(19) United States
(12) Patent Application Publication
(10) Pub. No.: US 2023/0303630 A1
(43) Pub. Date: Sep. 28, 2023

(54) ADENO-ASSOCIATED VIRUS COMPOSITIONS HAVING PREFERRED EXPRESSION LEVELS

(71) Applicants: Capsida, Inc., Thousand Oaks, CA (US); California Institute of Technology, Pasadena, CA (US)

(72) Inventors: Viviana Gradinaru, Pasadena, CA (US); Nicholas C. Flytzanis, Thousand Oaks, CA (US); Nicholas S. Goeden, Thousand Oaks, CA (US); Karl Beutner, La Canada Flintridge, CA (US)

(21) Appl. No.: 18/022,056

(22) PCT Filed: Aug. 20, 2021
(86) PCT No.: PCT/US2021/046904

§ 371 Date: Feb. 17, 2023

Related U.S. Application Data
(60) Provisional application No. 63/068,614, filed on Aug. 21, 2020.

(51) Int. Cl. C07K 14/005

(52) U.S. Cl. CPC C07K 14/005; C12N 2750/14122

(57) ABSTRACT

Described herein are compositions and kits comprising recombinant adeno-associated viruses (rAAVs) with tropisms showing increased viral transduction in the CNS. The rAAV compositions described herein encapsidate a transgene, such as a therapeutic nucleic acid. Gene therapy using the rAAVs is described. Also described are methods of treating CNS-related diseases and conditions.

### CROSS-REFERENCE TO RELATED APPLICATIONS

This application claims the benefit of U.S. Provisional Application No. 63/068,614, the content of which is incorporated herein in its entirety.

### STATEMENT REGARDING FEDERALLY SPONSORED RESEARCH OR DEVELOPMENT

This invention was made with government support under Grant Nos. NS087949 & NS111369 awarded by the National Institutes of Health. The government has certain rights in the invention.

### BACKGROUND

Recombinant adeno-associated viruses (rAAVs) are widely used as vectors for gene delivery in therapeutic applications because of their ability to transduce both dividing and non-dividing cells, their long-term persistence as episomal DNA in infected cells, and their low immunogenicity. These characteristics make them appealing for applications in therapeutic applications, such as gene therapy. However, there is a need to significantly improve the performance of existing AAV serotypes to selectively and efficiently express in distinct cell-types, upon systemic delivery to a subject. This need is especially acute when the AAV must be expressed in the central nervous system (CNS).

Systemic delivery of existing AAV serotypes show limited transduction of certain cell types and organs, and non-specific, overlapping tropisms in others. This leads to several complications in gene therapy applications, including but not limited to off-target effects due to transduction of unimpacted organs and cell types (for example, the liver).

### SUMMARY OF THE INVENTION

Disclosed herein are rAAVs with engineered specificity into the capsid structure through iterative rounds of selection in non-human primates (NHPs), yielding variants with tropisms having an increased specificity and transduction efficiency when measured in the CNS.

The present invention provides rAAVs with widespread transduction to the CNS.

The present invention provides, in an aspect, a peptide insertion comprising or consisting of an amino-acid sequence set forth in any one of Tables 1 and 4-30, FIG. 4 and/or Formulas I-XXXIII, as defined below in greater detail.

Another aspect of the invention is a modified capsid protein wherein the AAV capsid protein, with a peptide insertion comprising or consisting of an amino-acid sequence set forth in any one of Tables 1 and 4-30, FIG. 4 and/or Formulas I-XXXIII, as defined below in greater detail, is characterized by increased CNS transduction in a subject.

The present disclosure moreover includes pharmaceutical compositions comprising rAAVs with a peptide insertion comprising or consisting of an amino-acid sequence set forth in any one of Tables 1 and 4-30, FIG. 4 and/or Formulas I-XXXIII, as defined below in greater detail, and a pharmaceutically acceptable excipient.

Aspects disclosed herein provide methods of treating a disease or condition in a subject comprising administering a therapeutically effective amount of a pharmaceutical formulation comprising the AAV capsid protein or the AAV capsid of the present disclosure. In some embodiments, the disease or the condition is a disease or a condition of the CNS, neurons or spinal column of the subject. Relatedly, the invention includes use of the rAAVs in the manufacture of a medicament for treating or preventing the disease or medical condition.

Other aspects of the invention will be apparent from the detailed description and claims that follow.

### BRIEF DESCRIPTION OF THE FIGURES

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows staining against the HA tag fused to hFXN transcripts virally expressed in the macaque brain. Robust and broad expression was achieved by a pool of eight viruses throughout the brain. Stained sections from each coronal block of the brain were imaged in their entirety at a 4× magnification (FIG. 1A). Sub-regions identified within various major brain areas, the four main cortical lobes, hippocampus, caudate, putamen, thalamus and midbrain, were imaged at a 10× magnification across a z-thickness of 25 m (FIG. 1B).

FIG. 2. shows a 3-dimensional scatter plot of the distribution of engineered rAAV sequences in liver, spinal cord or brain tissue after administration of a viral library to marmosets and next-generation sequencing of the variants pulled out from tissue.

FIG. 3 shows the result of further refinement of the data in the scatter plot in FIG. 2 focusing on the expression of the sequences that express in the spinal cord.

FIG. 4 shows AAV capsid protein insertion amino acid sequences and DNA sequences encoding the amino acid sequences which were found in the non-human primate CNS after two rounds of selection of an engineered AAV library.

FIG. 5 shows the expression achieved by the eight AAV variants from the pool in FIG. 1 throughout the macaque brain (FIG. 5A), spinal cord (FIG. 5B) and liver (FIG. 5C). The relative viral genomes and transcript expression levels of each of the barcoded viruses were normalized to those of AAV9 and averaged across two animals.

FIG. 6 shows the biodistribution of an AAV variant from the pool in FIG. 1 throughout the cynomolgus maccaques, including portions of the CNS (brain, and spinal cord), dorsal root ganglia and liver. The transcript expression levels of the viruses were normalized to GAPDH for the three animals.

### DETAILED DESCRIPTION OF THE DISCLOSURE

In one aspect the disclosure provides rAAVs with high expression levels in the CNS.

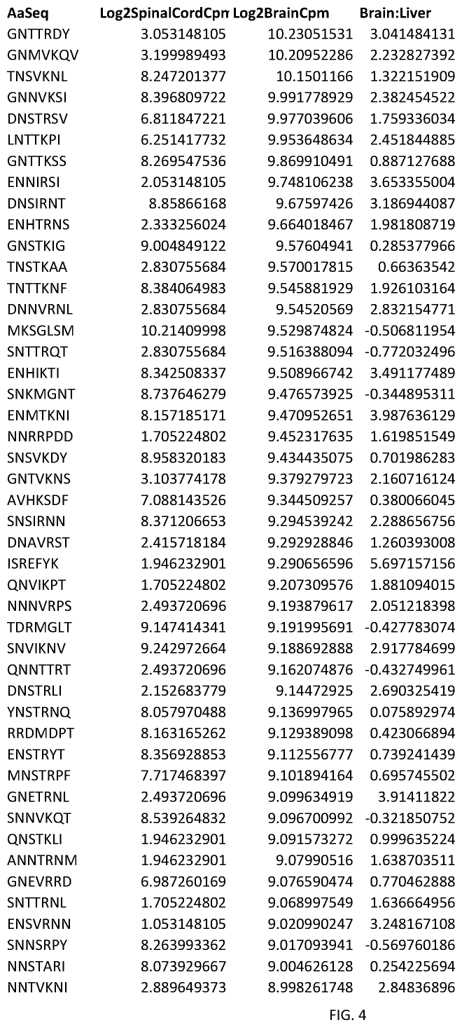

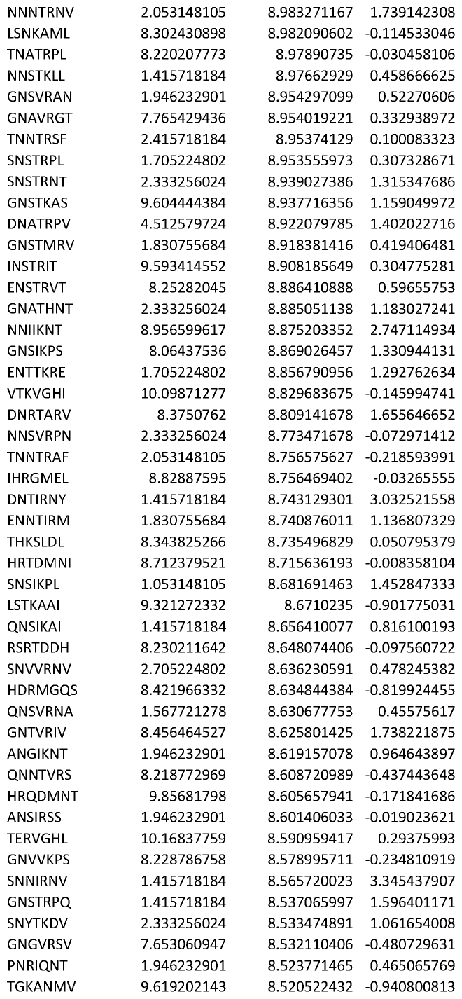

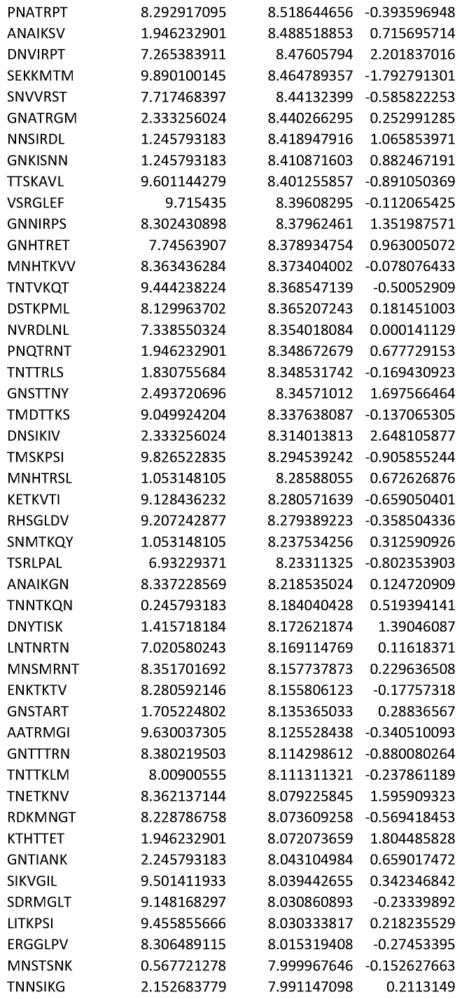

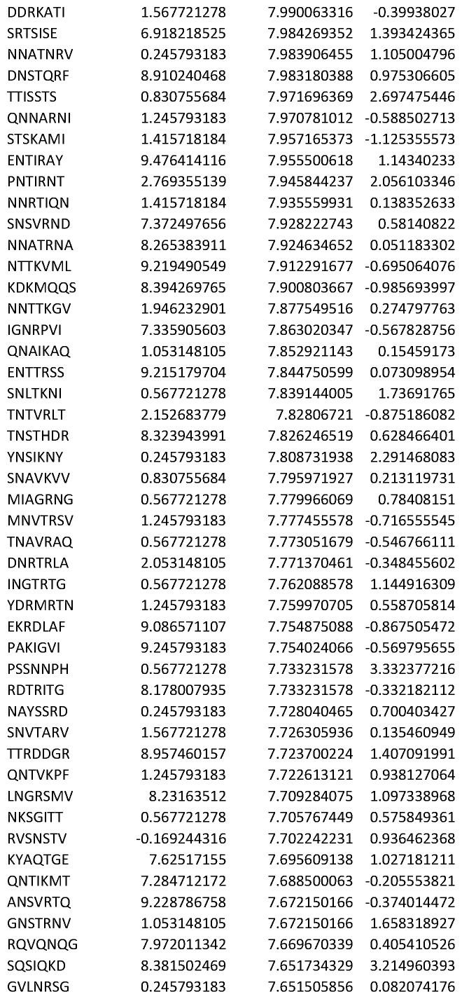

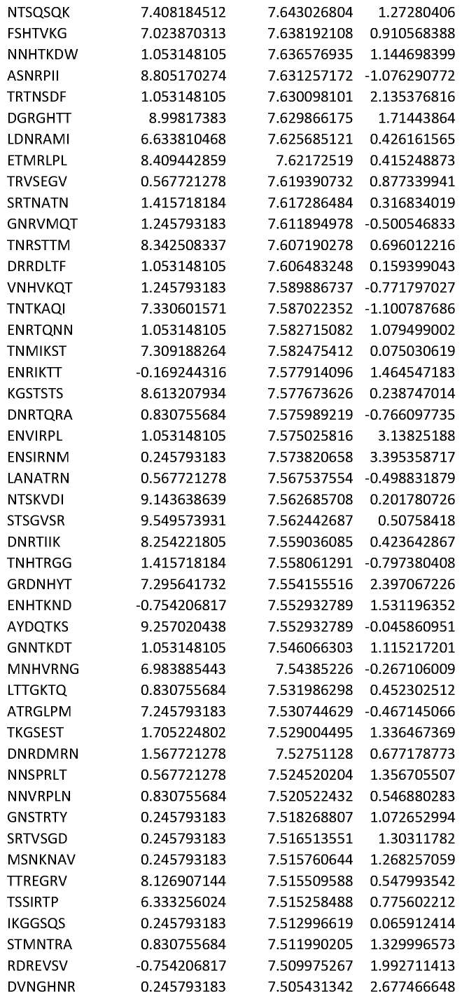

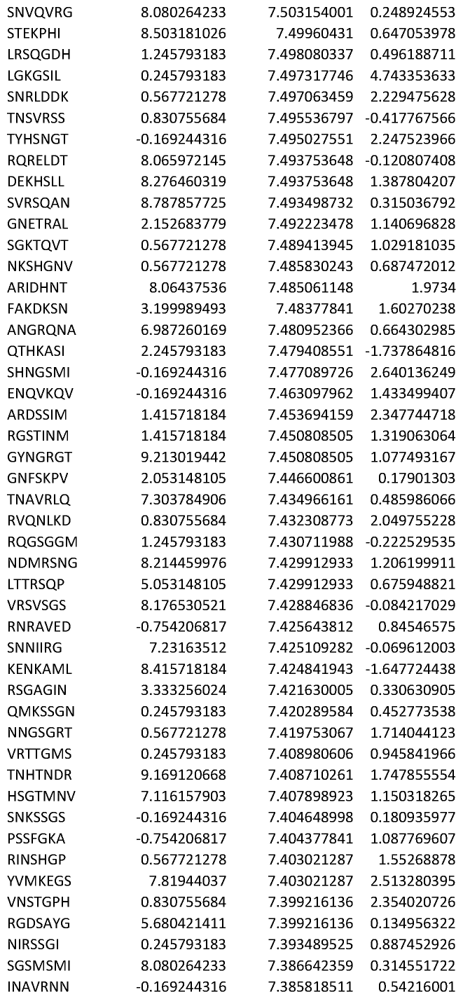

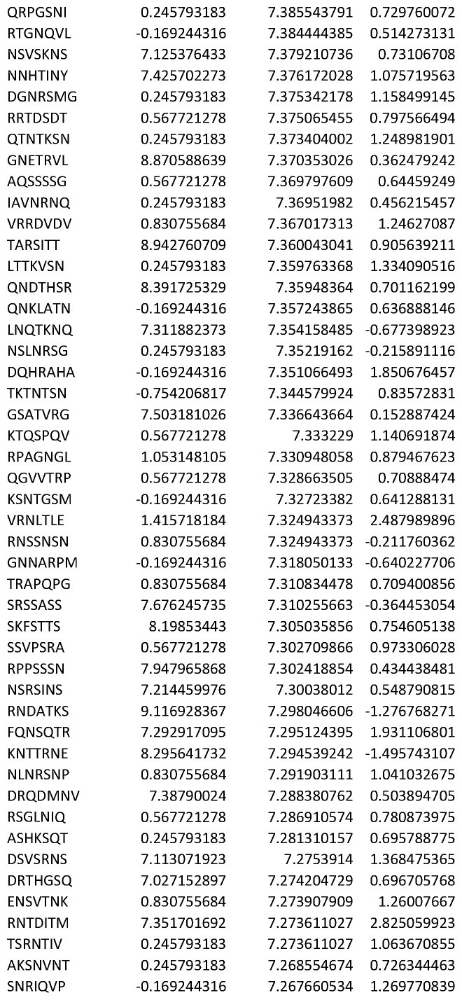

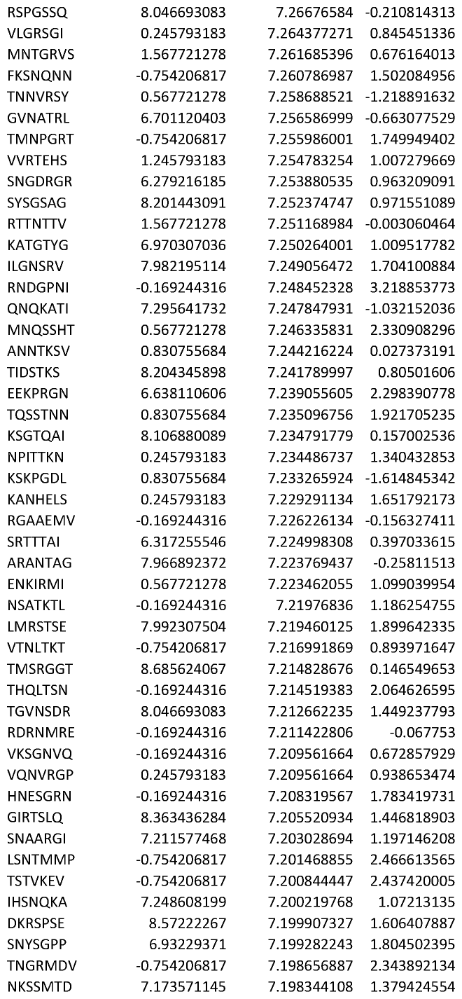

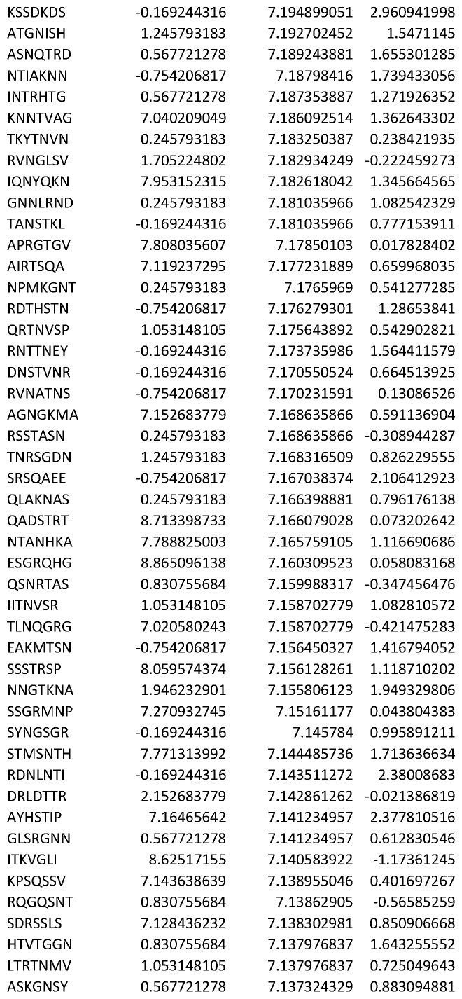

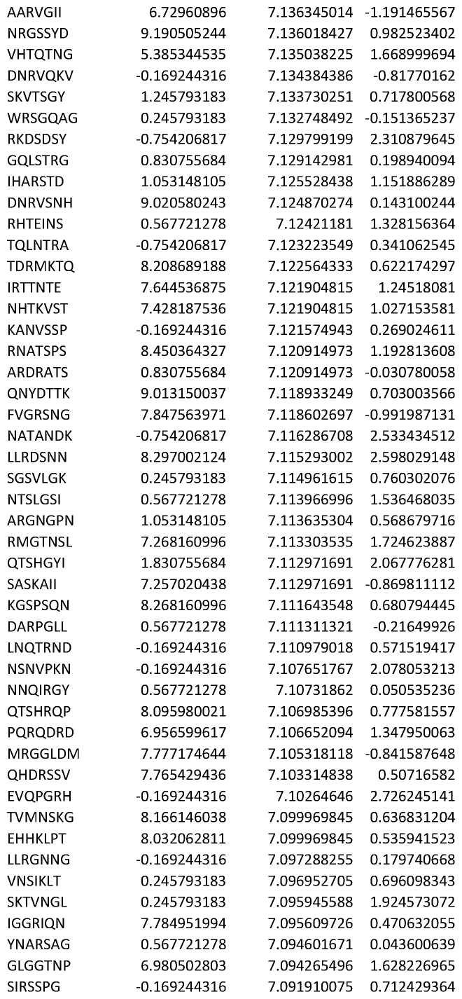

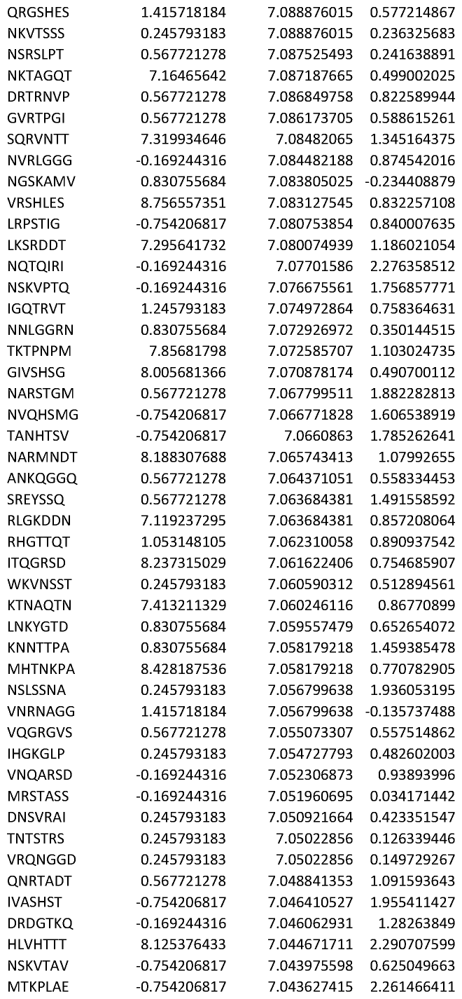

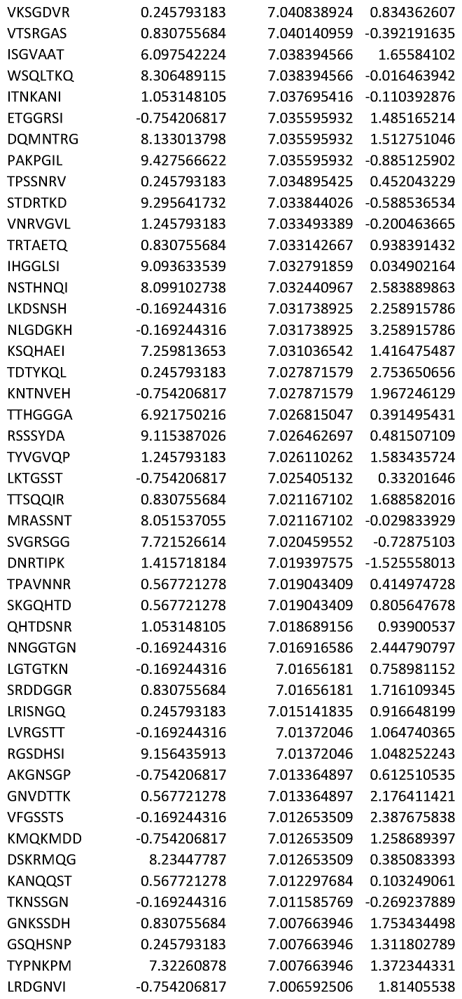

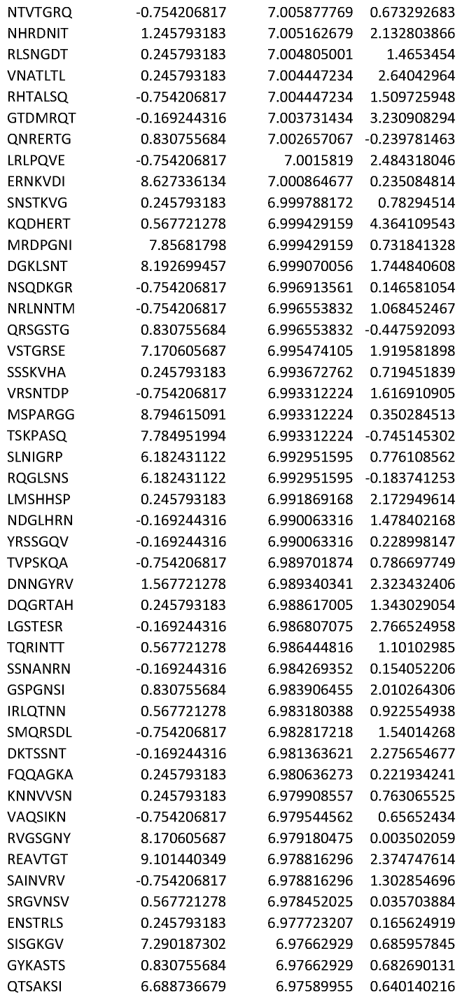

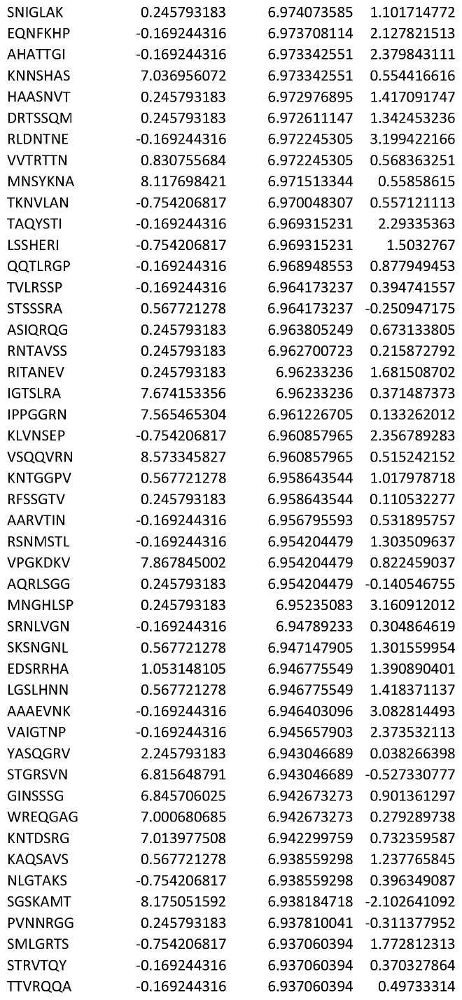

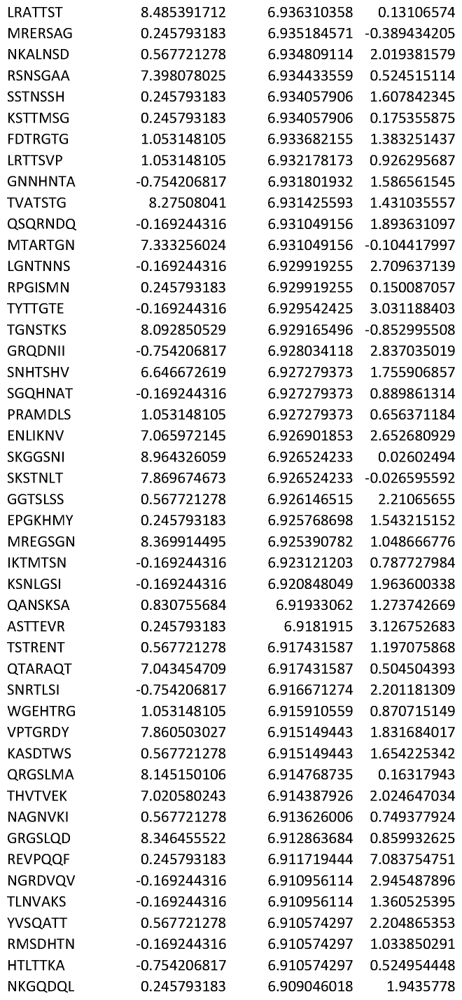

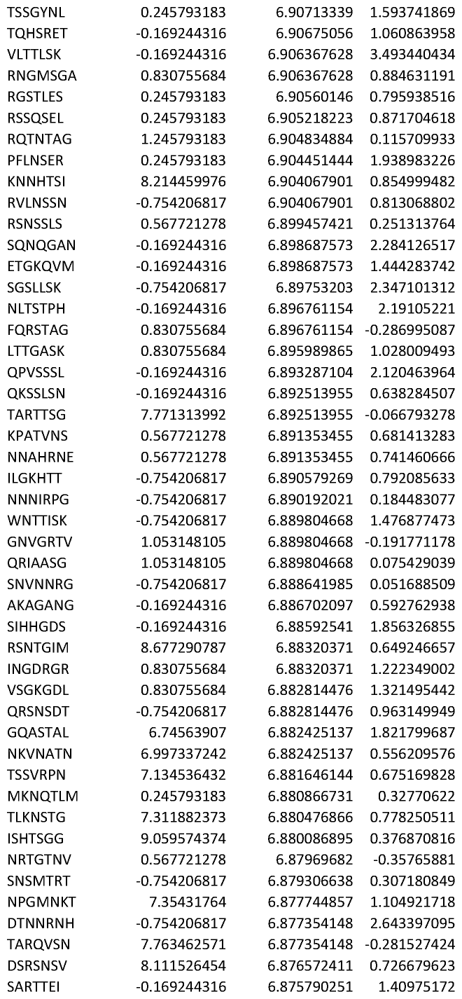

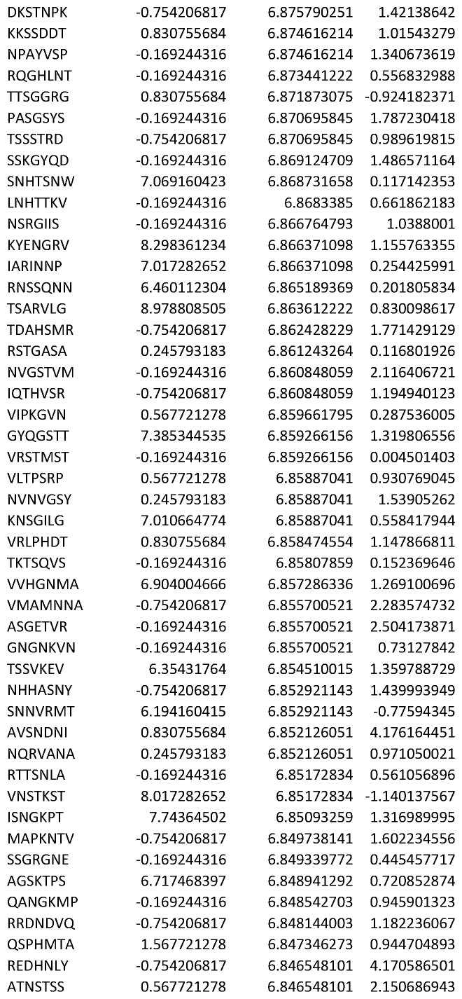

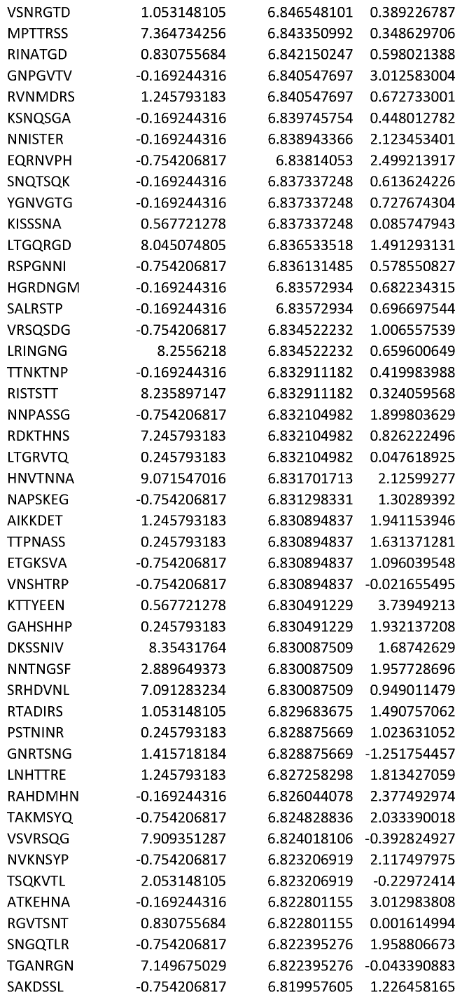

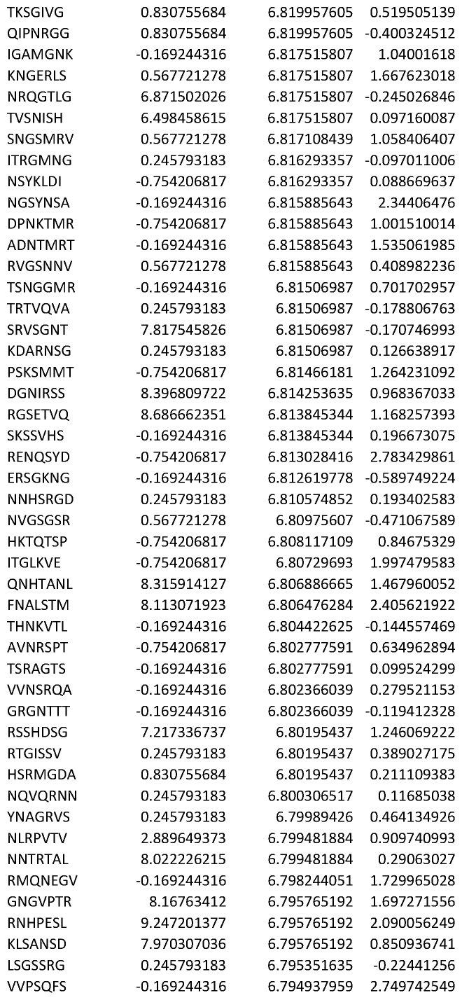

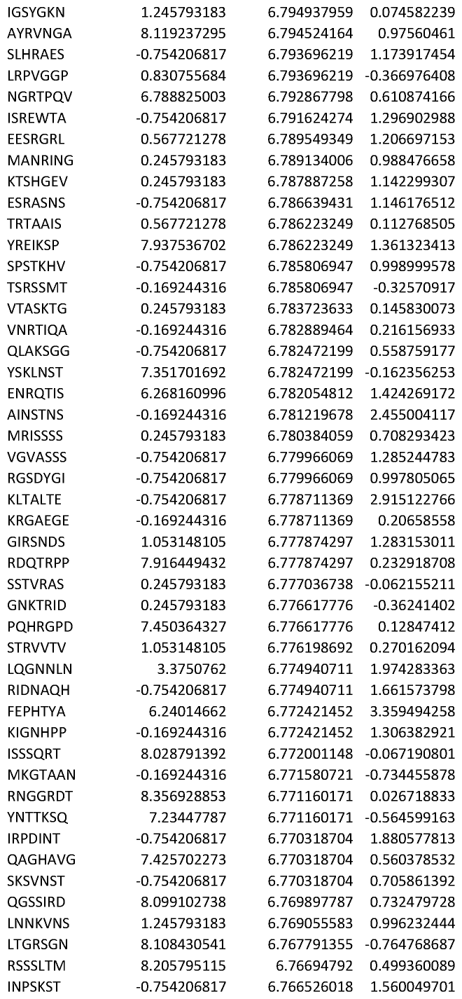

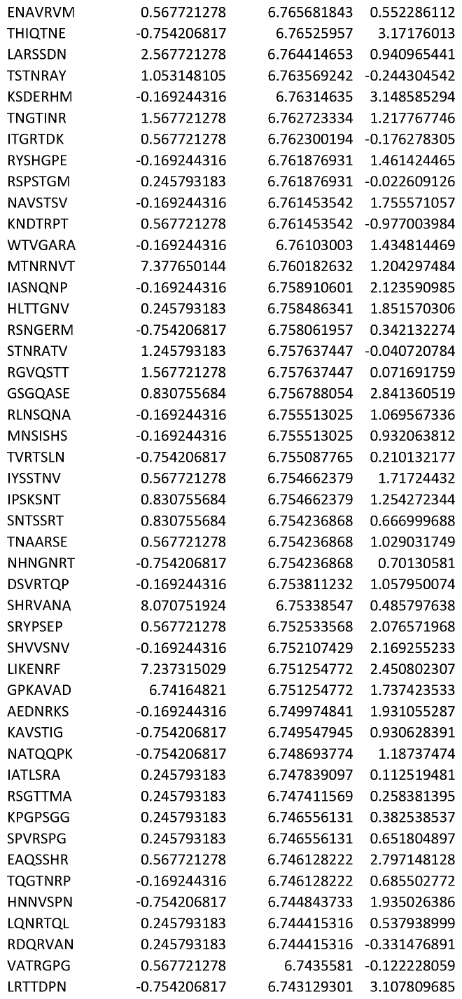

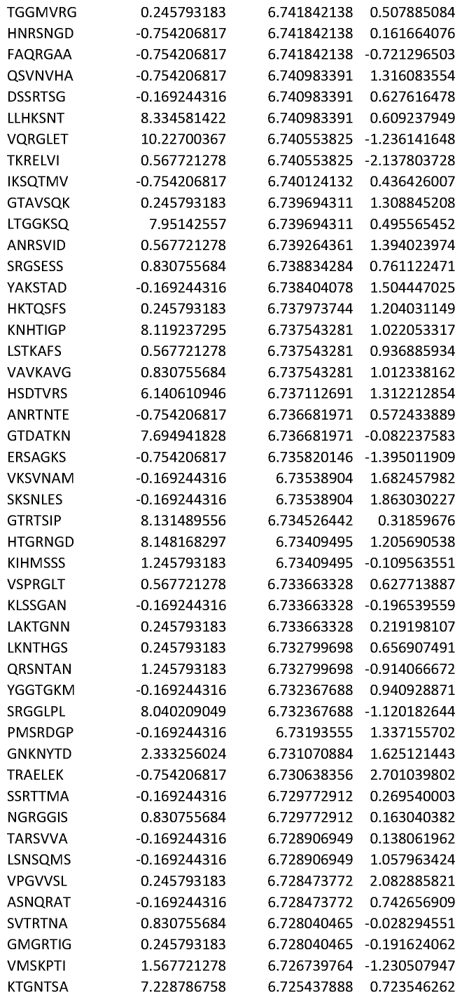

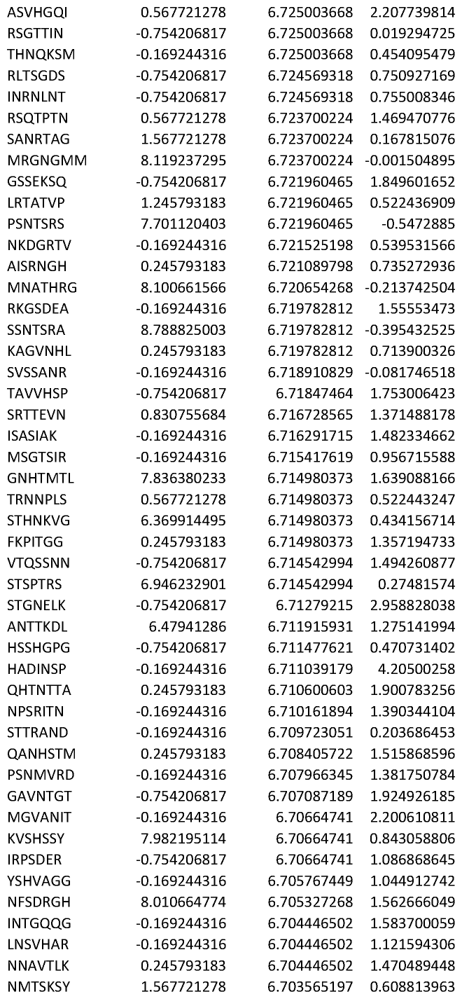

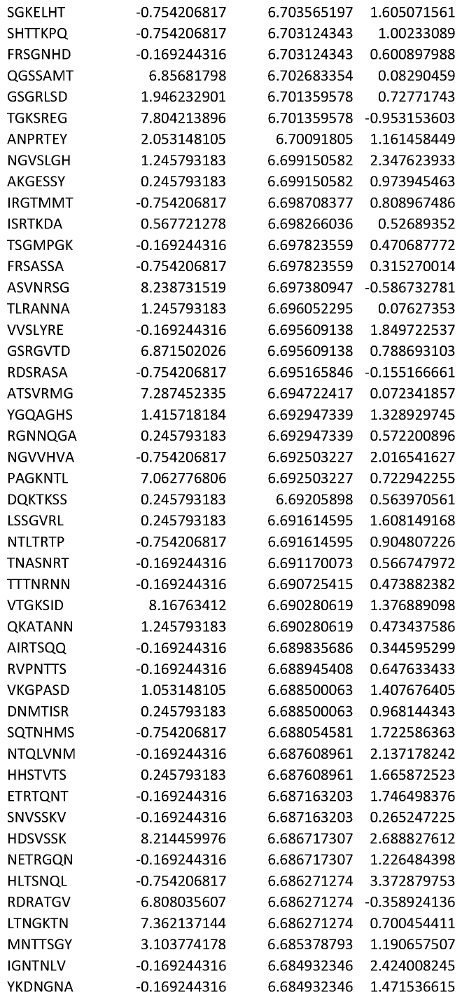

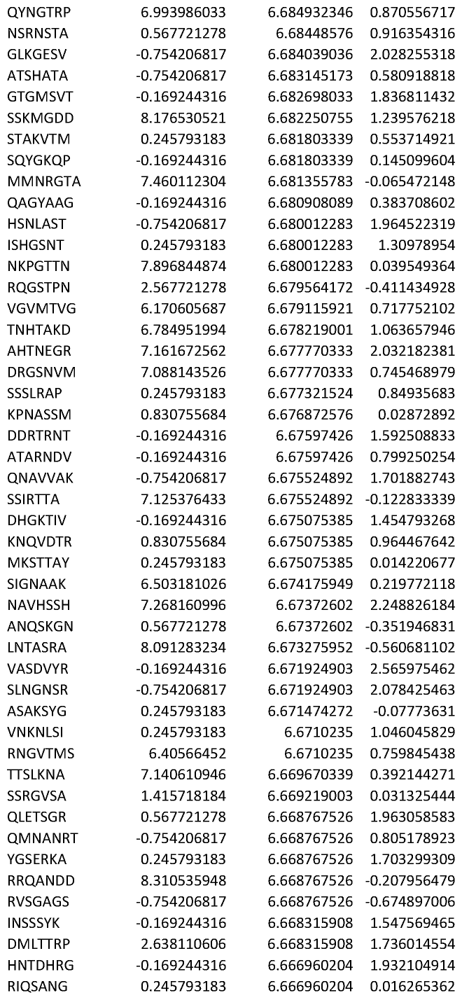

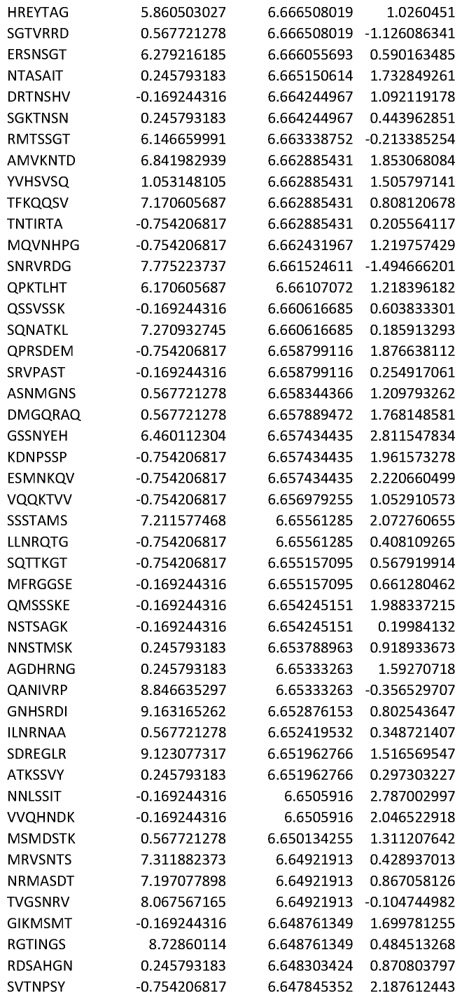

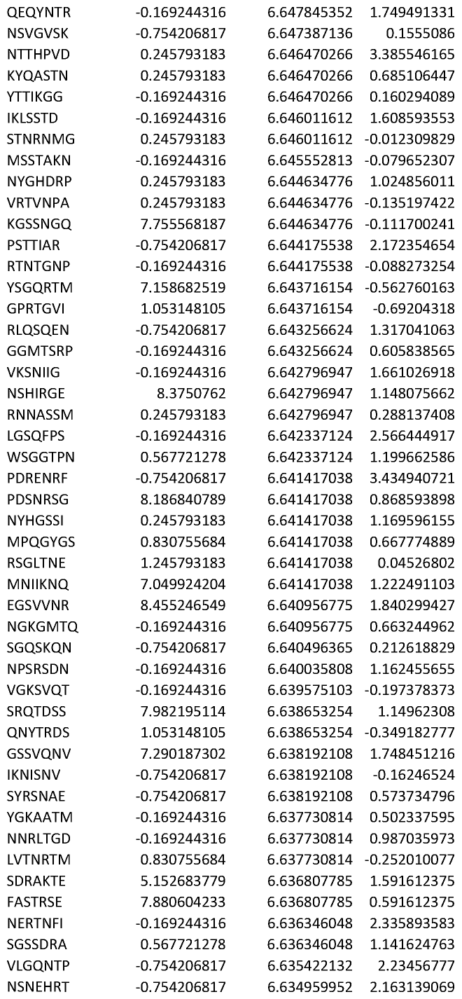

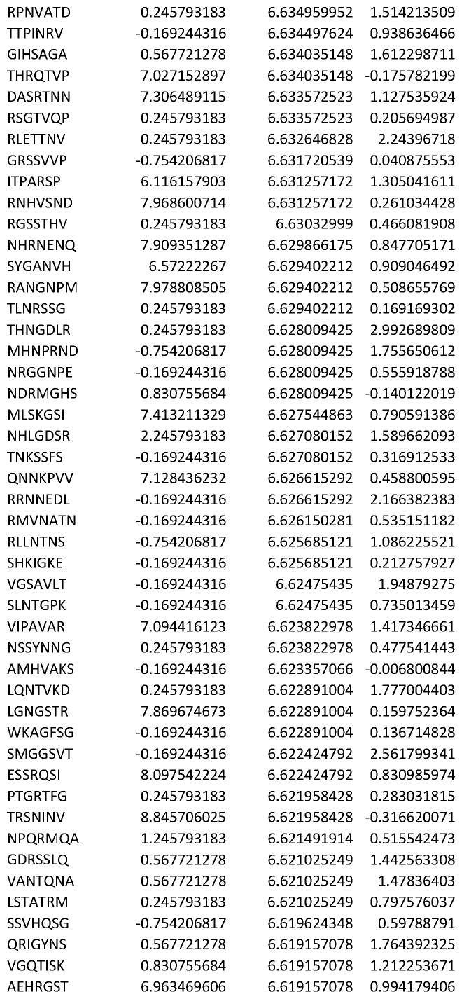

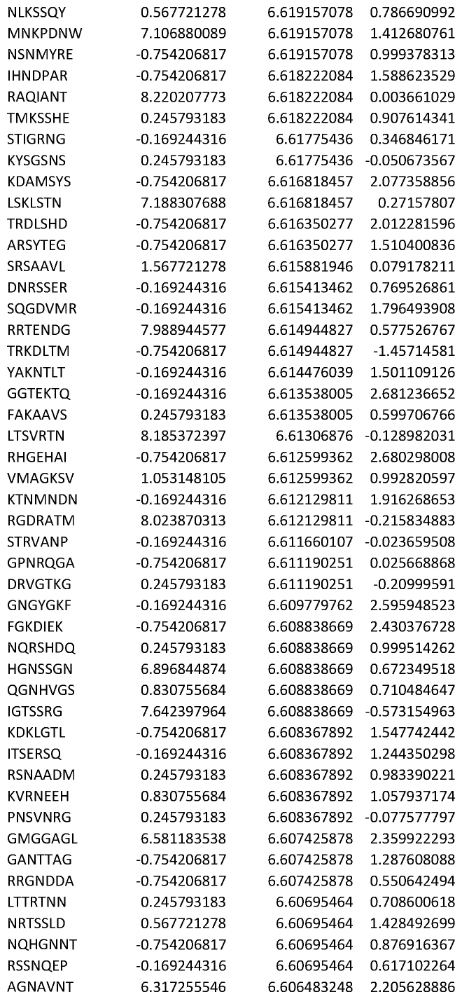

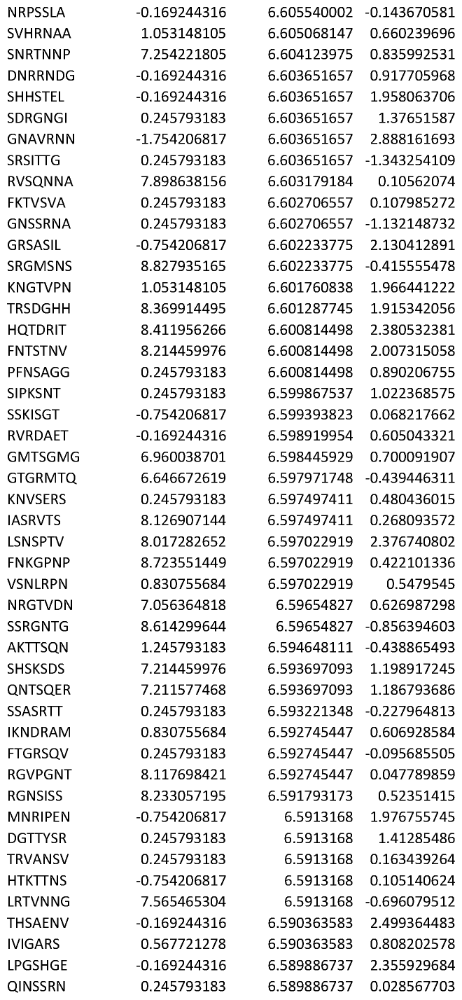

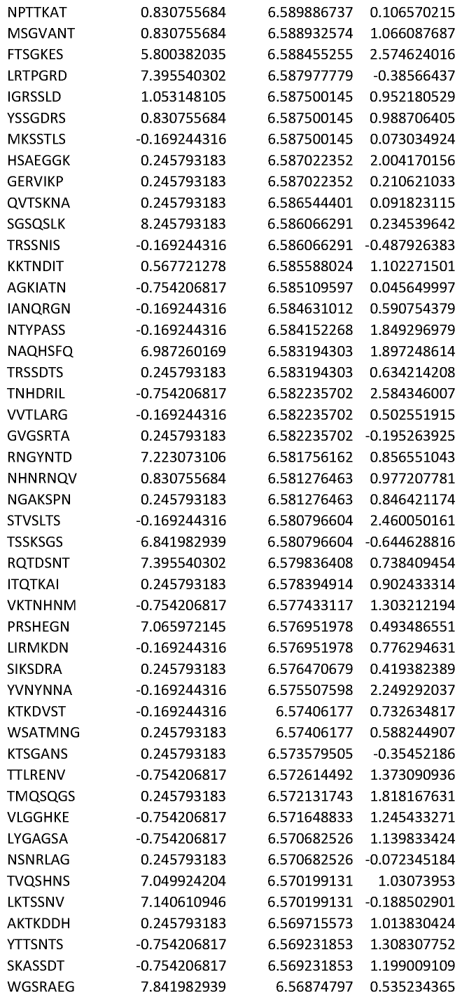

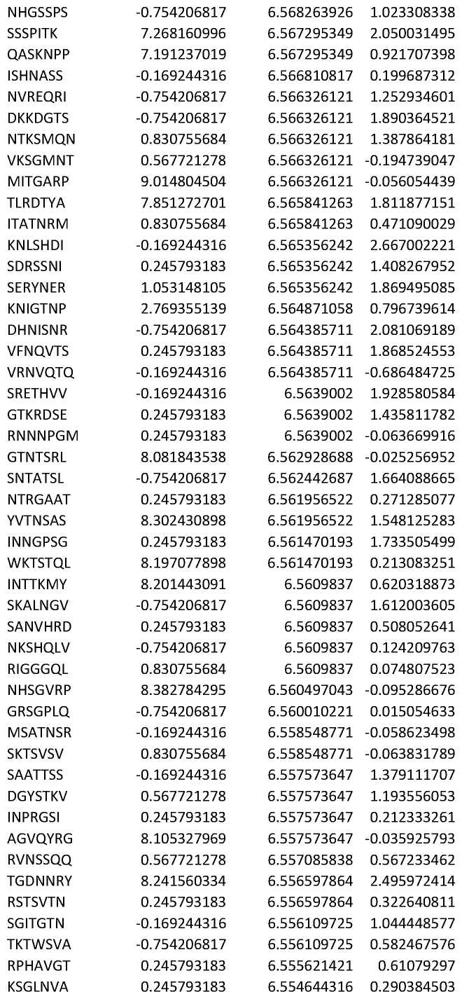

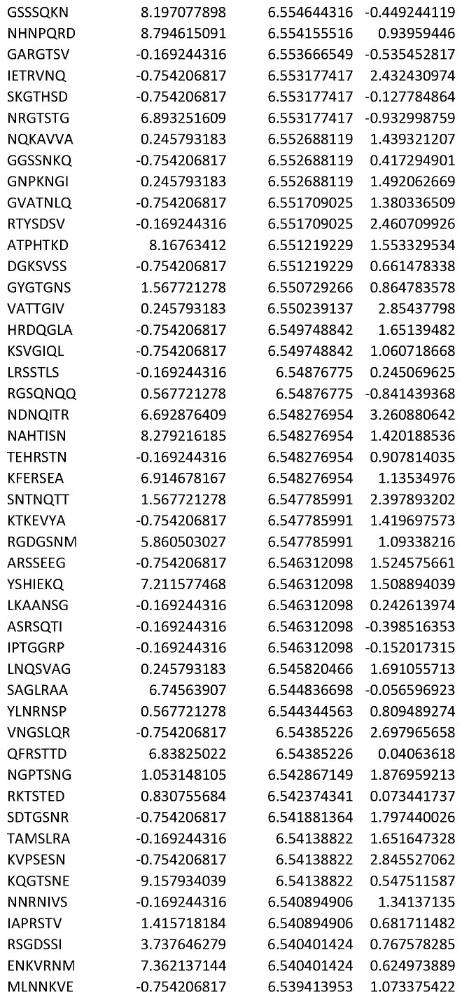

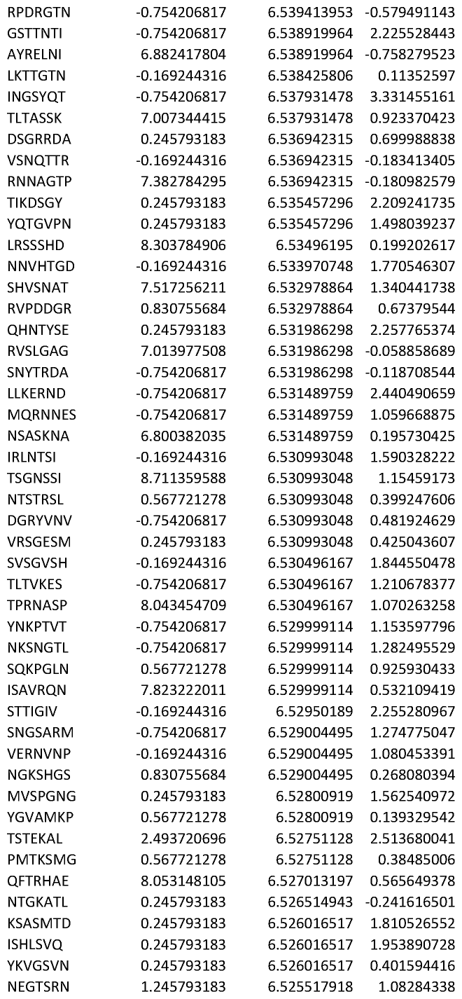

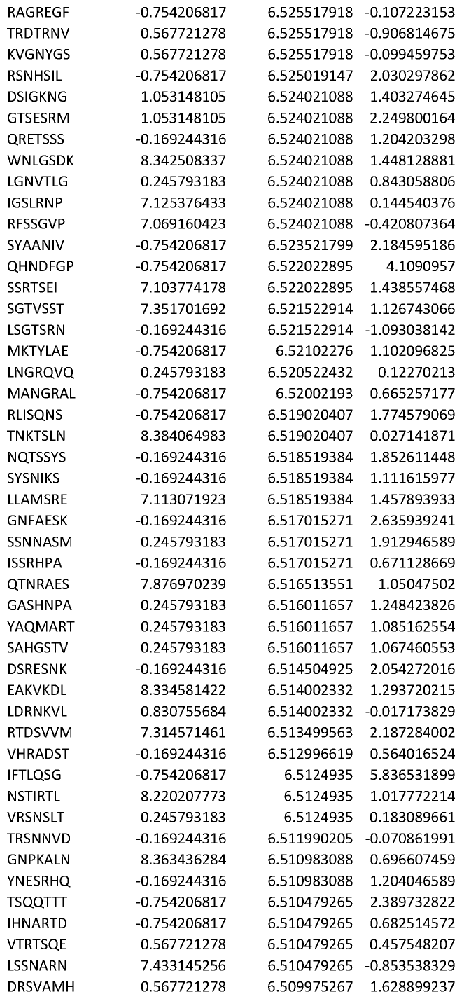

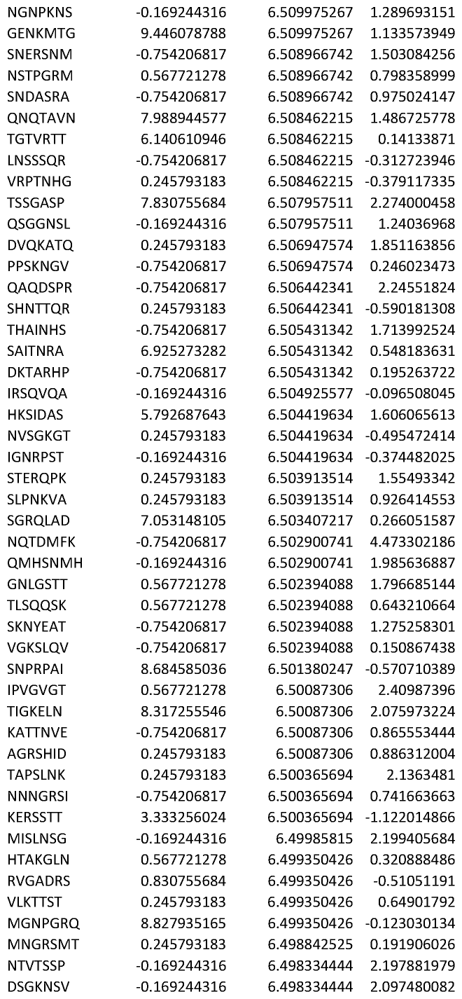

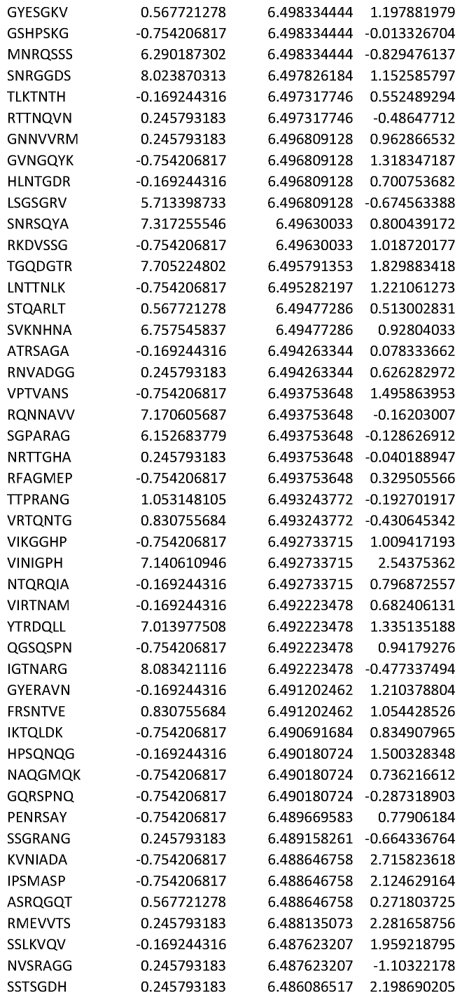

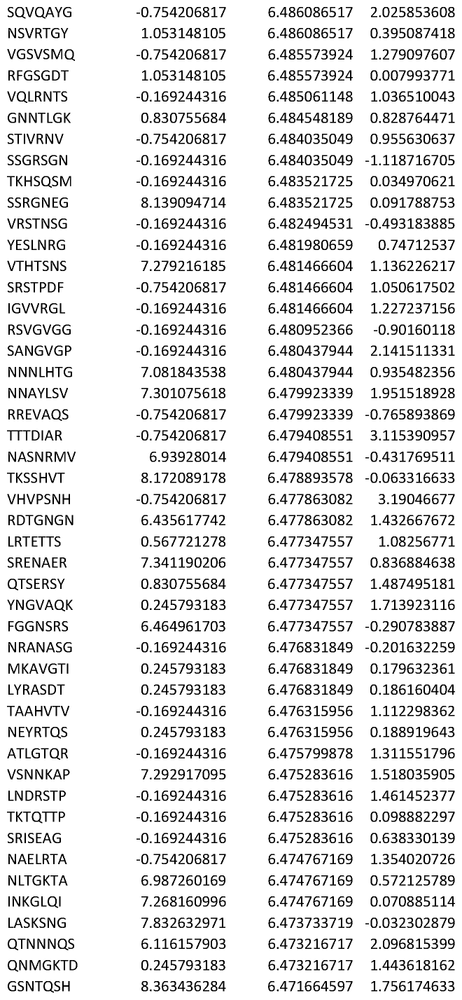

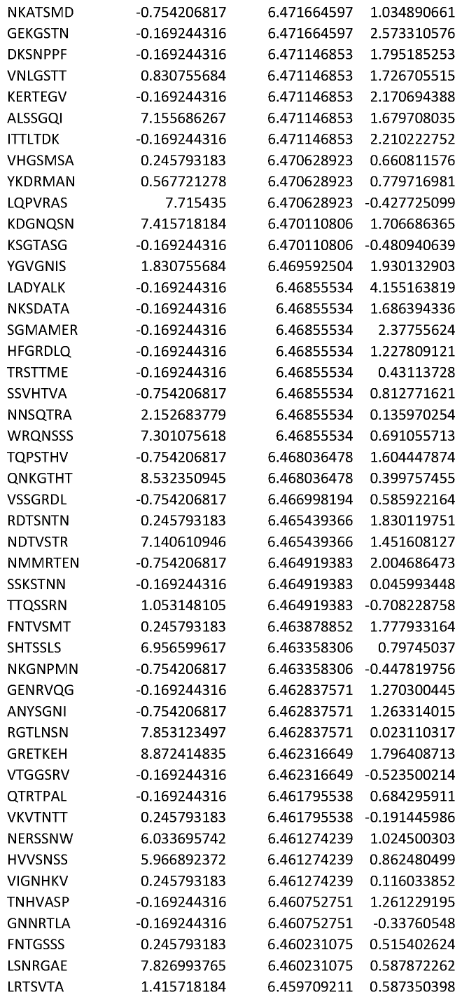

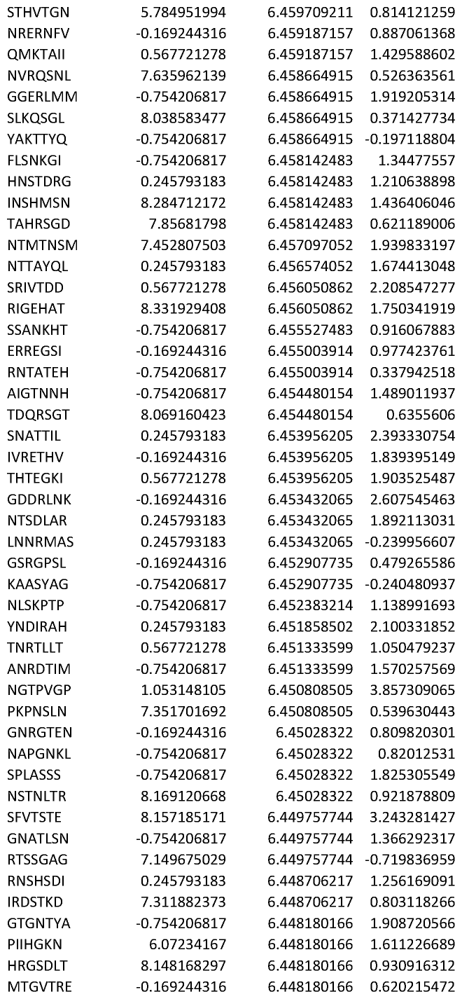

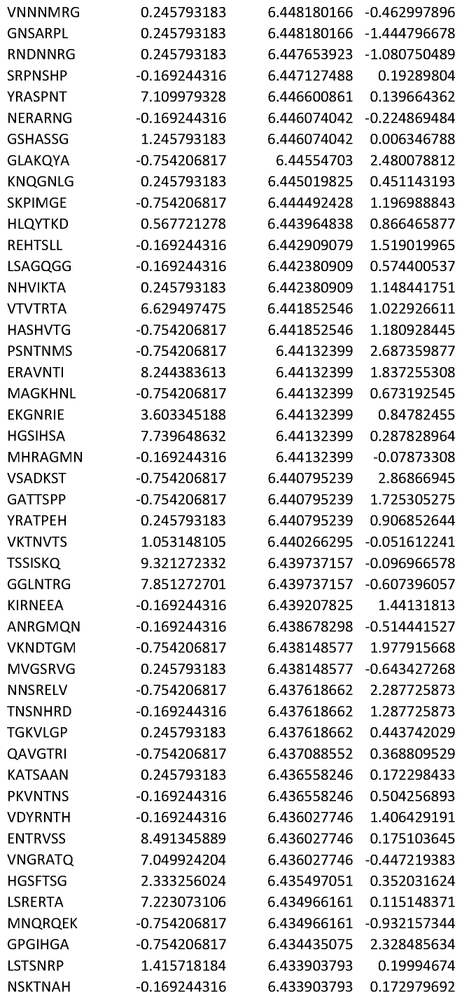

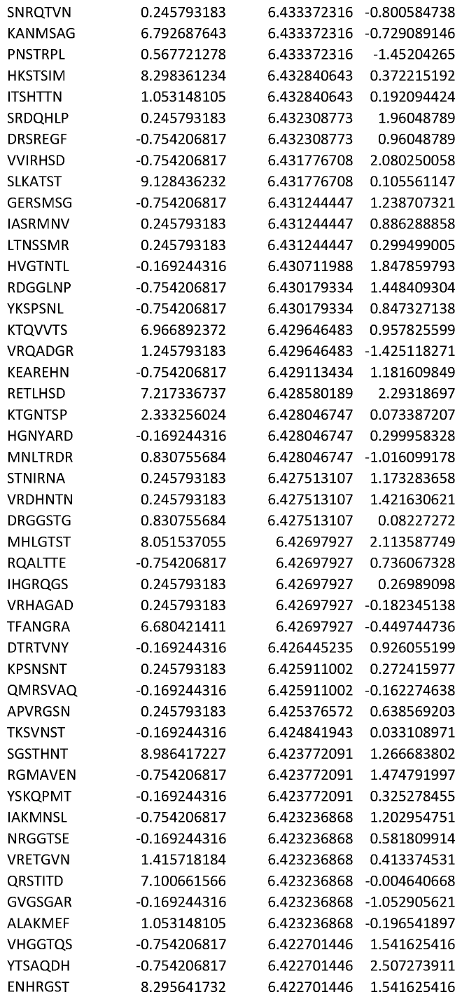

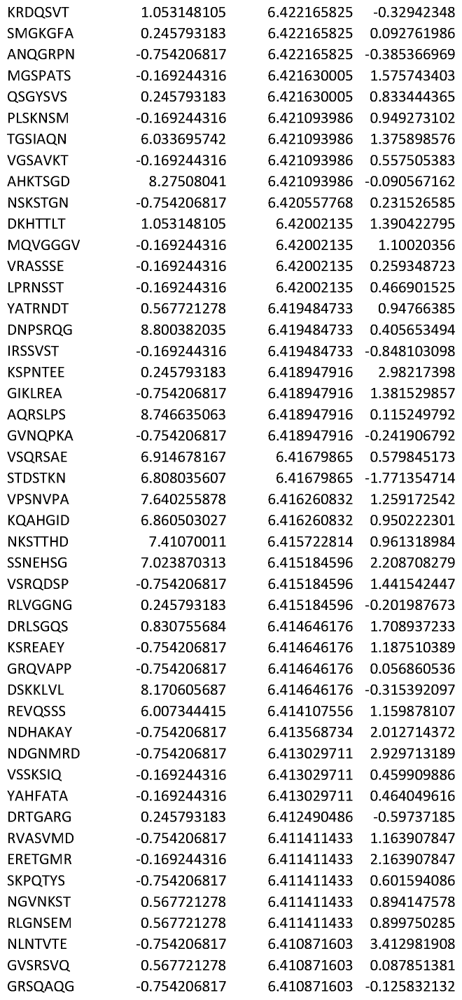

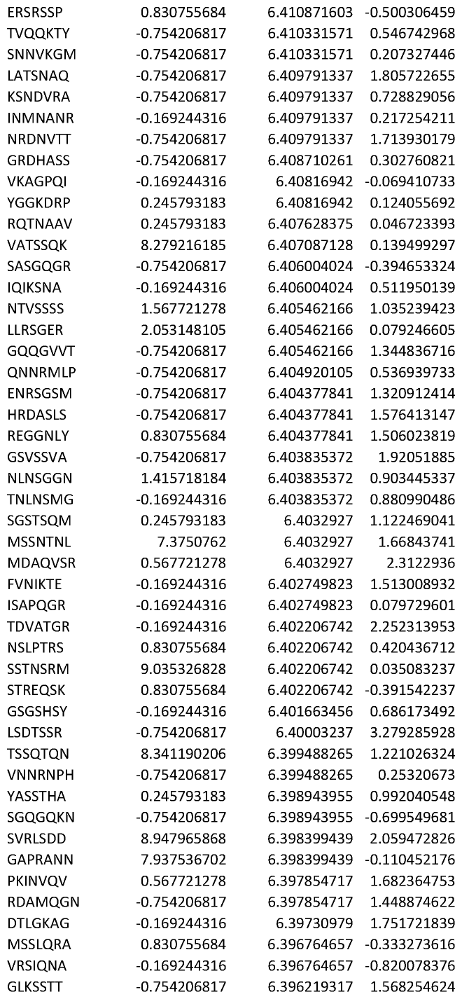

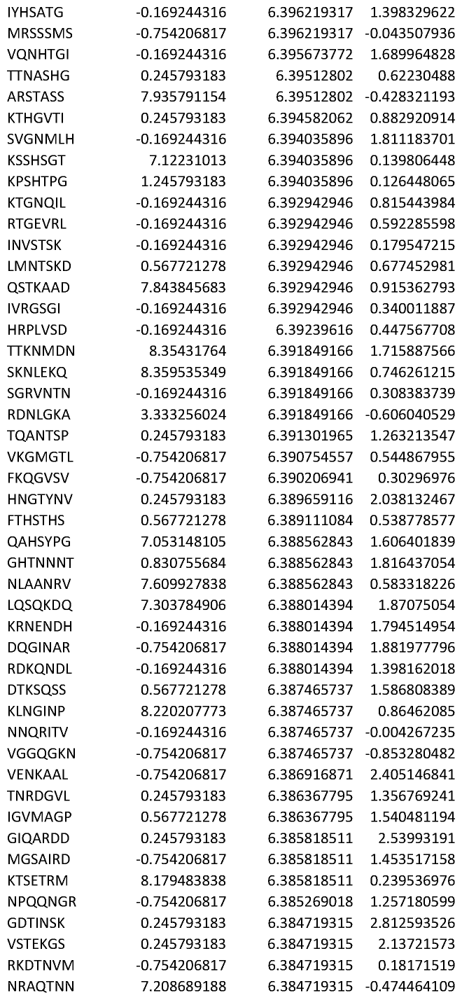

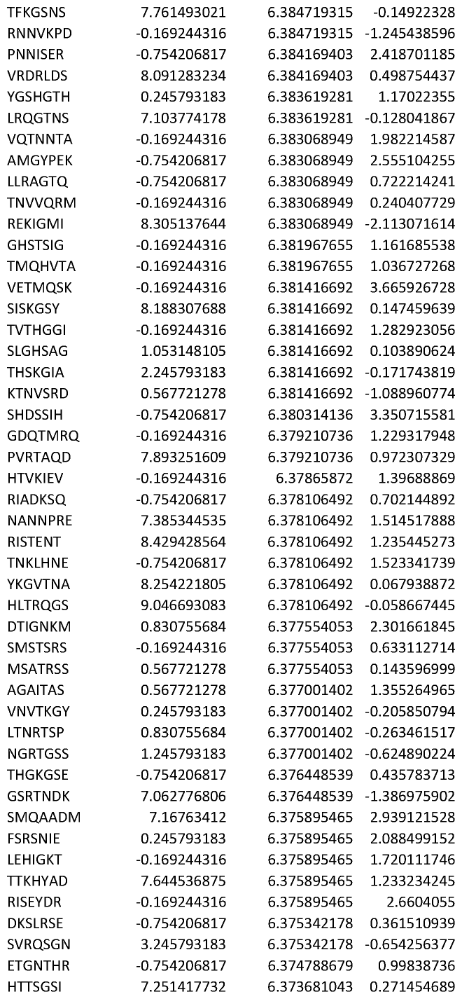

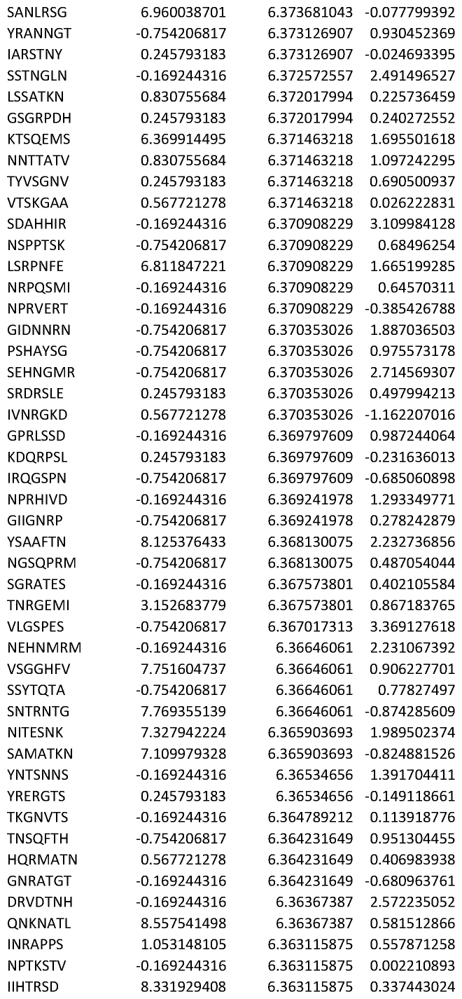

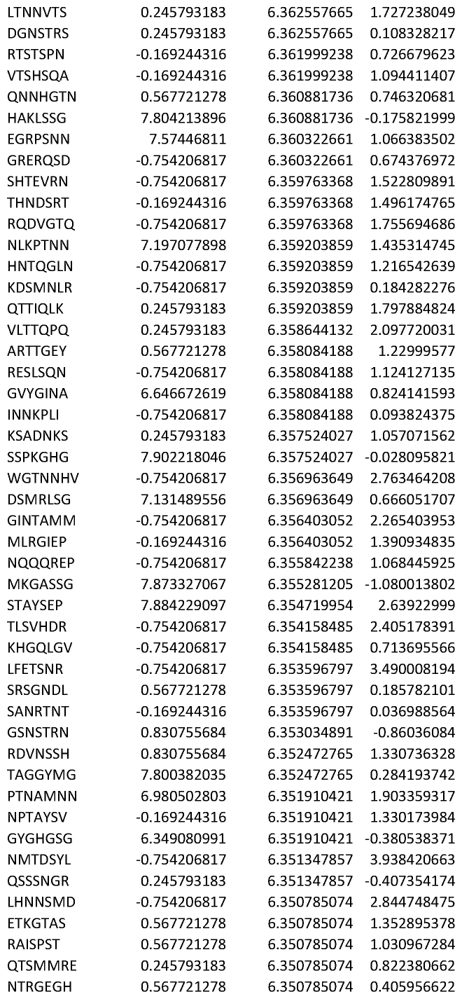

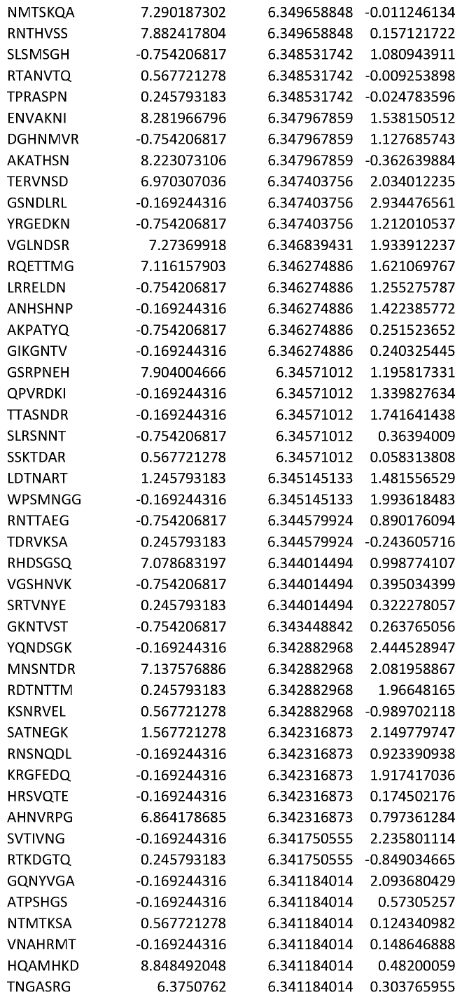

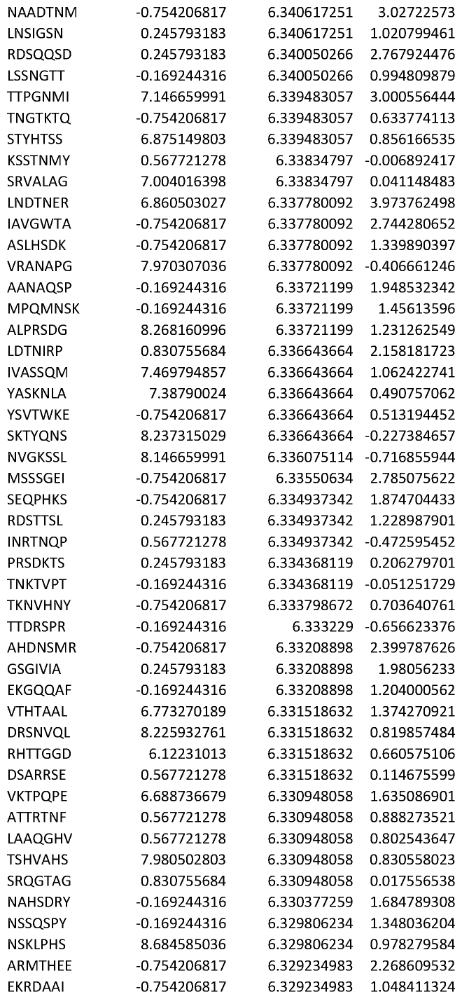

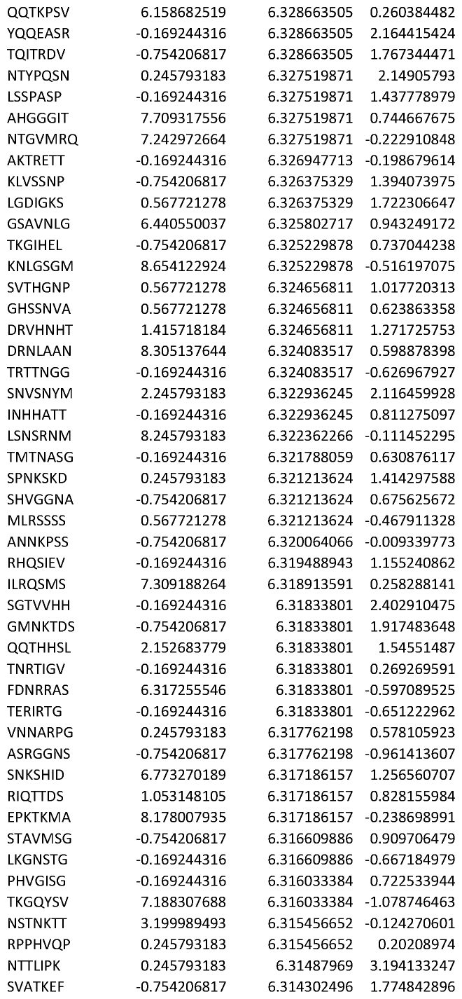

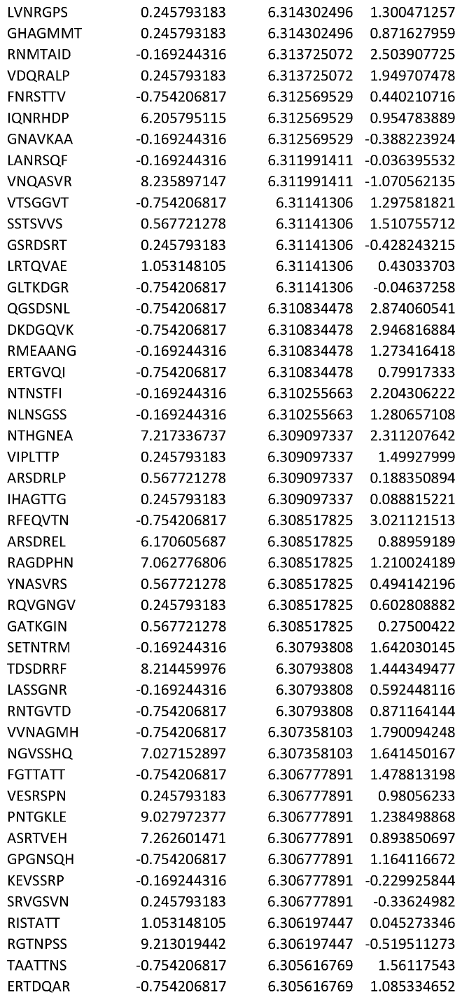

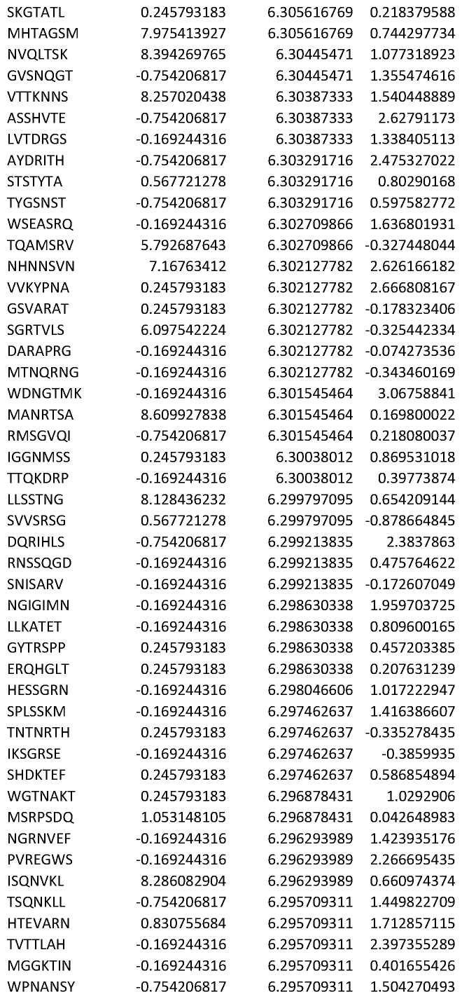

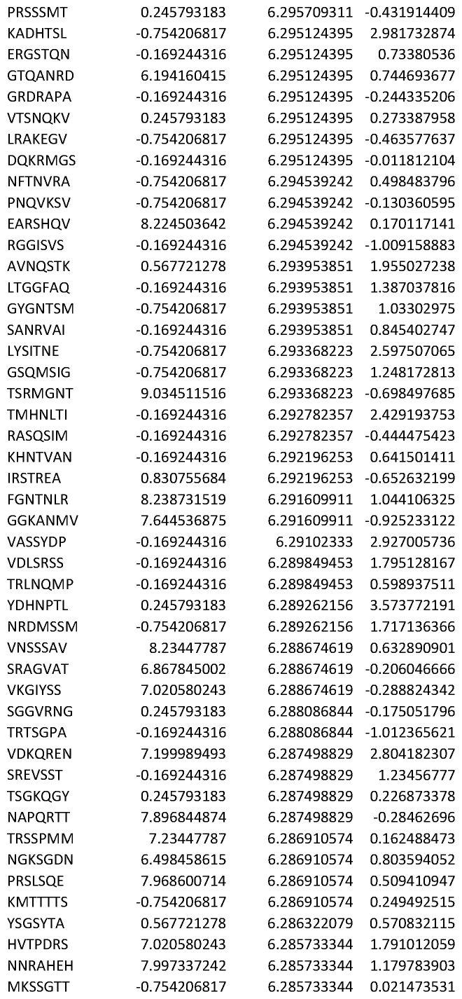

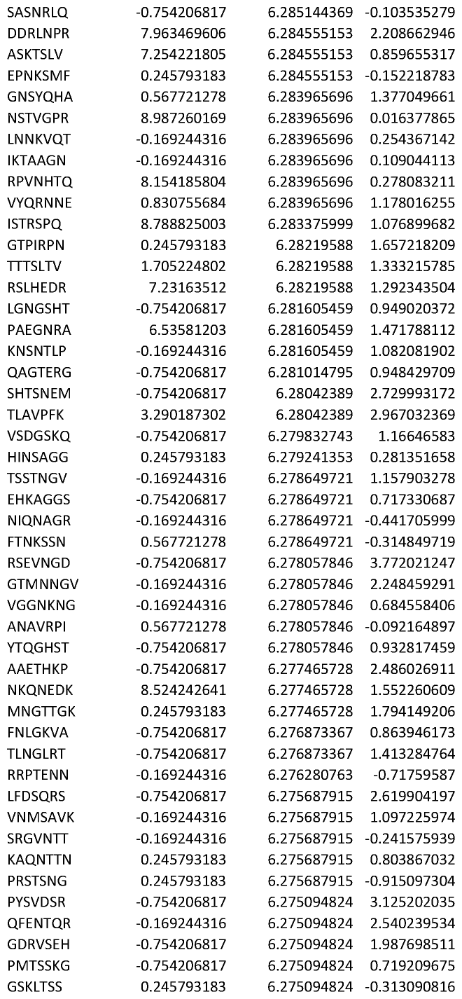

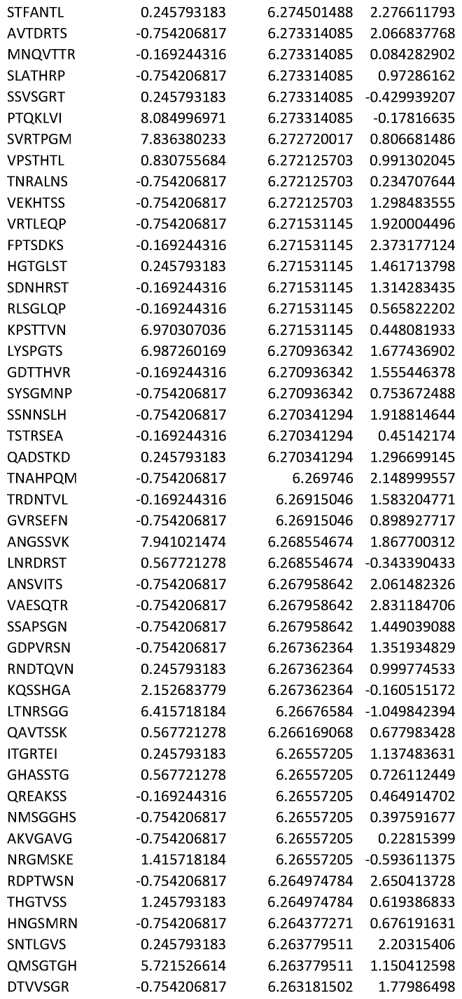

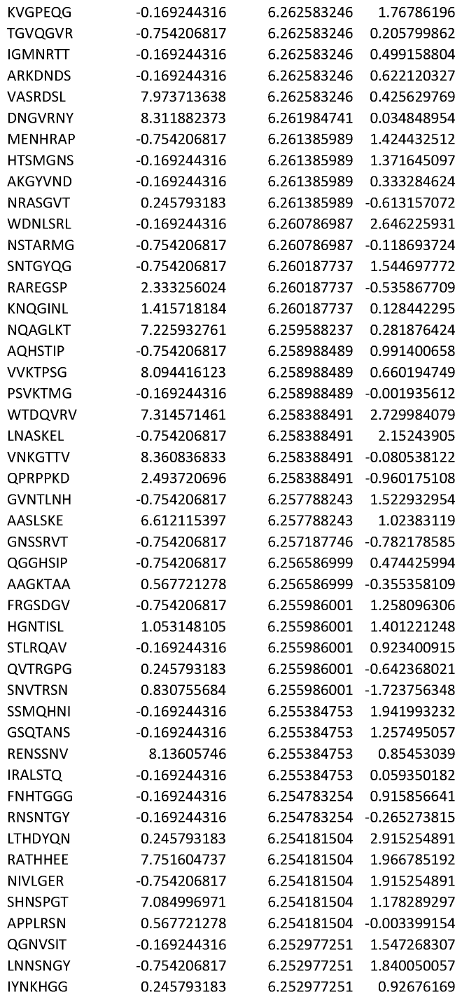

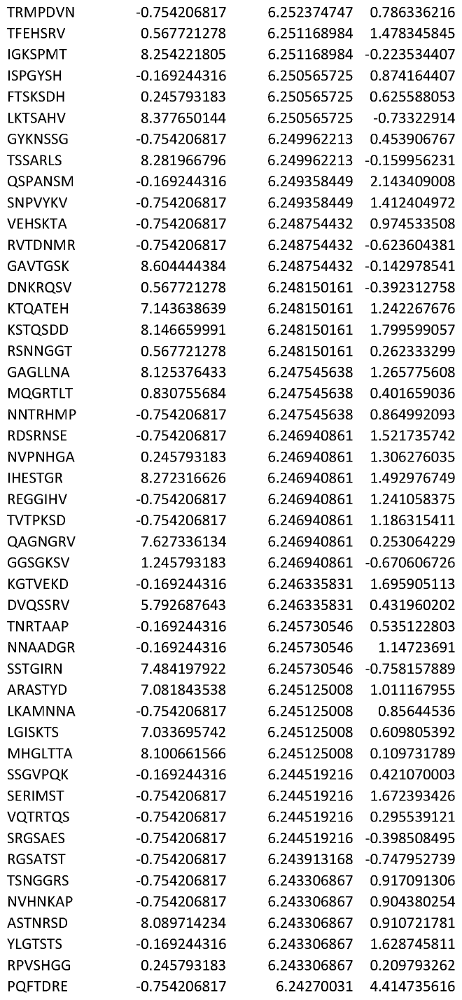

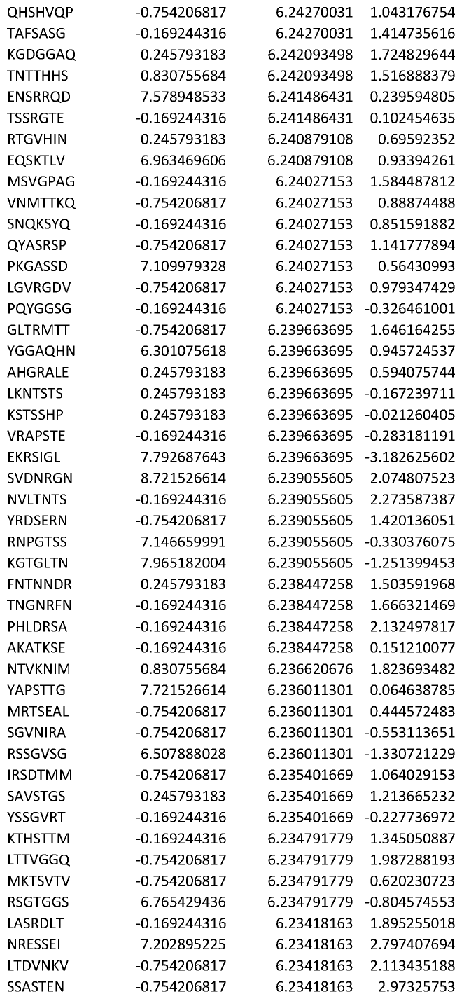

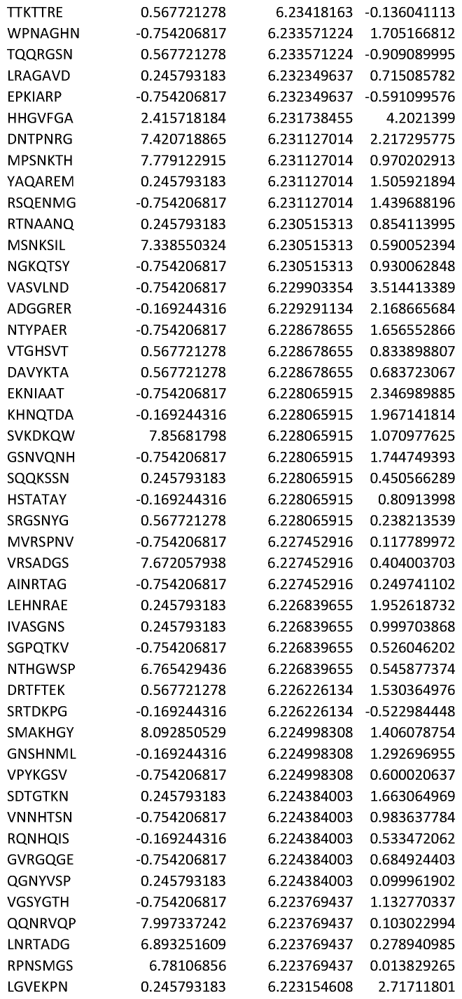

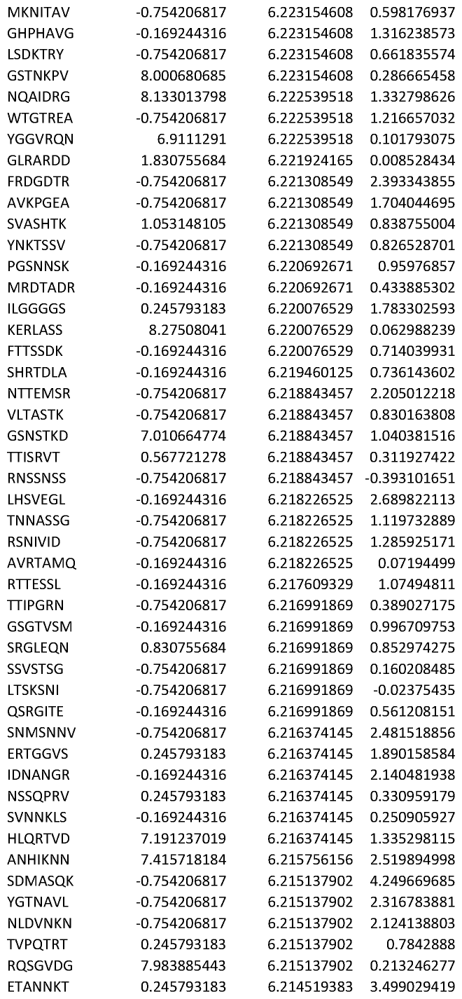

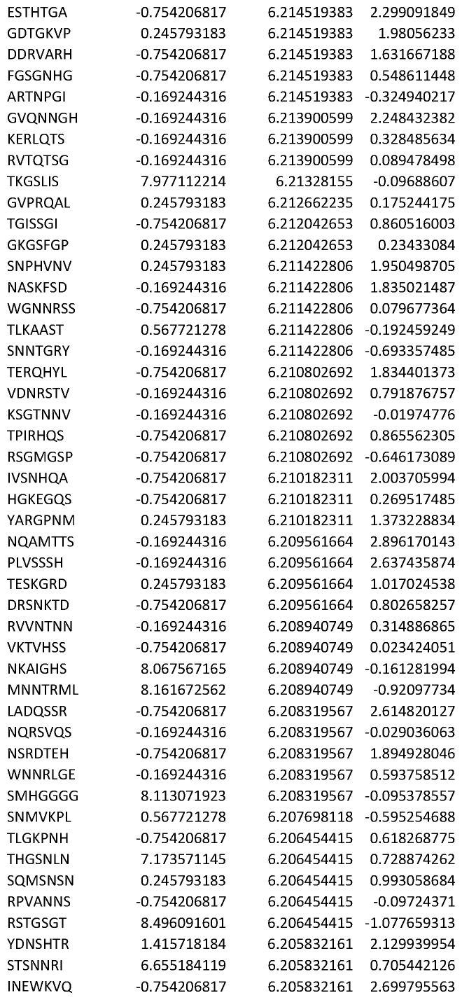

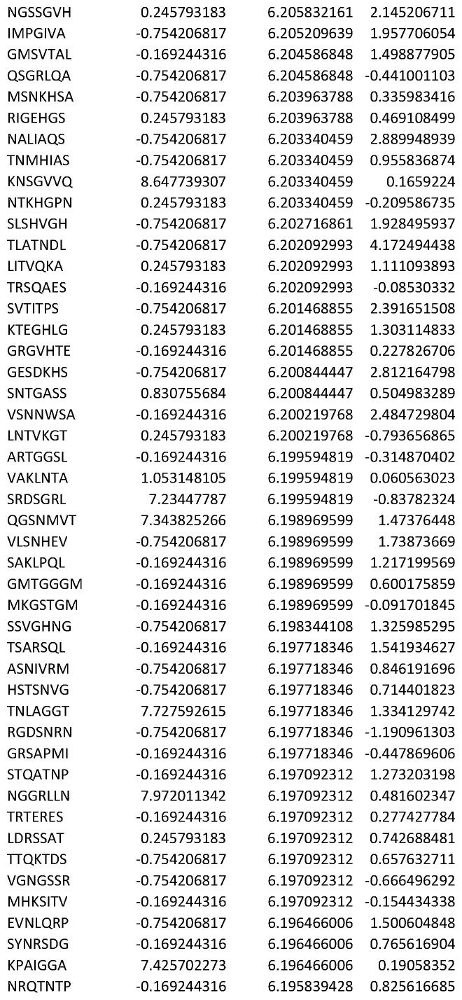

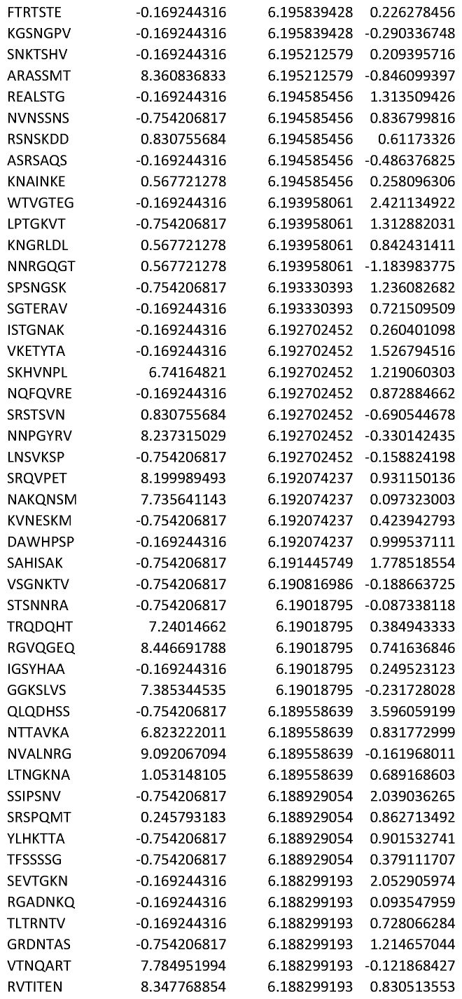

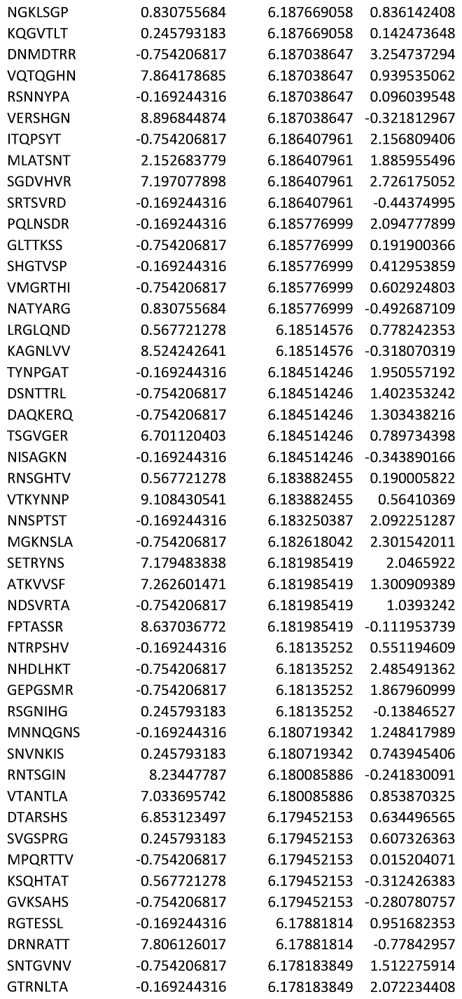

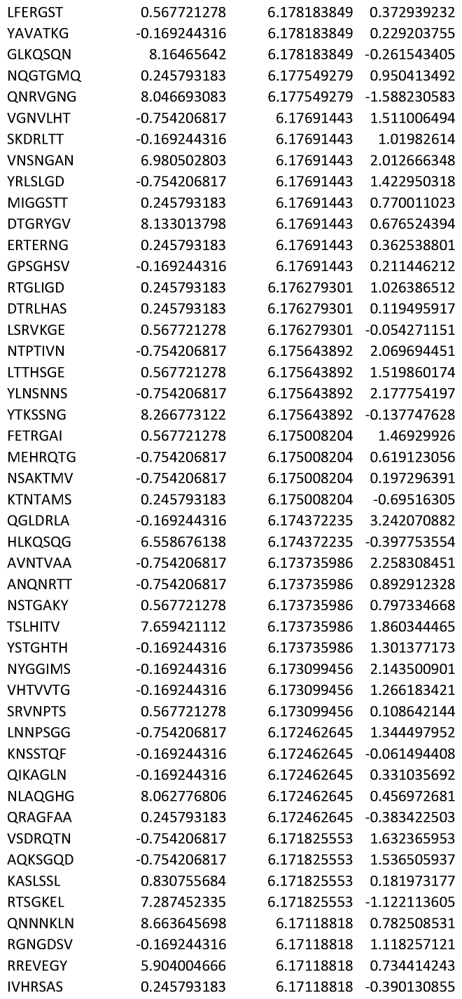

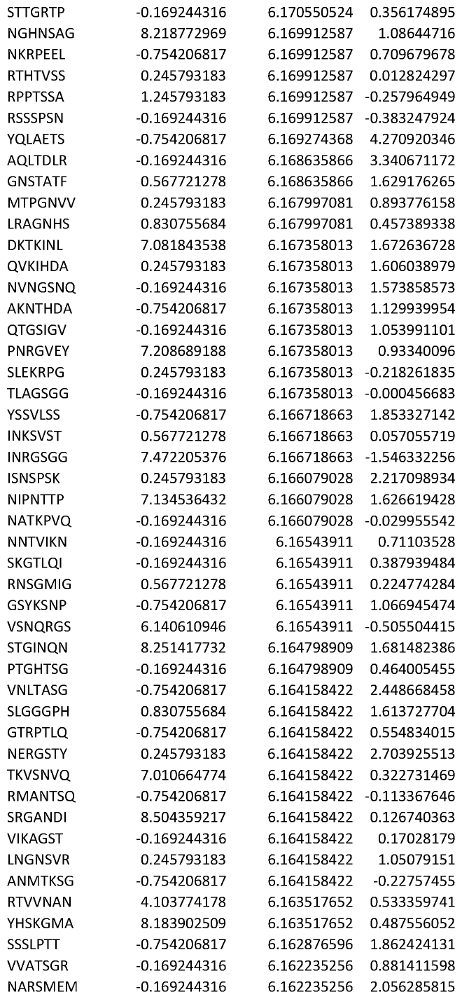

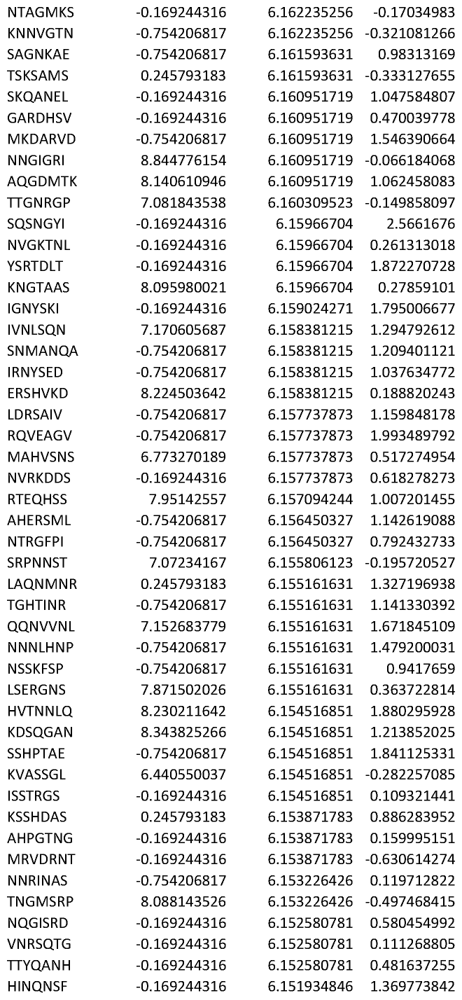

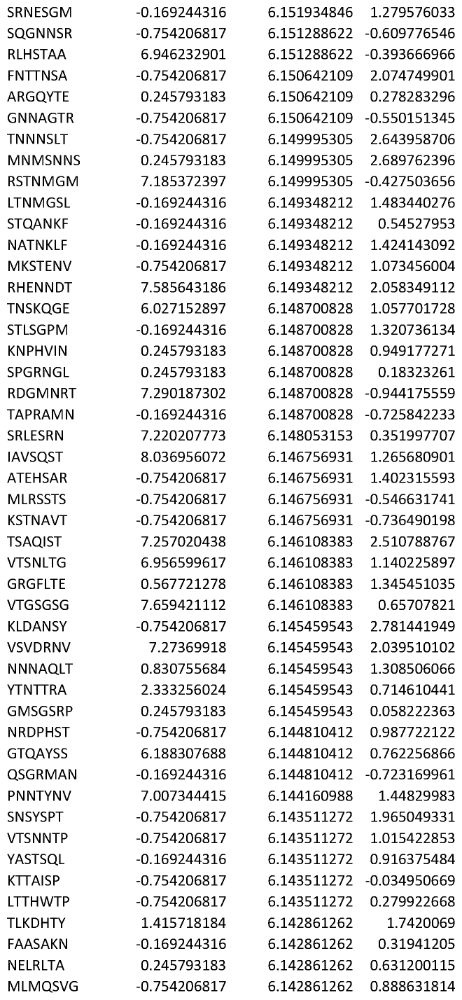

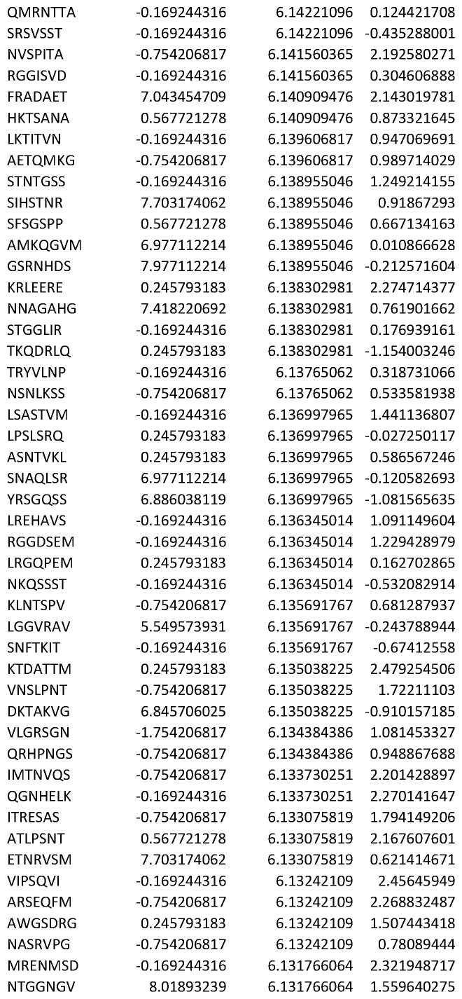

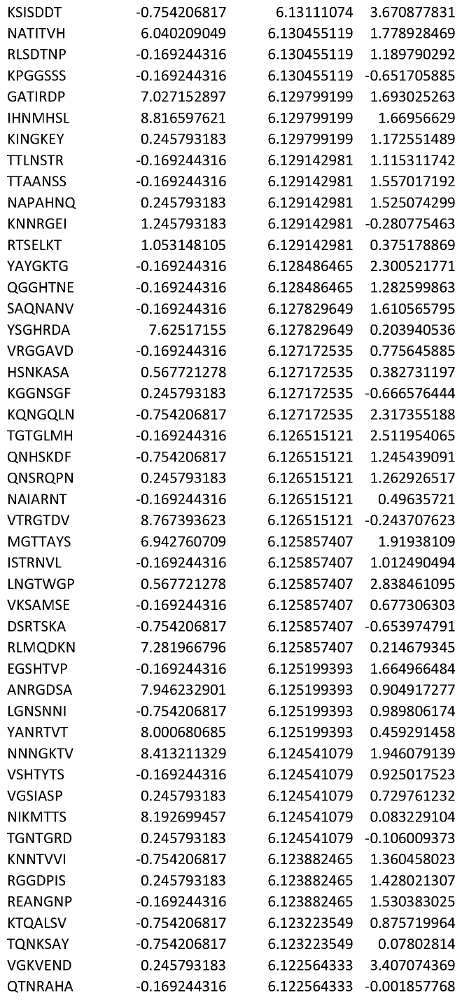

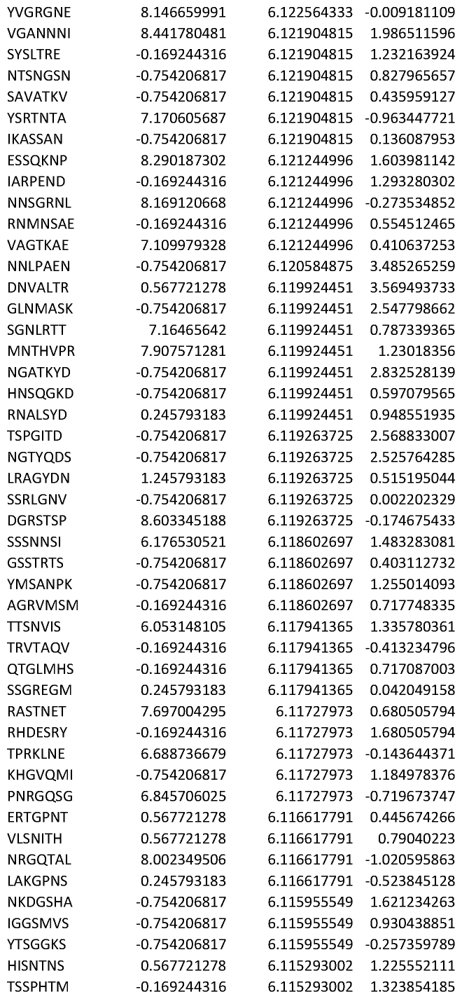

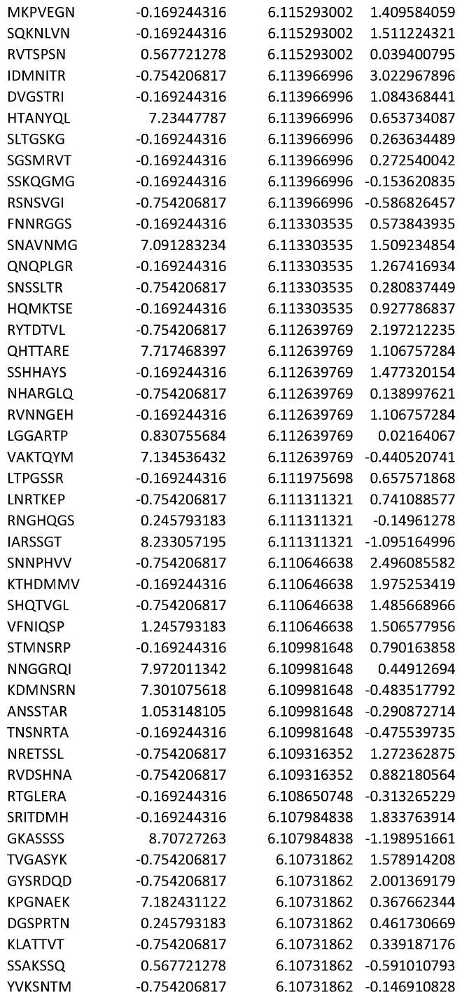

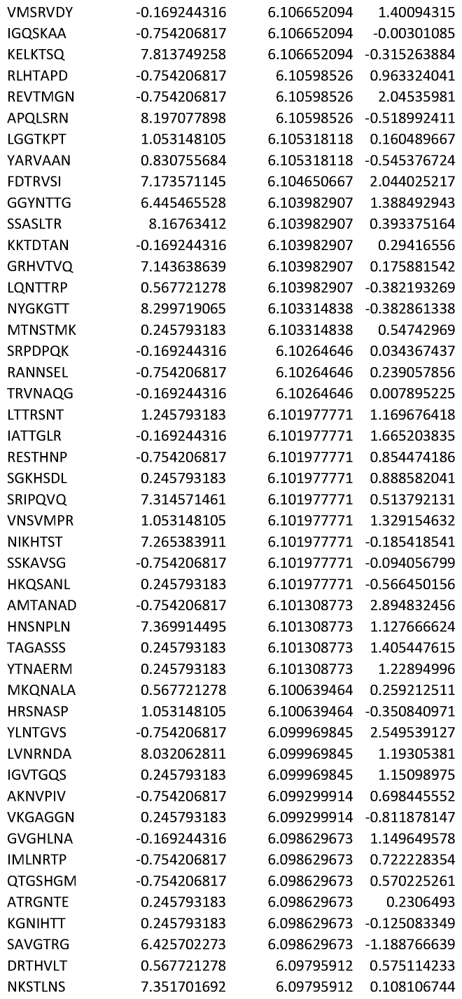

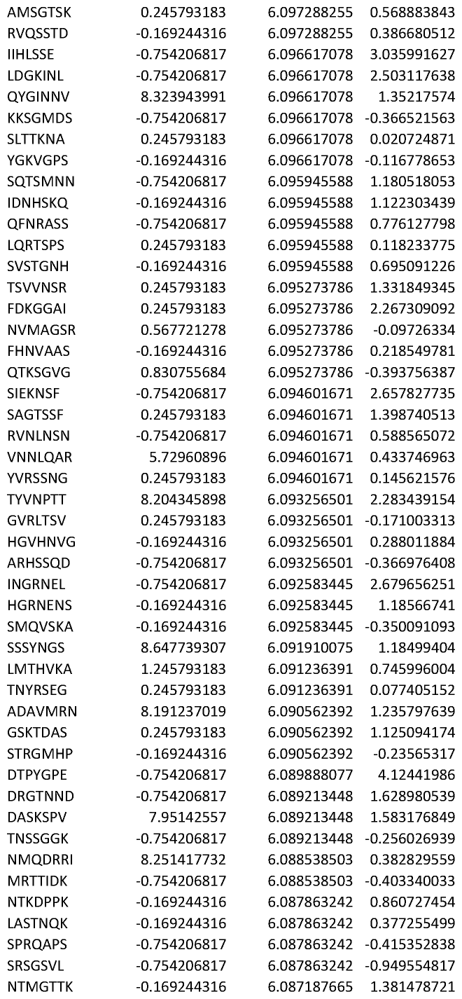

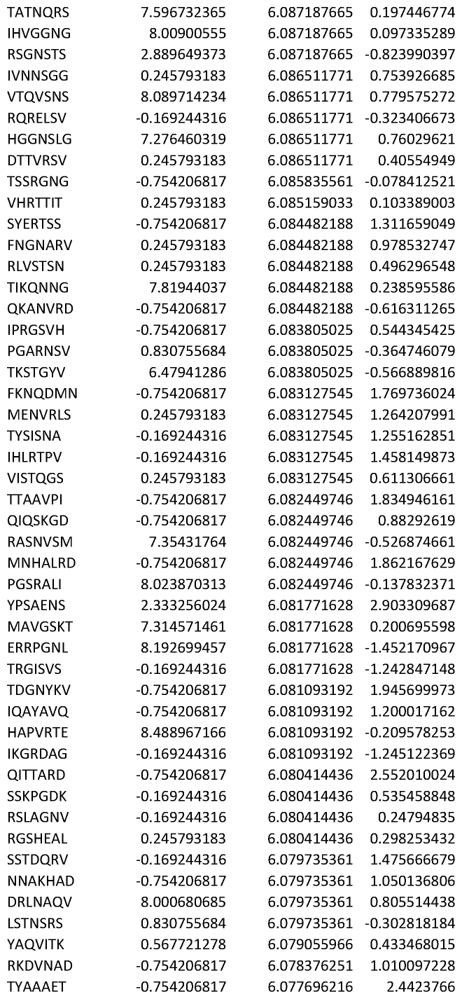

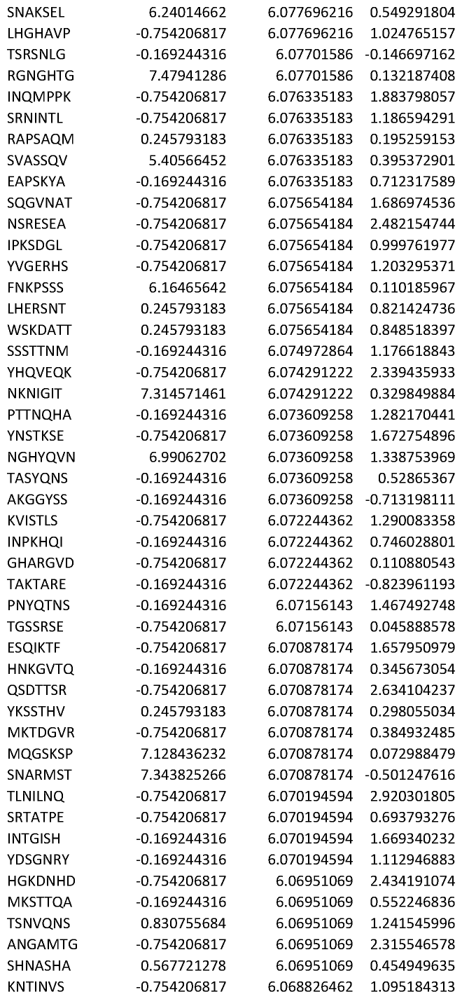

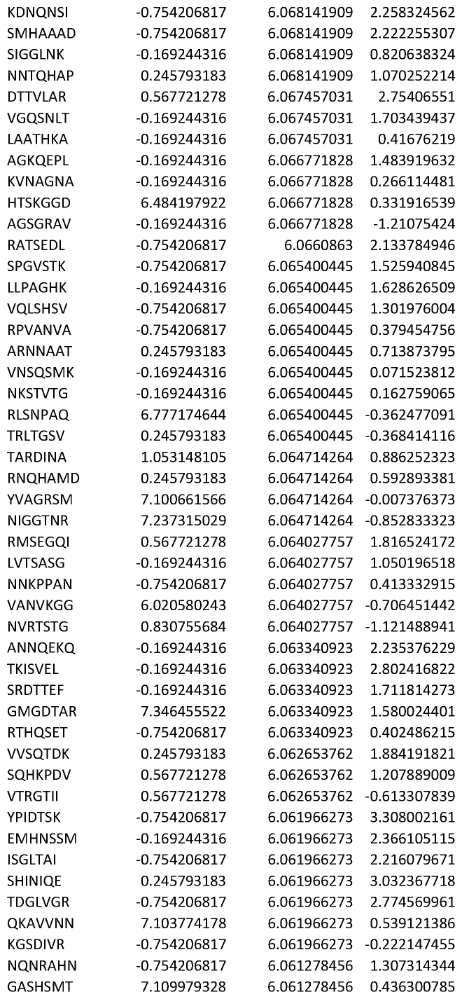

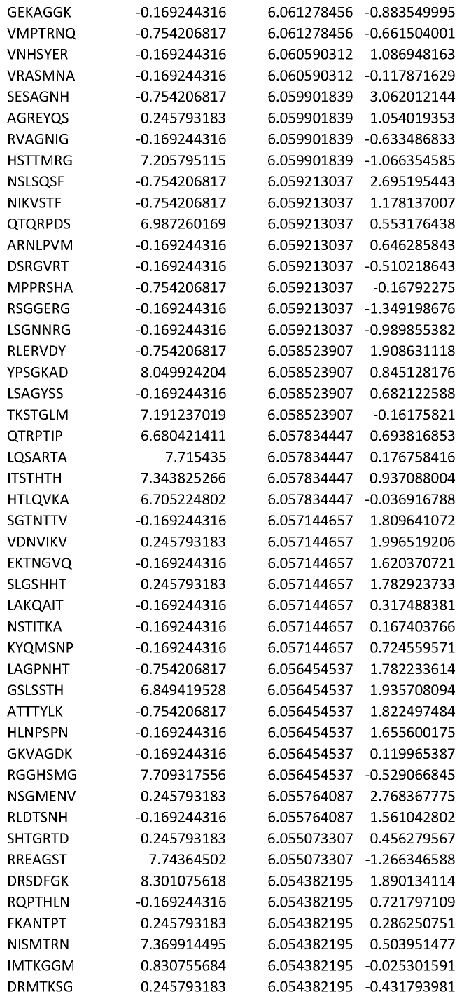

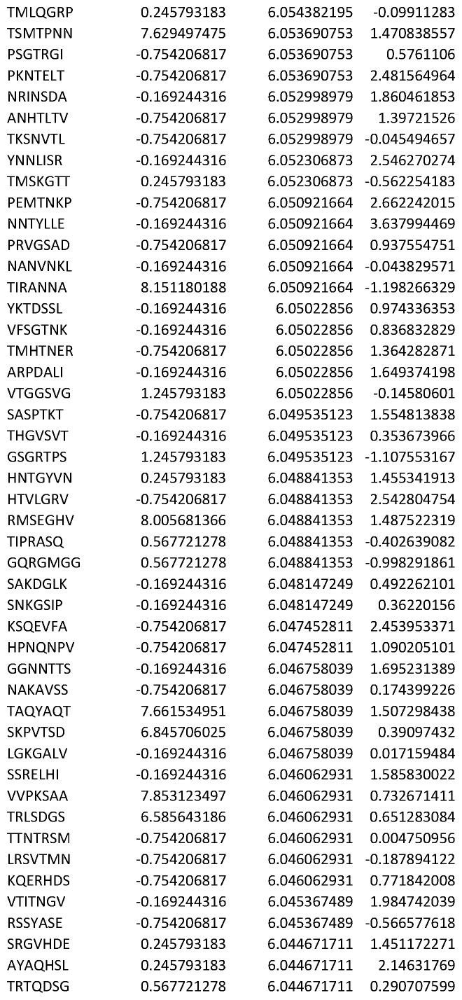

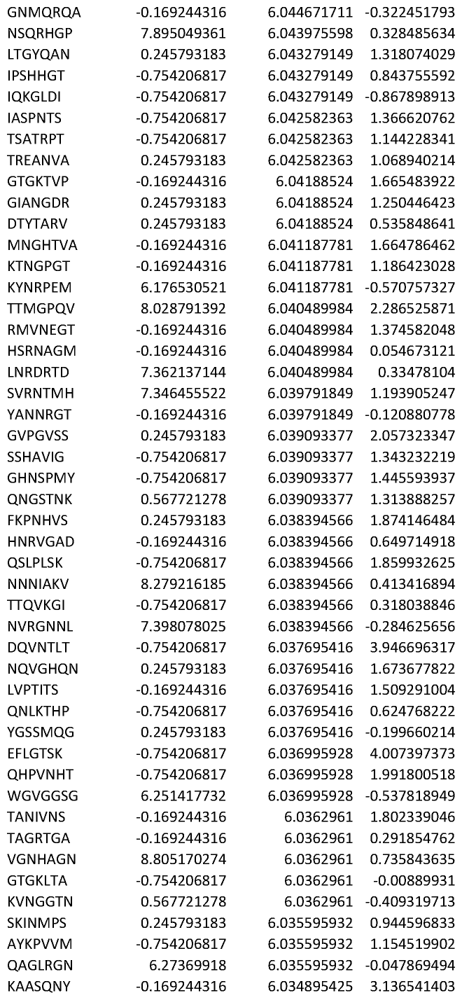

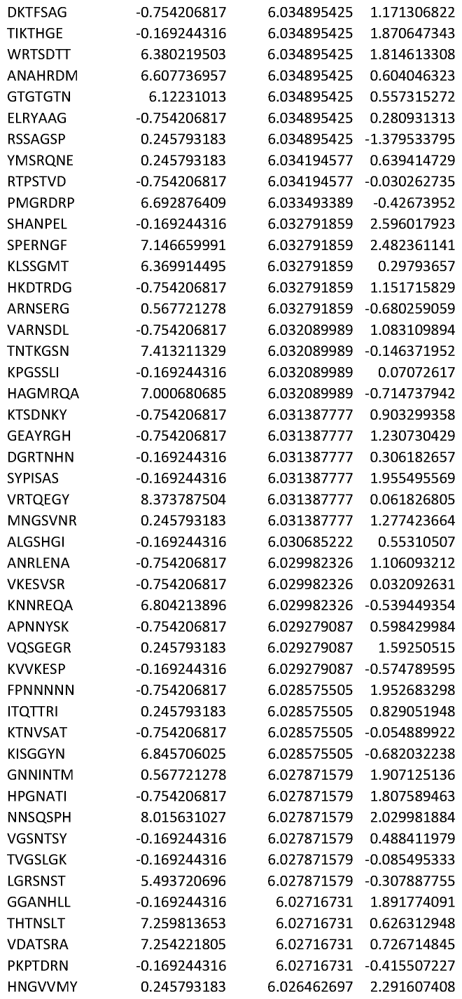

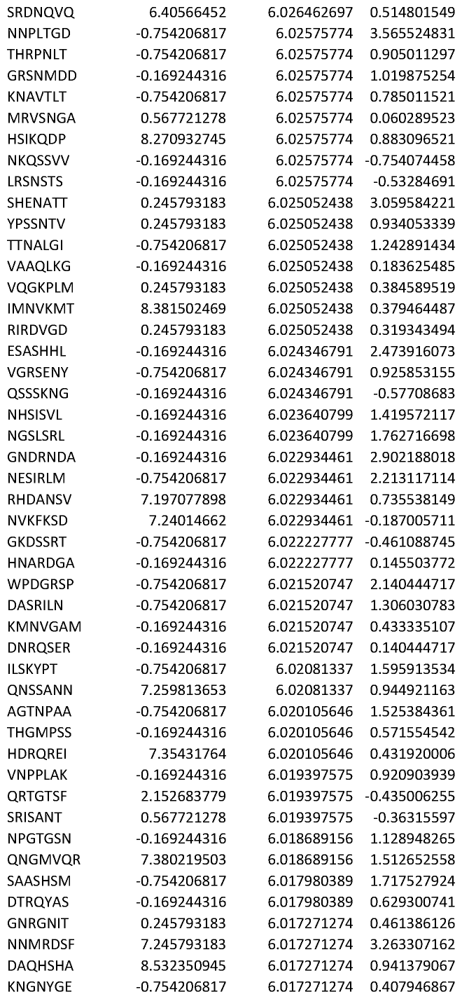

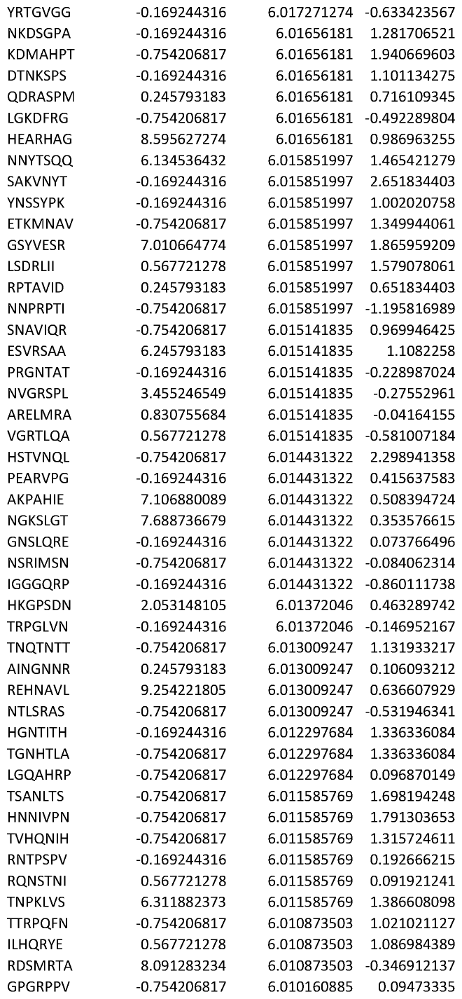

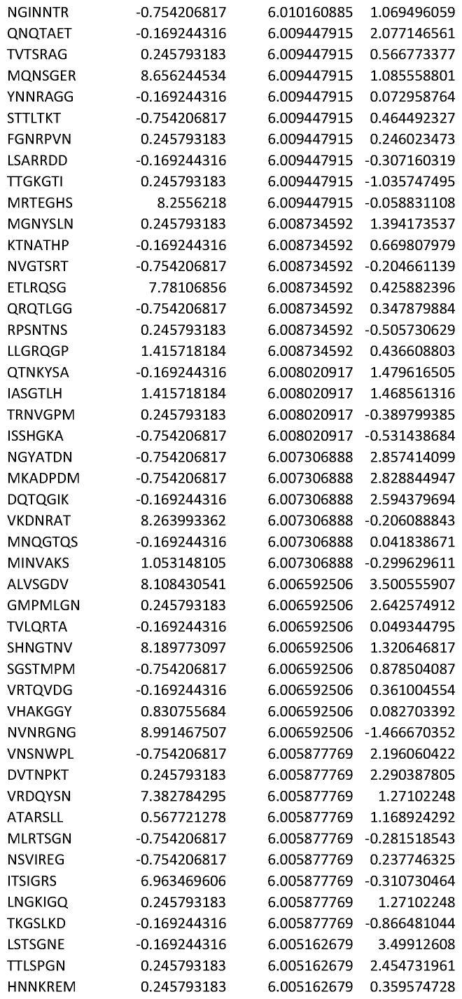

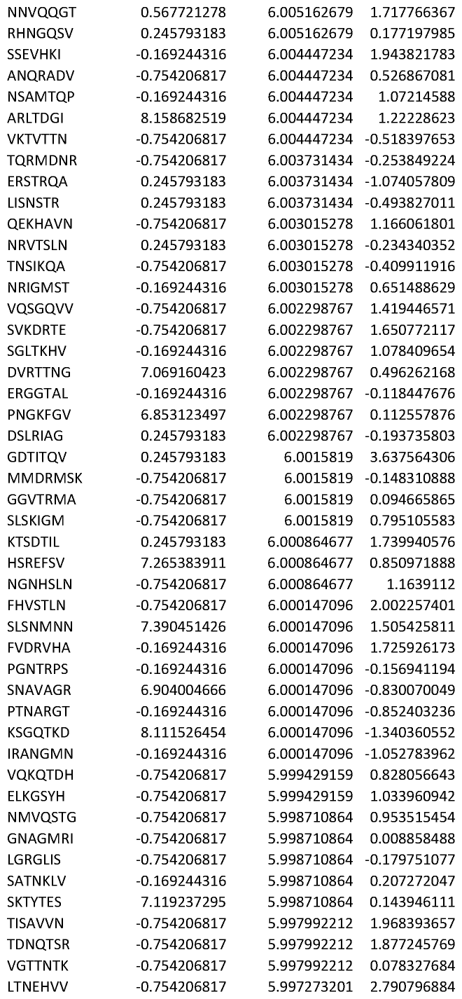

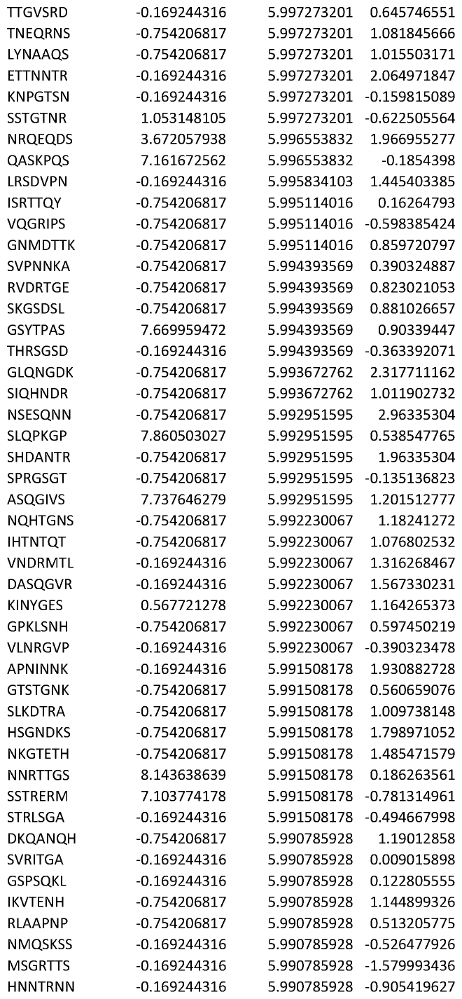

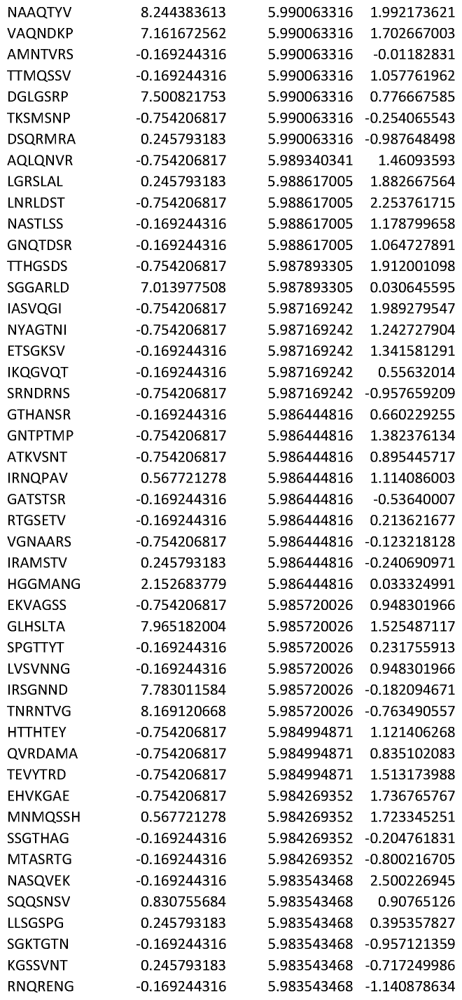

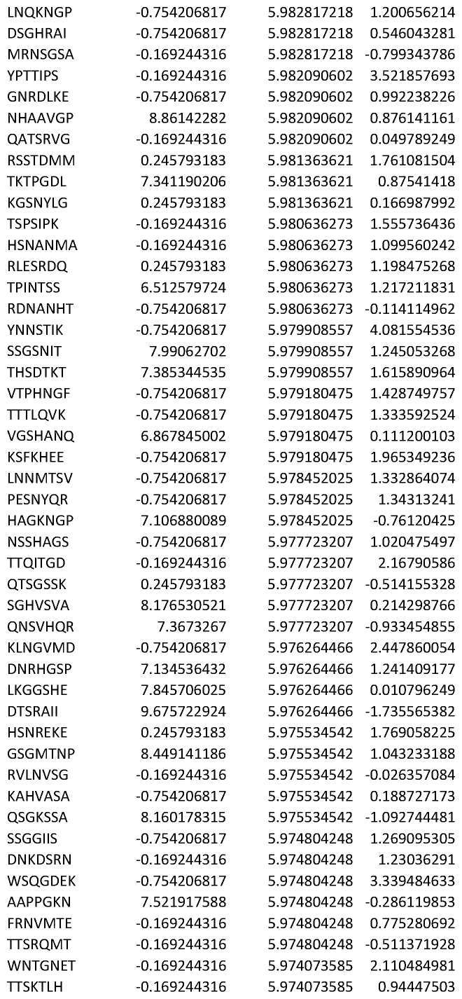

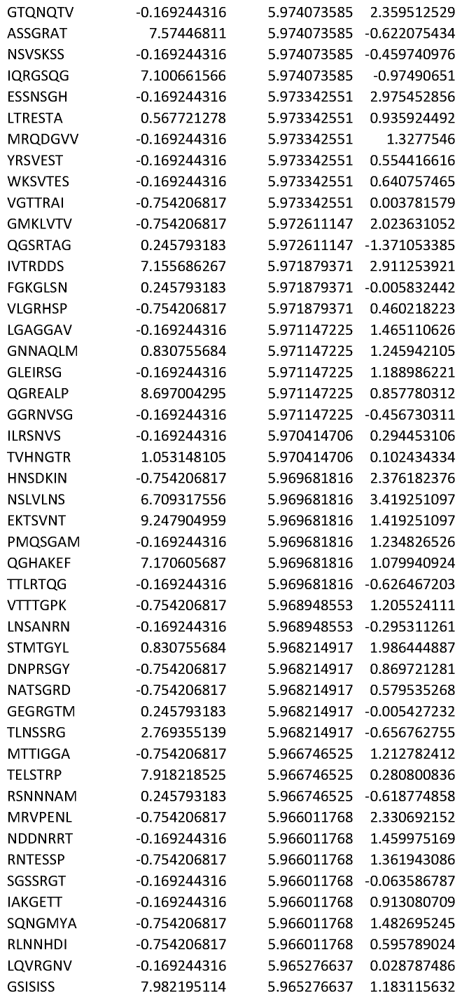

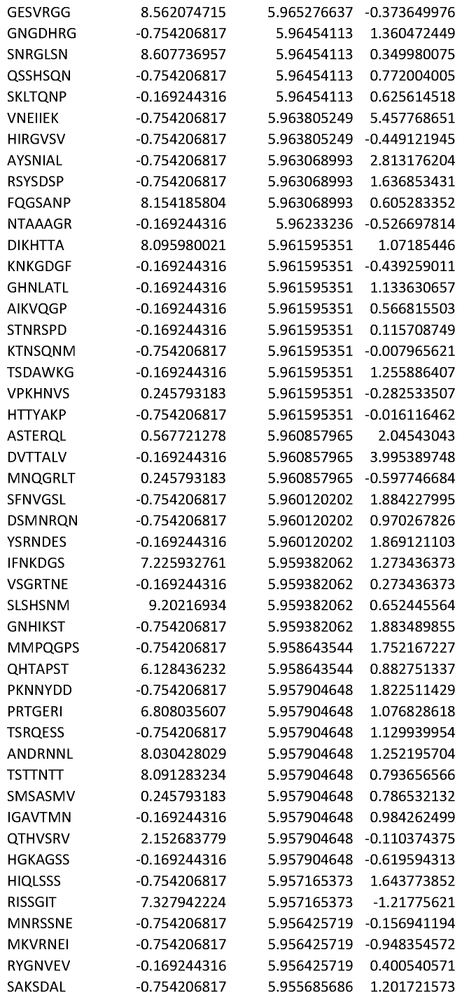

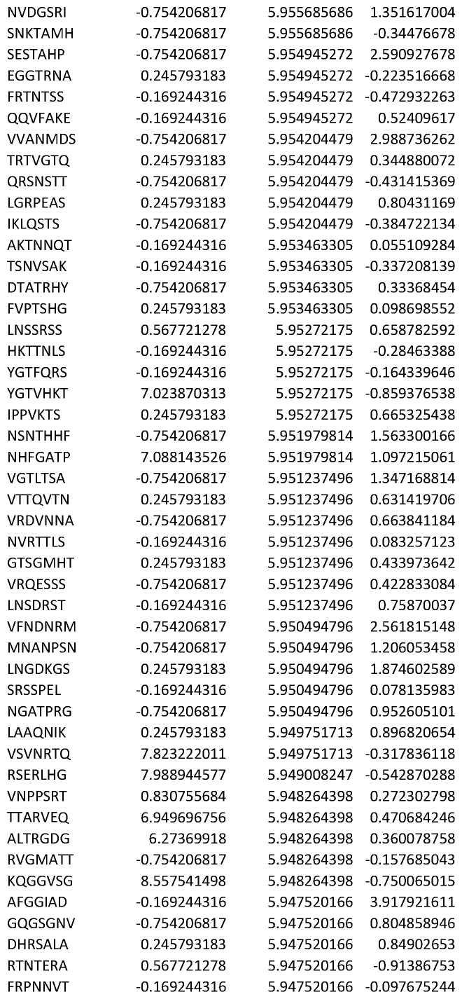

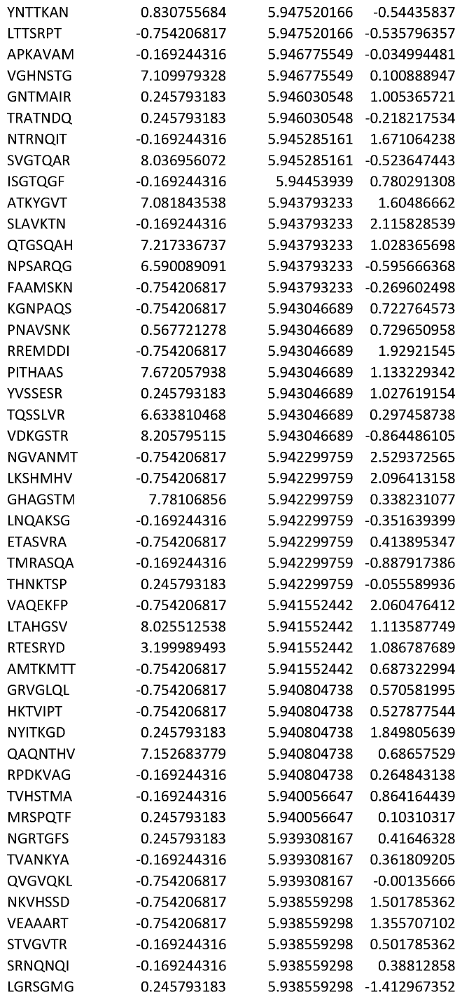

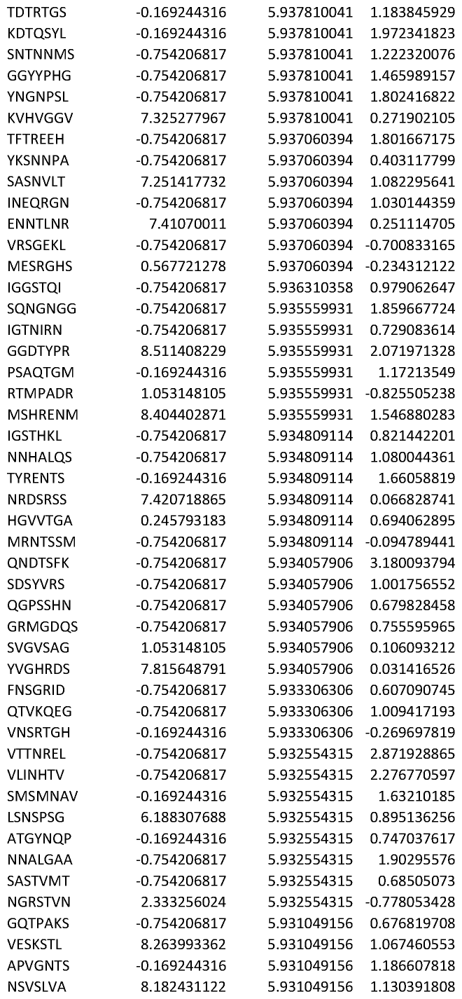

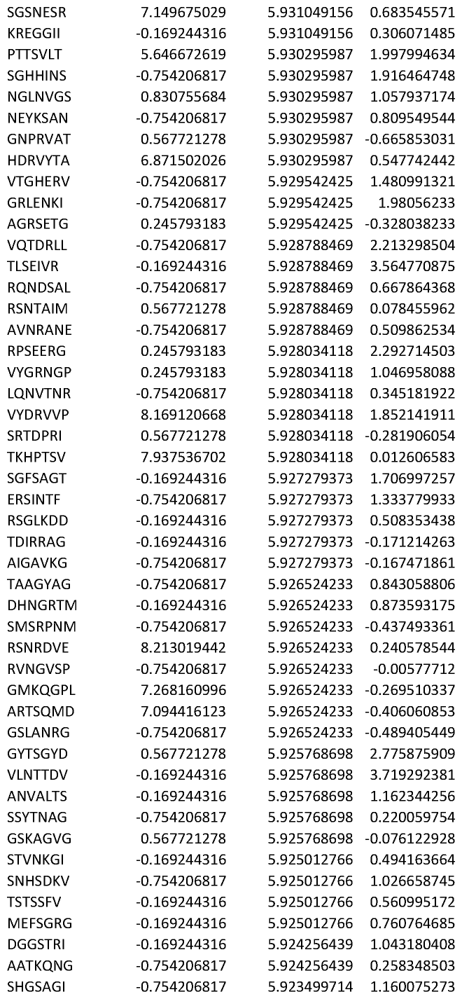

In one aspect, the disclosure provides rAAVs with a peptide insertion comprising or consisting of an amino-acid sequence set forth in any one of Tables 1 and 4-30, FIG. 4 and/or Formulas I-XXXIII, as defined below in greater detail.

Some aspects disclosed herein provide AAV capsids comprising an AAV capsid protein comprising an insertion sequence of Formula I

(I)
(SEQ ID NO: 2)
X¹-X²-X³-X⁴-X⁵-X⁶-X⁷

wherein: X¹ is an amino acid selected from I, L, M and V;
X² is an amino acid selected from A, S and T;
X³ is an amino acid selected from K and R;
X⁴ is an amino acid selected from D, E, N and Q;
X⁵ is an amino acid selected from F, W and Y;
X⁶ is an amino acid selected from F, W and Y; and
X⁷ is an amino acid selected from K and R.

Aspects disclosed herein provide AAV capsids comprising an AAV capsid protein comprising an insertion sequence of Formula II

(II)
(SEQ ID NO: 3)
X⁸-X⁹-X¹⁰-X¹¹-X¹²-P-X¹³

wherein: X⁸ is an amino acid selected from I, L, M and V;
X⁹ is an amino acid selected from D, E, N, and Q;
X¹⁰ is an amino acid selected from A, S and T;
X¹¹ is an amino acid selected from A, S and T;
X¹² is an amino acid selected from K and R; and
X³ is an amino acid selected from I, L, M and V.

Aspects disclosed herein provide AAV capsids comprising an AAV capsid protein comprising an insertion sequence of Formula III

(III)
(SEQ ID NO: 4)
X¹⁴-X¹⁵-H-X¹⁶-X¹⁷-X¹⁸-X¹⁹

wherein: X¹⁴ is an amino acid selected from D, E, N and Q;
X¹⁵ is an amino acid selected from D, E, N and Q;
X¹⁶ is an amino acid selected from A, S and T;
X¹⁷ is an amino acid selected from K and R;
X¹⁸ is an amino acid selected from D, E, N and Q; and
X¹⁹ is an amino acid selected from D, E, N and Q.

Aspects disclosed herein provide AAV capsids with greater expression in brain comprising an AAV capsid protein comprising an insertion sequence of Formula IV

(IV)
(SEQ ID NO: 5)
X²⁰-X²¹-X²²-X²³-X²⁴-X²⁵-X²⁶

wherein: X²⁰ is an amino acid selected from A, I, G, P, H, N, S, R and Y;
X²¹ is an amino acid selected from Q, N, S, T, F, L, A and E;
X²² is an amino acid selected from T, S, G, R, N, and D;
X²³ is an amino acid selected from D, E, S, T, G, I, M, H and N;
X²⁴ is an amino acid selected from I, L, F, R, T, S, N and Q;
X²⁵ is an amino acid selected from A, L, Q, G, K, S, P and Y; and
X²⁶ is an amino acid selected from D, K, H, M, Y, T, L, and I;
   Provided X²² is not S when X²⁴ is R or S; further provided X²¹ is not S when X²³ is S or when X²⁵ is S; further provided X²⁵ is not S when X²⁴ is T or F or when X²⁶ is L; further provided X²³ is not T when X²⁴ is Q or when X²⁵ is P; further provided X²² is not G when X²⁰ is S or when X²⁶ is M; further provided X²⁵ is not L when X²³ is S or when X²⁶ is T or K; further provided X²² is not T when X²⁴ is S or when X²⁵ is P; further provided X²⁴ is not S when X²² is D or R; further provided X²⁵ is not G when X²² is G or T; further provided X²⁰ is not G when X²⁵ is P; further provided X²⁵ is not A or X²³ is T when X²⁶ is T; further provided X²⁰ is not Y when X²² is A; further provided X²⁰ is not R when X²³ is D; further provided X²¹ is not L when X²⁴ is L; further provided X²¹ is not T when X²³ is H; further provided X²¹ is not N when X²² is N; further provided X²³ is not G when X²⁶ is H; further X²² is not R when X²³ is I; and further provided X²⁵ is not Q when X²⁰ is P.

In some embodiments, the insertion sequence comprises a sequence of Formula IV wherein X²² is R.

In some embodiments, the insertion sequence as described in Table 4, is selected from AFGGIAD (SEQ ID NO: 37), ISREFYK (SEQ ID NO: 38), GTDMRQT (SEQ ID NO: 39), HLTSNQL (SEQ ID NO: 40), PSSNNPH (SEQ ID NO: 41), NARSTGM (SEQ ID NO: 42), SNRTLSI (SEQ ID NO: 43), SQSIQKD (SEQ ID NO: 44), REDHNLY (SEQ ID NO: 45) and YQNDSGK (SEQ ID NO: 46).

Aspects disclosed herein provide AAV capsids with greater enrichment in the BRAIN over that found in the LIVER comprising an AAV capsid protein comprising an insertion sequence of Formula V

(V)
(SEQ ID NO: 6)
X²⁷-X²⁸-X²⁹-X³⁰-X³¹-X³²-X³³

wherein: X²⁷ is an amino acid selected from I, G, L, T, V, D, S and N;
X²⁸ is an amino acid selected from D, A, L, I, H, Y, F and N;
X²⁹ is an amino acid selected from S, T, M, E, V, L, I and N;
X³⁰ is an amino acid selected from P, G, L, I, V, E and D;
X³¹ is an amino acid selected from T, E, S, G, I, M, Q and N;
X³² is an amino acid selected from P, S, M, H, I, V, E and D; and
X³³ is an amino acid selected from G, L, K, H, T and D;
   provided X²⁷ is not S when X³² is S; further provided X²⁷ is not T when X²⁹ is I or S; further provided X²⁷ is not V when X²⁹ is S; further provided X²⁷ is not L when X³¹ is N; further provided X²⁸ is not N when X³² is P; further provided X²⁹ is not V when X³⁰ is P; further provided X²⁹ is not N when X³⁰ is V; further provided X³⁰ is not G when X³¹ is Q; further provided X²⁹ is not S when X³² is P; further provided X³¹ is not T when X³² is S or V; and further provided X³² is not S when X³³ is K or L.

In some embodiments, the insertion sequence comprises a sequence of Formula V wherein X²⁷ is I or L.

In some embodiments, the insertion sequence as described in Table 7, is selected from IDVDTPT (SEQ ID NO: 47), GASGEDL (SEQ ID NO: 48), LDNLSVT (SEQ ID NO: 49), TLMEGMK (SEQ ID NO: 50), VNEIIEK (SEQ ID NO: 51), LHLGMID (SEQ ID NO: 52), DHEVTDH (SEQ ID NO: 53), SYIPGHK (SEQ ID NO: 54), NIEDNMG (SEQ ID NO: 55) and IFTLQSG (SEQ ID NO: 56).

Aspects disclosed herein provide AAV capsids having greater enrichment in the BRAIN over that found in the SPINAL CORD comprising an AAV capsid protein comprising an insertion sequence of Formula VI

(VI)
(SEQ ID NO: 7)
X³⁴-X³⁵-X³⁶-X³⁷-X³⁸-X³⁹-X⁴⁰

wherein: X³⁴ is an amino acid selected from T, K, N, A, V and L;
X³⁵ is an amino acid selected from T, S, A, L, P and N;
X³⁶ is an amino acid selected from T, S, I, A, N and P;
X³⁷ is an amino acid selected from S, T, D, E, N, V, I and L;
X³⁸ is an amino acid selected from S, T, K, R, P, V, L, A and G;
X³⁹ is an amino acid selected from N, T, S, K, D, E and G; and
X⁴⁰ is an amino acid selected from S, T, K, N, Q, D, L and E;
   Provided X⁴⁰ is not S when X³⁴ is A or N or when X³⁵ is N; further provided X³⁹ is not S when X³⁴ is T or L; further provided X⁴⁰ is not N or when X³⁵ is A or when X³⁶ is S; further provided X³⁶ is not S when X³⁹ is T or when X⁴⁰ is L; further provided X³⁵ is not S when X³⁹ is G or when X⁴⁰ is D or K; further provided X³⁸ is not S when X³⁴ is V or when X⁴⁰ is K; further provided X³⁵ is not P when X³⁶ is P or when X³⁷ is L; further provided X³⁹ is not T when X³⁴ is not L or when X³⁶ is A; further provided X³⁷ is not S when X³⁶ is A or N; further provided X³⁷ is not V when X³⁴ is T or K; further provided X³⁵ is not T when X³⁴ is K or when X³⁹ is K; further provided X³⁴ is not V when X³⁵ is A or when X⁴⁰ is Q; further provided X³⁴ is not L when X³⁶ is P; further provided X³⁴ is not A when X³⁸ is P; further provided X³⁵ is not N when X³⁶ is T; and further provided X³⁷ is not T when X³⁹ is N.

In some embodiments, the insertion sequence comprises a sequence of Formula VI wherein X⁴¹ is L, X⁴³ is T, and X⁴⁷ is V.

In some embodiments, the insertion sequence as described in Table 8, is selected from TTISSTS (SEQ ID NO: 57), KSSDKDS (SEQ ID NO: 58), NSNVPKN (SEQ ID NO: 59), AAAEVNK (SEQ ID NO: 60), VLTTLSK (SEQ ID NO: 61), VTTNREL (SEQ ID NO: 62), NPTVANT (SEQ ID NO: 63), TLNILNQ (SEQ ID NO: 64), NNPLTGD (SEQ ID NO: 65) and LSTSGNE (SEQ ID NO: 66).

Aspects disclosed herein provide AAV capsids having greater enrichment in the BRAIN over that found in the LIVER and SPINAL CORD comprising an AAV capsid protein comprising an insertion sequence of Formula VII

(VII)
(SEQ ID NO: 8)
X⁴¹-X⁴²-X⁴³-X⁴⁴-X⁴⁵-X⁴⁶-X⁴⁷

wherein: X⁴¹ is an amino acid selected from Q, G, A, S, C, E, P and L;
X⁴² is an amino acid selected from D, P, H, S, G, V, L and N;
X⁴³ is an amino acid selected from N, E, Q, S, T, V, G and D;
X⁴⁴ is an amino acid selected from G, T, S, M, Y and E;
X⁴⁵ is an amino acid selected from P, F, T, K, E, M, A and G;
X⁴⁶ is an amino acid selected from V, E, D, M, K, S and Y; and
X⁴⁷ is an amino acid selected from R, K, N, A, T, V and W;
   Provided X⁴¹ is not G when X⁴⁶ is S; further provided X⁴¹ is not S when X⁴⁶ is Y or S; further provided X⁴¹ is not A when X⁴⁵ is A; further provided X⁴¹ is not P when X⁴³ is N; further provided X⁴² is not P when X⁴⁶ is S; further provided X⁴² is not S when X⁴⁶ is D; further provided X⁴² is not H when X⁴⁷ is K; further provided X⁴³ is not S when X⁴⁴ is G; further provided X⁴³ is not G when X⁴⁵ is P; further provided X⁴⁴ is not T when X⁴⁷ is T; further provided X⁴⁴ is not S when X⁴⁶ is V; and further provided X⁴⁵ is not G when X⁴⁷ is V.

In some embodiments, the insertion sequence comprises a sequence of Formula VII wherein X⁴¹ is A. In some embodiments, the insertion sequence comprises a sequence of Formula VII wherein X⁴³ is D. In some embodiments, the insertion sequence comprises a sequence of Formula VII wherein X⁴¹ is L, X⁴³ is T, and X⁴⁷ is V. In some embodiments, the insertion sequence comprises a sequence of Formula VII wherein X⁴⁶ is E or D, and X⁴⁷ is K or R.

In some embodiments, the insertion sequence as described in Table 9, is selected from QVDGPVR (SEQ ID NO: 67), GDNGFYK (SEQ ID NO: 68), APVTGEN (SEQ ID NO: 69), SNDMTEK (SEQ ID NO: 70), CNEEMKA (SEQ ID NO: 71), ENQSAST (SEQ ID NO: 72), PHSEGDN (SEQ ID NO: 73), LSTETMV (SEQ ID NO: 74), AGDYKEW (SEQ ID NO: 75) and ALGEEST (SEQ ID NO: 76).

Aspects disclosed herein provide AAV capsids having greater enrichment in the SPINAL CORD comprising an AAV capsid protein comprising an insertion sequence of Formula VIII

(VIII)
(SEQ ID NO: 9)
X⁴⁸-X⁴⁹-X⁵⁰-X⁵¹-X⁵²-X⁵³-X⁵⁴

wherein: X⁴⁸ is an amino acid selected from E, S, G, A, N, and P;
X⁴⁹ is an amino acid selected from D, S, K, N, I and L;
X⁵⁰ is an amino acid selected from N, S, T, G, V, A and R;
X⁵¹ is an amino acid selected from L, T, G, N, D, R and A;
X⁵² is an amino acid selected from S, A, P, E, I, T and M;
X⁵³ is an amino acid selected from Y, F, T, N, G, E, P and Q; and
X⁵⁴ is an amino acid selected from V, I, D, A, Y, N, E and T;
   Provided X⁵² is not S when X⁴⁹ is L or S; further provided X⁴⁸ is not S when X⁴⁹ is K, further provided X⁴⁸ is not S, when X⁵² is T or when X⁵³ is P; further provided X⁴⁸ is not P when X⁵³ is N, further provided X⁴⁸ is not G when X⁵³ is T, further provided X⁴⁹ is not S when X⁵² is M or X⁵¹ is N; further provided X⁴⁹ is not N when X⁵³ is T; further provided X⁵⁰ is not G when X⁵¹ is L, further provided X⁴⁹ is not N when X⁵⁴ is V, and further provided X⁵³ is not N when X⁵⁴ is A.

In some embodiments, the insertion sequence comprises a sequence of Formula VIII wherein X⁴⁸ is E or S. In some embodiments, the insertion sequence comprises a sequence of Formula VIII wherein X⁴⁹ is D.

In some embodiments, the insertion sequence as described in Table 5, is selected from EDNLSYV (SEQ ID NO: 77), SDSTAFI (SEQ ID NO: 78), SSNGPTD (SEQ ID NO: 79), EKTNEND (SEQ ID NO: 80), SNTDSGT (SEQ ID NO: 81), GIGTSEA (SEQ ID NO: 82), AIVAAGY (SEQ ID NO: 83), NLANIPN (SEQ ID NO: 84), PLRTTQE (SEQ ID NO: 85) and SDRRMNT (SEQ ID NO: 86).

Aspects disclosed herein provide AAV capsids having greater enrichment in the SPINAL CORD over that found in the LIVER comprising an AAV capsid protein comprising an insertion sequence of Formula IX

(IX)
(SEQ ID NO: 10)
X⁵⁵-X⁵⁶-X⁵⁷-X⁵⁸-X⁵⁹-X⁶⁰-X⁶¹

wherein: X⁵⁵ is an amino acid selected from N, E, M, G, S, P, D, C and V;
X⁵⁶ is an amino acid selected from Q, L, A, I, G, R, T, S, and V;
X⁵⁷ is an amino acid selected from K, N, V, L, G, A and E;
X⁵⁸ is an amino acid selected from P, T, G, M, S and E;
X⁵⁹ is an amino acid selected from D, S, A, P, R, I, M, Q and L;
X⁶⁰ is an amino acid selected from A, M, E, P, T, V, L and Q; and
X⁶¹ is an amino acid selected from K, P, T, M, H, N and Y;
   Provided X⁵⁵ is not V when X⁵⁶ is G or L or when X⁵⁷ is N; further provided X⁵⁵ is not P when X⁵⁷ is K or when X⁵⁸ is P; further provided X⁵⁸ is not S when X⁵⁵ is S or E or when X⁶⁰ is A; further provided X⁵⁷ is not L when X⁵⁹ is R or when X⁶¹ is P; further provided X⁵⁷ is not G when X⁵⁹ is L or when X⁶¹ is P; further provided X⁶¹ is not T when X⁵⁷ is A or G; further provided X⁵⁹ is not P when X⁵⁶ is R or when X⁶¹ is M; further provided X⁵⁹ is not S when X⁵⁷ is A or when X⁶¹ is K; further provided X⁵⁵ is not D when X⁵⁶ is V; further provided X⁵⁵ is not N when X⁵⁷ is V; further provided X⁵⁸ is not T when X⁵⁶ is T; further provided X⁵⁷ is not E when X⁶¹ is H; further provided X⁵⁶ is not Q when X⁶⁰ is not P; and further provided X⁵⁸ is not G when X⁶¹ is not P.

In some embodiments, the insertion sequence comprises a sequence of Formula IX wherein X⁵⁹ is S.

In some embodiments, the insertion sequence as described in Table 12, is selected from NSEPDAN (SEQ ID NO: 87), ELGTAEM (SEQ ID NO: 88), STLEMPH (SEQ ID NO: 89), VQVGSMT (SEQ ID NO: 90), PTNMPPT (SEQ ID NO: 91), DAVSRVP (SEQ ID NO: 92), CGKTILT (SEQ ID NO: 93), MVNELTP (SEQ ID NO: 94), NIAEQPK(SEQ ID NO: 95) and GREPSQY (SEQ ID NO: 96).

Aspects disclosed herein provide AAV capsids having a greater enrichment in the SPINAL CORD over that found in BRAIN comprising an AAV capsid protein comprising an insertion sequence of Formula X

(X)
(SEQ ID NO: 11)
X⁶²-X⁶³-X⁶⁴-X⁶⁵-X⁶⁶-X⁶⁷-X⁶⁸

wherein: X⁶² is an amino acid selected from D, T, K, M, I, A, G and N;
   X⁶³ is an amino acid selected from Q, N, T, P, L, I, G and Y;
   X⁶⁴ is an amino acid selected from T, S, M, G, A and F;
   X⁶⁵ is an amino acid selected from N, T, H, G and P;
   X⁶⁶ is an amino acid selected from S, D, Q, N and R;
   X⁶⁷ is an amino acid selected from T, G, A, Y, E, D, K and N; and
   X⁶⁸ is an amino acid selected from H, A, F, Y, P, N, I and V;
      provided X⁶⁴ is not S when X⁶² is T; further provided X⁶⁵ is not N or T when X⁶⁶ is R; further provided X⁶³ is not P when X⁶² is T or M; further provided X⁶² is not G when X⁶⁵ is N; further provided X⁶⁵ is not G when X⁶⁷ is T; further provided X⁶³ is not Y when X⁶⁷ is A; further provided X⁶⁴ is not S when X⁶⁸ is N; and further provided X⁶⁴ is not T when X⁶⁶ is N.

In some embodiments, the insertion sequence comprises a sequence of Formula X wherein X⁶⁵ is N. In some embodiments, the insertion sequence comprises a sequence of Formula X wherein X⁶⁶ is S. In some embodiments, the insertion sequence comprises a sequence of Formula X wherein X⁶³ is Q or N.

In some embodiments, the insertion sequence as described in Table 11, is selected from DQTNSTH (SEQ ID NO: 97), MQMNSGA (SEQ ID NO: 98), NTMNSYP (SEQ ID NO: 99), ILSNQAF (SEQ ID NO: 100), GYSTSEV (SEQ ID NO: 101), ANSHDKI (SEQ ID NO: 102), GPGTSDN (SEQ ID NO: 103), TGFNNKI (SEQ ID NO: 104), DIAGRNP (SEQ ID NO: 105) and KQSPSNY (SEQ ID NO: 106).

Aspects disclosed herein provide AAV capsids having greater enrichment in the SPINAL CORD over that found in the LIVER and BRAIN comprising an AAV capsid protein comprising an insertion sequence of Formula XI

(XI)
(SEQ ID NO: 12)
X⁶⁹-X⁷⁰-X⁷¹-X⁷²-X⁷³-X⁷⁴-X⁷⁵

wherein: X⁶⁹ is an amino acid selected from S, G, M, Q, H, V, I, A and E;
   X⁷⁰ is an amino acid selected from T, E, N, H, A, L, D, and R;
   X⁷¹ is an amino acid selected from H, M, D, E, and A;
   X⁷² is an amino acid selected from D, K, F, G, L, N, and T;
   X⁷³ is an amino acid selected from R, D, V, S, T, G, N, and H;
   X⁷⁴ is an amino acid selected from D, M, S, R, T, and G; and
   X⁷⁵ is an amino acid selected from F, M, T, T, H, W, and P;
      Provided X⁷¹ is not A or M when X⁷⁴ is S; further provided X⁷² is not G or T when X⁷⁴ is T; further provided X⁷⁰ is not R when X⁷³ is V or when X⁶⁹ is Q; further provided X⁷³ is not R when X⁶⁹ is I or when X⁷¹ is M; further provided X⁷⁴ is not E when X⁶⁹ is S or when X⁷² is L; further provided X⁷³ is not S when X⁷⁰ is L or when X⁶⁹ is G; further provided X⁷⁰ is not H when X⁷³ is G; further provided X⁷¹ is not A when X⁷⁴ is D; further provided X⁷¹ is not H when X⁷² is L; further provided X⁷² is not T when X⁷⁴ is R; and further provided X73 is not T when X⁷⁴ is G.

In some embodiments, the insertion sequence comprises a sequence of Formula XI wherein X⁷¹ is D or E. In some embodiments, the insertion sequence comprises a sequence of Formula XI wherein X⁷² is K.

In some embodiments, the insertion sequence as described in Table 10, is selected from STHDRDF (SEQ ID NO: 107), GEMKDMS (SEQ ID NO: 108), MNDFVSL (SEQ ID NO: 109), QHDGSML (SEQ ID NO: 110), HADLRDG (SEQ ID NO: 111), GLEFTRH (SEQ ID NO: 112), VDANGTW (SEQ ID NO: 113), IEEKNGT (SEQ ID NO: 114), ARDTDDA (SEQ ID NO: 115) and ETDKHGP (SEQ ID NO: 116).

Aspects disclosed herein provide AAV capsids having improved enrichment in both the BRAIN and in the SPINAL CORD comprising an AAV capsid protein comprising an insertion sequence of Formula XII

(XII)
(SEQ ID NO: 13)
X⁷⁶-X⁷⁷-X⁷⁸-X⁷⁹-X⁸⁰-X⁸¹-X⁸²

wherein: X⁷⁶ is an amino acid selected from S, G, P, V, A and E;
   X⁷⁷ is an amino acid selected from N, G, A, L, V, D, and K;
   X⁷⁸ is an amino acid selected from I, N, Q, T, S, E, and G;
   X⁷⁹ is an amino acid selected from G, P, F, K, S, Q, N, and T;
   X⁸⁰ is an amino acid selected from K, R, T, S, Y, G, V and N;
   X⁸¹ is an amino acid selected from H, E, S, T, V and N; and
   X⁸² is an amino acid selected from I, N, L, H, K, D, Y, and T;
      provided X⁷⁷ is not L when X⁷⁸ is I or when X⁸⁰ is G or when X⁸² is T; further provided X⁷⁶ is not S when X⁷⁸ is G or when X⁷⁹ is S; further provided X⁷⁶ is not P when X⁷⁷ is V or when X⁸⁰ is S; further provided X⁷⁷ is not A when X⁷⁹ is P or when X⁸² is I; further provided X⁷⁸ is not S when X⁷⁹ is G or when X⁸¹ is S; further provided X⁸¹ is not S when X⁷⁹ is G or when X⁸⁰ is S; further provided X⁸¹ is not N when X⁷⁷ is N or when X⁸⁰ is T; further provided X⁸¹ is not T when X⁸² is L; further provided X⁷⁹ is not N when X⁸¹ is V; further provided X⁷⁷ is not G when X⁸⁰ is R; and further provided X⁷⁶ is not V when X⁷⁸ is T.

In some embodiments, the insertion sequence comprises a sequence of Formula XII wherein X⁷⁶ is S. In some embodiments, the insertion sequence comprises a sequence of Formula XII wherein X⁷⁷ is A, L or V. In some embodiments, the insertion sequence comprises a sequence of Formula XII wherein X⁸¹ is N.

In some embodiments, the insertion sequence as described in Table 16, is selected from SDIGKTH (SEQ ID NO: 117), PNEGGHN (SEQ ID NO: 118), AGNPGVI (SEQ ID NO: 119), VVGSTVL (SEQ ID NO: 120), GAITNNY (SEQ ID NO: 121), SLNNVTN (SEQ ID NO: 122), EKTSVNT (SEQ ID NO: 123), SLSQYEK (SEQ ID NO: 124), GAQFRSD (SEQ ID NO: 125) and VASKSNH (SEQ ID NO: 126).

Aspects disclosed herein provide AAV capsids having improved enrichment in the SPINAL CORD AND BRAIN over that found in the LIVER comprising an AAV capsid protein comprising an insertion sequence of Formula XIII

(XIII)
(SEQ ID NO: 14)
X⁸³-X⁸⁴-X⁸⁵-X⁸⁶-X⁸⁷-X⁸⁸-X⁸⁹

wherein: X⁸³ is an amino acid selected from F, I, A, N, E, D, N, Q, K and T;
   X⁸⁴ is an amino acid selected from G, T, I, W, S, D, A, and H;
   X⁸⁵ is an amino acid selected from E, D, T, I, N, M, and S;
   X⁸⁶ is an amino acid selected from I, N, P, E, D, H, V, and A;
   X⁸⁷ is an amino acid selected from T, R, V, H, G, A, and K;
   X⁸⁸ is an amino acid selected from P, I, A, Q, E, K, G, and H; and
   X⁸⁹ is an amino acid selected from G, V, H, E, S, K, A, P, and N;
      Provided X⁸⁴ is not S when X⁸⁵ is S or N or when X⁸³ is F; further provided X⁸⁵ is not T when X⁸⁷ is R or X⁸⁴ is S; further provided X⁸⁴ is not H when X⁸⁸ or X⁸⁶ is P; further provided X⁸³ is not A when X⁸⁷ is R; further provided X⁸⁶ is not A when X⁸⁹ is P; further provided X⁸⁴ is not T when X⁸⁹ is S; further provided X⁸⁵ is not S when X⁸⁷ is G; further provided X⁸⁵ is not T when X⁸⁴ is A or when X⁸⁷ is G; further provided X⁸³ is not T when X⁸⁴ is G or W; further provided X⁸³ is not E when X⁸⁴ is A; further provided X⁸⁶ is not V when X⁸⁸ is Q; and further provided X⁸⁸ is not P when X⁸⁹ is V.

In some embodiments, the insertion sequence comprises a sequence of Formula XIII wherein X⁸⁵ is D. In some embodiments, the insertion sequence comprises a sequence of Formula XIII wherein X⁸⁶ is N.

In some embodiments, the insertion sequence as described in Table 29, is selected from FGEITPG (SEQ ID NO: 127), ITDNRIV (SEQ ID NO: 128), AITPVAH (SEQ ID NO: 129), NGIERQE (SEQ ID NO: 130), EWNNHES (SEQ ID NO: 131), DSMDGKK (SEQ ID NO: 132), NDNNAGA (SEQ ID NO: 133), KDDHKEP (SEQ ID NO: 134), QADVGAN (SEQ ID NO: 135) and THSAVHH (SEQ ID NO: 136).

Aspects disclosed herein provide AAV capsids having an improved enrichment in the SPINAL CORD comprising an AAV capsid protein comprising an insertion of Formula XIV

(SEQ ID NO: 15)
X⁹⁰-X⁹¹-X⁹²-X⁹³-X⁹⁴-X⁹⁵-X⁹⁶
(XIV)

wherein: X⁹⁰ is an amino acid selected from E, N, D, T, S, I, N, and K;
   X⁹¹ is an amino acid selected from G, S, Q, I, L, P, and V;
   X⁹² is an amino acid selected from K, D, T, S, A and Q;
   X⁹³ is an amino acid selected from L, P, A, T, S and N;
   X⁹⁴ is an amino acid selected from H, P, I, A, S, T, Q, E and R;
   X⁹⁵ is an amino acid selected from V, A, T, S, G, N, Q and E; and
   X⁹⁶ is an amino acid selected from I, T, N, R, H, and Y;
      Provided X⁹⁰ is not S when X⁹² is T or when X⁹³ is S or when X⁹¹ is G; further provided X⁹² is not S or A or X⁹³ is not A when X⁹⁶ is T; further provided X⁹² is not A when X⁹³ is A or when X⁹⁵ is Q; further provided X⁹⁰ is not N when X⁹⁵ is G; further provided X⁹⁰ is not T when X⁹⁴ is A; further provided X⁹⁰ is not D when X⁹⁶ is N; further provided X⁹² is not S when X⁹⁴ is S; further provided X⁹⁵ is not S when X⁹⁶ is I; further provided X⁹¹ is not V when X⁹² is Q; further provided X⁹² is not T when X⁹⁶ is H; further provided X⁹¹ is not S when X⁹⁰ is I or T; and further provided X⁹⁴ is not S when X⁹⁰ is D or when X⁹³ is T.

In some embodiments, the insertion sequence comprises a sequence of Formula XIV wherein X⁹¹ is G, I, L or V. In some embodiments, the insertion sequence comprises a sequence of Formula XIV wherein X⁹³ is N.

In some embodiments, the insertion sequence as described in Table 14, is selected from EGKNEVI (SEQ ID NO: 137), NSDNHNI (SEQ ID NO: 138), DQKLPAT (SEQ ID NO: 139), TITPITN (SEQ ID NO: 140), ILTASER (SEQ ID NO: 141), IGTTQTN (SEQ ID NO: 142), SPATASH (SEQ ID NO: 143), SVDNRGN (SEQ ID NO: 144), NVSSRSN (SEQ ID NO: 145) and KSQATQY (SEQ ID NO: 146).

Aspects disclosed herein provide AAV capsids having improved enrichment in the SPINAL CORD over the LIVER comprising an AAV capsid protein comprising an insertion sequence of Formula XV

(SEQ ID NO: 16)
X⁹⁷-X⁹⁸-X⁹⁹-X¹⁰⁰-X¹⁰¹-X¹⁰²-X¹⁰³
(XV)

wherein: X⁹⁷ is an amino acid selected from D, G, A, M, I, N and T;
   X⁹⁸ is an amino acid selected from N, T, I, V, F, P, R and G;
   X⁹⁹ is an amino acid selected from G, E, M, A, I, F, S and V;
   X¹⁰⁰ is an amino acid selected from V, I, A, L, K, G, S, E, and D;
   X¹⁰¹ is an amino acid selected from K, V, I, A, G, Y, E and T;
   X¹⁰² is an amino acid selected from E, S, D, N, K, P, A and G; and
   X¹⁰³ is an amino acid selected from K, R, A, V, I, G and L;
      Provided X¹⁰² is not S when X⁹⁷ is T or when X¹⁰¹ is T; further provided X⁹⁷ is not N when X¹⁰⁰ is G or S or when X⁹⁸ is N; further provided X⁹⁷ is not G when X⁹⁸ is R or when X⁹⁹ is G; further provided X⁹⁹ is not S when X¹⁰¹ is T or A or when X⁹⁸ is G; further provided X⁹⁸ is not R when X¹⁰³ is A; further provided X¹⁰⁰ is not G when X¹⁰² is P; further provided X¹⁰⁰ is not S when X¹⁰² is A; further provided X⁹⁹ is not A when X¹⁰⁰ is L; further provided X⁹⁷ is not M when X¹⁰¹ is A or when X⁹⁸ is I or when X¹⁰² is S; further provided X¹⁰¹ is not T when X¹⁰³ is V; further provided X⁹⁷ is not I when X⁹⁸ is G or when X¹⁰⁰ is A; further provided X⁹⁸ is not T when X⁹⁷ is A or T; further provided X⁹⁸ is not P when X⁹⁹ is G; and further provided X¹⁰⁰ is not V when X¹⁰¹ is E or when X⁹⁸ is V.

In some embodiments, the insertion sequence comprises a sequence of Formula XV wherein X¹⁰⁰ is G, A, I or L.

In some embodiments, the insertion sequence as described in Table 18, is selected from DNGVKEK (SEQ ID NO: 147), GTELVSR (SEQ ID NO: 148), AIMKIDA (SEQ ID NO: 149), AFAGANV (SEQ ID NO: 150), MNFAGPI (SEQ ID NO: 151), GVSSIDK (SEQ ID NO: 152), IVSEYAG (SEQ ID NO: 153), NPIAESR (SEQ ID NO: 154), NREDTKL (SEQ ID NO: 155) and TGVIEGL (SEQ ID NO: 156).

Aspects disclosed herein provide AAV capsids having improved enrichment in the SPINAL CORD over that found in BRAIN comprising an AAV capsid protein comprising an insertion sequence of Formula XVI

(SEQ ID NO: 17)
X¹⁰⁴-X¹⁰⁵-X¹⁰⁶-X¹⁰⁷-X¹⁰⁸-X¹⁰⁹-X¹¹⁰
(XVI)

wherein: X¹⁰⁴ is an amino acid selected from N, Q, K, M, T, L, I, V and G;
   X¹⁰⁵ is an amino acid selected from G, E, S, T, A, Q and H;
   X¹⁰⁶ is an amino acid selected from S, T, N, K, R, I and L;
   X¹⁰⁷ is an amino acid selected from T, S, A, N, E, R and G;
   X¹⁰⁸ is an amino acid selected from D, R, K, T, S, P, A and V;
   X¹⁰⁹ is an amino acid selected from H, G, N, P, V, T, S and F; and
   X¹¹⁰ is an amino acid selected from D, S, T, I, A, L, F and Y;
      Provided X¹⁰⁴ is not V when X¹⁰⁵ is A or when X¹⁰⁶ is S; further provided X¹⁰⁴ is not N when X¹⁰⁸ is D or P; further provided X¹⁰⁴ is not L when X¹⁰⁵ is A or S; further provided X¹⁰⁵ is not E when X¹⁰⁴ is M or V; further provided X¹⁰⁵ is not S when X¹⁰⁴ is Q or when X¹¹⁰ is A; further provided X¹⁰⁵ is not T when X¹⁰⁷ is A or when X¹¹⁰ is A; further provided X¹⁰⁵ is not H when X¹⁰⁸ is S or when X¹⁰⁹ is P; further provided X¹⁰⁶ is not S when X¹⁰⁴ is G or when X¹⁰⁵ is S; further provided X¹⁰⁶ is not T when X¹⁰⁴ is L or when X¹⁰⁹ is V; further provided X¹⁰⁶ is not R when X¹⁰⁴ is L or when X¹⁰⁵ is A; further provided X¹⁰⁸ is not A when X¹⁰⁹ is F or when X¹¹⁰ is T; further provided X¹⁰⁸ is not V when X¹⁰⁹ is G or when X¹¹⁰ is L; further provided X¹¹⁰ is not L when X¹⁰⁵ is G or S; further provided X¹¹⁰ is not S when X¹⁰⁵ is S or when X¹⁰⁸ is S; further provided X¹¹⁰ is not T when X¹⁰⁶ is N or when X¹⁰⁹ is H; further provided X¹⁰⁴ is not T when X¹⁰⁸ is P; further provided X¹⁰⁵ is not Q when X¹⁰⁸ is T; and further provided X¹⁰⁷ is not S when X¹⁰⁹ is S.

In some embodiments, the insertion sequence comprises a sequence of Formula XVI wherein X¹⁰⁴ is G and X¹⁰⁵ is S. In some embodiments, the insertion sequence comprises a sequence of Formula XVI wherein X¹⁰⁵ is S. In some embodiments, the insertion sequence comprises a sequence of Formula XVI wherein X¹⁰⁹ is S.

In some embodiments, the insertion sequence as described in Table 20, is selected from IGNTDHD (SEQ ID NO: 157), LEISTTS (SEQ ID NO: 158), VSLAPSI (SEQ ID NO: 159), GSKSTFF (SEQ ID NO: 160), NASNASA (SEQ ID NO: 161), QQNNSSL (SEQ ID NO: 162), MHTERGT (SEQ ID NO: 163), KSRSVND (SEQ ID NO: 164), GSLGKPT (SEQ ID NO: 165) and TTNRTVY (SEQ ID NO: 166).

Aspects disclosed herein provide AAV capsids having improved enrichment in the SPINAL CORD over that found in the LIVER and BRAIN comprising an AAV capsid protein comprising an insertion sequence of Formula XVII

(SEQ ID NO: 18)
X¹¹¹-X¹¹²-X¹¹³-X¹¹⁴-X¹¹⁵-X¹¹⁶-X¹¹⁷
(XVII)

wherein: X¹¹¹ is an amino acid selected from N, H, T, S, G, A, I, Y and F;
   X¹¹² is an amino acid selected from N, E, G, L, I, P and S;
   X¹¹³ is an amino acid selected from G, S, T, R and E;
   X¹¹⁴ is an amino acid selected from S, E, D, N, V and L;
   X¹¹⁵ is an amino acid selected from S, V, I, R, K, H, D, Q and P;
   X¹¹⁶ is an amino acid selected from T, S, G, E, D, I and V; and
   X¹¹⁷ is an amino acid selected from S, Y, P, A, V, L, Q and M;
      Provided X¹¹¹ is not Y when X¹¹² is E or I; further provided X¹¹¹ is not N when X¹¹² is P or when X¹¹³ is T; further provided X¹¹¹ is not G when X¹¹² is L or when X¹¹⁷ is S; further provided X¹¹¹ is not H when X¹¹⁷ is V; further provided X¹¹¹ is not T when X¹¹⁵ is P; further provided X¹¹¹ is not A when X¹¹² is G; further provided X¹¹¹ is not S when X¹¹⁶ is V; further provided X¹¹² is not N when X¹¹³ is T or when X¹¹⁴ is N; further provided X¹¹² is not S when X¹¹³ is G or when X¹¹⁶ is T; further provided X¹¹³ is not S when X¹¹⁴ is N or V; further provided X¹¹³ is not R when X¹¹⁵ is H or when X¹¹⁷ is L; further provided X¹¹⁴ is not S when X¹¹⁶ is S or when X¹¹⁷ is L; further provided X¹¹⁶ is not S when X¹¹⁷ is S; further provided X¹¹⁴ is not L when X¹¹⁵ is S; and further provided X¹¹⁵ is not R when X¹¹⁶ is T.

In some embodiments, the insertion sequence comprises a sequence of Formula XVII wherein X¹¹³ is T. In some embodiments, the insertion sequence comprises a sequence of Formula XVII wherein X¹¹³ is G. In some embodiments, the insertion sequence comprises a sequence of Formula XVII wherein X¹¹³ is S.

In some embodiments, the insertion sequence as described in Table 22, is selected from HNGVSIL (SEQ ID NO: 167), NESSVTS (SEQ ID NO: 168), TGTEIGY (SEQ ID NO: 169), SLSDREY (SEQ ID NO: 170), GPGEHSP (SEQ ID NO: 171), TSTSDIA (SEQ ID NO: 172), ASRDSDV (SEQ ID NO: 173), YNSLQGQ (SEQ ID NO: 174), FIENKVA (SEQ ID NO: 175) and IGTLPTM (SEQ ID NO: 176).

Aspects disclosed herein provide AAV capsids having significant enrichment in both the BRAIN and in the SPINAL CORD comprising an AAV capsid protein comprising an insertion of Formula XVIII

(SEQ ID NO: 19)
X¹¹⁸-X¹¹⁹-X¹²⁰-X¹²¹-X¹²²-X¹²³-X¹²⁴
(XVIII)

wherein: X¹¹⁸ is an amino acid selected from H, E, N, S, T and V;
   X¹¹⁹ is an amino acid selected from G, T, D, S and V;
   X¹²⁰ is an amino acid selected from S, P, A, N and D;
   X¹²¹ is an amino acid selected from N, D, K, S, G, A, I and P;
   X¹²² is an amino acid selected from A, I, V, L, H, N, S and T;
   X¹²³ is an amino acid selected from R, D, A, I, H, T, Q, F and P; and
   X¹²⁴ is an amino acid selected from D, R, K, G, S, F, V, P and Y;
      Provided X¹¹⁹ is not S when X¹²⁰ is S or when X¹²² is A or when X¹¹⁸ is T; further provided X¹¹⁹ is not V when X¹¹⁸ is S or when X¹²⁰ is S; further provided X¹²¹ is not S when X¹²² is S or when X¹¹⁹ is D or G; further provided X¹²⁰ is not D when X¹²³ is R; further provided X¹¹⁹ is not T when X¹¹⁸ is V or N; further provided X¹²⁰ is not P when X¹²¹ is A or when X¹²³ is R; further provided X¹²¹ is not I when X¹²² is N; further provided X¹¹⁸ is not T when X¹²³ is P; further provided X¹¹⁸ is not V when X¹¹⁹ is G or D; further X¹¹⁹ is not D when X¹²⁴ is P; and further provided X¹¹⁸ is not H when X¹²⁰ is N.

In some embodiments, the insertion sequence comprises a sequence of Formula XVIII wherein X¹¹⁸ is N and X¹¹⁹ is D. In some embodiments, the insertion sequence comprises a sequence of Formula XVIII wherein X¹¹⁸ is E and X¹¹⁹ is T. In some embodiments, the insertion sequence comprises a sequence of Formula XVIII wherein X¹¹⁹ is S.

In some embodiments, the insertion sequence as described in Table 17, is selected from HGSDIRD (SEQ ID NO: 177), ETPNHDG (SEQ ID NO: 178), NDSGAAS (SEQ ID NO: 179), ETASVHF (SEQ ID NO: 180), NDNANTK (SEQ ID NO: 181), SSNALQV (SEQ ID NO: 182), SGANHFS (SEQ ID NO: 183), TGSPNIP (SEQ ID NO: 184), VSNISRY (SEQ ID NO: 185) and NVDKTPR (SEQ ID NO: 186).

Aspects disclosed herein provide AAV capsids having significant enrichment in the SPINAL CORD and BRAIN over that found in the LIVER comprising an AAV capsid protein comprising an insertion sequence of Formula XIX

(SEQ ID NO: 20)
X¹²⁵-X¹²⁶-X¹²⁷-X¹²⁸-X¹²⁹-X¹³⁰-X¹³¹
(XIX)

wherein: X¹²⁵ is an amino acid selected from P, G, K, E, T and A;
   X¹²⁶ is an amino acid selected from R, T, G, N, P and V;
   X¹²⁷ is an amino acid selected from D, Q, E, N, V, I, A and P;
   X¹²⁸ is an amino acid selected from L, I, V, N, D, Q, K and S;
   X¹²⁹ is an amino acid selected from N, D, E, G, S, T and I;
   X¹³⁰ is an amino acid selected from D, N, Q, F, T, G, L and V; and
   X¹³¹ is an amino acid selected from P, M, I, G, T, H and K;
      Provided X¹²⁵ is not P when X¹²⁷ is I or when X¹²⁶ is V; further provided X¹²⁶ is not P when X¹²⁵ is E or G; further provided X¹²⁹ is not S when X¹²⁶ is R or when X¹³⁰ is T; further provided X¹²⁸ is not S when X¹³¹ is T or when X¹²⁶ is P; further provided X¹²⁶ is not G when X¹²⁵ is G or when X¹²⁷ is P; further provided X¹²⁷ is not A when X¹²⁸ is L; further provided X¹²⁵ is not K when X¹²⁶ is T; and further provided X¹²⁵ is not T when X¹²⁷ is N.

In some embodiments, the insertion sequence comprises a sequence of Formula XIX wherein X¹²⁵ is P. In some embodiments, the insertion sequence comprises a sequence of Formula XIX wherein X¹²⁸ is Q.

In some embodiments, the insertion sequence as described in Table 30, is selected from PRDLNDP (SEQ ID NO: 187), GTQNDVM (SEQ ID NO: 188), KGVDGDI (SEQ ID NO: 189), ENPSSNG (SEQ ID NO: 190), KGDVTFT (SEQ ID NO: 191), PPNQDQH (SEQ ID NO: 192), TPANELK (SEQ ID NO: 193), GNEQITG (SEQ ID NO: 194), EVIKETG (SEQ ID NO: 195) and ATVINGT (SEQ ID NO: 196).

Aspects disclosed herein provide AAV capsids having improved enrichment in the SPINAL CORD comprising an AAV capsid protein comprising an insertion of Formula XX

(SEQ ID NO: 21)
X¹³²-X¹³³-X¹³⁴-X¹³⁵-X¹³⁶-X¹³⁷-X¹³⁸
(XX)

wherein: X¹³² is an amino acid selected from P, Y, N, S, T and A;
   X¹³³ is an amino acid selected from H, E, S, T, N, G and A;
   X¹³⁴ is an amino acid selected from N, R, D, S, F, L and Y;
   X¹³⁵ is an amino acid selected from L, A, D, E, P, Q, K and S;
   X¹³⁶ is an amino acid selected from L, D, Q, N, R, Y and T;
   X¹³⁷ is an amino acid selected from N, Q, T, S, L and V; and
   X¹³⁸ is an amino acid selected from N, S, T, L and A;
      Provided X¹³⁵ is not S when X¹³⁷ is S; further provided X¹³³ is not T when X¹³⁶ is T or when X¹³² is S; further provided X¹³² is not N when X¹³³ is E or when X¹³⁴ is R; further provided X¹³³ is not H when X¹³⁷ is T or when X¹³² is S; further provided X¹³³ is not A when X¹³⁴ is N or when X¹³⁵ is A; further provided X¹³⁴ is not S when X¹³⁵ is A or when X¹³⁶ is R; further provided X¹³⁴ is not L when X¹³⁷ is L or when X¹³² is A; further provided X¹³³ is not S when X¹³² is P or when X¹³⁶ is T; further provided X¹³² is not Y when X¹³⁶ is N; and further provided X¹³² is not T when X¹³⁴ is F.

In some embodiments, the insertion sequence comprises a sequence of Formula XX wherein X¹³⁶ is N.

In some embodiments, the insertion sequence as described in Table 15, is selected from THNDLLN (SEQ ID NO: 197), PERAQVS (SEQ ID NO: 198), YESLTQN (SEQ ID NO: 199), SERPDTL (SEQ ID NO: 200), TNDANTL (SEQ ID NO: 201), SSNEYST (SEQ ID NO: 202), NTFSRNN (SEQ ID NO: 203), YNLQLNS (SEQ ID NO: 204), AGYPNSA (SEQ ID NO: 205) and NADKNNL (SEQ ID NO: 206).

Aspects disclosed herein provide AAV capsids having significant enrichment in the SPINAL CORD over the LIVER comprising an AAV capsid protein comprising an insertion sequence of Formula XXI

(SEQ ID NO: 22)
X¹³⁹-X¹⁴⁰-X¹⁴¹-X¹⁴²-X¹⁴³-X¹⁴⁴-X¹⁴⁵
(XXI)

wherein: X¹³⁹ is an amino acid selected from H, N, S, R, L, V and A;
   X¹⁴⁰ is an amino acid selected from H, E, D, N, K, S, L and A;
   X¹⁴¹ is an amino acid selected from N, A, L, V, E, D and P;
   X¹⁴² is an amino acid selected from D, S, G, K, L and M;
   X¹⁴³ is an amino acid selected from N, E, Q, R, S and M;
   X¹⁴⁴ is an amino acid selected from P, T, S, H, Y, I and V; and
   X¹⁴⁵ is an amino acid selected from E, D, P, G, V, L and A;
      Provided X¹⁴³ is not R when X¹⁴² is S or D or when X¹⁴⁴ is T or when X¹⁴⁵ is E; further provided X¹⁴² is not S when X¹⁴³ is S or when X¹³⁹ is A; further provided X¹⁴¹ is not A when X¹⁴⁴ is S or when X¹³⁹ is S or R; further provided X¹⁴³ is not N when X¹⁴⁰ is A and when X¹³⁹ is R; further X¹⁴² is not G when X¹⁴⁰ is N or when X¹⁴³ is E; further provided X¹⁴⁰ is not H when X¹³⁹ is H or L; further provided X¹⁴¹ is not L when X¹⁴⁴ is H; further provided X¹⁴¹ is not P when X¹⁴⁵ is A; further provided X¹⁴⁰ is S when X¹⁴² is K; further provided X¹³⁹ is not V when X¹⁴¹ is V.

In some embodiments, the insertion sequence comprises a sequence of Formula XXI wherein X¹³⁹ is V. In some embodiments, the insertion sequence comprises a sequence of Formula XXI wherein X¹⁴⁰ is E. In some embodiments, the insertion sequence comprises a sequence of Formula XXI wherein X¹⁴¹ or X¹⁴² is D.

In some embodiments, the insertion sequence as described in Table 19, is selected from NHNDSVE (SEQ ID NO: 207), LEASNTA (SEQ ID NO: 208), VDNDNPL (SEQ ID NO: 209), VELGSSP (SEQ ID NO: 210), VNEKESV (SEQ ID NO: 211), SAVDMSA (SEQ ID NO: 212), RLDLQHD (SEQ ID NO: 213), HEDKSVA (SEQ ID NO: 214), RSPGQIG (SEQ ID NO: 215) and AKEMRYA (SEQ ID NO: 216).

Aspects disclosed herein provide AAV capsids having significant enrichment in the SPINAL CORD over that found in BRAIN comprising an AAV capsid protein comprising an insertion sequence of Formula XXII

(SEQ ID NO: 23)
X¹⁴⁶-X¹⁴⁷-X¹⁴⁸-X¹⁴⁹-X¹⁵⁰-X¹⁵¹-X¹⁵²
(XXII)

wherein: X¹⁴⁶ is an amino acid selected from M, N, Q, T, S, Y and I;
   X¹⁴⁷ is an amino acid selected from V, G, I, D, Q, T and S;
   X¹⁴¹ is an amino acid selected from N, A, L, M, T, S and P;
   X¹⁴⁹ is an amino acid selected from V, A, S, K, R, Q, N and G;
   X¹⁵⁰ is an amino acid selected from N, G, V, L, I, S and K;
   X¹⁵¹ is an amino acid selected from F, S, T, M, N, P, L, G and V; and
   X¹⁵² is an amino acid selected from K, S, T, P, A, M, N, E and Y;
      Provided X¹⁴⁶ is not T when X¹⁴⁷ is T or when X¹⁴⁸ is L or when X¹⁵⁰ is L; further provided X¹⁴⁶ is not I when X¹⁴⁸ is N or when X¹⁵⁰ is I; further provided X¹⁴⁶ is not N when X¹⁴⁷ is V or when X¹⁵¹ is P; further provided X¹⁴⁶ is not S when X¹⁴⁸ is S, L or N or when X¹⁵⁰ is V; further provided X¹⁴⁷ is not S when X¹⁴⁸ is A or when X¹⁴⁹ is S or when X¹⁵⁰ is V; further provided X¹⁴⁷ is not T when X¹⁴⁶ is S or when X¹⁵² is A; further provided X¹⁴⁷ is not V when X¹⁵⁰ is I or when X¹⁵¹ is T; further provided X¹⁴⁸ is not S when X¹⁴⁷ is D or S or when X¹⁵¹ is G; further provided X¹⁵⁰ is not S when X¹⁴⁸ is T or when X¹⁵¹ is S; further provided X¹⁵⁰ is not N when X¹⁴⁷ is I or when X¹⁴⁸ is T; further provided X¹⁴⁶ is not Y when X¹⁵⁰ is K or when X¹⁵² is S; further provided X¹⁵⁰ is not L when X¹⁴⁶ is S or when X¹⁵¹ is N; further provided X¹⁵⁰ is not G when X¹⁴⁷ is S or when X¹⁵² is T; further provided X¹⁴⁷ is not D when X¹⁴⁹ is G or when X¹⁵⁰ is G; further provided X¹⁴⁹ is not R when X¹⁴⁶ is T or when X¹⁵² is P; and further provided X¹⁵² is not S when X¹⁴⁷ is G or when X¹⁴⁸ is P or when X¹⁵¹ is V.

In some embodiments, the insertion sequence comprises a sequence of Formula XXII wherein X¹⁴⁸ is N.

In some embodiments, the insertion sequence as described in Table 21, is selected from MVNVNVK (SEQ ID NO: 217), NTLASFS (SEQ ID NO: 218), IGAKGSP (SEQ ID NO: 219), NITSVTA (SEQ ID NO: 220), ITMRSMM (SEQ ID NO: 221), MDNQSNN (SEQ ID NO: 222), YQSGLLE (SEQ ID NO: 223), TGANIGY (SEQ ID NO: 224), QDNSKLS (SEQ ID NO: 225) and SSPAKPT (SEQ ID NO: 226).

Aspects disclosed herein provide AAV capsids having significant enrichment in the SPINAL CORD over that found in the LIVER and BRAIN comprising an AAV capsid protein comprising an insertion of Formula XXIII

(SEQ ID NO: 24)
X¹⁵³-X¹⁵⁴-X¹⁵⁵-X¹⁵⁶-X¹⁵⁷-X¹⁵⁸-X¹⁵⁹
(XXIII)

wherein: X¹⁵³ is an amino acid selected from Q, P, W, M, S, R, D, V and I;
   X¹⁵⁴ is an amino acid selected from E, D, W, L, P, H, Y, G and S;
   X¹⁵⁵ is an amino acid selected from N, G, H, F and D;
   X¹⁵⁶ is an amino acid selected from D, E, P, H, R, T, N and G;
   X¹⁵⁷ is an amino acid selected from L, H, Q, G, P, Y, T, S and R;
   X¹⁵¹ is an amino acid selected from V, T, S, P, H, N and G; and
   X¹⁵⁹ is an amino acid selected from S, T, H, A, L and E;
      Provided X¹⁵⁵ is not N when X¹⁵⁴ is L or when X¹⁵⁹ is L or when X¹⁵⁸ is N or V; further provided X¹⁵⁵ is not G when X¹⁵⁶ is G or when X¹⁵⁸ is P or when X¹⁵⁹ is A or when X¹⁵⁴ is L; further provided X¹⁵⁵ is not D when X¹⁵⁸ is S; further provided X¹⁵⁵ is not H when X¹⁵⁴ is S or when X¹⁵⁹ is S; further provided X¹⁵⁷ is not S when X¹⁵⁹ is A or when X¹⁵⁴ is G; further provided X¹⁵⁸ is not G when X¹⁵⁴ is L or when X¹⁵³ is M; further provided X¹⁵³ is not S when X¹⁵⁴ is P or S; further provided X¹⁵⁴ is not S when X¹⁵⁷ is R; further provided X¹⁵³ is not P when X¹⁵⁶ is N; further provided X¹⁵³ and X¹⁵⁴ are not both D; and further provided X¹⁵³ is not V when X¹⁵⁴ is Y.

In some embodiments, the insertion sequence comprises a sequence of Formula XXIII wherein X¹⁵⁴ is E. In some embodiments, the insertion sequence comprises a sequence of Formula XXIII wherein X¹⁵⁹ is E. In some embodiments, the insertion sequence comprises a sequence of Formula XXIII wherein X¹⁵⁹ is S or T.

In some embodiments, the insertion sequence as described in Table 23, is selected from QEGNLVS (SEQ ID NO: 227), PDNTTTS (SEQ ID NO: 228), WSGTLVH (SEQ ID NO: 229), MLHGHHL (SEQ ID NO: 230), VWHDQSA (SEQ ID NO: 231), IPFPGPE (SEQ ID NO: 232), SHHHPTT (SEQ ID NO: 233), RYDERNA (SEQ ID NO: 234), IGNRYPT (SEQ ID NO: 235) and DEDRSGE (SEQ ID NO: 236).

Aspects disclosed herein provide AAV capsids comprising an AAV capsid protein comprising an insertion sequence of Formula XXIV

(SEQ ID NO: 25)
X¹⁶⁰-X¹⁶¹-T-T-K
(XXIV)

wherein: X¹⁶⁰ is an amino acid selected from L, I, A, S, T and E; and X¹⁶¹ is an amino acid selected from N and H.

In some embodiments, the insertion sequence comprises a sequence of Formula XXIV wherein X¹⁶⁰ is L and X¹⁶¹ is N.

Aspects disclosed herein provide AAV capsids comprising an AAV capsid protein comprising an insertion sequence of Formula XXIVa

(SEQ ID NO: 26)
X¹⁶⁰-X¹⁶¹-T-T-K-X¹⁶²
(XXIVa)

wherein X¹⁶⁰ is an amino acid selected from L, I, A, S, T and E; X¹⁶¹ is an amino acid selected from N and H; and X¹⁶² is an amino acid selected from P, L, M, N, R, S and D.

In some embodiments, the insertion sequence comprises a sequence of Formula XXIV wherein X¹⁶⁰ is L, X¹⁶¹ is N and X¹⁶² is S or P.

Aspects disclosed herein provide AAV capsids comprising an AAV capsid protein comprising an insertion of Formula XXIVb

(SEQ ID NO: 27)
X¹⁶⁰-X¹⁶¹-T-T-K-X¹⁶²-X¹⁶³
(XXIVb)

wherein X¹⁶⁰ is an amino acid selected from L, I, A, S, T and E; X¹⁶¹ is an amino acid selected from N and H; X¹⁶² is an amino acid selected from M, P, N, R, S and D; and X¹⁶³ is an amino acid selected from P, I, Y, F, Q, E, S and L.

In some embodiments, the insertion sequence comprises a sequence of Formula XXIV wherein X¹⁶⁰ is L, X¹⁶¹ is N, X¹⁶² is S or P and X¹⁶³ is I.

In some embodiments, the insertion sequence is selected from ANTTKDL (SEQ ID NO: 237), INTTKMY (SEQ ID NO: 238), TNTTKNF (SEQ ID NO: 239), ENTTKRE (SEQ ID NO: 240), LNTTKPI (SEQ ID NO: 241), SHTTKPQ (SEQ ID NO: 242) and GNTTKSS (SEQ ID NO: 243).

Aspects disclosed herein provide AAV capsids comprising an AAV capsid protein comprising an insertion sequence of Formula XXV

(SEQ ID NO: 28)
E-N-H-X¹⁶⁴-X¹⁶⁵-X¹⁶⁶-X¹⁶⁷
(XXV)

Wherein X¹⁶⁴ is an amino acid selected from I, L, A, G, S, T, K and R;
X¹⁶⁵ is an amino acid selected from K, R, I, L, A and G;
X¹⁶⁶ is an amino acid selected from T, N, Q and S; and
X¹⁶⁷ is an amino acid selected from I, L, A, G, E, D, S and T.

In some aspects, the AAV capsid protein comprises an insertion sequence of Formula XXV wherein X¹⁶⁴ is an amino acid selected from I, L, A, G, S, T and R; X¹⁶⁵ is an amino acid selected from K, R and G; X¹⁶⁶ is an amino acid selected from T, N and S; and X¹⁶⁷ is an amino acid selected from I, A, E, D, S and T. In some aspects, X¹⁶⁴ is an amino acid selected from I, T and R; X¹⁶⁵ is an amino acid selected from K and R; X¹⁶⁶ is an amino acid selected from T, N and S; and X¹⁶⁷ is an amino acid selected from I, D, S and T.

In some embodiments, the insertion sequence is selected from ENHIKTI (SEQ ID NO: 244), ENHTRNS (SEQ ID NO: 245), ENHTKND (SEQ ID NO: 246) and ENHRGST (SEQ ID NO: 247).

Aspects disclosed herein provide AAV capsids comprising an AAV capsid protein comprising an insertion sequence of Formula XXVI

(SEQ ID NO: 29)
X¹⁶⁸-S-R-E-X¹⁶⁹-X¹⁷⁰-X¹⁷¹
(XXVI)

wherein X¹⁶⁸ is an amino acid selected from D, H, I, K, M and N;
   X¹⁶⁹ is an amino acid selected from F, S, W, A, L and R;
   X¹⁷⁰ is an amino acid selected from K, N, S, Y, L, T, E and D; and
   X¹⁷¹ is an amino acid selected from I, K, V, Y, A, T and S.

In some aspects, the AAV capsid protein comprises an insertion sequence of Formula XXVI wherein X¹⁶⁸ is an amino acid selected from D, I and K; X¹⁶⁹ is an amino acid selected from F, S, W, A and L; X¹⁷⁰ is an amino acid selected from K, N, Y, L, T, E and D; and X¹⁷¹ is an amino acid selected from I, K, Y, A and T.

In some embodiments, the insertion sequence is selected from DSRESNK (SEQ ID NO: 248), HSREFSV (SEQ ID NO: 249), ISREFYK (SEQ ID NO: 38), ISRESLY (SEQ ID NO: 250), ISREWTA (SEQ ID NO: 251), KSREAEY (SEQ ID NO: 252), KSRELDT (SEQ ID NO: 253) and NSRESEA (SEQ ID NO: 254).

Aspects disclosed herein provide AAV capsids comprising an AAV capsid protein comprising an insertion sequence of Formula XXVII

(SEQ ID NO: 30)
X¹⁷²-N-X¹⁷³-X¹⁷⁴-X¹⁷⁵-X¹⁷⁶-X¹⁷⁷
(XXVII)

wherein X¹⁷² is an amino acid selected from G, T, D, L and E;
   X¹⁷³ is an amino acid selected from T, S, M, N and H;
   X¹⁷⁴ is an amino acid selected from V, T and I;
   X¹⁷⁵ is an amino acid selected from R and K;
   X¹⁷⁶ is an amino acid selected from D, Q, N, S and P; and
   X¹⁷⁷ is an amino acid selected from I, V, Y, L, T and S;
      Provided X¹⁷² is not T when X¹⁷⁴ is T or X¹⁷³ is N; further provided X¹⁷⁵ is not R when X¹⁷⁶ is P or when X¹⁷¹ is L; further provided X¹⁷¹ is not E when X¹⁷³ is M.

In some embodiments, the insertion sequence comprises a sequence of Formula XXVII wherein X¹⁷² is G. In some embodiments, the insertion sequence comprises a sequence of Formula XXVII wherein X¹⁷³ is T. In some embodiments, the insertion sequence comprises a sequence of Formula XXVII wherein X¹⁷⁴ is N. In some embodiments, the insertion sequence comprises a sequence of Formula XXVII wherein X¹⁷⁶ is S.

In some embodiments, the insertion sequence as described in Table 24 is selected from GNTTRDY (SEQ ID NO: 255), GNMVKQV (SEQ ID NO: 256), TNSVKNL (SEQ ID NO: 257), GNNVKSI (SEQ ID NO: 258), DNSTRSV (SEQ ID NO: 259), LNTTKPI (SEQ ID NO: 241), GNTTKSS (SEQ ID NO: 243), ENNIRSI (SEQ ID NO: 260), DNSIRNT (SEQ ID NO: 261) and ENHTRNS (SEQ ID NO: 245).

Aspects disclosed herein provide AAV capsids having the best expression in the BRAIN of the insertions expressed in the one spinal cord group comprising an AAV capsid protein comprising an insertion sequence of Formula XXVIII

(SEQ ID NO: 31)
X¹⁷⁸-N-X¹⁷⁹-X¹⁸⁰-X¹⁸¹-X¹⁸²-X¹⁸³
(XXVIII)

wherein X¹⁷⁸ is an amino acid selected from N, Q, A, S, G and E;
   X¹⁷⁹ is an amino acid selected from R, V, S, N and T;
   X¹⁸⁰ is an amino acid selected from R, I, T and V;
   X¹⁸¹ is an amino acid selected from M, P, R and K;
   X¹⁸² is an amino acid selected from D, L, N, R, A and P; and
   X¹⁸³ is an amino acid selected from D, T, I, M, L, N and V;
      Provided X¹⁷⁸ is not S when X¹⁸¹ is K or when X¹⁷⁹ is R; further provided X¹⁸¹ is not R when X¹⁸⁰ is I or when X¹⁸² is L or when X¹⁸³ is T; and further X¹⁸⁰ is not T when X¹⁸² is A.

In some embodiments, the insertion sequence comprises a sequence of Formula XXVIII wherein X¹⁷⁸ is N, and X¹⁸³ is L. In some embodiments, the insertion sequence comprises a sequence of Formula XXVIII wherein X¹⁷⁹ is T, and X¹⁸³ is L. In some embodiments, the insertion sequence comprises a sequence of Formula XXVIII wherein X¹⁷⁹ is T, X¹⁸² is N, and X¹⁸³ is L.

In some embodiments, the insertion sequence as described in Table 27, is selected from NNRRPDD (SEQ ID NO: 262), QNVIKPT (SEQ ID NO: 263), QNSTKLI (SEQ ID NO: 264), ANNTRNM (SEQ ID NO: 265), SNTTRNL (SEQ ID NO: 266), ENSVRNN (SEQ ID NO: 267), NNSTKLL (SEQ ID NO: 268), GNSVRAN (SEQ ID NO: 269), SNSTRPL (SEQ ID NO: 270) and GNSTMRV (SEQ ID NO: 271).

Aspects disclosed herein provide AAV capsids having the best expression in the BRAIN of the insertions expressed in another spinal cord group comprising an AAV capsid protein comprising an insertion sequence of Formula XXIX

(SEQ ID NO: 32)
X¹⁸⁴-X¹⁸⁵-X¹⁸⁶-X¹⁸⁷-X¹⁸⁸-X¹⁸⁹-X¹⁹⁰
(XXIX)

wherein X¹⁸⁴ is an amino acid selected from G, T, M, S, A and Y;
   X¹⁸⁵ is an amino acid selected from K, V, N and D;
   X¹⁸⁶ is an amino acid selected from S, K, V, R, T and H;
   X¹⁸⁷ is an amino acid selected from M, G, V, I, T and K;
   X¹⁸⁸ is an amino acid selected from K, L, R, S and G;
   X¹⁸⁹ is an amino acid selected from N, S, D, I and L; and
   X¹⁹⁰ is an amino acid selected from F, M, T, Y, N, G, V and Q;
      provided X¹⁸⁵ is not N when X¹⁸⁴ is M or A or when X¹⁸⁶ is H; further provided X¹⁸⁵ is not V when X¹⁸⁴ is T; further provided X¹⁸⁵ is not D when X¹⁸⁴ is Y; further provided X¹⁸⁶ is not S when X¹⁹⁰ is V; further provided X¹⁸⁶ and X¹⁹⁰ are not both T; further provided X¹⁸⁶ is not R when X¹⁸⁸ is S; and further provided X¹⁸⁷ is not V when X¹⁸⁸ is R.

In some embodiments, the insertion sequence comprises a sequence of Formula XXIX wherein X¹⁸⁵ is N. In some embodiments, the insertion sequence comprises a sequence of Formula XXIX wherein X¹⁸⁶ is S. In some embodiments, the insertion sequence comprises a sequence of Formula XXIX wherein X¹⁸⁹ is N.

In some embodiments, the insertion sequence as described in Table 28, is selected from GNSTKIG (SEQ ID NO: 272), TNTTKNF (SEQ ID NO: 239), MKSGLSM (SEQ ID NO: 273), SNKMGNT (SEQ ID NO: 274), SNSVKDY (SEQ ID NO: 275), AVHKSDF (SEQ ID NO: 276), SNSIRNN (SEQ ID NO: 277), TDRMGLT (SEQ ID NO: 278), SNVIKNV (SEQ ID NO: 279) and YNSTRNQ (SEQ ID NO: 280).

Aspects disclosed herein provide AAV capsids having the best expression in the BRAIN of the insertions expressed in the brain and the spinal cord comprising an AAV capsid protein comprising an insertion sequence of Formula XXX

(SEQ ID NO: 33)
X¹⁹¹-X¹⁹²-X¹⁹³-X¹⁹⁴-X¹⁹⁵-X¹⁹⁶-X¹⁹⁷
(XXX)

wherein X¹⁹¹ is an amino acid selected from G, D, N, T, L, S, I, Q and F;
   X¹⁹² is an amino acid selected from G, S, V, N and R;
   X¹⁹³ is an amino acid selected from E, V, R, T, S, N and H;
   X¹⁹⁴ is an amino acid selected from I, D, L, N, S, V, R and T;
   X¹⁹⁵ is an amino acid selected from L, P, R, I, K and V;
   X¹⁹⁶ is an amino acid selected from R, P, N, A, T, S, V, M and K; and
   X¹⁹⁷ is an amino acid selected from D, T, L, N, E, I and G;
      Provided X¹⁹¹ is not S when X¹⁹² is G or when X¹⁹³ is N; further provided X¹⁹² is not V when X¹⁹¹ is L or T; further provided X¹⁹² is not R when X¹⁹³ is S or when X¹⁹⁶ is A; further provided X¹⁹² is not G when X¹⁹¹ is Q or when X¹⁹⁵ is L; further provided X¹⁹² is not S when X¹⁹⁵ is L or R; further provided X¹⁹³ is not T when X¹⁹¹ is T or when X¹⁹⁴ is T or V; further provided X¹⁹² is not N when X¹⁹³ is N or when X¹⁹⁴ is T or when X¹⁹⁷ is G; further provided X¹⁹³ is not S when X¹⁹¹ is L or N; further provided X¹⁹⁴ is not N when X¹⁹¹ is S or F; further provided X¹⁹⁵ is not P when X¹⁹¹ is L or when X¹⁹³ is V; further provided X¹⁹⁷ is not T when X¹⁹³ is S or when X¹⁹⁵ is I; further provided X¹⁹⁷ is not E when X¹⁹² is V or when X¹⁹³ is R; and further X¹⁹⁴ is not S when X¹⁹⁵ is V.

In some embodiments, the insertion sequence comprises a sequence of Formula XXX wherein X¹⁹² is N. In some embodiments, the insertion sequence comprises a sequence of Formula XXX wherein X¹⁹⁵ is R.

In some embodiments, the insertion sequence as described in Table 26, is selected from GNEVRRD (SEQ ID NO: 281), DNVIRPT (SEQ ID NO: 282), NVRDLNL (SEQ ID NO: 283), TSRLPAL (SEQ ID NO: 284), LNTNRTN (SEQ ID NO: 285), SRTSISE (SEQ ID NO: 286), SNSVRND (SEQ ID NO: 287), IGNRPVI (SEQ ID NO: 288), QNTIKMT (SEQ ID NO: 289) and FSHTVKG (SEQ ID NO: 290).

Aspects disclosed herein provide AAV capsids having greater expression in the BRAIN and low expression in the spinal cord comprising an AAV capsid protein comprising an insertion sequence of Formula XXXI

(SEQ ID NO: 34)
X¹⁹⁸-X¹⁹⁹-X²⁰⁰-X²⁰¹-X²⁰²-X²⁰³-X²⁰⁴
(XXXI)

wherein X¹⁹⁸ is an amino acid selected from R, E, M, S, N, L, T and G;
   X¹⁹⁹ is an amino acid selected from N, S and R;
   X²⁰⁰ is an amino acid selected from D, S, N and A;
   X²⁰¹ is an amino acid selected from M, S, K, V and T;
   X²⁰² is an amino acid selected from D, R, A and K;
   X²⁰³ is an amino acid selected from P, Y, Q, R, M, A and G; and
   X²⁰⁴ is an amino acid selected from F, T, L, Y, I and S;
      Provided X¹⁹⁹ is not S when X²⁰¹ is S; further provided X¹⁹⁸ is not S when X²⁰⁰ is S; further provided X²⁰⁰ is not N when X¹⁹⁸ is T or G; further provided X¹⁹⁸ is not N when X²⁰⁴ is T; further provided X²⁰² is not R when X²⁰³ is Q; and further provided X¹⁹⁸ is not T when X²⁰⁰ is D.

In some embodiments, the insertion sequence comprises a sequence of Formula XXXI wherein X¹⁹⁹ is N. In some embodiments, the insertion sequence comprises a sequence of Formula XXXI wherein X²⁰⁰ is N. In some embodiments, the insertion sequence comprises a sequence of Formula XXXI wherein X²⁰¹ is T. In some embodiments, the insertion sequence comprises a sequence of Formula XXXI wherein X²⁰² is R.

In some embodiments, the insertion sequence as described in Table 25, is selected from RRDMDPT (SEQ ID NO: 291), ENSTRYT (SEQ ID NO: 292), MNSTRPF (SEQ ID NO: 293), SNNVKQT (SEQ ID NO: 294), SNNSRPY (SEQ ID NO: 295), NNSTARI (SEQ ID NO: 296), LSNKAML (SEQ ID NO: 297), TNATRPL (SEQ ID NO: 298), GNAVRGT (SEQ ID NO: 299) and GNSTKAS (SEQ ID NO: 300).

Aspects disclosed herein provide AAV capsids having greater enrichment in both the BRAIN and in the SPINAL CORD comprising an AAV capsid protein comprising an insertion sequence of Formula XXXII

(SEQ ID NO: 35)
X²⁰⁵-X²⁰⁶-X²⁰⁷-X²⁰⁸-X²⁰⁹-X²¹⁰-X²¹¹
(XXXII)

wherein X²⁰⁵ is an amino acid selected from E, L, I, V, N, G, S, and F
   X²⁰⁶ is an amino acid selected from Q, L, D, T, I, and S;
   X²⁰⁷ is an amino acid selected from S, R, G, K and N;
   X²⁰⁸ is an amino acid selected from H, D, N, Q, S, E and T;
   X²⁰⁹ is an amino acid selected from G, S, R, I, N, A and Q;
   X²¹⁰ is an amino acid selected from S, N, R, E, T, M and Q; and
   X²¹¹ is an amino acid selected from K, N, V, R, S, and F;
      Provided X²⁰⁶ is not L when X²⁰⁵ is N or when X²⁰⁸ is T or when X²¹⁰ is S; further provided X²⁰⁶ is not S when X²⁰⁵ is G or when X²⁰⁹ is N; further provided X²⁰⁷ is not G when X²⁰⁵ is L or N; further provided X²⁰⁷ is not S when X²⁰⁸ is S or when X²¹⁰ is T; further provided X²¹¹ is not S when X²⁰⁷ is R or when X²⁰⁹ is G or when X²¹⁰ is S; further provided X²⁰⁵ is not S when X²⁰⁷ is N; further provided X²⁰⁶ is not N when X²⁰⁸ is S; further provided X²⁰⁶ is not T when X²¹¹ is V; and further provided X²⁰⁹ is not A when X²¹⁰ is Q.

In some embodiments, the insertion sequence comprises a sequence of Formula XXXII wherein X²¹¹ is N. In some embodiments, the insertion sequence comprises a sequence of Formula XXXII wherein X²⁰⁵ is N. In some embodiments, the insertion sequence comprises a sequence of Formula XXXII wherein X²⁰⁸ is S.

In some embodiments, the insertion sequence as described in Table 6, is selected from EQSHGSK (SEQ ID NO: 301), LLRDSNN (SEQ ID NO: 302), ILGNSRV (SEQ ID NO: 303), VDKQREN (SEQ ID NO: 304), NDNQITR (SEQ ID NO: 305), GTNSSTS (SEQ ID NO: 306), LIKENRF (SEQ ID NO: 307), SSSTAMS (SEQ ID NO: 308), FQNSQTR (SEQ ID NO: 309) and NTSQSQK (SEQ ID NO: 310).

Aspects disclosed herein provide AAV capsids having greater enrichment in both SPINAL CORD and BRAIN over that found in the LIVER comprising an AAV capsid protein comprising an insertion sequence of Formula XXXIII

(SEQ ID NO: 36)
X²¹²-X²¹³-X²¹⁴-X²¹⁵-X²¹⁶-X²¹⁷-X²¹⁸
(XXXIII)

wherein X²¹² is an amino acid selected from T, A, S, E, N, L and F;
   X²¹³ is an amino acid selected from Q, L, E, N, P and S;
   X²¹⁴ is an amino acid selected from P, V, Y, M, H, E, D and L;
   X²¹⁵ is an amino acid selected from T, S, G, I, T, V and H;
   X²¹⁶ is an amino acid selected from M, G, T, K, Q, P, N, L and T;
   X²¹⁷ is an amino acid selected from E, D, K, N, T, S, N and Y; and
   X²¹⁸ is an amino acid selected from N, V, H, I, R, S, and A;
      Provided X²¹² is not A when X²¹³ is S or when X²¹⁵ is T; further provided X²¹² is not T when X²¹⁴ is H or when X²¹⁸ is V; further provided X²¹⁸ is not S when X²¹⁵ is T or when X²¹⁷ is S; further provided X²¹² is not L when X²¹⁴ is P; further provided X²¹² is not S when X²¹³ is L; further provided X²¹³ is not N when X²¹⁸ is A; further provided X²¹⁴ is not V when X²¹⁸ is R; further provided X²¹⁴ is not L when X²¹⁸ is N; further provided X²¹⁴ is not D when X²¹⁶ is M; further provided X²¹⁵ is not S when X²¹⁶ is L; and further provided X²¹⁶ is not T when X²¹⁷ is T.

In some embodiments, the insertion sequence comprises a sequence of Formula XXXIII wherein X²¹³ is N. In some embodiments, the insertion sequence comprises a sequence of Formula XXXIII wherein X²¹⁵ is T, In some embodiments, the insertion sequence comprises a sequence of Formula XXXIII wherein X²¹⁶ is T.

In some embodiments, the insertion sequence as described in Table 13, is selected from TQPTMEN (SEQ ID NO: 311), ALVSGDV (SEQ ID NO: 312), SEYGTKH (SEQ ID NO: 313), ENMTKNI (SEQ ID NO: 314), ENHIKTI (SEQ ID NO: 244), NNVSQEI (SEQ ID NO: 315), TPEGPSN (SEQ ID NO: 316), LNDTNER (SEQ ID NO: 317), NSLVLNS (SEQ ID NO: 318) and FEPHTYA (SEQ ID NO: 319).

In some embodiments, the insertion sequence is represented by the peptide sequences listed in Table 1.

TABLE 1

| Sequence | SEQ ID NO | Sequence | SEQ ID NO |
|---|---|---|---|
| AAAEVNK | 60 | AFAGANV | 150 |
| AFGGIAD | 37 | AGDYKEW | 75 |
| AGNPGVI | 119 | AGYPNSA | 205 |
| AIMKIDA | 149 | AITPVAH | 129 |
| AIVAAGY | 83 | AKEMRYA | 216 |
| ALGEEST | 76 | ALVSGDV | 312 |
| ANSHDKI | 102 | APVTGEN | 69 |
| ARDTDDA | 115 | ASRDSDV | 173 |
| ATVINGT | 196 | CGKTILT | 93 |
| CNEEMKA | 71 | DAVSRVP | 92 |
| DEDRSGE | 236 | DHEVTDH | 53 |
| DIAGRNP | 105 | DNGVKEK | 147 |
| DQKLPAT | 139 | DQTNSTH | 97 |
| DSMDGKK | 132 | EDNLSYV | 77 |
| EGKNEVI | 137 | EKTNEND | 80 |
| EKTSVNT | 123 | ELGTAEM | 88 |
| ENHIKTI | 244 | ENMTKNI | 314 |
| ENPSSNG | 190 | ENQSAST | 72 |
| EQSHGSK | 301 | ETASVHF | 180 |
| ETDKHGP | 116 | ETPNHDG | 178 |
| EVIKETG | 195 | EWNNHES | 131 |
| FEPHTYA | 319 | FGEITPG | 127 |
| FIENKVA | 175 | FQNSQTR | 309 |
| GAITNNY | 121 | GAQFRSD | 125 |
| GASGEDL | 48 | GDNGFYK | 68 |
| GEMKDMS | 108 | GIGTSEA | 82 |
| GLEFTRH | 112 | GNEQITG | 194 |
| GPGEHSP | 171 | GPGTSDN | 103 |
| GREPSQY | 96 | GSKSTFF | 160 |
| GSLGKPT | 165 | GTDMRQT | 39 |
| GTELVSR | 148 | GTNSSTS | 306 |
| GTQNDVM | 188 | GVSSIDK | 152 |
| GYSTSEV | 101 | HADLRDG | 111 |
| HEDKSVA | 214 | HGSDIRD | 177 |
| HLTSNQL | 40 | HNGVSIL | 167 |
| IDVDTPT | 47 | IEEKNGT | 114 |
| IFTLQSG | 56 | IGAKGSP | 219 |
| IGNRYPT | 235 | IGNTDHD | 157 |
| IGTLPTM | 176 | IGTTQTN | 142 |
| ILGNSRV | 303 | ILSNQAF | 100 |
| ILTASER | 141 | IPFPGPE | 232 |
| ISREFYK | 38 | ITDNRIV | 128 |
| ITMRSMM | 221 | IVSEYAG | 153 |
| KDDHKEP | 134 | KGDVTFT | 191 |
| KGVDGDI | 189 | KQSPSNY | 106 |
| KSQATQY | 146 | KSRSVND | 164 |
| KSSDKDS | 58 | LDNLSVT | 49 |
| LEASNTA | 208 | LEISTTS | 158 |
| LHLGMID | 52 | LIKENRF | 307 |
| LLRDSNN | 302 | LNDTNER | 317 |
| LSTETMV | 74 | LSTSGNE | 66 |
| MDNQSNN | 222 | MHTERGT | 163 |
| MLHGHHL | 230 | MNDFVSL | 109 |
| MNFAGPI | 151 | MQMNSGA | 98 |
| MVNELTP | 94 | MVNVNVK | 217 |
| NADKNNL | 206 | NARSTGM | 42 |
| NASNASA | 161 | NDNANTK | 181 |
| NDNNAGA | 133 | NDNQITR | 305 |
| NDSGAAS | 179 | NESSVTS | 168 |
| NGIERQE | 130 | NHNDSVE | 207 |
| NIAEQPK | 95 | NIEDNMG | 55 |
| NITSVTA | 220 | NLANIPN | 84 |
| NNPLTGD | 65 | NNVSQEI | 315 |
| NPIAESR | 154 | NPTVANT | 63 |
| NREDTKL | 155 | NSDNHNI | 138 |
| NSEPDAN | 87 | NSLVLNS | 318 |
| NSNVPKN | 59 | NTFSRNN | 203 |
| NTLASFS | 218 | NTMNSYP | 99 |
| NTSQSQK | 310 | NVDKTPR | 186 |
| NVSSRSN | 145 | PDNTTTS | 228 |
| PERAQVS | 198 | PHSEGDN | 73 |
| PLRTTQE | 85 | PNEGGHN | 118 |
| PPNQDQH | 192 | PRDLNDP | 187 |
| PSSNNPH | 41 | PTNMPPT | 91 |
| QADVGAN | 135 | QDNSKLS | 225 |
| QEGNLVS | 227 | QHDGSML | 110 |
| QQNNSSL | 162 | QVDGPVR | 67 |
| REDHNLY | 45 | RLDLQHD | 213 |
| RSPGQIG | 215 | RYDERNA | 234 |
| SAVDMSA | 212 | SDIGKTH | 117 |
| SDRRMNT | 86 | SDSTAFI | 78 |
| SERPDTL | 200 | SEYGTKH | 313 |
| SGANHFS | 183 | SHHHPTT | 233 |
| SLNNVTN | 122 | SLSDREY | 170 |
| SLSQYEK | 124 | SNDMTEK | 70 |
| SNRTLSI | 43 | SNTDSGT | 81 |
| SPATASH | 143 | SQSIQKD | 44 |
| SSNALQV | 182 | SSNEYST | 202 |
| SSNGPTD | 79 | SSPAKPT | 226 |
| SSSTAMS | 308 | STHDRDF | 107 |
| STLEMPH | 89 | SVDNRGN | 144 |
| SYIPGHK | 54 | TGANIGY | 224 |
| TGFNNKI | 104 | TGSPNIP | 184 |
| TGTEIGY | 169 | TGVIEGL | 156 |
| THNDLLN | 197 | THSAVHH | 136 |
| TITPITN | 140 | TLMEGMK | 50 |
| TLNILNQ | 64 | TNDANTL | 201 |
| TPANELK | 193 | TPEGPSN | 316 |
| TQPTMEN | 311 | TSTSDIA | 172 |
| TTISSTS | 57 | TTNRTVY | 166 |
| VASKSNH | 126 | VDANGTW | 113 |
| VDKQREN | 304 | VDNDNPL | 209 |
| VELGSSP | 210 | VLTTLSK | 61 |
| VNEIIEK | 51 | VNEKESV | 211 |
| VQVGSMT | 90 | VSLAPSI | 159 |
| VSNISRY | 185 | VTTNREL | 62 |
| VVGSTVL | 120 | VWHDQSA | 231 |
| WSGTLVH | 229 | YESLTQN | 199 |
| YNLQLNS | 204 | YNSLQGQ | 174 |
| YQNDSGK | 46 | YQSGLLE | 223 |
| LNTTKPI | 241 | ENHTKND | 246 |
| GNTTRDY | 255 | GNMVKQV | 256 |
| TNSVKNL | 257 | GNNVKSI | 258 |
| DNSTRSV | 259 | | |
| GNTTKSS | 243 | ENNIRSI | 260 |
| DNSIRNT | 261 | ENHTRNS | 245 |
| NNRRPDD | 262 | QNVIKPT | 263 |
| QNSTKLI | 264 | ANNTRNM | 265 |
| SNTTRNL | 266 | ENSVRNN | 267 |
| NNSTKLL | 268 | GNSVRAN | 269 |
| SNSTRPL | 270 | GNSTMRV | 271 |
| GNSTKIG | 272 | TNTTKNF | 239 |
| MKSGLSM | 273 | SNKMGNT | 274 |
| SNSVKDY | 275 | AVHKSDF | 276 |
| SNSIRNN | 277 | TDRMGLT | 278 |
| SNVIKNV | 279 | YNSTRNQ | 280 |
| GNEVRRD | 281 | DNVIRPT | 282 |
| NVRDLNL | 283 | TSRLPAL | 284 |
| LNTNRTN | 285 | SRTSISE | 286 |
| SNSVRND | 287 | IGNRPVI | 288 |
| QNTIKMT | 289 | FSHTVKG | 290 |
| RRDMDPT | 291 | ENSTRYT | 292 |
| MNSTRPF | 293 | SNNVKQT | 294 |
| SNNSRPY | 295 | NNSTARI | 296 |
| LSNKAML | 297 | TNATRPL | 298 |
| GNAVRGT | 299 | GNSTKAS | 300 |
| IDAARPV | 320 | IDASKPI | 321 |
| IESSRPV | 322 | IESTKPI | 323 |
| INAARPL | 324 | INAARPM | 325 |
| IQATKPM | 326 | IQATKPV | 327 |
| LDSARPM | 328 | LDSARPV | 329 |
| MEASKPV | 330 | MEASRPI | 331 |
| MEASRPL | 332 | VNSSKPV | 333 |
| VNSSRPI | 324 | DDHARDN | 335 |
| DDHARDQ | 326 | DDHTKDQ | 337 |
| DDHTKED | 328 | DEHTKNE | 339 |
| DEHTKNN | 340 | DNHAKNQ | 341 |
| DNHAKQD | 342 | DQHTRDN | 343 |
| DQHTRDQ | 344 | EDHSRNN | 345 |
| EDHSRNQ | 346 | NEHTRDD | 347 |
| NEHTRDE | 348 | QNHTRQQ | 349 |
| QQHAKDD | 350 | IAKDWYR | 351 |
| IAKDYFK | 352 | ISRDFWR | 353 |
| ISRDFYK | 354 | LARQWWR | 355 |
| LARQWYK | 356 | LARQWYR | 357 |
| LARQYFK | 358 | LARQYFR | 359 |
| LARQYWK | 360 | MARNYFK | 361 |
| MARNYFR | 362 | MARNYYR | 363 |
| MARQFFR | 364 | VAKEWWK | 365 |
| VSRNYYR | 366 | | |

In some aspects, the insertion amino acid sequence is at least 71.4% identical to the amino acid sequence provided in Tables 1 and 4-30, FIG. 4 and/or Formulas I-XXXIII. In some aspects, the insertion amino acid sequence is at least 86.7% identical to the amino acid sequence provided in Tables 1 and 4-30, FIG. 4 and/or Formulas I-XXXIII.

Also disclosed herein are methods and kits for producing therapeutic recombinant AAV (rAAV) particles, as well as methods and pharmaceutical compositions or formulations comprising the rAAV particles, for the treatment of a disease or condition affecting the CNS.

Disclosed herein are AAV capsids engineered with desired tropisms, such as an increased viral transduction in the CNS. The AAV capsids can encapsidate a viral vector with a heterologous nucleic acid encoding, for example, a therapeutic gene expression product. Transduction of the heterologous nucleic acid in the CNS can be achieved upon systemic delivery to a subject of the AAV capsid of the present disclosure encapsidating a heterologous nucleic acid. The AAV capsids disclosed herein are advantageous for many applications of gene therapy to treat human disease, including, but not limited to, disorders of the central nervous system.

The recombinant AAV vectors comprising a nucleic acid sequence encoding the AAV capsid proteins of the present disclosure as also provided herein. For example, the viral vectors of the present disclosure comprise a nucleic acid sequence comprising the AAV viral Cap (Capsid) encoding VP1, VP2, and VP3, at least one of which is modified to produce the AAV capsid proteins of the present disclosure. The recombinant AAV vector provided can be derived from an AAV serotype (e.g., AAV9) or a variant AAV serotype including an insertion of the present invention.

### AAV Capsids

Provided herein are modified adeno-associated (AAV) virus capsid compositions useful for integrating a transgene into a target cell or environment (in a subject when they are administered systemically to the subject.

An rAAV comprises an AAV capsid that can be engineered to encapsidate a heterologous nucleic acid (e.g., therapeutic nucleic acid, gene editing machinery). The AAV capsid is made up of three AAV capsid protein monomers, VP1, VP2, and VP3. Sixty copies of these three VP proteins interact in a 1:1:10 ratio to form the viral capsid. VP1 covers the whole of VP2 protein in addition to a ˜137 amino acid N-terminal region (VP1u), VP2 covers the whole of VP3 in addition to ˜65 amino acid N-terminal region (VP1/2 common region). The three capsid proteins share a conserved amino acid sequence of VP3, which in some cases is the region beginning at amino acid position 138 (e.g., AA139-736).

While not wishing to be bound by theory, it is understood that a parent AAV capsid sequence comprises a VP1 region. In certain embodiments, a parent AAV capsid sequence comprises a VP1, VP2 and/or VP3 region, or any combination thereof. A parent VP1 sequence may be considered synonymous with a parent AAV capsid sequence.

The AAV VP3 structure contains highly conserved regions that are common to all serotypes, a core eight-stranded β-barrel motif (βB-βI) and a small α-helix (αA). The loop regions inserted between the β-strands consist of the distinctive HI loop between β-strands H and I, the DE loop between β-strands D and E, and nine variable regions (VRs), which form the top of the loops. These VRs, such as the AA588 loop, are found on the capsid surface and can be associated with specific functional roles in the AAV life cycle including receptor binding, transduction and antigenic specificity.

In some aspects, the rAAV variant of the present invention comprises an AAV capsid protein having a peptide insertion at the residues corresponding to amino acids 588-589 of the AAV9 native sequence of SEQ ID NO: 1.

The AAV capsids comprise AAV capsid proteins (e.g., VP1, VP2, and VP3), each with an insertion, such as in the 588 loop of a parental AAV capsid protein structure (AAV9 VP1 numbering). The 588 loop contains the site of heparan sulfate binding of AAV2 and is amenable to peptide display. The only known receptors for AAV9 is N-linked terminal galactose and AAV receptor (AAVR), but many indications point toward there being others. Modifications to AAV9 588 loop are shown herein to confer an increased specificity and transgene transduction in target in vivo environments.

The present invention provides, in an aspect, a peptide insertion at the AAV 588 loop comprising or consisting of an amino-acid sequence set forth in any one of Tables 1 and 4-30, FIG. 4 and/or Formulas I-XXXIII, as defined above.

Disclosed herein are AAV capsids comprising AAV capsid proteins with an insertion at the 588 loop that confer a desired tropism characterized by a higher efficiency and specificity for transduction in CNS cell types (e.g., brain endothelial cells, neurons, astrocytes). In particular, the AAV capsid proteins disclosed herein enable rAAV-mediated transduction of a heterologous nucleic acid (e.g., transgene) in the CNS of a subject. The AAV capsids of the present disclosure may be formulated as a pharmaceutical composition. In addition, the AAV capsids can be isolated and purified to be used for a variety of applications.

In some embodiments, the rAAV capsid of the present disclosure are generated using the methods disclosed herein. In some instances, the rAAV capsid is chimeric. In some instances, the rAAV, or variant AAV protein comprises therein, confer an increase in a localization of the rAAV within the target tissue, as compared to the parental AAV capsid or capsid protein.

### AAV Capsid Proteins

Disclosed herein are recombinant AAV (rAAV) capsids which comprise AAV capsid proteins that are engineered with a modified capsid protein (e.g., VP1, VP2, VP3). In some embodiments, the rAAV capsid proteins of the present disclosure are generated using the methods disclosed herein. In some embodiments, the AAV capsid proteins are used in the methods of delivering a therapeutic nucleic acid (e.g., a transgene) to a subject. In some instances, the rAAV capsid proteins have desired AAV tropisms rendering them particularly suitable for certain therapeutic applications, e.g., the treatment of a disease or disorder in a subject such as those disclosed herein.

The rAAV capsid proteins are engineered for optimized expression in the CNS, for example the brain, of a subject upon systemic administration of the rAAV to the subject. The rAAV capsid proteins are engineered to include the insertions provided in Tables 1 and 4-30, FIG. 4 and/or Formulas I-XXXIII. The rAAV capsid proteins including the insertions provided in Tables 1 and 4-30, FIG. 4 and/or Formulas I-XXXIII are engineered to achieve efficient transduction of an encapsidated transgene. In particular, the tropisms comprise at least one of an increased specificity and efficiency in the CNS of a subject.

The engineered AAV capsid proteins described herein have, in some cases, an insertion of an amino acid that is heterologous to the parental AAV capsid protein at amino acid positions in the 588 loop. In some embodiments, the amino acid is not endogenous to the parental AAV capsid protein at the amino acid position of the insertion. The amino acid may be a naturally occurring amino acid in the same or equivalent amino acid position as the insertion of the substitution in a different AAV capsid protein.

Generally, the insertion comprises a five-, six-, or seven-amino acid sequence (5-mer, 6-mer, or 7-mer, respectively) that is inserted or substituted at the 588 loop in a parental AAV capsid protein. Aspects provided herein provide amino acid insertions comprising seven amino acid polymer (7-mer) inserted at AA588-589, and may additionally include a substitution of one or two amino acids at amino acid positions flanking the 7-mer sequence (e.g., AA587-588 and/or AA589-590) to produce an eleven amino acid polymer (11-mer) at the 588 loop of a parental AAV capsid protein. The 7-mers described herein were advantageously generated using polymerase chain reaction (PCR) with degenerate primers, where each of the seven amino acids is encoded by a deoxyribose nucleic acid (DNA) sequence N-N-K. “N” is any of the four DNA nucleotides and K is guanine (G) or thymine (T). This method of generating random 7-mer amino acid sequences enables 1.28 billion possible combinations at the protein level.

The rAAV capsid proteins of the present disclosure comprise an insertion of an amino acid in an amino acid sequence of an AAV capsid protein. The AAV capsid, from which an engineered AAV capsid protein of the present disclosure is produced, is referred to as a “parental” AAV capsid. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562.

In some cases, the parental AAV is derived from an AAV with a serotype selected from AAV1, AAV2, AAV3, AAV4, AAV5, AAV6, AAV7, AAV8, AAV9, AAV10, AAV11 and AAV12. The AAV capsid protein that is “derived” from another may be a variant AAV capsid protein. A variant may include, for example, a heterologous amino acid in an amino acid sequence of the AAV capsid protein. The heterologous amino acid may be non-naturally occurring in the AAV capsid protein. The heterologous amino acid may be naturally occurring in a different AAV capsid protein. In some instances, the parental AAV capsid is described in US Pat Publication 2020/0165576 and U.S. Pat. App. Ser. No. 62/832,826 and PCT/US20/20778; the content of each of which is incorporated herein.

In some instances, the parental AAV is AAV9. In some instances, the amino acid sequence of the AAV9 capsid protein comprises SEQ ID NO: 1. The amino acid sequence of AAV9 VP1 capsid protein (>trIQ6JC40|Q6JC40_9VIRU Capsid protein VP1 OS=Adeno-associated virus 9 OX=235455 GN=cap PE=1 SV=1) is provided in SEQ ID NO: 1 (MAADGYLPDWLEDNLSEGIREWWALKPGAPQPKANQQHQDNARGLVLPGYKYL GPGNGLDKGEPVNAADAAALEHDKAYDQQLKAGDNPYLKYNHADAEFQERLKE DTSFGGNLGRAVFQAKKRLLEPLGLVEEAAKTAPGKKRPVEQSPQEPDSSAGIGKS GAQPAKKRLNFGQTGDTESVPDPQPIGEPPAAPSGVGSLTMASGGGAPVADNNEG ADGVGSSSGNWHCDSQWLGDRVITTSTRTWALPTYNNHLYKQISNSTSGGSSNDN AYFGYSTPWGYFDFNRFHCHFSPRDWQRLINNNWGFRPKRLNFKLFNIQVKEVTD NNGVKTIANNLTSTVQVFTDSDYQLPYVLGSAHEGCLPPFPADVFMIPQYGYLTLN DGSQAVGRSSFYCLEYFPSQMLRTGNNFQFSYEFENVPFHSSYAHSQSLDRLMNPLI DQYLYYLSKTINGSGQNQQTLKFSVAGPSNMAVQGRNYIPGPSYRQQRVSTTVTQ NNNSEFAWPGASSWALNGRNSLMNPGPAMASHKEGEDRFFPLSGSLIFGKQGTGR DNVDADKVMITNEEEIKTTNPVATESYGQVATNHQSAQAQAQTGWVQNQGILPG MVWQDRDVYLQGPIWAKIPHTDGNFHPSPLMGGFGMKHPPPQILIKNTPVPADPPT AFNKDKLNSFITQYSTGQVSVEIEWELQKENSKRWNPEIQYTSNYYKSNNVEFAVN TEGVYSEPRPIGTRYLTRNL). In some instances, the parental AAV capsid protein sequence is 70%, 75%, 80%, 85%, 90%, 91%, 92%, 93%, 94%, 95%, 96%, 97%, 98%, 99%, or 100% homologous to SEQ ID NO: 1.

AAV capsid proteins from native AAV serotypes, such as AAV9, with tropisms including the liver activate the innate immune response, which in come cases causes a severe inflammatory response in a subject, which can lead to multi-organ failure. By improving transduction efficiency of a native AAV serotype for a target in vivo tissue (e.g., brain) and additionally decreasing the specificity of the AAV capsid protein to the liver, the rAAV particles of the present disclosure reduce the immunogenic properties of AAV-mediated transgene delivery and prevent activation of the innate immune response.

In some instances, the parental AAV capsid protein comprises the entire VP1 region provided in SEQ ID NO: 1 (e.g., amino acids 1-736). In some instances, the parental AAV capsid protein comprises amino acids 217-736 in SEQ ID NO: 1, which is the common region found in VP1, VP2 and VP3 AAV9 capsid proteins. In some instances, the AAV capsid protein comprises amino acids 64-736 in SEQ ID NO: 1, which is the common region found in VP1 and VP2. The parental AAV capsid protein sequence may comprise amino acids selected from 1-736, 10-736, 20-736, 30-736, 40-736, 50-736, 60-736, 70-736, 80-736, 90-736, 100-736, 110-736, 120-736, 130-736, 140-736, 150-736, 160-736, 170-736, 180-736, 190-736, 200-736, 210-736, 220-736, 230-736, 240-736, 250-736, 260-736, 270-736, 280-736, 290-736, 300-736, 310-736, 320-736, 330-736, 340-736, 350-736, 360-736, 370-736, 380-736, 390-736, 400-736, 410-736, 420-736, 430-736, 440-736, and 450-736, from SEQ ID NO: 1. In some aspects, the rAAV variant comprises an AAV capsid protein comprising an amino acid sequence that is at least 98% identical to amino acid 217 to amino acid 736 of SEQ ID NO: 1. In some instances, the amino acid insertion is at a three (3)-fold axis of symmetry of a corresponding parental AAV capsid protein.

Disclosed herein are insertions of an amino acid sequence in an AAV capsid protein. Where the sequence numbering designation “588-589” is noted for AAV9, for example AAV VP1, the invention also includes insertions in similar locations in the other AAV serotypes. As used herein, “AA588-589” indicates that the insertion of the amino acid (or amino acid sequence) is immediately after an amino acid (AA) at position 588 and immediately before an AA at position 589 within an amino acid sequence of a parental AAV VP capsid protein (VP1 numbering). Amino acids 587-591 include a motif comprising “AQAQA” as set forth in SEQ ID NO: 1. Exemplary AAV capsid protein sequences are provided in Table 31. For example, GNTTRDY (SEQ ID NO: 255) is inserted at AA588-589 in an AAV9 capsid amino acid sequence, and provides variant C (SEQ ID NO: 376). It is envisioned that the insertions disclosed herein (Tables 1 and 4-30, FIG. 4 and/or Formulas I-XXXIII) may be inserted at AA588-589 in an amino acid sequence of a parental AAV9 capsid protein, a variant thereof, or equivalent amino acid position of a parental AAV of a different serotype (e.g., AAV1, AAV2, AAV3, and the like).

TABLE 31
Exemplary AAV Capsid Protein Sequences

| SEQ ID | | |
|---|---|---|
| NO: | Identifier | Sequence |
| 369 | AAV- | MAADGYLPDWLEDNLSEGIREWWALKPGAPQPKANQ |
| | PHP.eB | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSDGTLAVPFKAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 370 | AAV.CAP- | MAADGYLPDWLEDNLSEGIREWWALKPGAPQPKANQ |
| | A4 | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTIKD |
| | | NTPGRQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQAQAQTGWVQNQGILP |
| | | GMVWQDRDVYLQGPIWAKIPHTDGNFHPSPLMGGFGM |
| | | KHPPPQILIKNTPVPADPPTAFNKDKLNSFITQYSTGQVS |
| | | VEIEWELQKENSKRWNPEIQYTSNYYKSNNVEFAVNTE |
| | | GVYSEPRPIGTRYLTRNL |
| 371 | AAV.CAP- | MAADGYLPDWLEDNLSEGIREWWALKPGAPQPKANQ |
| | B2 | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTIQQ |
| | | GKQSVQTLKFSVAGPSNMAVQGRNYIPGPSYRQQRVS |
| | | TTVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSDGTLAVPFKAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 372 | AAV.CAP- | MAADGYLPDWLEDNLSEGIREWWALKPGAPQPKANQ |
| | B10 | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTIDG |
| | | AATKNQTLKFSVAGPSNMAVQGRNYIPGPSYRQQRVS |
| | | TTVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSDGTLAVPFKAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 373 | AAV.CAP- | MAADGYLPDWLEDNLSEGIREWWALKPGAPQPKANQ |
| | B22 | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTIDG |
| | | QSSKSQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSDGTLAVPFKAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 374 | Variant A | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQISREFYKAQAQTGWV |
| | | QNQGILPGMVWQDRDVYLQGPIWAKIPHTDGNFHPSPL |
| | | MGGFGMKHPPPQILIKNTPVPADPPTAFNKDKLNSFITQ |
| | | YSTGQVSVEIEWELQKENSKRWNPEIQYTSNYYKSNNV |
| | | EFAVNTEGVYSEPRPIGTRYLTRNL |
| 375 | Variant B | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQEDNLSYVAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 376 | Variant C | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQGNTTRDYAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 377 | Variant D | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQTNSVKNLAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 378 | Variant E | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQLNTTKPIAQAQTGWV |
| | | QNQGILPGMVWQDRDVYLQGPIWAKIPHTDGNFHPSPL |
| | | MGGFGMKHPPPQILIKNTPVPADPPTAFNKDKLNSFITQ |
| | | YSTGQVSVEIEWELQKENSKRWNPEIQYTSNYYKSNNV |
| | | EFAVNTEGVYSEPRPIGTRYLTRNL |
| 379 | Variant F | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQENHTKNDAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 380 | Variant G | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQNVRDLNLAQAQTGW |
| | | VQNQGILPGMVWQDRDVYLQGPIWAKIPHTDGNFHPSP |
| | | LMGGFGMKHPPPQILIKNTPVPADPPTAFNKDKLNSFIT |
| | | QYSTGQVSVEIEWELQKENSKRWNPEIQYTSNYYKSNN |
| | | VEFAVNTEGVYSEPRPIGTRYLTRNL |
| 381 | Variant H | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQQNSTKLIAQAQTGWV |
| | | QNQGILPGMVWQDRDVYLQGPIWAKIPHTDGNFHPSPL |
| | | MGGFGMKHPPPQILIKNTPVPADPPTAFNKDKLNSFITQ |
| | | YSTGQVSVEIEWELQKENSKRWNPEIQYTSNYYKSNNV |
| | | EFAVNTEGVYSEPRPIGTRYLTRNL |
| 382 | Variant I | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQSNVIKNVAQAQTGWV |
| | | QNQGILPGMVWQDRDVYLQGPIWAKIPHTDGNFHPSPL |
| | | MGGFGMKHPPPQILIKNTPVPADPPTAFNKDKLNSFITQ |
| | | YSTGQVSVEIEWELQKENSKRWNPEIQYTSNYYKSNNV |
| | | EFAVNTEGVYSEPRPIGTRYLTRNL |
| 383 | Variant J | MAADGYLPDWLEDNLSEGIREWWALKPGAPQPKANQ |
| | | QHQDNARGLVLPGYKYLGPGNGLDKGEPVNAADAAA |
| | | LEHDKAYDQQLKAGDNPYLKYNHADAEFQERLKEDTS |
| | | FGGNLGRAVFQAKKRLLEPLGLVEEAAKTAPGKKRPVE |
| | | QSPQEPDSSAGIGKSGAQPAKKRLNFGQTGDTESVPDPQ |
| | | PIGEPPAAPSGVGSLTMASGGGAPVADNNEGADGVGSS |
| | | SGNWHCDSQWLGDRVITTSTRTWALPTYNNHLYKQIS |
| | | NSTSGGSSNDNAYFGYSTPWGYFDFNRFHCHFSPRDWQ |
| | | RLINNNWGFRPKRLNFKLFNIQVKEVTDNNGVKTIANN |
| | | LTSTVQVFTDSDYQLPYVLGSAHEGCLPPFPADVFMIPQ |
| | | YGYLTLNDGSQAVGRSSFYCLEYFPSQMLRTGNNFQFS |
| | | YEFENVPFHSSYAHSQSLDRLMNPLIDQYLYYLSRTING |
| | | SGQNQQTLKFSVAGPSNMAVQGRNYIPGPSYRQQRVST |
| | | TVTQNNNSEFAWPGASSWALNGRNSLMNPGPAMASH |
| | | KEGEDRFFPLSGSLIFGKQGTGRDNVDADKVMITNEEEI |
| | | KTTNPVATESYGQVATNHQSAQNLANIPNAQAQTGWV |
| | | QNQGILPGMVWQDRDVYLQGPIWAKIPHTDGNFHPSPL |
| | | MGGFGMKHPPPQILIKNTPVPADPPTAFNKDKLNSFITQ |
| | | YSTGQVSVEIEWELQKENSKRWNPEIQYTSNYYKSNNV |
| | | EFAVNTEGVYSEPRPIGTRYLTRNL |

The insertions described herein may, in some cases, comprise a 7-mer insertion at AA588-589. It is envisioned that any 7-mer insertion disclosed herein in addition to a substitution with any amino acid at amino acid positions 587-590 may comprise an 11-mer.

Disclosed herein are AAV capsid proteins with an insertion described above in a parental AAV capsid protein that confers an increased efficiency or specificity for the CNS in a subject, even when delivered systemically. One of the many advantages of the AAV capsid proteins described herein is their ability to target tissue and cells within the CNS. The tissue can be the brain or the spinal cord. Non-limiting examples of CNS cells include a neuron and a glial cell. Glial cells can be selected from an oligodendrocyte, an ependymal cell, an astrocyte and a microglia.

In some instances, the AAV capsid protein comprises an insertion of at least or about five, six, or seven amino acids of an amino acid sequence of Tables 1 and 4-30, FIG. 4 and/or Formulas I-XXXIII at an amino acid position 588-589 in a parental AAV9 capsid protein (SEQ ID NO: 1). In some cases, the AAV capsid protein has an increased specificity for viral transduction in brain and or spinal cord.

The rAAV capsid proteins of the present disclosure may also have a substitution of an amino acid sequence at amino acid position 452-458 in a parental AAV9 capsid protein, or variant thereof, as described in WO2020068990. In some embodiments, the substitution of the amino acid sequence comprises KDNTPGR (SEQ ID NO: 367) at amino acid position 452-458 in the parental AAV9 capsid protein. In some embodiments, the substitution of the amino acid sequence comprises DGAATKN (SEQ ID NO: 368) at amino acid position 452-458 in the parental AAV9 capsid protein.

The rAAV capsid proteins described herein may be isolated and purified. The AAV may be isolated and purified by methods standard in the art such as by column chromatography, iodixanol gradients, or cesium chloride gradients. Methods for purifying AAV from helper virus are known in the art and may include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

In addition, the AAV capsid proteins disclosed herein, either isolated and purified, or not, may be formulated into a pharmaceutical formulation, which in some cases, further comprises a pharmaceutically acceptable carrier.

The rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein.

Peptide insertion sequences of the disclosure include sequences that have been modified in any way and for any reason, for example, to: (1) reduce susceptibility to proteolysis, (2) alter binding affinities, and (3) confer or modify other physicochemical or functional properties. For example, single or multiple amino acid substitutions (e.g., equivalent, conservative or non-conservative substitutions, deletions or additions) may be made in a sequence.

A conservative amino acid substitution refers to the substitution of an amino acid in an insertion sequence with a functionally similar amino acid having similar properties, e.g., size, charge, hydrophobicity, hydrophilicity, and/or aromaticity. The following six groups each contain amino acids that are conservative substitutions for one another are found in Table 2.

### TABLE 2

i. Alanine (A), Serine (S), and Threonine (T)
ii. Aspartic acid (D) and Glutamic acid (E)
iii. Asparagine (N) and Glutamine (Q)
iv. Arginine (R) and Lysine (K)
v. Isoleucine (I), Leucine (L), Methionine (M), and Valine (V)
vi. Phenylalanine (F), Tyrosine (Y), and Tryptophan (W)

Additionally, within the meaning of the term “equivalent amino acid substitution” as applied herein, one amino acid may be substituted for another, in one embodiment, within the groups of amino acids indicated herein below:
1. Amino acids with polar side chains (Asp, Glu, Lys, Arg, His, Asn, Gln, Ser, Thr, Tyr, and Cys,)
2. Amino acids with small nonpolar or slightly polar residues (Ala, Ser, Thr, Pro, Gly);
3. Amino acids with non-polar side chains (Gly, Ala, Val, Leu, Ile, Phe, Trp, Pro, and Met)
4. Amino acids with large, aliphatic, nonpolar residues (Met, Leu, Ile, Val, Cys, Norleucine (Nle), homocysteine)
5. Amino acids with aliphatic side chains (Gly, Ala Val, Leu, Ile)
6. Amino acids with cyclic side chains (Phe, Tyr, Trp, His, Pro)
7. Amino acids with aromatic side chains (Phe, Tyr, Trp)
8. Amino acids with acidic side chains (Asp, Glu)
9. Amino acids with basic side chains (Lys, Arg, His)
10. Amino acids with amide side chains (Asn, Gln)
11. Amino acids with hydroxy side chains (Ser, Thr)
12. Amino acids with sulphur-containing side chains (Cys, Met),
13. Neutral, weakly hydrophobic amino acids (Pro, Ala, Gly, Ser, Thr)
14. Hydrophilic, acidic amino acids (Gln, Asn, Glu, Asp), and
15. Hydrophobic amino acids (Leu, Ile, Val).

The following terms are used to describe the sequence relationships between two or more nucleic acids or nucleic acids or polypeptides: (a)“reference sequence,” (b) “comparison window,” (c)“sequence identity,” (d)“percentage of sequence identity,” and (e)“substantial identity.”

As used herein, “reference sequence” is a defined sequence used as a basis for sequence comparison. The reference sequence can be a nucleic acid sequence. A reference sequence may be a subset or the entirety of a specified sequence. For example, a reference sequence may be a segment of a full-length cDNA or of a genomic DNA sequence, or the complete cDNA or complete genomic DNA sequence, or a domain of a polypeptide sequence.

As used herein, “comparison window” refers to a contiguous and specified segment of a nucleic acid or an amino acid sequence, wherein the nucleic acid/amino acid sequence can be compared to a reference sequence and wherein the portion of the nucleic acid/amino acid sequence in the comparison window may comprise additions or deletions (i.e., gaps) compared to the reference sequence (which does not comprise additions or deletions) for optimal alignment of the two sequences. The comparison window can vary for nucleic acid and polypeptide sequences. Generally, for nucleic acids, the comparison window is at least 20 contiguous nucleotides in length, and optionally can be 30, 40, 50, 100 or more nucleotides. For amino acid sequences, the comparison window is at least about 10 amino acids, and can optionally be 15, 20, 30, 40, 50, 100 or more amino acids. Those of skill in the art understand that to avoid a high similarity to a reference sequence due to inclusion of gaps in the nucleic acid or amino acid sequence, a gap penalty is typically introduced and is subtracted from the number of matches.

Methods of alignment of nucleotide and amino acid sequences for comparison are well known in the art. The local homology algorithm (BESTFIT) of Smith and Waterman (1981) Adv. Appl. Math 2:482, may permit optimal alignment of compared sequences; by the homology alignment algorithm (GAP) of Needleman and Wunsch (1970) J. Mol. Biol. 48:443-453; by the search for similarity method (Tfasta and Fasta) of Pearson and Lipman (1988) Proc. Natl. Acad. Sci. USA 85:2444; by computerized implementations of these algorithms, including, but not limited to: CLUSTAL in the PC/Gene program by Intelligenetics, Mountain View, Calif., GAP, BESTFIT, BLAST, FASTA and TFASTA in the Wisconsin Genetics Software Package, Version 8 (available from Genetics Computer Group (GCG™ programs (Accelrys, Inc., San Diego, Calif.)). The CLUSTAL program is well described by Higgins and Sharp (1988) Gene 73:237-244; Higgins and Sharp (1989) CABIOS 5: 151-153; Corpet, et al. (1988) Nucleic Acids Res. 16: 10881-10890; Huang, et al. (1992) Computer Applications in the Biosciences 8: 155-165; and Pearson, et al. (1994) Meth. Mol. Biol. 24:307-331. An example of a good program to use for optimal global alignment of multiple sequences is PileUp (Feng and Doolittle (1987) J. Mol. Evol. 25:351-260, which is similar to the method described by Higgins and Sharp (1989) CABIOS 5: 151-153 (and is hereby incorporated by reference). The BLAST family of programs that can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., eds., Greene Publishing and Wiley-Interscience, New York (1995). An updated version of the BLAST family of programs includes the BLAST+ suite. (Camacho, C., et al. (2009 Dec. 15) BLAST+: architecture and applications. BMC Bioinformatics 10:421).

GAP uses the algorithm of Needleman and Wunsch (1970) J. Mol. Biol. 48:443-53, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP makes a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package are 8 and 2, respectively. The gap creation and gap extension penalties can be expressed as an integer selected from the group of integers consisting of from 0 to 100. Thus, for example, the gap creation and gap extension penalties can be 0, 1, 2, 3, 4, 5, 6, 7, 8, 9, 10, 15, 20, 30, 40, 50 or more.

GAP presents one member of the family of best alignments. There may be many members of this family. GAP displays four figures of merit for alignments: Quality, Ratio, Identity and Similarity. The Quality is the metric maximized in order to align the sequences. Ratio is the quality divided by the number of bases in the shorter segment.

“Percent Identity” is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see: Henikoff and Henikoff, (1989) Proc. Natl. Acad. Sci. USA 89: 10915).

Sequence identity/similarity values provided herein can refer to the value obtained using the BLAST+2.5.0 suite of programs using default settings (blast.ncbi.nlm.nih.gov) (Camacho, C., et al. (2009) BLAST+: architecture and applications. BMC Bioinformatics 10:421).

As those of ordinary skill in the art will understand, BLAST searches assume that proteins can be modeled as random sequences. However, many real proteins comprise regions of nonrandom sequences, which may be homopolymeric tracts, short-period repeats, or regions enriched in one or more amino acids. Such low-complexity regions may be aligned between unrelated proteins even though other regions of the protein are entirely dissimilar. A number of low-complexity filter programs can be employed to reduce such low-complexity alignments. For example, the SEG (Wooten and Federhen, (1993) Comput. Chem. 17: 149-63) and XNU (Ci-ayerie and States (1993) Comput. Chem. 17: 191-201) low-complexity filters can be employed alone or in combination.

The terms “substantial identity” and “substantially identical” indicate that a polypeptide or nucleic acid comprises a sequence with between 55-100% sequence identity to a reference sequence, with at least 55% sequence identity, or at least 60%, or at least 65%, or at least 70%, or at least 75%, or at least 80%, or at least 85%, or at least 90%, or at least 95%, or at least 99% sequence identity or any percentage of value within the range of 55-100% sequence identity relative to the reference sequence. The percent sequence identity may occur over a specified comparison window. Optimal alignment may be ascertained or conducted using the homology alignment algorithm of Needleman and Wunsch, supra.

For example, the insertion sequences may include, but are not limited to, sequences that are not exactly the same as the sequences disclosed herein, but which have, in addition to the substitutions explicitly described for various sequences listed herein, additional substitutions of amino acid residues which substantially do not impair the activity or properties of the sequences described herein, such as those predicted by homology software e.g. BLOSUM62 matrices. Examples of such conservative amino acid substitutions may include but are not limited to the sequences of Formulas I-III.

### AAV PARTICLES

The rAAV particles with the insertion sequences described herein have an increased transduction efficiency in the CNS. In some instances, the increased transduction efficiency comprises a 1-fold, 2-fold, 3-fold, 4-fold, 5-fold, 6-fold, 7-fold, 8-fold, 9-fold, 10-fold, 15-fold, 20-fold, 30-fold, 40-fold, 50-fold or 100-fold increase, or more. In some instances, the increased transduction efficiency is at least 2-fold. In some instances, the increased transduction efficiency is at least 4-fold. In some instances, the increased transduction efficiency is at least 8-fold.

The rAAV particles with the insertion sequences described herein have an increased expression efficiency or specificity in the CNS. Detecting whether a rAAV possesses more or less specificity for a target in vivo environment, includes measuring a level of gene expression product (e.g., RNA or protein) expressed from the heterologous nucleic acid encapsidated by the rAAV in a tissue sample obtained from a subject. Suitable methods for measuring expression of a gene expression product include next-generation sequencing (NGS) and quantitative polymerase chain reaction (qPCR).

The increased expression in the CNS is represented by the cpm values provided in Tables 4-30 and/or FIG. 4.

### Heterologous Nucleic Acids

Disclosed herein are therapeutic nucleic acids useful for the treatment or prevention of a disease or condition, or symptom of the disease or condition. In some embodiments, the therapeutic nucleic acids encode a therapeutic gene expression product. Non-limiting examples of gene expression products include proteins, polypeptides, peptides, enzymes, antibodies, antigen binding fragments, nucleic acid (RNA, DNA, antisense oligonucleotide, siRNA, and the like), and gene editing components, for use in the treatment, prophylaxis, and/or amelioration of the disease or disorder, or symptoms of the disease or disorder. In some instances, the therapeutic nucleic acids are placed in an organism, cell, tissue or organ of a subject by way of a rAAV, such as those disclosed herein.

Disclosed herein are rAAVs, each comprising a viral vector (e.g., a single stranded DNA molecule (ssDNA)). In some instances, the viral vector comprises two inverted terminal repeat (ITR) sequences that are about 145 bases each, flanking a transgene. In some embodiments, the transgene comprises a therapeutic nucleic acid, and in some cases, a promoter in cis with the therapeutic nucleic acid in an open reading frame (ORF). The promoter is capable of initiating transcription of therapeutic nucleic acid in the nucleus of the target cell. The ITR sequences can be from any AAV serotype. Non-limiting examples of AAV serotypes include AAV1, AAV2, AAV3, AAV4, AAV5, AAV6, AAV7, AAV8, AAV9, AAV10, AAV11, and AAV12. In some cases, an ITR is from AAV2. In some cases, an ITR is from AAV9.

Disclosed herein are transgenes that can comprise any number of nucleotides. In some cases, a transgene can comprise less than about 100 nucleotides. In some cases, a transgene can comprise at least about 100 nucleotides. In some cases, a transgene can comprise at least about 200 nucleotides. In some cases, a transgene can comprise at least about 300 nucleotides. In some cases, a transgene can comprise at least about 400 nucleotides. In some cases, a transgene can comprise at least about 500 nucleotides. In some cases, a transgene can comprise at least about 1000 nucleotides. In some cases, a transgene can comprise at least about 5000 nucleotides. In some cases, a transgene can comprise over 5,000 nucleotides. In some cases, a transgene can comprise between about 500 and about 5000 nucleotides. In some cases, a transgene comprises about 5000 nucleotides. In any of the cases disclosed herein, the transgene can comprise DNA, RNA, or a hybrid of DNA and RNA. In some cases, the transgene can be single stranded. In some cases, the transgene can be double stranded.

Disclosed herein are transgenes useful for modulating the expression or activity of a target gene or gene expression product thereof. In some instances, the transgene is encapsidated by an rAAV capsid protein of an rAAV particle described herein. In some instances, the rAAV particle is delivered to a subject to treat a disease or condition disclosed herein in the subject. In some instances, the delivery is systemic.

The transgenes disclosed herein are useful for expressing an endogenous gene at a level similar to that of a healthy or normal individual. This is particularly useful in the treatment of a disease or condition related to the underexpression, or lack of expression, of a gene expression product. In some embodiments, the transgenes disclosed herein are useful for overexpressing an endogenous gene, such that an expression level of the endogenous gene is above the expression level of a healthy or normal individual. Additionally, transgenes can be used to express exogenous genes (e.g., active agent such as an antibody, peptide, nucleic acid, or gene editing components). In some embodiments, the therapeutic gene expression product is capable of altering, enhancing, increasing, or inducing the activity of one or more endogenous biological processes in the cell. In some embodiments, the transgenes disclosed herein are useful for reducing expression of an endogenous gene, for example, a dominant negative gene. In some embodiments, the therapeutic gene expression product is capable of altering, inhibiting, reducing, preventing, eliminating, or impairing the activity of one or more endogenous biological processes in the cell. In some aspects, the increase of gene expression refers to an increase by at least about 20%, 30%, 40%, 50%, 60%, 70%, 80%, 85%, 90%, 95% and 100%. In one aspect, the protein product of the targeted gene may be increased by at least about 20%, 30%, 40%, 50%, 60%, 70%, 80%, 85%, 90%, 95% and 100%. In some aspects, the decrease of gene expression refers to an increase by at least about 20%, 30%, 40%, 50%, 60%, 70%, 80%, 85%, 90%, 95% and 100%. In one aspect, the protein product of the targeted gene may be decreased by at least about 20%, 30%, 40%, 50%, 60%, 70%, 80%, 85%, 90%, 95% and 100%.

When endogenous sequences (endogenous or part of a transgene) are expressed with a transgene, the endogenous sequences can be full-length sequences (wild-type or mutant) or partial sequences. The endogenous sequences can be functional. Non-limiting examples of the function of these full length or partial sequences include increasing the serum half-life of the polypeptide expressed by a transgene (e.g., therapeutic gene) and/or acting as a carrier.

A transgene can be inserted into an endogenous gene such that all, some or none of the endogenous gene is expressed. For example, a transgene as described herein can be inserted into an endogenous locus such that some (N-terminal and/or C-terminal to a transgene) or none of the endogenous sequences are expressed, for example as a fusion with a transgene. In other cases, a transgene (e.g., with or without additional coding sequences of the endogenous gene) is integrated into any endogenous locus, for example a safe-harbor locus. For example, a Frataxin (FXN) transgene can be inserted into an endogenous FXN gene. A transgene can be inserted into any gene, e.g., the genes as described herein.

At least one advantage of the present disclosure is that virtually any therapeutic nucleic acid may be used to express any therapeutic gene expression product. In some instances, the therapeutic gene expression product is a therapeutic protein or a peptide (e.g., antibody, antigen-binding fragment, peptide, or protein). In one embodiment the protein encoded by the therapeutic nucleic acid is between 50-5000 amino acids in length. In some embodiments the protein encoded is between 50-2000 amino acids in length. In some embodiments the protein encoded is between 50-1000 amino acids in length. In some embodiments the protein encoded is between 50-1500 amino acids in length. In some embodiments the protein encoded is between 50-800 amino acids in length. In some embodiments the protein encoded is between 50-600 amino acids in length. In some embodiments the protein encoded is between 50-400 amino acids in length. In some embodiments the protein encoded is between 50-200 amino acids in length. In some embodiments the protein encoded is between 50-100 amino acids in length. In some embodiments the peptide encoded is between 4-50 amino acids in length. In some embodiments, the protein encoded is a tetrapeptide, a pentapeptide, a hexapeptide, a heptapeptide, an octapeptide, a nonapeptide, or a decapeptide. In some embodiments, the protein encoded comprises a peptide of 2-30 amino acids, such as for example 5-30, 10-30, 2-25, 5-25, 10-25, or 10-20 amino acids. In some embodiments, the protein encoded comprises a peptide of at least 11, 12, 13, 14, 15, 17, 20, 25 or 30 amino acids, or a peptide that is no longer than 50 amino acids, e.g. no longer than 35, 30, 25, 20, 17, 15, 14, 13, 12, 11 or 10 amino acids.

Non-limiting examples of therapeutic protein or peptides include an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neuropilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinsase inhibitor, a proteolytic protein, a proteolytic protein inhibitor, a semaphoring, a semaphorin receptor, a serotonin transport protein, a serotonin uptake inhibitor, a serotonin receptor, a serpin, a serpin receptor, and a tumor suppressor. In certain embodiments, the therapeutic protein or peptide is selected from brain-derived neurotrophic factor (BDNF), ciliary neurotrophic factor (CNTF), macrophage colony-stimulating factor (CSF), epidermal growth factor (EGF), fibroblast growth factor (FGF), gonadotropin, interferon-gamma (IFN), insulin-like growth factor 1 (IFG-1), nerve growth factor (NGF), platelet-derived growth factor (PDGF), pigment epithelium-derived factor (PEDF), transforming growth factor (TGF), transforming growth factor-beta (TGF-B), tumor necrosis factor (TNF), vascular endothelial growth factor (VEGF), prolactin, somatotropin, X-linked inhibitor of apoptosis protein 1 (XIAP1), interleukin 1 (IL-1), IL-2, IL-3, IL-4, IL-5, IL-6, IL-7, IL-8, IL-9, IL-10, IL-10, viral IL-10, IL-11, IL-12, IL-13, IL-14, IL-15, IL-16, IL-17, and IL-18.

A therapeutic gene expression product can comprise gene editing components. Non-limiting examples of gene editing components include those required for CRISPR/Cas, artificial site-specific RNA endonuclease (ASRE), zinc finger endonuclease (ZFN), and transcription factor like effector nuclease (TALEN). In a non-limiting example, a subject having Huntington's disease is identified. The subject is then systemically administered a first amount of a rAAV encapsidating a viral vector encoding ZFN engineered to represses the transcription of the Huntingtin (HTT) gene. The rAAV will include a modified AAV capsid protein that includes an amino acid sequence provided in any one of Tables 1 and 4-30, FIG. 4 and/or Formulas I-XXXIII, so as to allow proper targeting of the ZFN to the nervous system, while reducing expression in off-target organs, such as the liver. If needed, the subject is administered a second or third dose of the rAAV, until a therapeutically effective amount of the ZFN is expressed in the subject's nervous system.

A therapeutic nucleic acid can comprise a non-protein coding gene e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. The non-protein coding gene may also encode a tRNA, rRNA, tmRNA, piRNA, double stranded RNA, snRNA, snoRNA, and/or long non-coding RNA (lncRNA). In some cases, the non-protein coding gene can modulate the expression or the activity of a target gene or gene expression product. For example, the RNAs described herein may be used to inhibit gene expression in the CNS. In some cases, inhibition of gene expression refers to an inhibition by at least about 20%, 30%, 40%, 50%, 60%, 70%, 80%, 85%, 90%, 95% and 100%. In some cases, the protein product of the targeted gene may be inhibited by at least about 20%, 30%, 40%, 50%, 60%, 70%, 80%, 85%, 90%, 95% and 100%. The gene can be either a wild type gene or a gene with at least one mutation. The targeted protein may be either a wild type protein or a protein with at least one mutation.

A therapeutic nucleic acid can modulate the expression or activity of a gene or gene expression product expressed from the gene that is implicated in a disease or disorder of the CNS. For example, the therapeutic nucleic acid, in some cases is a gene or a modified version of the gene described herein. In some instances, the gene or gene expression product is inhibited. In some instances, the gene or gene expression product is enhanced.

In another example, the therapeutic nucleic acid comprises an effector gene expression product such as a gene editing component specific to target a gene therein. Non-limited examples of genes include target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPCl, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMNI), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SODI), iduronate 2 sulfatase (hIDS), Glucosylceramidase Beta (GBA), fragile X mental retardation 1 (FMR1), NPC Intracellular Cholesterol Transporter 1 (NPCl), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26. In some instances, the gene or gene expression product is inhibited. In some instances, the gene or gene expression product is enhanced.

### AAV Vectors

Aspects disclosed herein comprise plasmid vectors comprising a nucleic acid sequence encoding the AAV capsids and AAV capsid proteins described herein. AAV vectors described herein are useful for the assembly of a rAAV and viral packaging of a heterologous nucleic acid. In addition, an AAV vector may encode a transgene comprising the heterologous nucleic acid.

An AAV vector can comprise a transgene, which in some cases encodes a heterologous gene expression product (e.g., therapeutic gene expression product, recombinant capsid protein, and the like). The transgene is in cis with two inverted terminal repeats (ITRs) flanking the transgene. The transgene may comprise a therapeutic nucleic acid encoding a therapeutic gene expression product. Due to the limited packaging capacity of the rAAV (˜5 kB), in some cases, a longer transgene may be split between two AAV vectors, the first with 3′ splice donor and the second with a 5′ splice acceptor. Upon co-infection of a cell, concatemers form, which are spliced together to express a full-length transgene.

A transgene is generally inserted so that its expression is driven by the endogenous promoter at the integration site, namely the promoter that drives expression of the endogenous gene into which a transgene is inserted. In some instances, a transgene comprises a promoter and/or enhancer, for example a constitutive promoter or an inducible or tissue/cell specific promoter. As a non-limiting example, the promoter may be CMV promoter, a CMV-β-Actin-intron-β-Globin hybrid promoter (CAG), CBA promoter, FRDA or FXN promoter, UBC promoter, GUSB promoter, NSE promoter, Synapsin promoter, MeCP2 promoter, GFAP promoter, H1 promoter, U6 promoter, NFL promoter, NFH promoter, SCN8A promoter, or PGK promoter. As a non-limiting example, promoters can be tissue-specific expression elements include, but are not limited to, human elongation factor 1α-subunit (EF1α), immediate-early cytomegalovirus (CMV), chicken β-actin (CBA) and its derivative CAG, the β glucuronidase (GUSB), and ubiquitin C (UBC). The transgene may include a tissue-specific expression elements for neurons such as, but not limited to, neuron-specific enolase (NSE), platelet-derived growth factor (PDGF), platelet-derived growth factor β-chain (PDGF-β), the synapsin (Syn), the methyl-CpG binding protein 2 (MeCP2), Ca2+/calmodulin-dependent protein kinase II (CaMKII), metabotropic glutamate receptor 2 (mGluR2), NFL, NFH, np32, PPE, Enk and EAAT2 promoters. The transgene may comprise a tissue-specific expression element for astrocytes such as, but not limited to, the glial fibrillary acidic protein (GFAP) and EAAT2 promoters. The transgene may comprise tissue-specific expression elements for oligodendrocytes such as, but not limited to, the myelin basic protein (MBP) promoter.

In some embodiments, the promoter is less than 1 kb. The promoter may have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800. The promoter may have a length between 200-300, 200-400, 200-500, 200-600, 200-700, 200-800, 300-400, 300-500, 300-600, 300-700, 300-800, 400-500, 400-600, 400-700, 400-800, 500-600, 500-700, 500-800, 600-700, 600-800 or 700-800. The promoter may provide expression of the therapeutic gene expression product for a period of time in targeted tissues such as, but not limited to, the CNS. Expression of the therapeutic gene expression product may be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or more than 65 years. Expression of the payload may be for 1-5 hours, 1-12 hours, 1-2 days, 1-5 days, 1-2 weeks, 1-3 weeks, 1-4 weeks, 1-2 months, 1-4 months, 1-6 months, 2-6 months, 3-6 months, 3-9 months, 4-8 months, 6-12 months, 1-2 years, 1-5 years, 2-5 years, 3-6 years, 3-8 years, 4-8 years or 5-10 years or 10-15 years, or 15-20 years, or 20-25 years, or 25-30 years, or 30-35 years, or 35-40 years, or 40-45 years, or 45-50 years, or 50-55 years, or 55-60 years, or 60-65 years.

An AAV vector can comprise a genome of a helper virus. Helper virus proteins are required for the assembly of a recombinant AAV (rAAV), and packaging of a transgene containing a heterologous nucleic acid into the rAAV. The helper virus genes are adenovirus genes E4, E2a and VA, that when expressed in the cell, assist with AAV replication. In some embodiments, an AAV vector comprises E2. In some embodiments, an AAV vector comprises E4. In some embodiments, an AAV vector comprises VA. In some instances, the AAV vector comprises one of helper virus proteins, or any combination.

The target gene or gene expression product for use in a transgene can be selected from ATP1A2, CACNAIA, SETD5, SHANK3, NF2, DNMT1, TCF4, RAI1, PEX1, ARSA, EIF2B5, EIF2B1, EIF2B2, NPCl, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMNI), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SODI), iduronate 2 sulfatase (hIDS), Glucosylceramidase Beta (GBA), fragile X mental retardation 1 (FMR1), NPC Intracellular Cholesterol Transporter 1 (NPCl), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX11β, PEX12, PEX13, PEX14, PEX16, PEX19, and PEX26.

An AAV vector can comprise a viral genome comprising a nucleic acid encoding the recombinant AAV (rAAV) capsid protein described herein. The viral genome can comprise a Replication (Rep) gene encoding a Rep protein, and Capsid (Cap) gene encoding an AAP protein in the first open reading frame (ORF1) or a Cap protein in the second open reading frame (ORF2). The Rep protein is selected from Rep78, Rep68, Rep52, and Rep40. In some instances, the Cap gene is modified encoding a modified AAV capsid protein described herein. A wild-type Cap gene encodes three proteins, VP1, VP2, and VP3. In some cases, VP1 is modified. In some cases, VP2 is modified. In some cases, VP3 is modified. In some cases, all three VP1-VP3 are modified. The AAV vector can comprise nucleic acids encoding wild-type Rep78, Rep68, Rep52, Rep40 and AAP proteins.

In some instances, the AAV9 VP1 gene provided in SEQ ID NO: 384 shown in Table 3, is modified to include any one of SEQ ID NOS: 37-366. The AAV vector described herein may be used to produce a variant AAV capsid by the methods described herein.

TABLE 3
VP1 Capsid Protein Nucleic Acid Sequences

| SEQ ID | | |
|---|---|---|
| NO: | Identifier | Sequence |
| 384 | AAV9 | ATGGCTGCCGATGGTTATCTTCCAGATTGGCTCGAGGACAACCTT |
| | >AY530579.1 | AGTGAAGGAATTCGCGAGTGGTGGGCTTTGAAACCTGGAGCCCCT |
| | Adeno- | CAACCCAAGGCAAATCAACAACATCAAGACAACGCTCGAGGTCT |
| | associated | TGTGCTTCCGGGTTACAAATACCTTGGACCCGGCAACGGACTCGA |
| | virus 9 | CAAGGGGGAGCCGGTCAACGCAGCAGACGCGGCGGCCCTCGAGC |
| | isolate hu.14 | ACGACAAGGCCTACGACCAGCAGCTCAAGGCCGGAGACAACCCG |
| | capsid | TACCTCAAGTACAACCACGCCGACGCCGAGTTCCAGGAGCGGCTC |
| | protein VP1 | AAAGAAGATACGTCTTTTGGGGGCAACCTCGGGCGAGCAGTCTTC |
| | (cap) gene, | CAGGCCAAAAAGAGGCTTCTTGAACCTCTTGGTCTGGTTGAGGAA |
| | complete cds | GCGGCTAAGACGGCTCCTGGAAAGAAGAGGCCTGTAGAGCAGTC |
| | | TCCTCAGGAACCGGACTCCTCCGCGGGTATTGGCAAATCGGGTG |
| | | CACAGCCCGCTAAAAAGAGACTCAATTTCGGTCAGACTGGCGACA |
| | | CAGAGTCAGTCCCAGACCCTCAACCAATCGGAGAACCTCCCGCAG |
| | | CCCCCTCAGGTGTGGGATCTCTTACAATGGCTTCAGGTGGTGGCG |
| | | CACCAGTGGCAGACAATAACGAAGGTGCCGATGGAGTGGGTAGT |
| | | TCCTCGGGAAATTGGCATTGCGATTCCCAATGGCTGGGGGACAGA |
| | | GTCATCACCACCAGCACCCGAACCTGGGCCCTGCCCACCTACAAC |
| | | AATCACCTCTACAAGCAAATCTCCAACAGCACATCTGGAGGATCT |
| | | TCAAATGACAACGCCTACTTCGGCTACAGCACCCCCTGGGGGTAT |
| | | TTTGACTTCAACAGATTCCACTGCCACTTCTCACCACGTGACTGGC |
| | | AGCGACTCATCAACAACAACTGGGGATTCCGGCCTAAGCGACTCA |
| | | ACTTCAAGCTCTTCAACATTCAGGTCAAAGAGGTTACGGACAACA |
| | | ATGGAGTCAAGACCATCGCCAATAACCTTACCAGCACGGTCCAGG |
| | | TCTTCACGGACTCAGACTATCAGCTCCCGTACGTGCTCGGGTCGG |
| | | CTCACGAGGGCTGCCTCCCGCCGTTCCCAGCGGACGTTTTCATGA |
| | | TTCCTCAGTACGGGTATCTGACGCTTAATGATGGAAGCCAGGCCG |
| | | TGGGTCGTTCGTCCTTTTACTGCCT |
| | | GGAATATTTCCCGTCGCAAATGCTAAGAACGGGTAACAACTTCCA |
| | | GTTCAGCTACGAGTTTGAGAACGTACCTTTCCATAGCAGCTACGC |
| | | TCACAGCCAAAGCCTGGACCGACTAATGAATCCACTCATCGACCA |
| | | ATACTTGTACTATCTCTCAAAGACTATTAACGGTTCTGGACAGAA |
| | | TCAACAAACGCTAAAATTCAGTGTGGCCGGACCCAGCAACATGGC |
| | | TGTCCAGGGAAGAAACTACATACCTGGACCCAGCTACCGACAAC |
| | | AACGTGTCTCAACCACTGTGACTCAAAACAACAACAGCGAATTTG |
| | | CTTGGCCTGGAGCTTCTTCTTGGGCTCTCAATGGACGTAATAGCTT |
| | | GATGAATCCTGGACCTGCTATGGCCAGCCACAAAGAAGGAGAGG |
| | | ACCGTTTCTTTCCTTTGTCTGGATCTTTAATTTTTGGCAAACAAGG |
| | | AACTGGAAGAGACAACGTGGATGCGGACAAAGTCATGATAACCA |
| | | ACGAAGAAGAAATTAAAACTACTAACCCGGTAGCAACGGAGTCC |
| | | TATGGACAAGTGGCCACAAACCACCAGAGTGCCCAAGCACAGGC |
| | | GCAGACCGGCTGGGTTCAAAACCAAGGAATACTTCCGGGTATGGT |
| | | TTGGCAGGACAGAGATGTGTACCTGCAAGGACCCATTTGGGCCAA |
| | | AATTCCTCACACGGACGGCAACTTTCACCCTTCTCCGCTGATGGG |
| | | AGGGTTTGGAATGAAGCACCCGCCTCCTCAGATCCTCATCAAAAA |
| | | CACACCTGTACCTGCGGATCCTCCAACGGCCTTCAACAAGGACAA |
| | | GCTGAACTCTTTCATCACCCAGTATTCTACTGGCCAAGTCAGCGTG |
| | | GAGATCGAGTGGGAGCTGCAGAAGGAAAACAGCAAGCGCTGGAA |
| | | CCCGGAGATCCAGTACACTTCCAACTATTACAAGTCTAATAATGT |
| | | TGAATTTGCTGTTAATACTGAAGGTGTATATAGTGAACCCCGCCC |
| | | CATTGGCACCAGATACCTGACTCGTAATCTGTAA |

### Methods of Producing rAAVs

Disclosed herein are methods of producing the AAV capsids comprising the AAV capsid proteins and viral vector encoding a therapeutic nucleic acid. The AAV capsid proteins are produced by introducing into a cell (e.g., immortalized stem cell) a first vector containing a transgene cassette flanked by inverted terminal repeat (ITR) sequences from a parental AAV virus (the transgene cassette has a promoter sequence that drives transcription of a heterologous nucleic acid in the nucleus of the target cell), a second vector encoding the AAV genome with a AAV capsid protein (encoding the AAV Rep gene as well as the modified Cap gene for the variant being produced), and a third vector encoding helper virus proteins, required for assembly of the AAV capsid structure and packaging of the transgene in the modified AAV capsid structure. The assembled AAV capsid can be isolated and purified from the cell using suitable methods known in the art. Tables 4-30 provide DNA sequences for using in the methods described herein.

The transgenes contained in a recombinant AAV (rAAV) vector and encapsidated by the AAV capsid proteins of the present disclosure are also provided herein. The transgenes disclosed herein are delivered to a subject for a variety of purposes, such as to treat a disease or condition in the subject. The transgene can be gene editing components that modulate the activity or expression of a target gene or gene expression product. Alternatively, the transgene is a gene encoding a therapeutic gene expression product that is effective to modulate the activity or expression of itself, or another target gene or gene expression product.

Aspects disclosed herein provide methods of manufacturing rAAV virus or virus particles comprising: (a) introducing into a cell a nucleic acid comprising: (i) first vector containing a transgene cassette flanked by inverted terminal repeat (ITR) sequences from a parental AAV virus (the transgene cassette has a promoter sequence that drives transcription of a heterologous nucleic acid in the nucleus of the target cell); (ii) a second vector encoding the AAV genome with a AAV capsid protein of the present invention; and (iii) a vector encoding helper virus proteins, required for assembly of the AAV capsid structure and packaging of the transgene in the modified AAV capsid structure; (b) expressing in the cell the AAV capsid protein described herein; (c) assembling an AAV particle comprising the AAV capsid proteins disclosed herein; and (d) packaging the AAV particle. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the Cap gene is derived from the deoxyribose nucleic acid (DNA) provided in any one of SEQ ID NOs: 6-10. In some instances, the 5′ ITR and the 3′ ITR are derived from an AAV2 serotype. In some instances, the 5′ ITR and the 3′ ITR are derived from an AAV5 serotype. In some instances, the 5′ ITR and the 3′ ITR are derived from an AAV9 serotype. In some instances, the first nucleic acid sequence and the second nucleic acid sequence are in trans. In some instances, the first nucleic acid sequence and the second nucleic acid sequence are in cis. In some instances, the first nucleic acid sequence, the second nucleic acid sequence and the third nucleic acid sequence, are in trans.

The Cap gene disclosed here comprises any one of SEQ ID NOS:385-654 from Tables 4-30, which are DNA sequences encoding the modified AAV capsid protein portions of the present disclosure.

In some instances, the methods comprise packing the first nucleic acid sequence encoding the therapeutic gene expression product such that it becomes encapsidated by the modified AAV capsid protein. In some embodiments, the rAAV particles are isolated, concentrated, and purified using suitable viral purification methods, such as those described herein.

In some cases, rAAVs of the present disclosure are generated using the methods described in Challis, R. C. et al. Nat. Protoc. 14, 379 (2019). Briefly, triple transfection of HEK293T cells (ATCC) using polyethylenimine (PEI) is performed, viruses are collected after 120 hours from both cell lysates and media and purified over iodixanol. In a non-limiting example, the rAAVs are generated by triple transfection of precursor cells (e.g., HEK293T) cells using a standard transfection protocol (e.g., PEI). Viral particles are harvested from the media after a period of time (e.g., 72 h post transfection) and from the cells and media at a later point in time (e.g., 120 h post transfection). Virus present in the media is concentrated by precipitation with 8% polyethylene glycol (PEG) and 500 mM sodium chloride and the precipitated virus is added to the lysates prepared from the collected cells. The viruses are purified over iodixanol (Optiprep, Sigma) step gradients (15%, 25%, 40% and 60%). Viruses are concentrated and formulated in PBS. Virus titers are determined by measuring the number of DNaseI-resistant vector genome copies (VGs) using qPCR and the linearized genome plasmid as a control.

The cell can be selected from a human, a primate, a murine, a feline, a canine, a porcine, an ovine, a bovine, an equine, an epine, a caprine and a lupine host cell. In some instances, the cell is a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some cases, the immortalized cell is a HEK293cell. In some instances, the cell is a differentiated cell. Based on the disclosure provided, it is expected that this system can be used in conjunction with any transgenic line expressing a recombinase in the target cell type of interest to develop AAV capsids that more efficiently transduce that target cell population.

### Methods of Treatment

Disclosed herein are methods of treating a disease or condition, or a symptom of the disease or condition, in a subject, comprising administrating of therapeutically effective amount of one or more compositions (e.g., rAAV particle, AAV vector, pharmaceutical composition) disclosed herein to the subject. In some embodiments, the composition is a rAAV capsid protein described herein. In some embodiments, the composition is an isolated and purified rAAV capsid protein described herein. In some embodiments, the rAAV particle encapsidates an AAV vector comprising a transgene (e.g., therapeutic nucleic acid). In some embodiments, the composition is a rAAV capsid protein described herein conjugated with a therapeutic agent disclosed herein. In some embodiments, the composition is a pharmaceutical composition comprising the rAAV particle and a pharmaceutically acceptable carrier. In some embodiments, the one or more compositions are administered to the subject alone (e.g., stand-alone therapy). In some embodiments, the composition is a first-line therapy for the disease or condition. In some embodiments, the composition is a second-line, third-line, or fourth-line therapy, for the disease or condition.

Recombinant adeno-associated virus (rAAV) mediated gene delivery leverages the AAV mechanism of viral transduction for nuclear expression of an episomal heterologous nucleic acid (e.g., a transgene, therapeutic nucleic acid). Upon delivery to a host in vivo environment, a rAAV will (1) bind or attach to cellular surface receptors on the target cell, (2) endocytose, (3) traffic to the nucleus, (4) uncoat the virus to release the encapsidated heterologous nucleic acid, (5) convert of the heterologous nucleic acid from single-stranded to double-stranded DNA as a template for transcription in the nucleus, and (6) transcribe of the episomal heterologous nucleic acid in the nucleus of the host cell (“transduction”). rAAVs engineered to have an increased specificity (binding to cellular surface receptors on the target cell) and transduction efficiency (transcription of the episomal heterologous nucleic acid in the host cell) are desirable for gene therapy applications.

Aspects disclosed herein provide methods of treating a disease or condition in a subject, the method comprising administering to the subject a therapeutically effective amount of the rAAV of the present disclosure, or the pharmaceutical formulation of the present disclosure, wherein the gene product is a therapeutic gene product. In some embodiments, the administering is by irracranial, intraventricular, intracerebroventricular, intravenous, intraarterial, intranasal, intrathecal, intracisternae magna, or subcutaneous.

Provided here, are methods of treating a disease or a condition associated with an aberrant expression or activity of a target gene or gene expression product thereof, the method comprising modulating the expression or the activity of a target gene or gene expression product in a subject by administering a rAAV encapsidating a heterologous nucleic acid of the present disclosure. In some instances, the expression or the activity of the target gene or gene expression product is decreased, relative to that in a normal (non-diseased) individual; and administering the rAAV to the subject is sufficient to increase the expression of the activity of the target gene or gene expression product. In some instances, the expression or the activity of the gene or gene expression product is increased, relative to that in a normal individual; and administering the rAAV to the subject is sufficient to decrease the expression or the activity of the target gene or gene expression product. In a non-limiting example, a subject diagnosed with Alzheimer's disease, which is caused, in some cases, by a gain-of-function of a Presenilin 1 and/or Presenilin 2 (encoded by the gene PSEN1 and PSEN2, respectively) is administered a rAAV disclosed herein encapsidating a therapeutic nucleic acid that is a silencing RNA (siRNA), or other RNAi with a loss-of-function effect on PSEN1 mRNA.

Also provided are methods of preventing a disease or condition disclosed herein in a subject comprising administering to the subject a therapeutically effective amount of an rAAV vector comprising a nucleic acid sequence encoding a therapeutic gene expression product described herein. The rAAV vector may be encapsidated in the modified capsid protein or rAAV viral particle described herein. In some instances, the therapeutic gene expression product is effective to modulate the activity or expression of a target gene or gene expression product.

Disclosed herein are methods of treating a disease or condition in a subject by administering a composition comprising a rAAV disclosed herein. An advantage of the rAAVs disclosed herein, is that the rAAV may be used to treat virtually any disease or condition that would benefit from a transgene therapy, including but not limited to spinal muscular atrophy (SMA), amyotrophic lateral sclerosis (ALS), Parkinson's disease, Pompe disease, mucopolysaccharidosis type II, fragile X syndrome, STXBP1 encephalopathy. Krabbe disease, Huntington's disease, Alzheimer's disease, Battens disease, lysosomal storage disorders, glioblastoma multiforme, Rett syndrome, Leber's congenital amaurosis, Late infantile neuronal ceroid lipofuscinosis (LINCL), chronic pain, stroke, spinal cord injury, traumatic brain injury and lysosomal storage disorders.

In some cases, the disease or condition is localized to a particular in vivo environment in the subject, e.g., the CNS. The compositions of the present disclosure are particularly useful for the treatment of the diseases or conditions described herein because they specifically or more efficiently target the in vivo environment and deliver a therapeutic nucleic acid engineered to modulate the activity or the expression of a target gene expression product involved with the pathogenesis or pathology of the disease or condition.

Provided herein are methods of treating a disease or a condition, or a symptom of the disease or condition, in a subject, comprising: (a) diagnosing a subject with a disease or a condition affecting a target in vivo environment; and (b) treating the disease or the condition by administering to the subject a therapeutically effective amount of a composition disclosed herein (e.g., rAAV particle, AAV vector, pharmaceutical composition), wherein the composition is engineered with an increased specificity for the target in vivo environment.

Disclosed herein are methods of treating a disease or a condition, or a symptom of the disease or the condition, afflicting a target in a subject comprising: (a) administering to the subject a composition (e.g., rAAV particle, AAV vector, pharmaceutical composition); and (b) expressing the therapeutic nucleic acid into a target in vivo environment in the subject with an increased specificity and/or transduction efficiency.

In some embodiments, methods further comprise reducing or ablating delivery of the heterologous nucleic acid in an off-target in vivo environment, such as the liver. In some embodiments, delivery is characterized by an increase in efficiency of transduction (e.g., of the heterologous nucleic acid) in the CNS.

In some embodiments, methods of treating a disease or condition affecting the CNS comprise administering a rAAV particle to a CNS in a subject, the rAAV particle comprising an rAAV capsid protein comprising an insertion of about, five, six, or seven amino acids of an amino acid sequence provided in Tables 1 and 4-30, FIG. 4 and/or Formulas I-XXXIII, at an amino acid position 588-589 in a parental AAV capsid protein. In some embodiments, the parental AAV capsid protein is AAV9 capsid protein (for e.g., provided in SEQ ID NO: 1.

Also provided are methods of modulating a target gene expression product, the methods comprising administering to a subject in need thereof a composition (e.g., rAAV particle, AAV vector, pharmaceutical composition) disclosed herein. For example, methods provided herein comprise administering to a subject a rAAV with a rAAV capsid protein encapsidating a viral vector comprising a heterologous nucleic acid that modulates the expression or the activity of the target gene expression product.

The term “normal individual” refers to an individual that is not afflicted with the disease or the condition characterized by the variation in expression or activity of the gene or gene expression product thereof.

In some embodiments, the disease or condition of the CNS is selected from Absence of the Septum Pellucidum, Acid Lipase Disease, Acid Maltase Deficiency, Acquired Epileptiform Aphasia, Acute Disseminated Encephalomyelitis, Attention Deficit-Hyperactivity Disorder (ADHD), Adie's Pupil, Adie's Syndrome, Adrenoleukodystrophy, Agenesis of the Corpus Callosum, Agnosia, Aicardi Syndrome, Aicardi-Goutieres Syndrome Disorder, AIDS-Neurological Complications, Alexander Disease, Alpers' Disease, Alternating Hemiplegia, Alzheimer's Disease, Amyotrophic Lateral Sclerosis (ALS), Anencephaly, Aneurysm, Angelman Syndrome, Angiomatosis, Anoxia, Antiphospholipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Atrial Fibrillation and Stroke, Attention Deficit-Hyperactivity Disorder, Autism Spectrum Disorder, Autonomic Dysfunction, Back Pain, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Birth Injuries, Brachial Plexus Injuries, Bradbury-Eggleston Syndrome, Brain and Spinal Tumors, Brain Aneurysm, Brain Injury, Brown-Sequard Syndrome, Bulbospinal Muscular Atrophy, Cerebral Autosomal Dominant Arteriopathy with Subcortical Infarcts and Leukoencephalopathy (CADASIL), Canavan Disease, Carpal Tunnel Syndrome, Causalgia, Cavernomas, Cavernous Angioma, Cavernous Malformation, Central Cervical Cord Syndrome, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysms, Cerebral Arteriosclerosis, Cerebral Atrophy, Cerebral Beriberi, Cerebral Cavemous Malformation, Cerebral Gigantism, Cerebral Hypoxia, Cerebral Palsy, Cerebro-Oculo-Facio-Skeletal Syndrome (COFS), Charcot-Marie-Tooth Disease, Charcot-Marie-Tooth syndrome, classical rhizomelic chondrodysplasia punctata (RCDP), Chiari Malformation, Cholesterol Ester Storage Disease, Chorea, Choreoacanthocytosis, Chronic Inflammatory Demyelinating Polyneuropathy (CIDP), Chronic Orthostatic Intolerance, Chronic Pain, Cockayne Syndrome, Cockayne Syndrome Type II, Coffin Lowry Syndrome, Colpocephaly, Coma, Complex Regional Pain Syndrome, Congenital Facial Diplegia, Congenital Myasthenia, Congenital Myopathy, Congenital Vascular Cavernous Malformations, Corticobasal Degeneration, Cranial Arteritis, Craniosynostosis, Cree encephalitis, Creutzfeldt-Jakob Disease, Cumulative Trauma Disorders, Cushing's Syndrome, Cytomegalic Inclusion Body Disease, Cytomegalovirus Infection, Dancing Eyes-Dancing Feet Syndrome, Dandy-Walker Syndrome, Dawson Disease, Deafness, De Morsier's Syndrome, Dejerine-Klumpke Palsy, Dementia, Dementia-Multi-Infarct, Dementia-Semantic, Dementia-Subcortical, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Duchenne muscular dystrophy, Dysautonomia, Dysgraphia, Dyslexia, Dysphagia, Dyspraxia, Dyssynergia Cerebellaris Myoclonica, Dyssynergia Cerebellaris Progressiva, Dystonias, Early Infantile Epileptic Encephalopathy, Empty Sella Syndrome, Encephalitis, Encephalitis Lethargica, Encephaloceles, Encephalopathy, Encephalopathy (familial infantile), Encephalotrigeminal Angiomatosis, Epilepsy, Epileptic Hemiplegia, Erb's Palsy, Erb-Duchenne and Dejerine-Klumpke Palsies, Essential Tremor, Extrapontine Myelinolysis, Fabry Disease, Fahr's Syndrome, Fainting, Familial Dysautonomia, Familial Hemangioma, Familial Idiopathic Basal Ganglia Calcification, Familial Periodic Paralyses, Familial Spastic Paralysis, Farber's Disease, Febrile Seizures, Fibromuscular Dysplasia, Fisher Syndrome, Floppy Infant Syndrome, Foot Drop, Fragile X syndrome, Friedreich's Ataxia, Frontotemporal Dementia (FTD), Gaucher Disease, Generalized Gangliosidoses, Gerstmann's Syndrome, Gerstmann-Straussler-Scheinker Disease, Giant Axonal Neuropathy, Giant Cell Arteritis, Giant Cell Inclusion Disease, glioblastoma, Globoid Cell Leukodystrophy, Glossopharyngeal Neuralgia, Glycogen Storage Disease, Guillain-Barre Syndrome, Hallervorden-Spatz Disease, Head Injury, Headache, Hemicrania Continua, Hemifacial Spasm, Hemiplegia Alterans, Hereditary Neuropathies, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydrocephalus—Normal Pressure, Hydromyelia, Hypercortisolism, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease (IRD), Infantile Spasms, Inflammatory Myopathies, Iniencephaly, Intestinal Lipodystrophy, Intracranial Cysts, Intracranial Hypertension, Isaacs' Syndrome, Joubert Syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kliiver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Femoral Cutaneous Nerve Entrapment, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus-Neurological Sequelae, Lyme Disease—Neurological Complications, Machado-Joseph Disease, Macrencephaly, Maple syrup urine disease, Megalencephaly, Melkersson-Rosenthal Syndrome, Meningitis, Meningitis and Encephalitis, Menkes Disease, Menkes syndrome, Meralgia Paresthetica, Metachromatic Leukodystrophy, Microcephaly, Migraine, Miller Fisher Syndrome, Mini Stroke, Mitochondrial Myopathy, Moebius Syndrome, Monomelic Amyotrophy, Motor Neuron Diseases, Moyamoya Disease, Mucolipidoses, Mucopolysaccharidosis, Mucopolysaccharidosis II, Multi-Infarct Dementia, Multifocal Motor Neuropathy, Multiple Sclerosis, Multiple System Atrophy, Multiple System Atrophy with Orthostatic Hypotension, Muscular Dystrophy, Myasthenia-Congenital, Myasthenia Gravis, Myelinoclastic Diffuse Sclerosis, Myoclonic Encephalopathy of Infants, Myoclonus, Myopathy, Myopathy-Congenital, Myopathy-Thyrotoxic, Myotonia, Myotonia Congenita, Myotonic dystrophy, Narcolepsy, Neuroacanthocytosis, Neurodegeneration with Brain Iron Accumulation, Neurofibromatosis, Neuroleptic Malignant Syndrome, Neurological Complications of AIDS, Neurological Complications of Lyme Disease, Neurological Consequences of Cytomegalovirus Infection, Neurological Manifestations of Pompe Disease, Neurological Sequelae Of Lupus, Neuromyelitis Optica, Neuromyotonia, Neuronal Ceroid Lipofuscinosis, Neuronal Migration Disorders, Neuropathy-Hereditary, Neurosarcoidosis, Neurosyphilis, Neurotoxicity, Nevus Cavernosus, Niemann-Pick Disease, O'Sullivan-McLeod Syndrome, Occipital Neuralgia, Ohtahara Syndrome, Olivopontocerebellar Atrophy, Opsoclonus Myoclonus, Orthostatic Hypotension, Overuse Syndrome, Pain-Chronic, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Pena Shokeir II Syndrome, Perineural Cysts, Periodic Paralyses, Peripheral Neuropathy, Periventricular Leukomalacia, Persistent Vegetative State, Pervasive Developmental Disorders, Phenylketonuria, Phytanic Acid Storage Disease, Pick's Disease, Pinched Nerve, Piriformis Syndrome, Pituitary Tumors, Polymyositis, Pompe Disease, Porencephaly, Post-Polio Syndrome, Postherpetic Neuralgia, Postinfectious Encephalomyelitis, Postural Hypotension, Postural Orthostatic Tachycardia Syndrome, Postural Tachycardia Syndrome, Prader-Willi syndrome, Primary Dentatum Atrophy, Primary Lateral Sclerosis, Primary Progressive Aphasia, Prion Diseases, Progressive Hemifacial Atrophy, Progressive Locomotor Ataxia, Progressive Multifocal Leukoencephalopathy, Progressive Sclerosing Poliodystrophy, Progressive Supranuclear Palsy, Prosopagnosia, Pseudo-Torch syndrome, Pseudotoxoplasmosis syndrome, Pseudotumor Cerebri, Psychogenic Movement, Ramsay Hunt Syndrome I, Ramsay Hunt Syndrome II, Rasmussen's Encephalitis, Reflex Sympathetic Dystrophy Syndrome, Refsum Disease, Refsum Disease—Infantile, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Sacral Nerve Root Cysts, Saint Vitus Dance, Salivary Gland Disease, Sandhoff Disease, Schilder's Disease, Schizencephaly, Seitelberger Disease, Seizure Disorder, Semantic Dementia, Septo-Optic Dysplasia, Severe Myoclonic Epilepsy of Infancy (SMEI), Shaken Baby Syndrome, Shingles, Shy-Drager Syndrome, Sjogren's Syndrome, Sleep Apnea, Sleeping Sickness, Sotos Syndrome, Spasticity, Spina Bifida, Spinal Cord Infarction, Spinal Cord Injury, Spinal Cord Tumors, Spinal Muscular Atrophy, Spinocerebellar ataxia, Spinocerebellar Atrophy, Spinocerebellar Degeneration, Steele-Richardson-Olszewski Syndrome, Stiff-Person Syndrome, Striatonigral Degeneration, Stroke, Sturge-Weber Syndrome, STXBP1 encephalopathy, Subacute Sclerosing Panencephalitis, Subcortical Arteriosclerotic Encephalopathy, Short-lasting, Unilateral, Neuralgiform (SUNCT) Headache, Swallowing Disorders, Sydenham Chorea, Syncope, Syphilitic Spinal Sclerosis, Syringohydromyelia, Syringomyelia, Systemic Lupus Erythematosus, Tabes Dorsalis, Tangier disease, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tic Douloureux, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vascular Erectile Tumor, Vasculitis Syndromes of the Central Nervous Systems, Von Economo's Disease, Von Hippel-Lindau Disease (VHL), Von Hippel-Lindau syndrome, Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson Disease, Wolman's Disease, X-Linked Spinal and Bulbar Muscular Atrophy and Zellweger syndrome.

In some embodiments, the pharmaceutical formulation comprises a therapeutic nucleic acid encoding a therapeutic gene expression product. In some instances, the therapeutic gene expression product is effective to modulate an activity or an expression of a target gene or gene expression product selected from ATP1A2, CACNAIA, SETD5, SHANK3, NF2, DNMT1, TCF4, RAI1, PEX1, ARSA, EIF2B5, EIF2B1, EIF2B2, NPCl, ADAR, MFSD8, STXBP1, PRICKLE2, PRRT2, IDUA, STX1B, Sarcoglycan Alpha (SGCA), glutamic acid decarboxylase 65 (GAD65), glutamic acid decarboxylase 67 (GAD67), CLN2, Nerve Growth Factor (NGF), glial cell derived neurotrophic factor (GDNF), Survival Of Motor Neuron 1, STXBP1, Telomeric (SMNI), Factor X (FIX), Retinoid Isomerohydrolase (RPE65), sarco/endoplasmic reticulum Ca2+-ATPase (SERCA2a), Glucocerebrosidase (GCase), galactocerebrosidase (GALC), CDKL5, Frataxin (FXN), Huntingtin (HTT), methyl-CpG binding protein 2 (MECP2), a peroxisomal biogenesis factor (PEX), progranulin (GRN), an antitubulin agent, copper-zinc superoxide dismutase (SODI), iduronate 2 sulfatase (hIDS), Glucosylceramidase Beta (GBA), fragile X mental retardation 1 (FMR1), NPC Intracellular Cholesterol Transporter 1 (NPCl), SCN1A, C9orf72, NPS3 and a NLRP3 inflammasome. In some embodiments, the peroxisomal biogenesis factor (PEX) is selected from PEX1, PEX2, PEX3, PEX4, PEX5, PEX6, PEX7, PEX10, PEX110, PEX12, PEX13, PEX14, PEX16, PEX19, and PEX26.

In some aspects, other examples of genes involved in CNS diseases or disorders include MAPT, IDUA, SNCA, ATXN2, Ube3a, GNS, HGSNAT, NAGLU, SGSH, CLN1, CLN3, CLN4, CLN5, CLN6, CLN7, CLN8, CTSD, ABCD1, HEXA, HEXB, ASM, ASPA, GLB1, AADC, MFN2, GNAO1, SYNGAP1, GRIN2A, GRIN2B, KCNQ2, EPM2A, NHLRC1, SLC6A1, SLC13A5, SURF1, GBE1, ATXN1, ATXN3, and ATXN7.

In some instances, the therapeutic gene expression product comprises gene editing components. In some instances, the gene editing components are selected from an artificial site-specific RNA endonuclease (ASRE), a zinc finger endonuclease (ZFN), a transcription factor like effector nuclease (TALEN), a clustered regularly interspaced short palindromic repeats (CRISPR)/Cas enzyme, and a CRISPR)/Cas guide RNA.

In some instances, the expression of a gene or expression or activity of a gene expression product is inhibited by the administration of the composition to the subject. In some instances, the expression of a gene or the expression or the activity of a gene expression product is enhanced by the administration of the composition to the subject.

### Formulations, Dosages, and Routes of Administration

Disclosed herein are methods comprising delivering a rAAV particle encapsidating a heterologous nucleic acid to the CNS in a subject, the rAAV particle comprising (i) an increased transduction of the heterologous nucleic acid in the CNS, wherein the rAAV particle has an rAAV capsid protein comprising an insertion of five, six, or seven amino acids of an amino acid sequence provided in Tables 1 and 4-30, FIG. 4 and/or Formulas I-XXXIII, at an amino acid position 588-589 in a parental AAV capsid protein.

In general, methods disclosed herein comprise administering a therapeutic rAAV composition by systemic administration. In some instances, methods comprise administering a therapeutic rAAV composition by intravenous (“i.v.”) administration. One may administer therapeutic rAAV compositions by additional routes, such as subcutaneous injection, intramuscular injection, intradermal injection, transdermal injection, percutaneous administration, intranasal administration, intralymphatic injection, rectal administration intragastric administration, intraocular administration, intracerebroventricular administration, intrathecally, intracisternal, or any other suitable parenteral administration. Routes, dosage, time points, and duration of administrating therapeutics may be adjusted. In some embodiments, administration of therapeutics is prior to, or after, onset of either, or both, acute and chronic symptoms of the disease or condition. Other routes of delivery to the CNS include, but are not limited to intracranial administration, lateral cerebroventricular administration, and endovascular administration.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration may be changed, or an additional agent may be administered to the subject, along with the therapeutic rAAV composition. In some embodiments, as a patient is started on a regimen of a therapeutic rAAV composition, the patient is also weaned off (e.g., step-wise decrease in dose) a second treatment regimen.

In some cases, a dose of the pharmaceutical composition may comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, or 2×10¹⁷. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, or 3×10¹⁷. In some cases, the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, or 4×10¹⁷. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, or 5×10¹⁷. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, or 6×10¹⁷. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, or 7×10¹⁷. In some cases, the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, or 8×10¹⁷. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, or 9×10¹⁷.

Disclosed herein, in some embodiments are formulations of pharmaceutically-acceptable excipients and carrier solutions suitable for delivery of the rAAV compositions described herein, as well as suitable dosing and treatment regimens for using the particular compositions described herein in a variety of treatment regimens. In some embodiments, the amount of therapeutic gene expression product in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In some embodiments, the pharmaceutical forms of the rAAV-based viral compositions suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

In some cases, for administration of an injectable aqueous solution, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions may be advantageous for systemic administration, for example by intravenous or intrathecal administration.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of rAAV compositions and time of administration of such compositions will be within the purview of the skilled artisan having benefit of the present teachings. It is likely, however, that the administration of therapeutically-effective amounts of the disclosed compositions may be achieved by a single administration, example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

In certain embodiments, the data obtained from cell culture assays and animal studies are used in formulating the therapeutically effective daily dosage range and/or the therapeutically effective unit dosage amount for use in mammals, including humans. In certain embodiments, the dosage range and/or the unit dosage amount varies within this range depending upon the dosage form employed and the route of administration utilized.

### Combination Therapies

A therapeutic rAAV may be used alone or in combination with an additional therapeutic agent (together, “therapeutic agents”). In some cases, a therapeutic rAAV as used herein is administered alone. The therapeutic agent may be administered together or sequentially in a combination therapy. The combination therapy may be administered within the same day, or may be administered one or more days, weeks, months, or years apart.

The additional therapeutic agent can comprise a small molecule. The additional therapeutic agent can comprise an antibody, or antigen-binding fragment. The additional therapeutic agent can include lipid nanoparticle-based therapies, anti-sense oligonucleotide therapies, as well as other viral therapies.

The additional therapeutic agent can comprise a cell-based therapy. Exemplary cell-based therapies include without limitation immune effector cell therapy, chimeric antigen receptor T-cell (CAR-T) therapy, natural killer cell therapy and chimeric antigen receptor natural killer (NK) cell therapy. Either NK cells, or CAR-NK cells, or a combination of both NK cells and CAR-NK cells can be used in combination with the methods disclosed herein. In some embodiments, the NK cells and CAR-NK cells are derived from human induced pluripotent stem cells (iPSC), umbilical cord blood, or a cell line. The NK cells and CAR-NK cells can comprise a cytokine receptor and a suicide gene. The cell-based therapy can comprise a stem cell therapy. The stem cell therapy may be embryonic or somatic stem cells. The stem cells may be isolated from a donor (allogeneic) or isolated from the subject (autologous). The stem cells may be expanded adipose-derived stem cells (eASCs), hematopoietic stem cells (HSCs), mesenchymal stem (stromal) cells (MSCs), or induced pluripotent stem cells (iPSCs) derived from the cells of the subject.

### KITS

Disclosed herein are kits comprising compositions disclosed herein. Also disclosed herein are kits for the treatment or prevention of a disease or conditions of the CNS. In some instances, the disease or condition is cancer, a pathogen infection, pulmonary disease or condition, neurological disease, muscular disease, or an immune disorder, such as those described herein.

In one embodiment, a kit can include a therapeutic or prophylactic composition containing an effective amount of a composition of a rAAV particle encapsidating a recombinant AAV vector encoding a therapeutic nucleic acid (e.g., therapeutic nucleic acid) and a recombinant AAV (rAAV) capsid protein of the present disclosure. In another embodiment, a kit can include a therapeutic or prophylactic composition containing an effective amount of cells modified by the rAAV described herein (“modified cell”), in unit dosage form that express therapeutic nucleic acid. In some embodiments, a kit comprises a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In some instances, the kit further comprises a cell. In some instances, the cell is mammalian. In some instances, the cell is immortalized. In some instances, the immortalized cell is an embryonic stem cell. In some instances, the embryonic stem cell is a human embryonic stem cell. In some instances, the human embryonic stem cell is a human embryonic kidney 293 (HEK-293) cell. In some instances, the kit further comprises an AAV vector comprising a heterologous nucleic acid encoding a therapeutic gene expression product. In some instances, the AAV vector is an episome.

In some cases, rAAV are provided together with instructions for administering the rAAV to a subject having or at risk of developing the disease or condition (e.g., disease of the CNS). Instructions can generally include information about the use of the composition for the treatment or prevention of the disease or condition.

In some cases, the instructions include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. In some cases, instructions provide procedures for administering the rAAV to the subject alone. In some instances, the instructions provide that the rAAV is formulated for systemic delivery.

### Definitions

The terminology used herein is for the purpose of describing particular cases only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” should be assumed to mean an acceptable error range for the particular value.

As used herein “consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure, such as compositions for treating skin disorders like acne, eczema, psoriasis, and rosacea.

The terms “homologous,” “homology,” or “percent homology” are used herein to generally mean an amino acid sequence or a nucleic acid sequence having the same, or similar sequence to a reference sequence. Percent homology of sequences can be determined using the most recent version of BLAST, as of the filing date of this application.

The terms “increased,” or “increase” are used herein to generally mean an increase by a statically significant amount. In some embodiments, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms, “decreased” or “decrease” are used herein generally to mean a decrease by a statistically significant amount. In some embodiments, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease.

The terms “subject” is any organism. In some instances, the organism is a mammal. Non-limiting examples of mammal include, any member of the mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human. The term “animal” as used herein comprises human beings and non-human animals. In one embodiment, a “non-human animal” is a mammal, for example a rodent such as rat or a mouse. In one embodiment, a “non-human primate” is a mammal, for example a monkey. In some instances, the subject is a patient, which as used herein, may refer to a subject diagnosed with a particular disease or disorder.

The term “gene,” as used herein, refers to a segment of nucleic acid that encodes an individual protein or RNA (also referred to as a “coding sequence” or “coding region”), optionally together with associated regulatory region such as promoter, operator, terminator and the like, which may be located upstream or downstream of the coding sequence.

The term “adeno-associated virus,” or “AAV” as used herein refers to the adeno-associated virus or derivatives thereof. Non-limited examples of AAV's include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), avian AAV, bovine AAV, canine AAV, equine AAV, primate AAV, non-primate AAV, and ovine AAV. In some instances, the AAV is described as a “Primate AAV,” which refers to AAV that infect primates. Likewise an AAV may infect bovine animals (e.g., “bovine AAV”, and the like). In some instances, the AAV is wildtype, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a transgene, therapeutic nucleic acid, viral genome). In some instances, the AAV capsid of the instant disclosure is a modified AAV capsid, relative to a corresponding parental AAV capsid protein.

The term “tropism” as used herein refers to a quality or characteristic of the AAV capsid that may include specificity for, and/or an increase or a decrease in efficiency of, expressing the encapsidated genetic information into an in vivo environment, relative to a second in vivo environment. An in vivo environment, in some instances, is a cell-type. An in vivo environment, in some instances, is an organ or organ system.

The term “AAV vector” as used herein refers to nucleic acid polymer encoding genetic information related to the virus. The AAV vector may be a recombinant AAV vector (rAAV), which refers to an AAV vector generated using recombinatorial genetics methods. In some instances, the rAAV vector comprises at least one heterologous polynucleotide (e.g. a polynucleotide other than a wild-type or naturally occurring AAV genome such as a transgene).

The term “AAV particle” as used herein refers to an AAV virus, virion, AAV capsid protein or component thereof. In some cases, the AAV particle is modified relative to a parental AAV particle.

The term “gene product” of “gene expression product” refers to an expression product of a polynucleotide sequence such as, for e.g., a polypeptide, peptide, protein or RNA, including interfering RNA (e.g., siRNA, miRNA, shRNA) and messenger RNA (mRNA).

The term “heterologous” as used herein refers to a genetic element (e.g., coding region) or gene expression product (e.g., RNA, protein) that is derived from a genotypically distinct entity from that of the rest of the entity to which it is being compared.

The term “endogenous” as used herein refers to a genetic element (e.g., coding region) or gene expression product (e.g., RNA, protein) that is naturally occurring in or associated with an organism or a particular cell within the organism.

The terms “treat,” “treating,” and “treatment” as used herein refers to alleviating or abrogating a disorder, disease, or condition; or one or more of the symptoms associated with the disorder, disease, or condition; or alleviating or eradicating a cause of the disorder, disease, or condition itself. Desirable effects of treatment can include, but are not limited to, preventing occurrence or recurrence of disease, alleviation of symptoms, diminishing any direct or indirect pathological consequences of the disease, preventing metastasis, decreasing the rate of disease progression, amelioration or palliation of the disease state and remission or improved prognosis.

The term “therapeutically effective amount” refers to the amount of a compound or therapy that, when administered, is sufficient to prevent development of, or alleviate to some extent, one or more of the symptoms of a disorder, disease, or condition of the disease; or the amount of a compound that is sufficient to elicit biological or medical response of a cell, tissue, system, animal, or human that is being sought by a researcher, veterinarian, medical doctor, or clinician.

The term “pharmaceutically acceptable carrier,” “pharmaceutically acceptable excipient,” “physiologically acceptable carrier,” or “physiologically acceptable excipient” refers to a pharmaceutically acceptable material, composition, or vehicle, such as a liquid or solid filler, diluent, excipient, solvent, or encapsulating material. A component can be “pharmaceutically acceptable” in the sense of being compatible with the other ingredients of a pharmaceutical formulation. It can also be suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st Edition; Lippincott Williams & Wilkins: Philadelphia, P A, 2005; Handbook of Pharmaceutical Excipients, 5th Edition; Rowe et al., Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, F L, 2004).

The term “pharmaceutical composition” refers to a mixture of a compound disclosed herein with other chemical components, such as diluents or carriers. The pharmaceutical composition can facilitate administration of the compound to an organism. Multiple techniques of administering a compound exist in the art including, but not limited to, systemic administration.

Non-limiting examples of “sample” include any material from which nucleic acids and/or proteins can be obtained. As non-limiting examples, this includes whole blood, peripheral blood, plasma, serum, saliva, mucus, urine, semen, lymph, fecal extract, cheek swab, cells or other bodily fluid or tissue, including but not limited to tissue obtained through surgical biopsy or surgical resection. Alternatively, a sample can be obtained through primary patient derived cell lines, or archived patient samples in the form of preserved samples, or fresh frozen samples.

The term “in vivo” is used to describe an event that takes place in a subject's body.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

The term “CNS” or “central nervous system” means a tissue selected from brain, thalamus, cortex, putamen, lateral ventricles, medulla, the pons, the amygdala, the motor cortex, caudate, hypothalamus, striatum, ventral midbrain, neocortex, basal ganglia, hippocampus, cerebrum, cerebellum, brain stem, and spinal cord. The brain includes a variety of cortical and subcortical areas, including the frontal, temporal, occipital and parietal lobes.

The term “systemic delivery” is defined as a route of administration of medication or other substance into a circulatory system so that the entire body is affected, Administration can take place via enteral administration (absorption of the drug through the gastrointestinal tract) or parenteral administration (generally injection, infusion, or implantation). “Circulatory system” includes both blood or cerebrospinal fluid circulatory systems. Examples of systemic administration for the CNS include intraarterial, intravenous or intrathecal injection. Other examples include administration to the cerebrospinal fluid at any location, in the spine (i.e. but not limited to lumbar) or brain (i.e. but not limited to cisterna magna). The terms “systemic administration” and “systemic delivery” are used interchangeably.

The section headings used herein are for organizational purposes only and are not to be construed as limiting the subject matter described.

### EXAMPLES

### Example 1

### Method of Identifying the Modified Capsid Proteins in Marmosets

Of primary concern for the therapeutic applicability of engineered adeno-associated viruses (AAVs) is how well their transduction profiles translate to human application. While previous engineering efforts have focused on in vitro or in vivo rodent screening platforms due to the ease and flexibility of their use, screening efforts directly in non-human primates (NHPs) are much more likely to identify viruses that translate. We chose marmosets, a new world NHP, for our engineering efforts. We focused our engineering efforts on a region of the AAV9 capsid surface located at amino acid position 588, one of the most exposed loops on the capsid surface that is a variable region between natural AAV serotypes and has a role in receptor binding. Insertion of peptides between positions 588 and 589 has been studied in the past by us, and others, and has resulted in novel receptor binding (AAV-PHP.B/AAV-PHP.eB binding of Ly6a on rodent brain endothelium to facilitate blood-brain barrier crossing and high transduction of the brain) and drastically altered capsid tropism. We chose to create a library of viral capsid by performing a random 7 amino acid insertion at this site within AAV9, hoping for novel tropism toward the NHP CNS.

Plasmids. The first-round viral DNA library was generated by amplification of a section of the AAV9 capsid genome between amino acids 450-599 using NNK degenerate primers (Integrated DNA Technologies, Inc., IDT) to insert seven random amino acids between amino acids 588 and 589 with all possible variations. The resulting library inserts were then introduced into the rAAV-ΔCap-in-cis-Lox plasmid via Gibson assembly as previously described (Deverman et al., Nat Biotechnol. 2016 February; 34(2): 204-209). The resulting capsid DNA library, rAAV-Cap-in-cis-Lox, contained a diversity of ˜1.28 billion variants at the amino acid level. The second round viral DNA library was generated similarly to the first round, but instead of NNK degenerate primers inserted at the 588, a synthesized oligo pool (Twist Biosicence) was used to generate only selected variants. This second-round DNA library contained a diversity of 33,287 variants at the amino acid level, and 66,574 variants at the DNA level (the 33,287 pulled out of the first round and a codon-modified version of each).

The AAV2/9 REP-AAP-ΔCAP plasmid transfected into HEK293T cells to provide the Rep gene for library viral production prevents production of a wild-type AAV9 capsid during viral library production after a plausible recombination event between this plasmid co-transfected with rAAV-ΔCap-in-cis-Lox containing the library inserts.

Viral production. Recombinant AAVs were generated according to established protocols. Briefly, immortalized HEK293T cells (ATCC) were quadruple transfected with four vectors using polyethylenimine (PEI). The first vector was the rAAV-Cap-in-cis-Lox library flanked by inverted terminal repeat (ITR) sequences from a parental AAV virus. The second vector was the AAV2/9 REP-AAP-ΔCAP plasmid. The third vector contains nucleic acids encoding helper virus proteins needed for viral assembly and packaging of the heterologous nucleic acid into the modified capsid structure. The fourth is a pUC-18 plasmid included to achieve the right PEI/DNA ratio for optimal transfection efficiency. Only 10 ng of rAAV-Cap-in-cis-Lox library DNA was transfected (per 150 mm plate) to decrease the likelihood of multiple library DNAs entering the same cell. Viral particles are harvested from the cells and media after 60 h post transfection. Virus present in the media is concentrated by precipitation with 8% polyethylene glycol and 500 mM sodium chloride and the precipitated virus is added to the lysates prepared from the collected cells. The viruses are purified over iodixanol (Optiprep, Sigma) step gradients (15%, 25%, 40%, and 60%). Viruses are concentrated and formulated in PBS. Virus titers are determined by measuring the number of DNaseI-resistant vector genome copies (VGs) using qPCR and the linearized genome plasmid as a control.

Animals. Marmoset (Callithrix jacchus) procedures were approved by ACUC of the National Institutes of Mental Health. Marmosets were born and raised in NIMH colonies and housed in family groups under standard conditions of 27° C. temperature and 50% humidity. They were fed ad libitum and received enrichment as part of the primate enrichment program for NHPs at the NIH. For AAV infusions, animals were screened for endogenous neutralizing antibodies (Nab). None of the animals that were screened showed any detectible blocking reaction at 1:5 dilution of serum (Penn Vector Core, University of Pennsylvania). They were then housed individually for several days and acclimated to a new room before injections. Four adult males were used for the library screening, two each for first and second round libraries. The day before infusion the animals' food was removed. Animals were anesthetized with isoflurane in oxygen, the skin over the femoral vein was shaved and sanitized with an isopropanol scrub, and the virus was infused over several minutes. Anesthesia was withdrawn and the animals were monitored until they became active, upon which they were returned to their cages. Activity and behavior were closely monitored over the next three days, with daily observations thereafter.

DNA/RNA recovery and sequencing. Round 1 and round 2 viral libraries were injected into marmosets at a dose of 2×10¹² vg/animal and rAAV genomes were recovered four weeks post injection. Animals were euthanized and brain (both round 1 and round 2), spinal cord (round 2 only) and liver (round 2 only) were recovered, snap frozen, and placed into long-term storage at −80° C. For round 1, the brain was separated into four coronal sections, and for round 2, six coronal sections. 100 mg of each brain section, spinal cord, and liver was homogenized in Trizol (Life Technologies, 15596) using a BeadBug (Benchmark Scientific, D1036) and viral DNA was isolated according to the manufacturers recommended protocol. Recovered viral DNA was treated with RNase, underwent restriction digestion with SmaI (found within the ITRs) to improve later rAAV genome recovery by PCR, and purified with a Zymo DNA Clean and Concentrator kit (D4033). Viral genomes were enriched by 25 cycles of PCR amplification with primers flanking the 588-589 insertion site in the capsid genome using 50% of the total extracted viral DNA as a template. After Zymo DNA purification, samples were diluted 1:100 and each dilution further amplified around the library variable region with 10 cycles of PCR. Subsequently, samples were further amplified using NEBNext Dual Index Primers for Illumina sequencing (New England Biolabs, E7600) for 10 more cycles. The amplification products were run on a 2% low-melting point agarose gel (ThermoFisher Scientific, 16520050) for better separation and recovery of the 210 bp band.

For the second round library only, packaged viral library DNA was isolated from the injected viral library by digestion of the viral capsid and purification of the contained ssDNA. These viral genomes were amplified by two PCR amplification steps, like the viral DNA extracted from tissue, to add adapters and indices for Illumina next-generation sequencing, and purified after gel electrophoresis. This viral library DNA, along with the viral DNA extracted from tissue, was sent for deep sequencing using an Illumina HiSeq 2500 system (Millard and Muriel Jacobs Genetics and Genomics Laboratory, Caltech).

NGS data alignment and processing. Raw fastq files from NGS runs were processed with custom-built scripts (https://github.com/GradinaruLab/protfarm). For the first round library, the pipeline to process these datasets involved filtering to remove low-quality reads, utilizing a quality score for each sequence, and eliminating bias from PCR-induced mutations or high GC-content. The filtered dataset was then aligned by a perfect string match algorithm and trimmed to improve the alignment quality. Read counts for each sequence were pulled out and displayed by tissue, at which point all sequences found in the brain were compiled for formation of the second round library.

For the second round library read counts by tissue were similarly tabulated. Then, a read count of 1 was added to each sequence to remove 0 values, all brain regions for each sequence were summed together, and the read sequences for each codon replicate of a given 7-mer amino acid sequence were summed together to give a single value for each peptide insertion. Finally, the data was log 2 counts per million (Cpm) normalized.

Tissue preparation and immunohistochemistry. Marmosets were euthanized (Euthanasia, VetOne) and perfused with 1×PBS. One hemisphere of the brain is cut into coronal blocks (4 for first round library, 6 for second round library), and along with sections of the spinal cord and liver (second round library only) were flash frozen in 2-methylbutane (Sigma Aldrich, M32631) chilled with dry ice.

### Example 2

### Method of Identifying Modified Capsid Proteins in Macaques

To assess how the top CNS transducing variants from our viral libraries performed compared to their parent, AAV9, we performed a pooled virus experiment in young Rhesus Macaques.

Plasmids. One rAAV genome was used in this study. pAAV-CAG-hFXN-HA utilizes an ssAAV genome containing an HA-tagged human frataxin (hFXN) protein under control of the synthetic CAG promoter and harboring a unique 12 bp sequence in the 3′UTR to differentiate different capsids packaging the same transgene.

Viral production. Recombinant AAVs were generated according to established protocols. Briefly, immortalized HEK293T cells (ATCC) were triple transfected with three vectors using polyethylenimine (PEI). The first vector contains a transgene cassette flanked by inverted terminal repeat (ITR) sequences from a parental AAV virus. The transgene cassette has a promoter sequence that drives transcription of a heterologous nucleic acid in the nucleus of the target cell. The second vector contains nucleic acids encoding the AAV Rep gene as well as the modified Cap gene for the variant being produced. The modified Cap gene comprises any one of SEQ ID NOS: 37-366, which are the DNA sequences encoding the modified AAV capsid proteins of the present disclosure. The modified CAP gene, in some cases, comprises any one of SEQ ID NOS: 385-654, which are the DNA sequences encoding the full-length VP1 protein with the insertions at amino acid positions 588-589. The third vector contains nucleic acids encoding helper virus proteins needed for viral assembly and packaging of the heterologous nucleic acid into the modified capsid structure. Viral particles are harvested from the media after 72 h post transfection and from the cells and media at 120 h post transfection. Virus present in the media is concentrated by precipitation with 8% polyethylene glycol and 500 mM sodium chloride and the precipitated virus is added to the lysates prepared from the collected cells. The viruses are purified over iodixanol (Optiprep, Sigma) step gradients (15%, 25%, 40%, and 60%). Viruses are concentrated and formulated in PBS. Virus titers are determined by measuring the number of DNaseI-resistant vector genome copies (VGs) using qPCR and the linearized genome plasmid as a control.

Animals. Rhesus Macaque (Macaca mulatta) procedures were performed at the CNPCR and approved by the UC Davis IACUC. Monkeys were born within the CNPRC colony of a mother screened and found negative for NAbs against AAV9 and raised as a separate family unit from the rest of the colony under standard conditions. Two infants aged approximately 5.5 mo old were used for the pooled injection study. Animals were fasted overnight prior to injection. At time of procedure, monkeys were sedated and the dorsal aspect of the lumbosacral spine was shaved and prepped with 70% isopropyl alcohol. The monkeys were placed in the prone position and the needle of the injection assembly introduced between L4-L5 and slowly advanced until cerebrospinal fluid (CSF) was aspirated. Pooled virus (0.5 mL) formulated in sterile PBS was injected followed by a sterile saline flush immediately afterward. After dosing, the monkeys were placed in the ventral recumbency position while recovering from anesthesia. General wellbeing was confirmed twice daily throughout the extent of the study.

DNA/RNA recovery and sequencing. A pool of viruses (AAV9, AAV-PHP.eB, AAV.CAP-A4, AAV.CAP-B2, AAV.CAP-B10, AAV.CAP-B22, and variants of the current invention) packaging CAG-hFXN-HA with unique 12 bp barcodes were injected into two 5.5 mo old macaques. After four weeks, animals were euthanized, one hemisphere of the brain was split into eight even thickness coronal sections, and along with samples of the spinal cord and liver were snap frozen. 100 mg slices from each coronal brain section as well as from the spinal cord and liver were homogenized in Trizol (Life Technologies, 15596) using a BeadBug (Benchmark Scientific, D1036) and total DNA and RNA were recovered according to the manufacturer's recommended protocol. Recovered DNA was treated with RNase, underwent restriction digestion with SmaI, and purified with a Zymo DNA Clean and Concentrator Kit (D4033). Recovered RNA was treated with DNase, and cDNA was generated from the mRNA using Superscript III (Thermo Fisher Scientific, 18080093) and oligo(dT) primers according to the manufacturer's recommended protocol. Barcoded FXN transcripts were recovered from both the DNA and cDNA libraries, as well as the injected pool, using primers that bound around the barcoded region on the 3′UTR of the transcripts and Q5 DNA polymerase in five reactions using 50 ng of DNA, cDNA or viral DNA, each, as a template. After Zymo DNA purification, samples were diluted 1:100 and further amplified around the barcode region using primers to attach adapters for Illumina next-generation sequencing. After cleanup, these products were further amplified using NEBNext Dual Index Primers for Illumina sequencing (New England Biolabs, E7600) for ten cycles. The amplification products were run on a 2% low-melting point agarose gel (ThermoFisher Scientific, 16520050) for better separation and recovery of the 210 bp band. All indexed samples were sent for deep sequencing similar to previous.

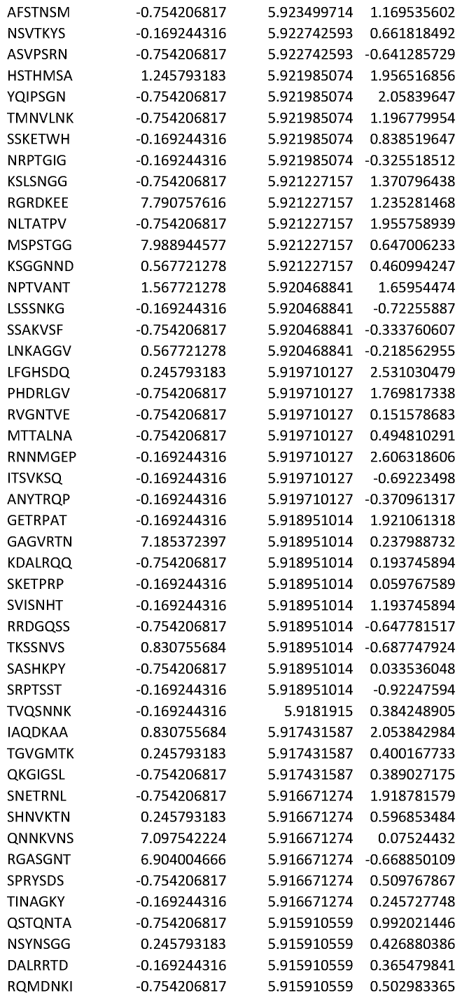

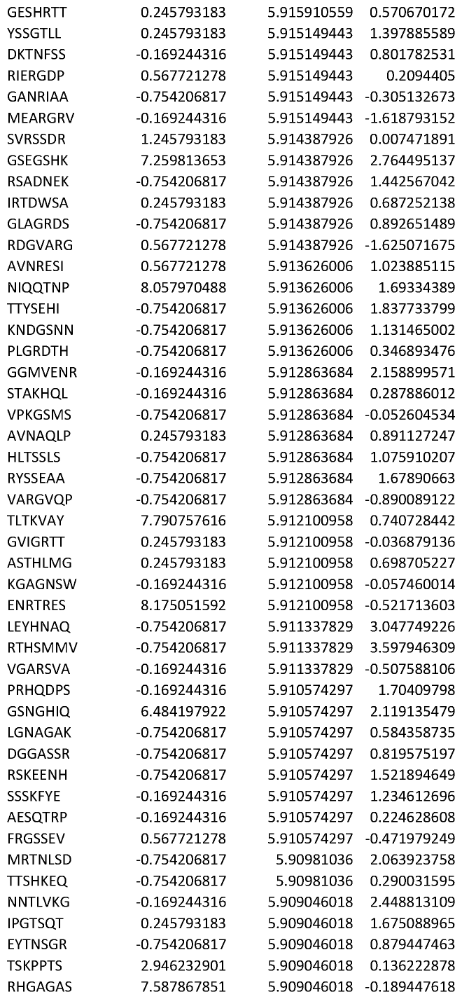

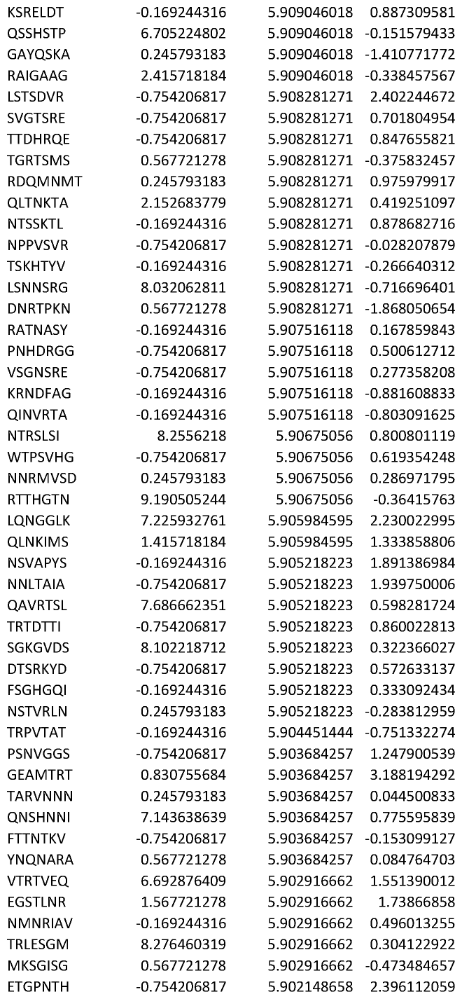

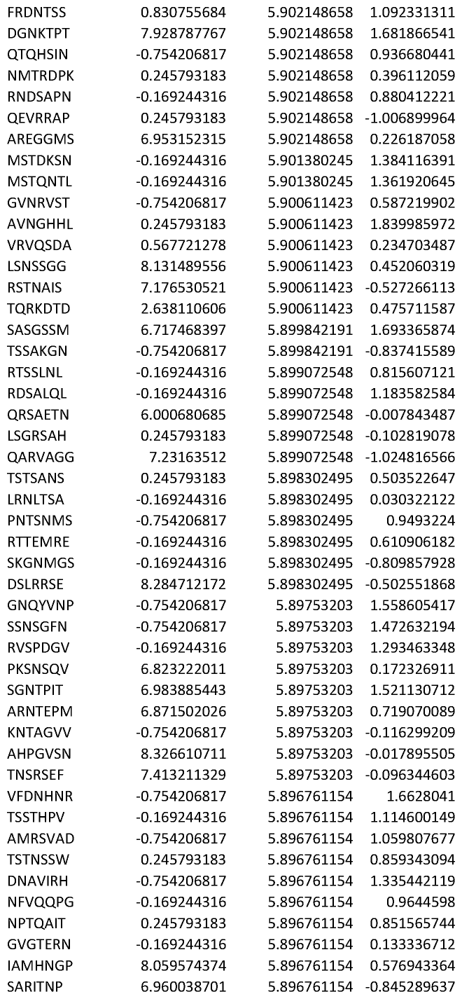

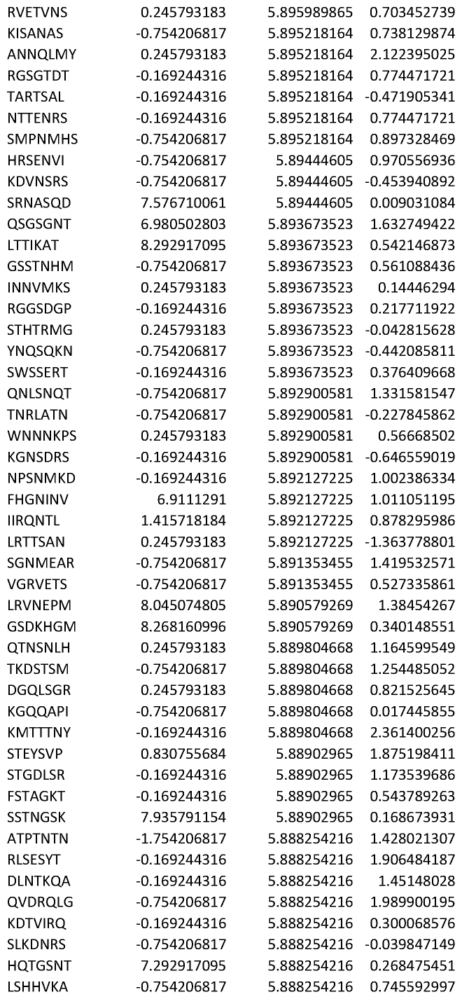

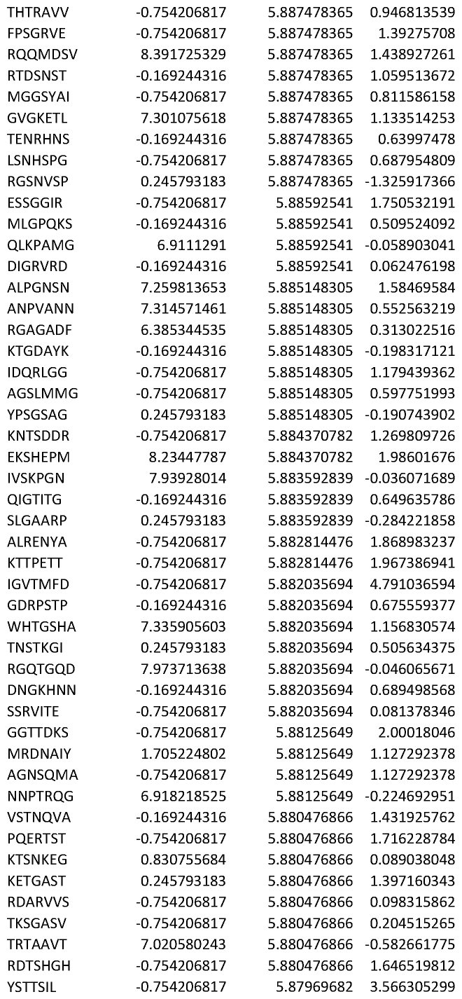

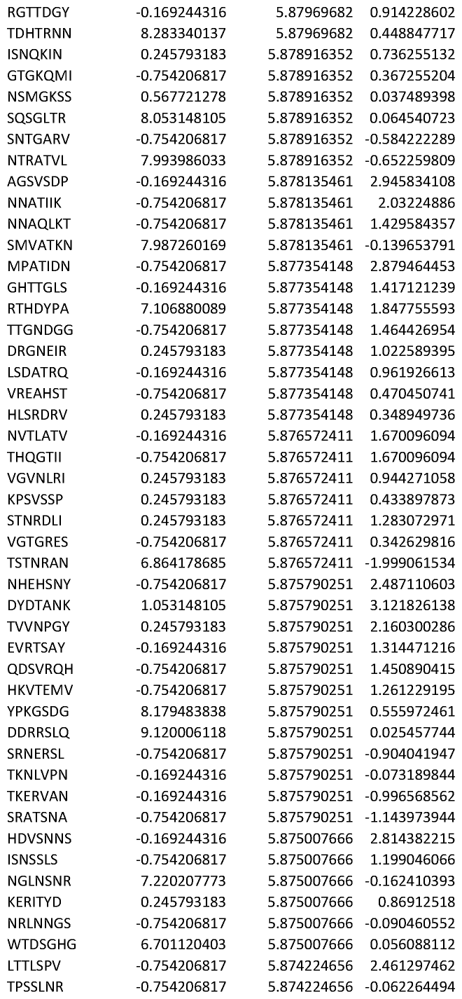

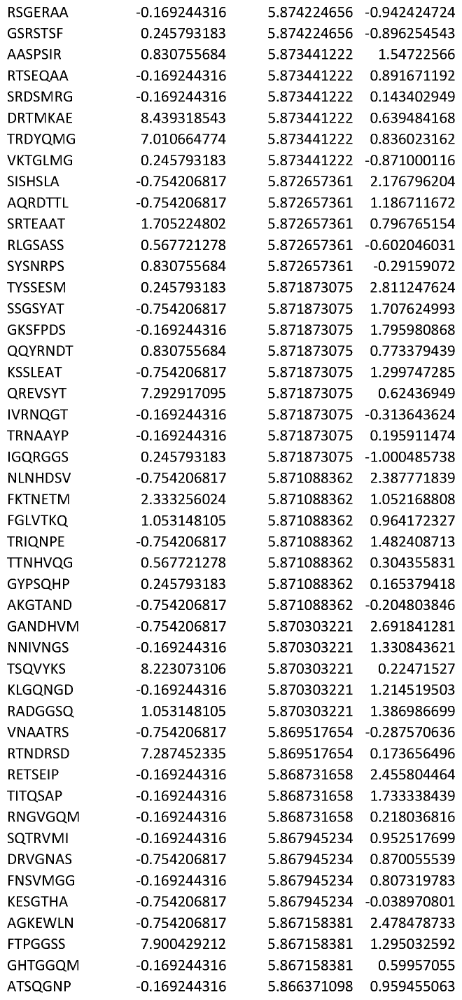

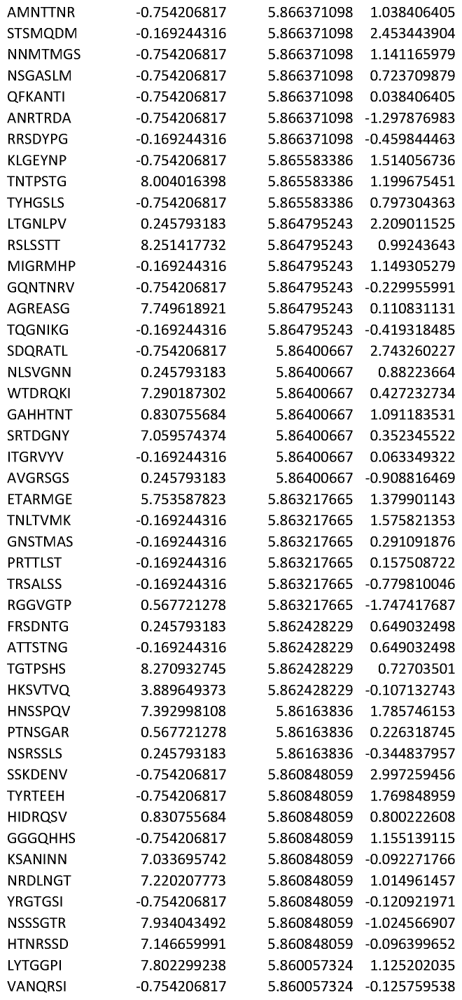

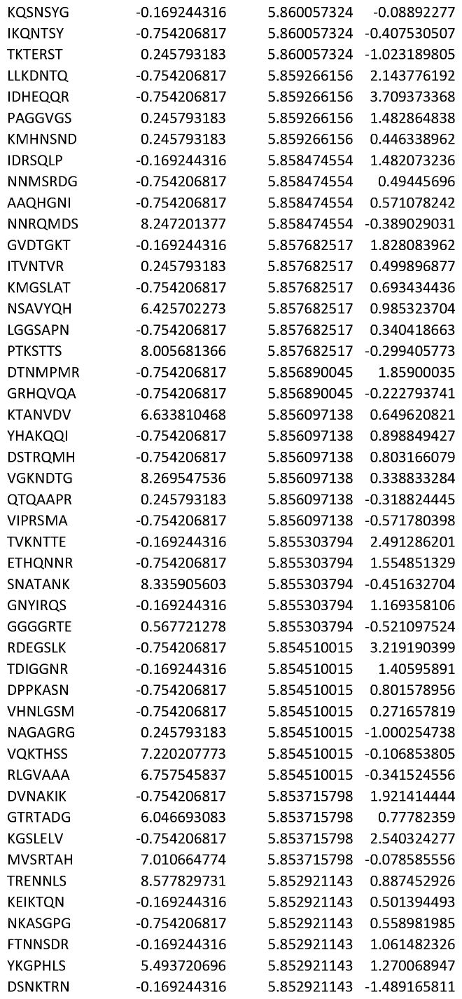

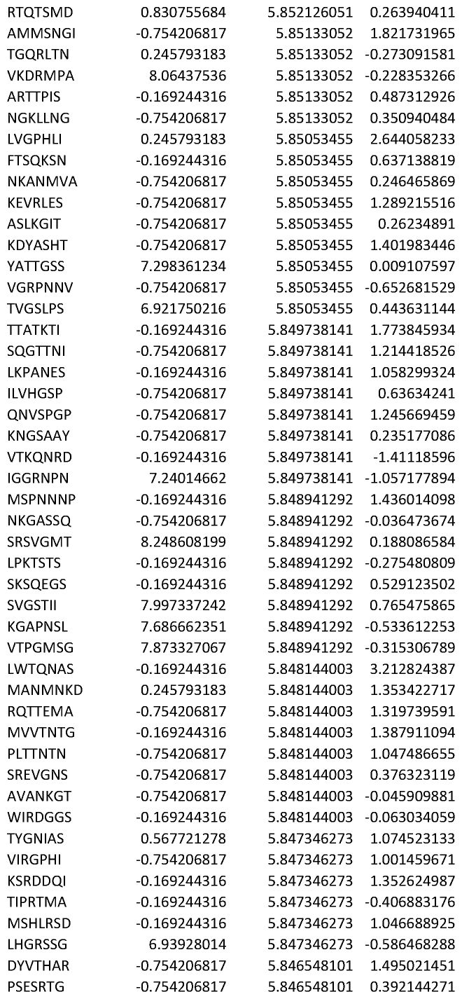

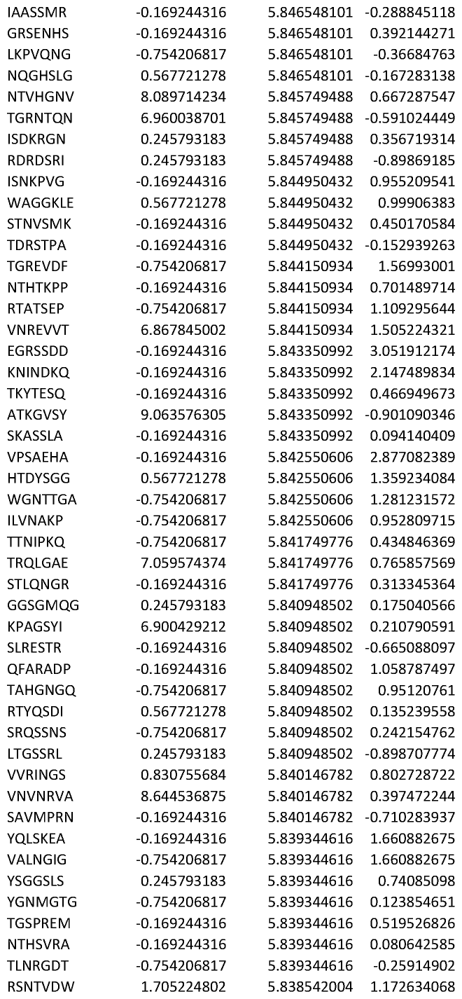

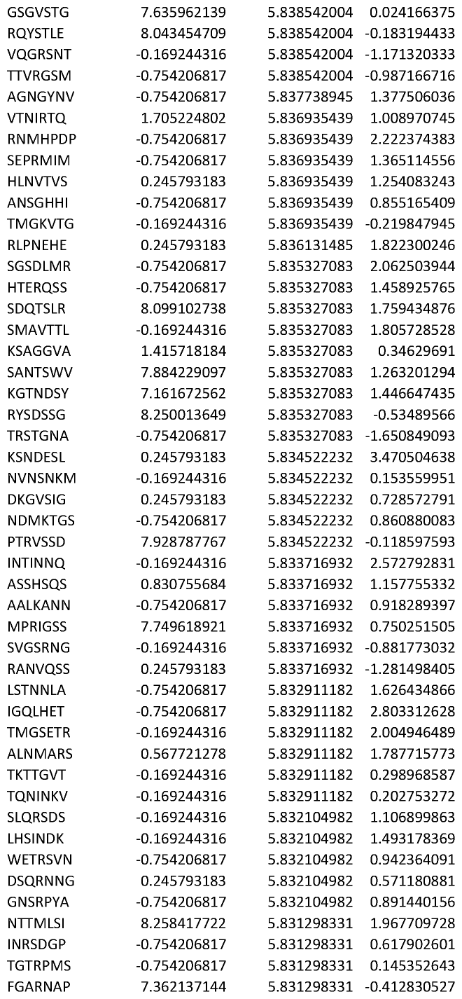

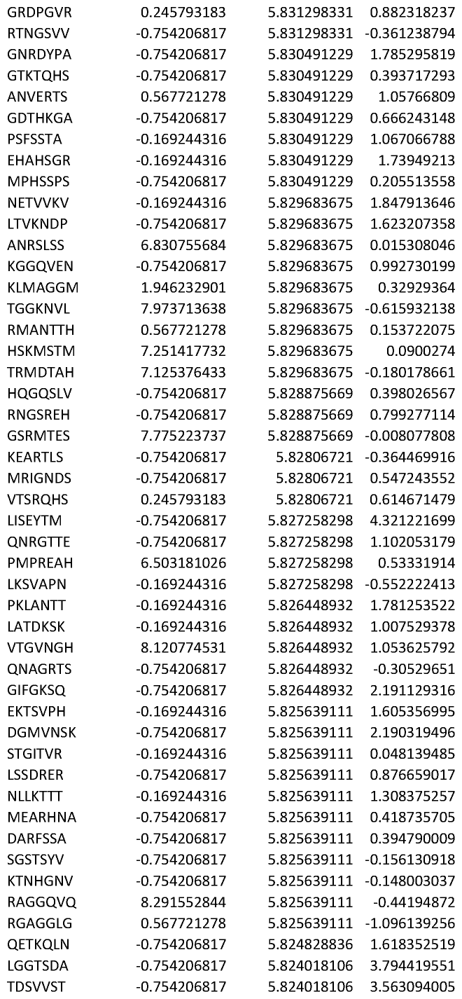

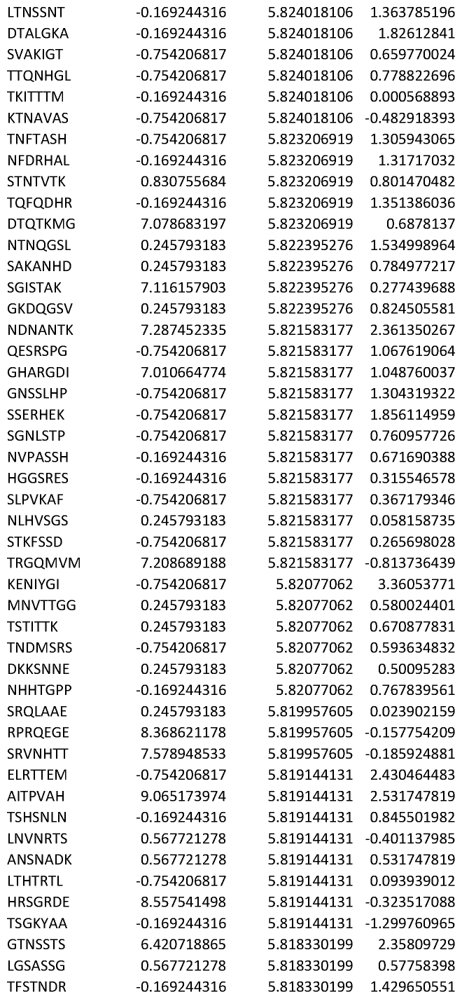

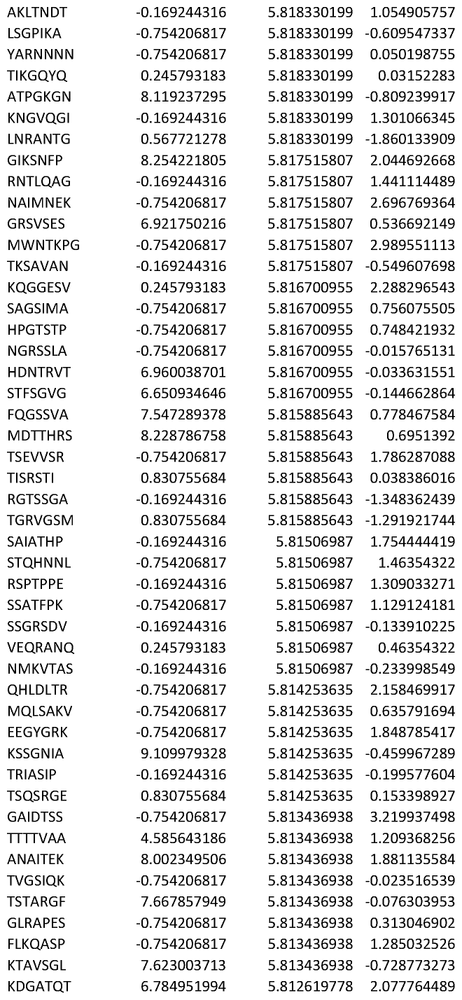

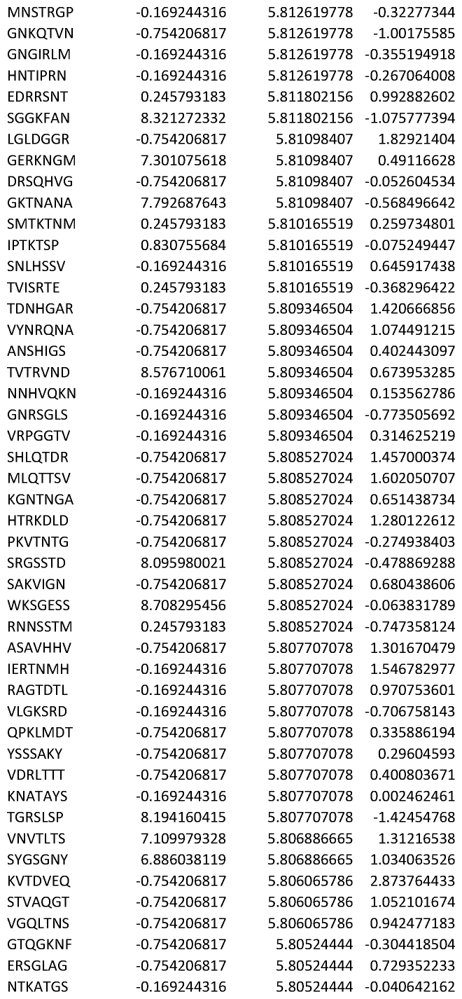

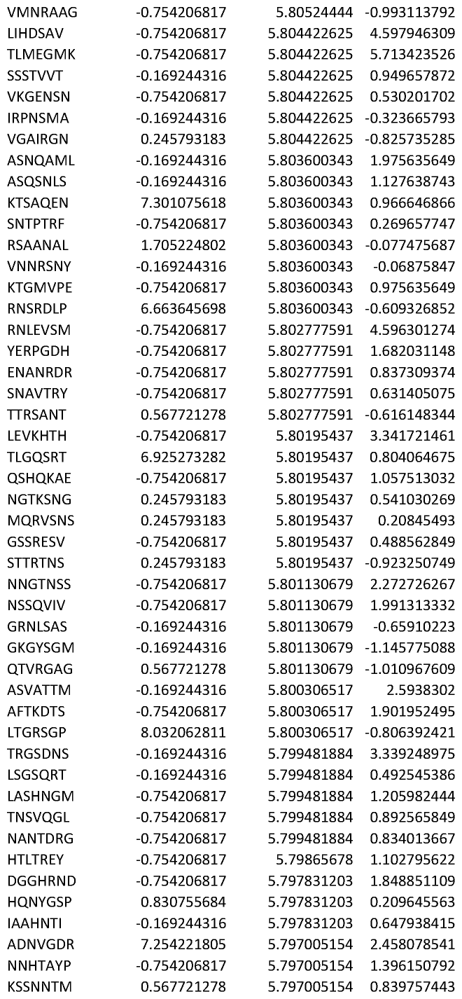

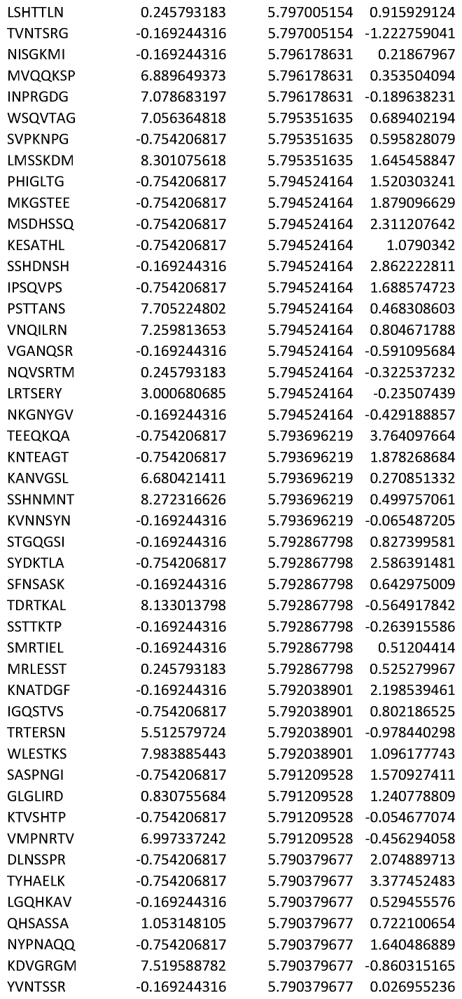

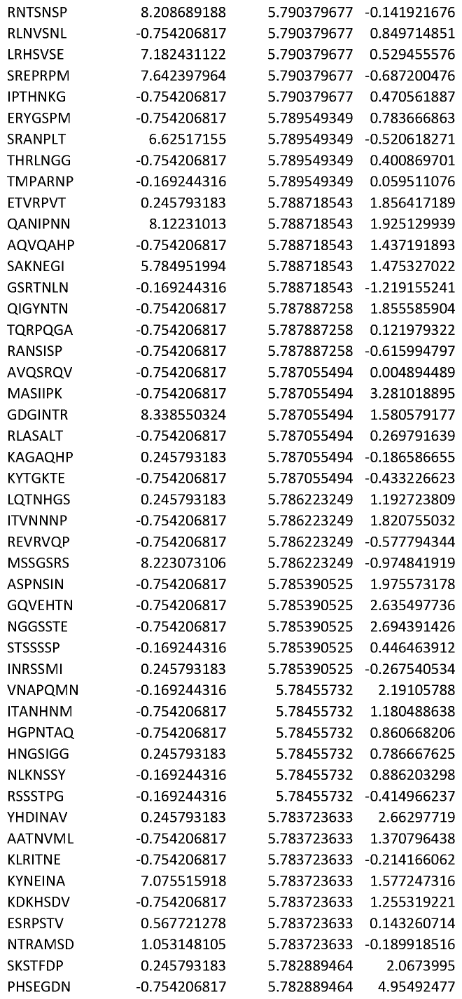

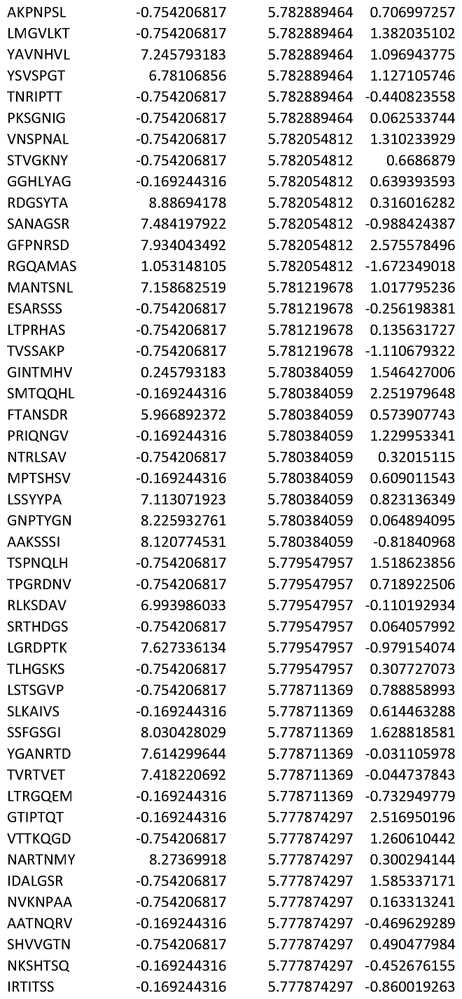

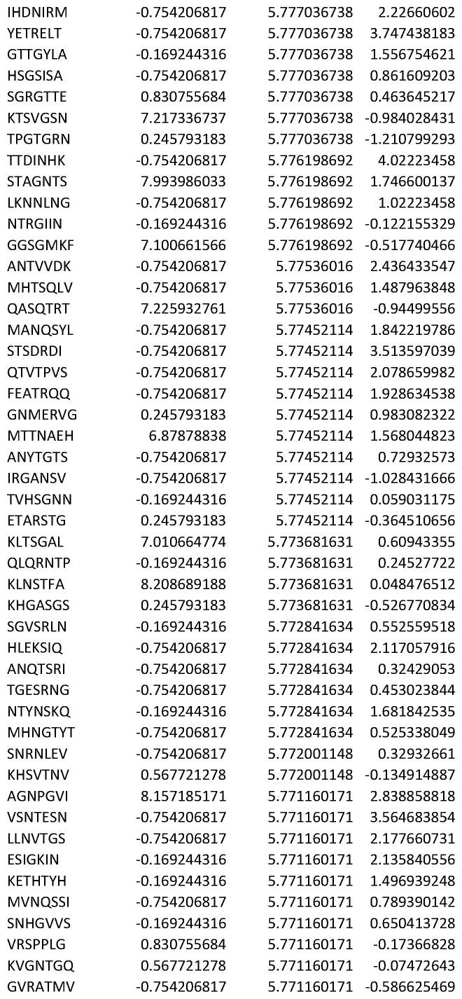

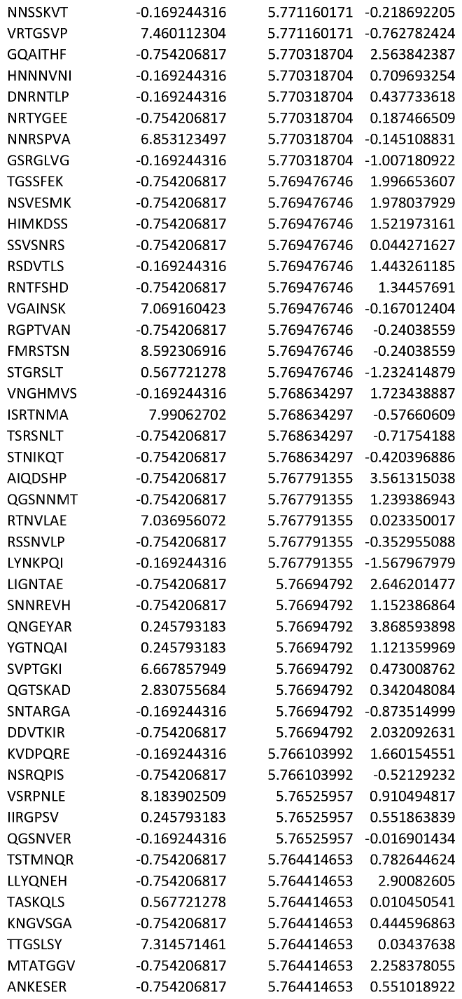

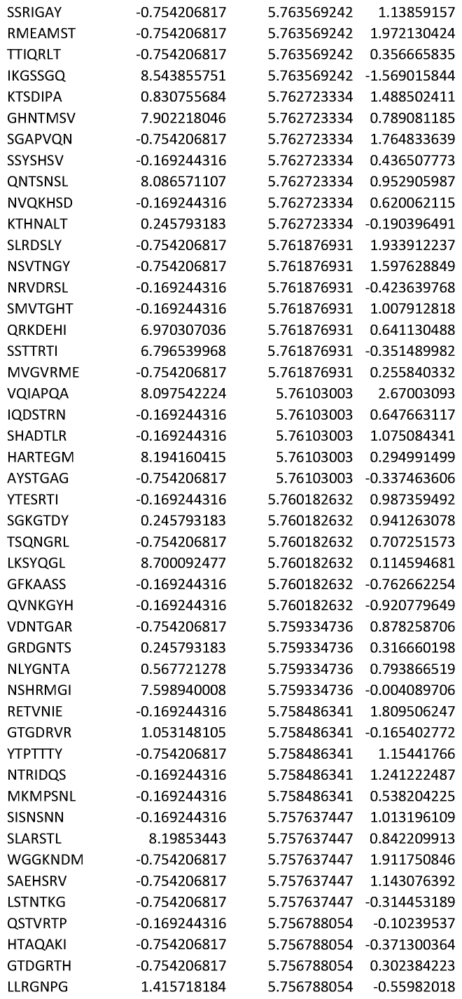

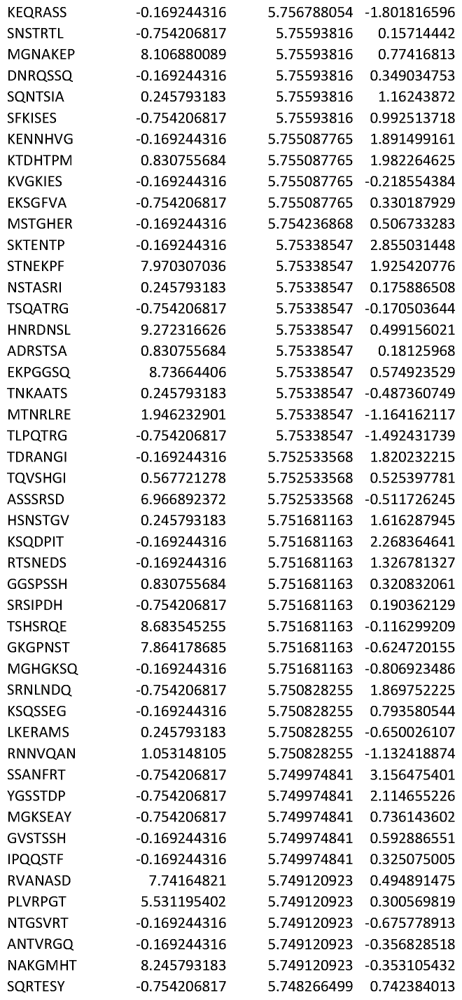

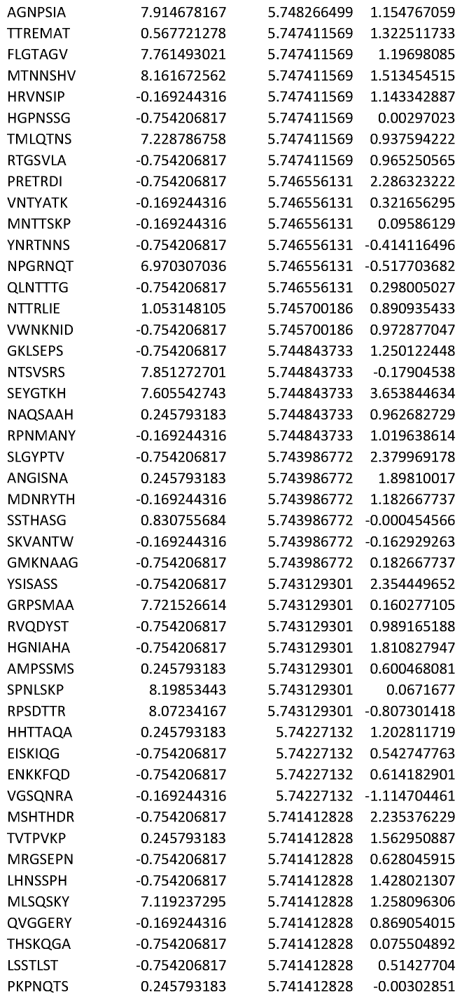

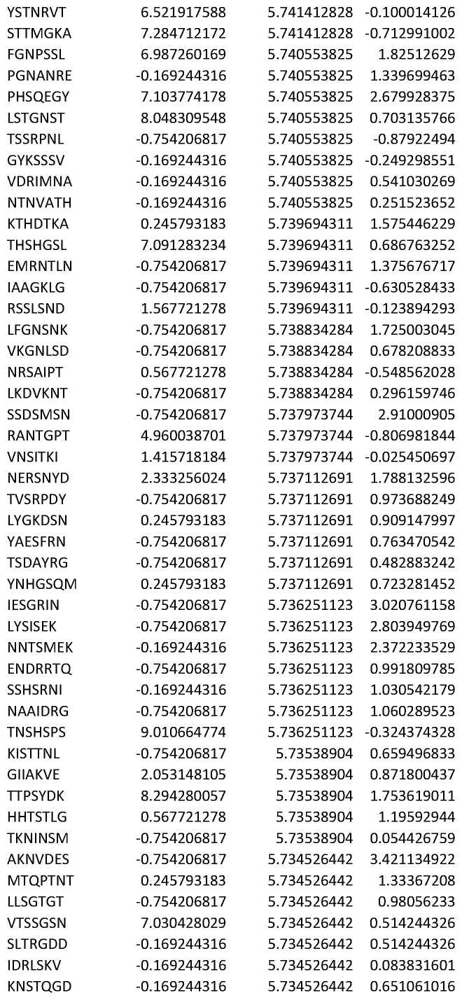

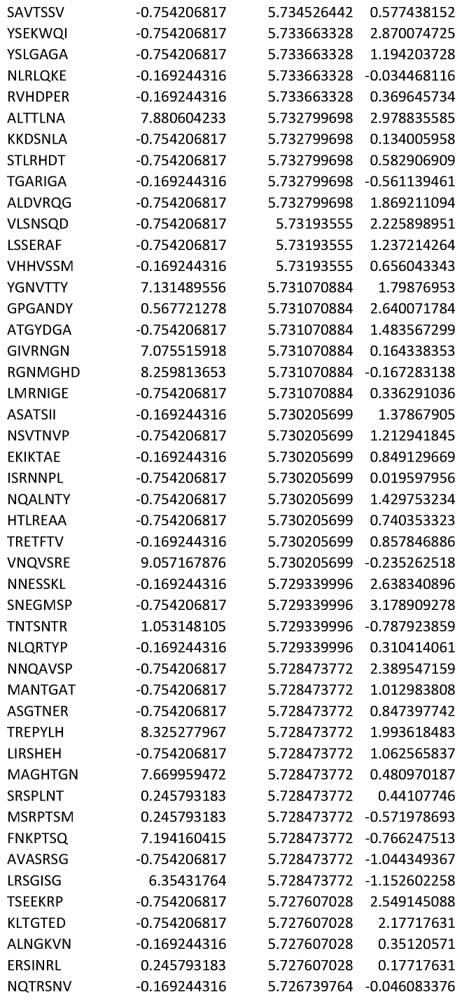

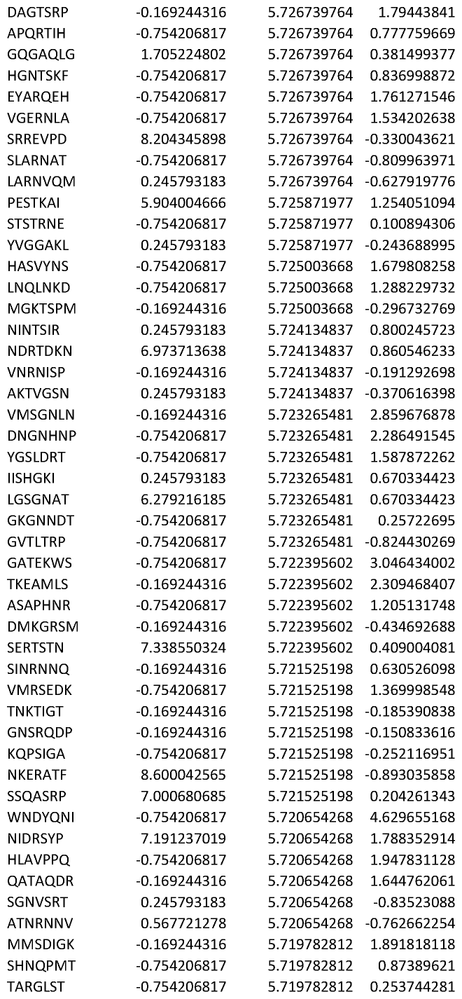

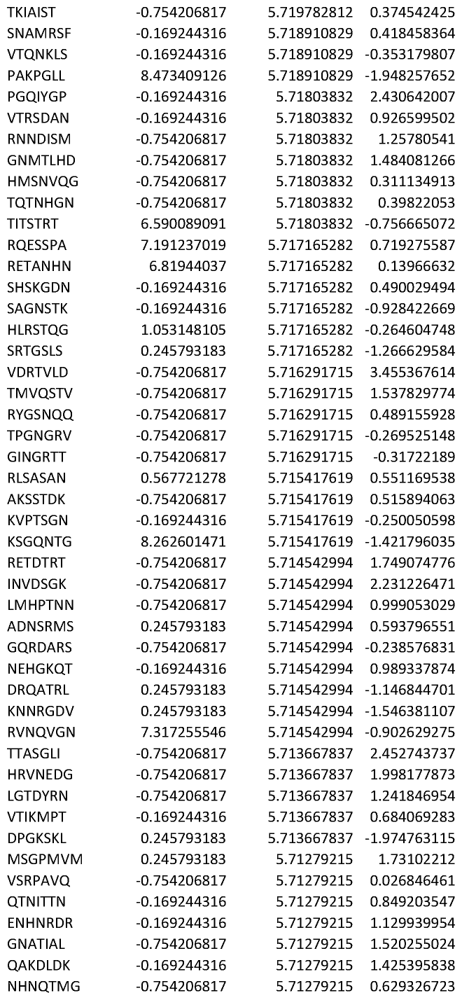

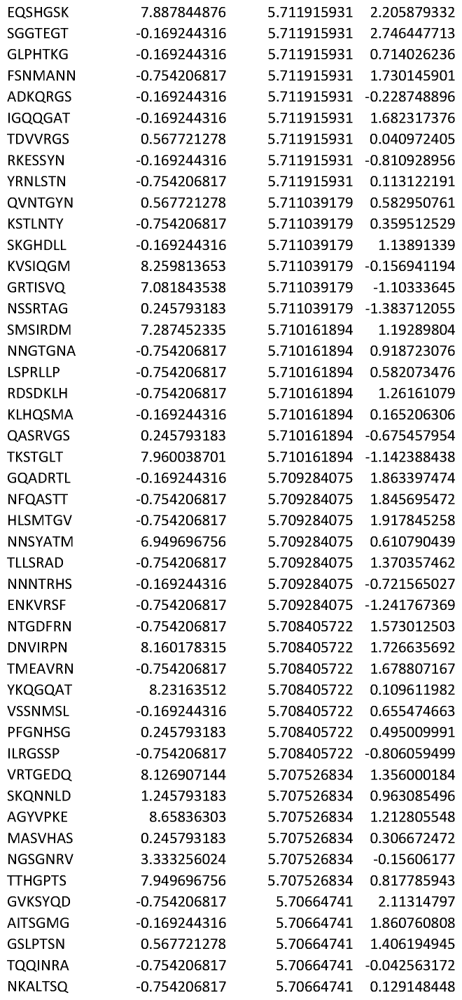

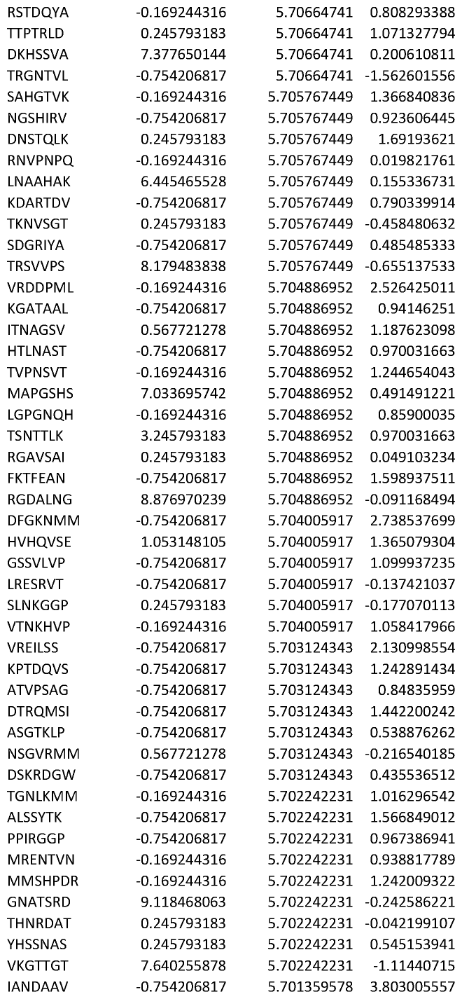

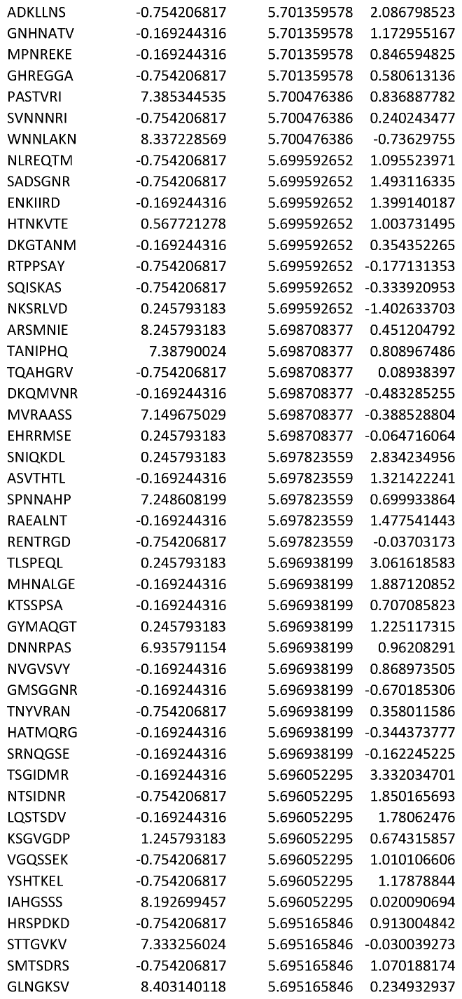

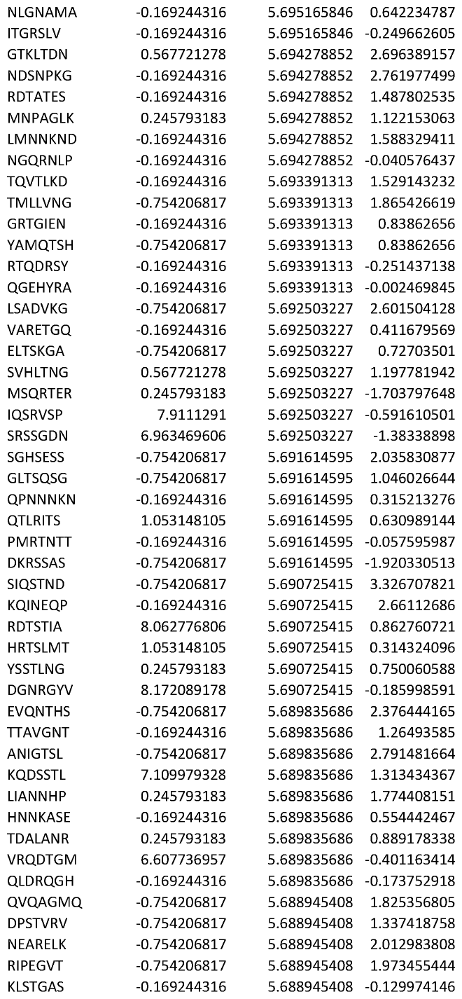

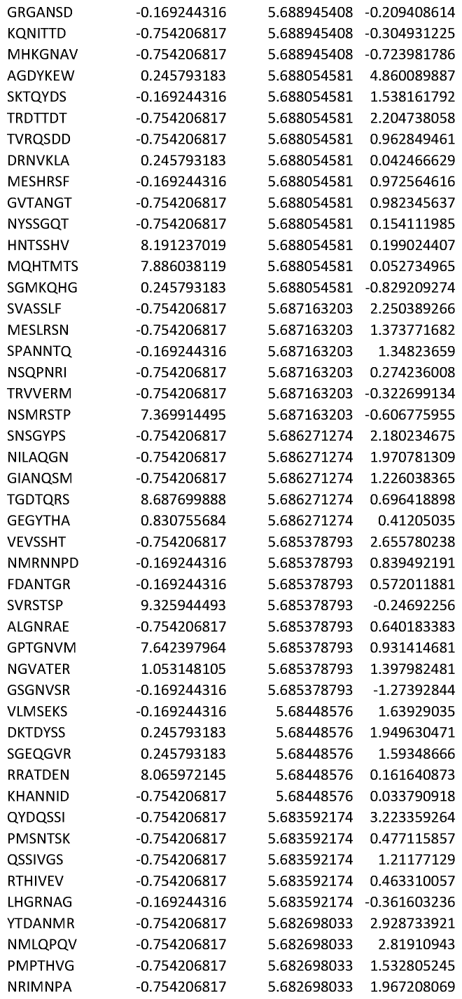

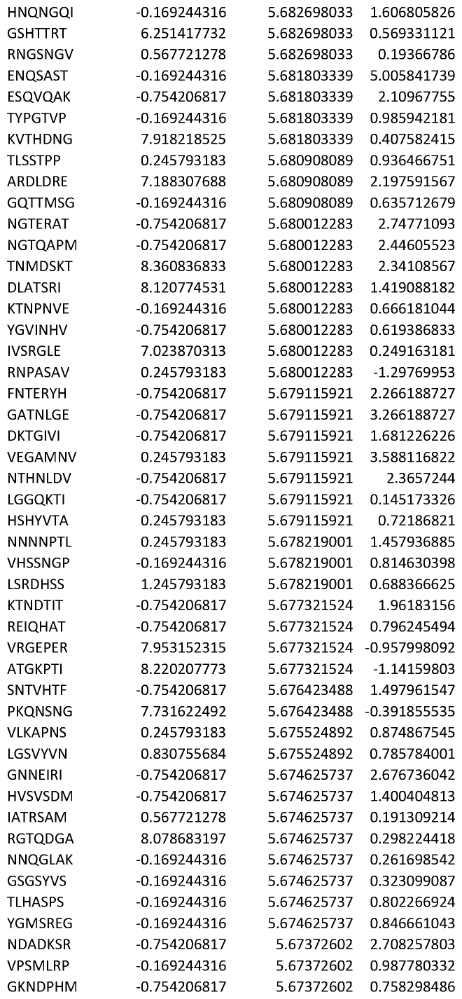

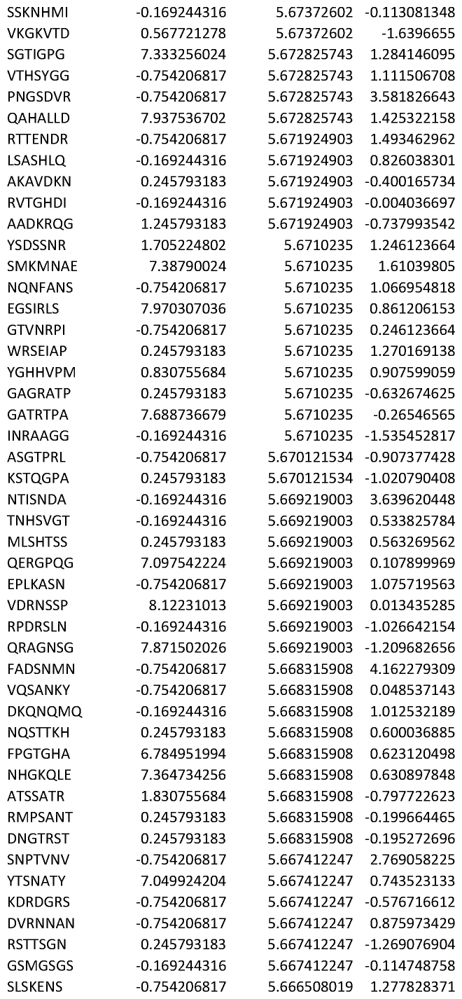

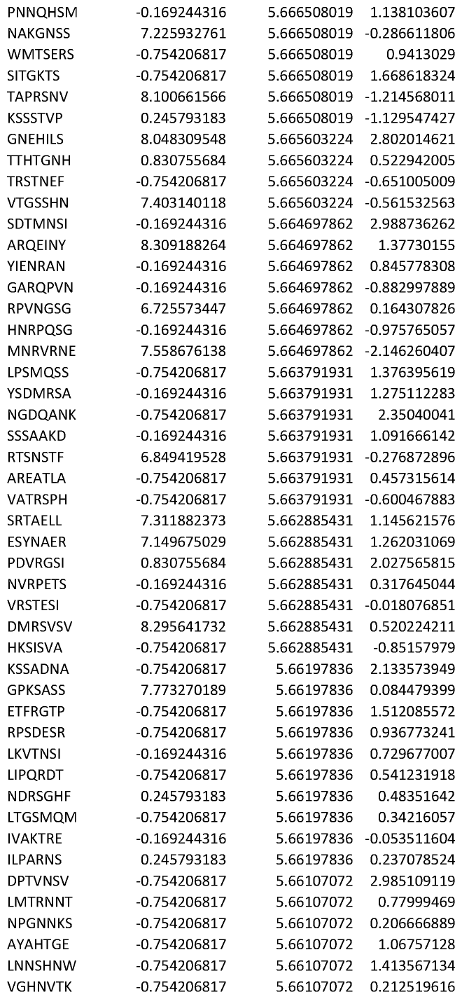

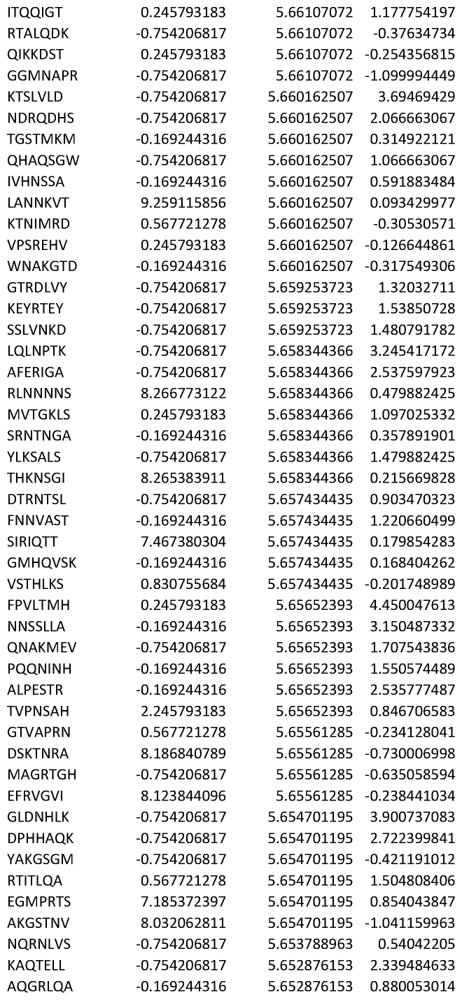

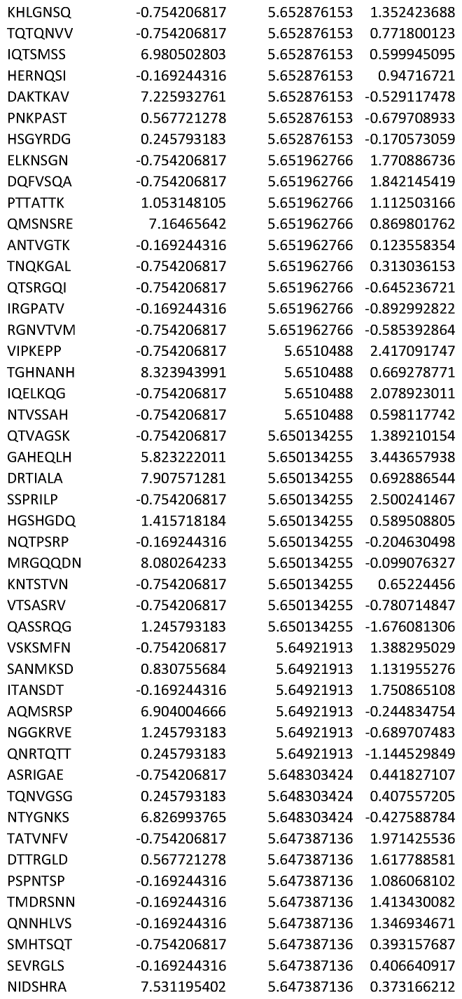

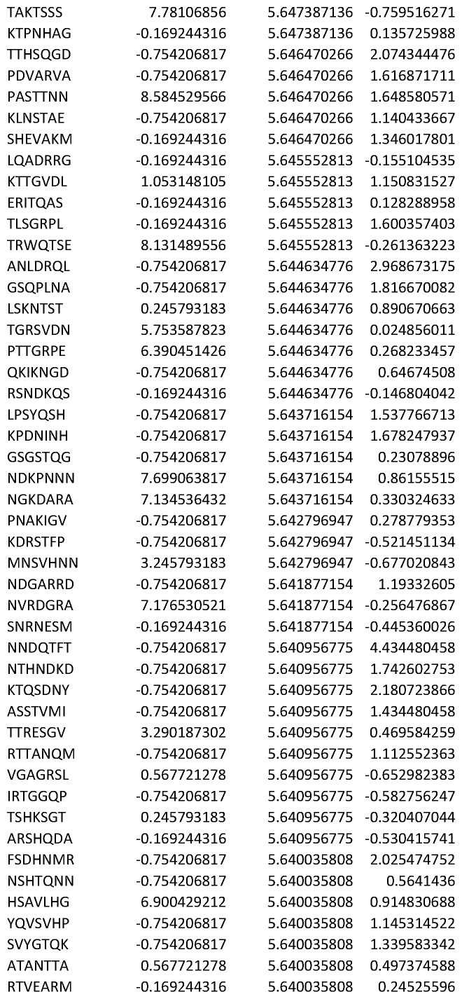

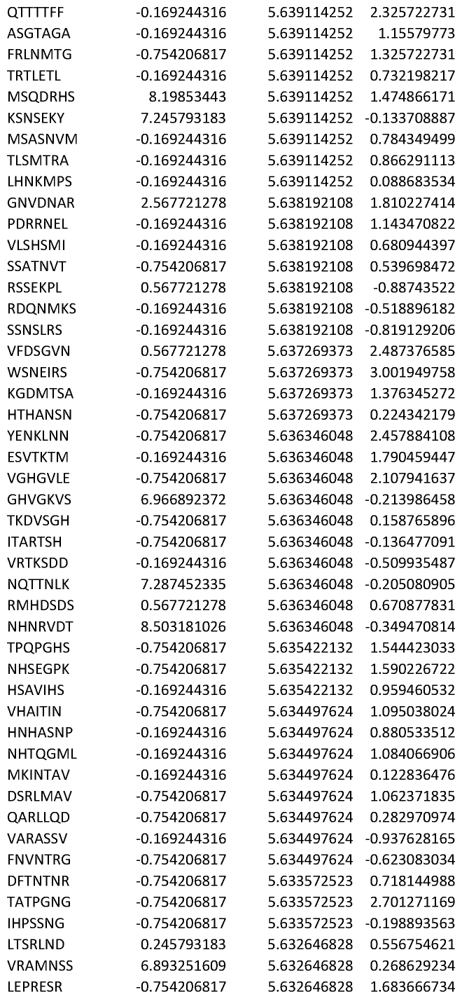

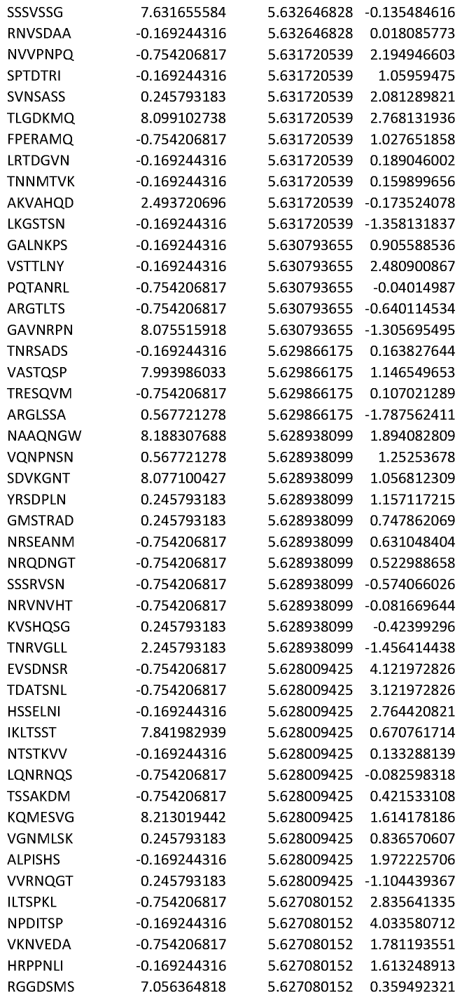

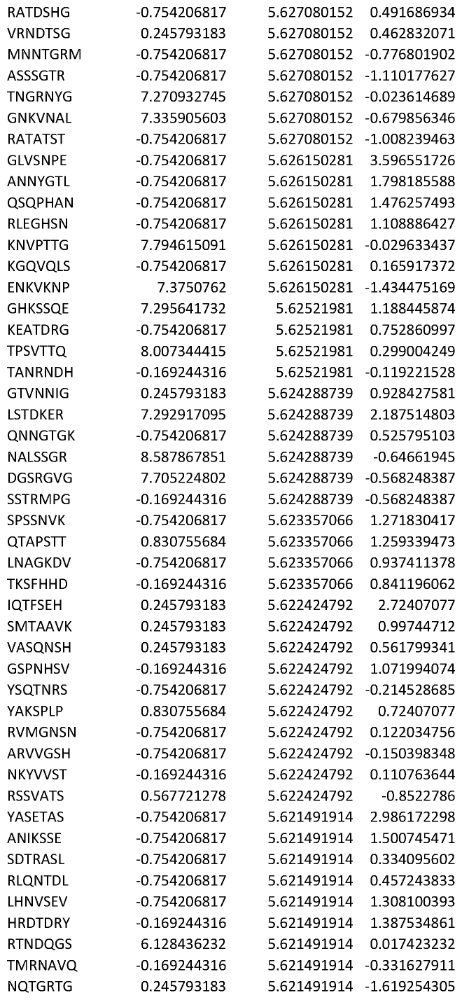

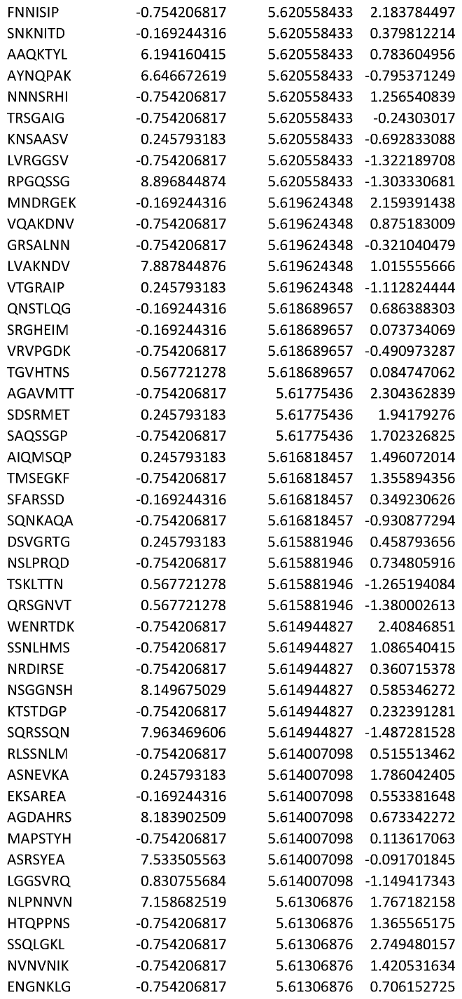

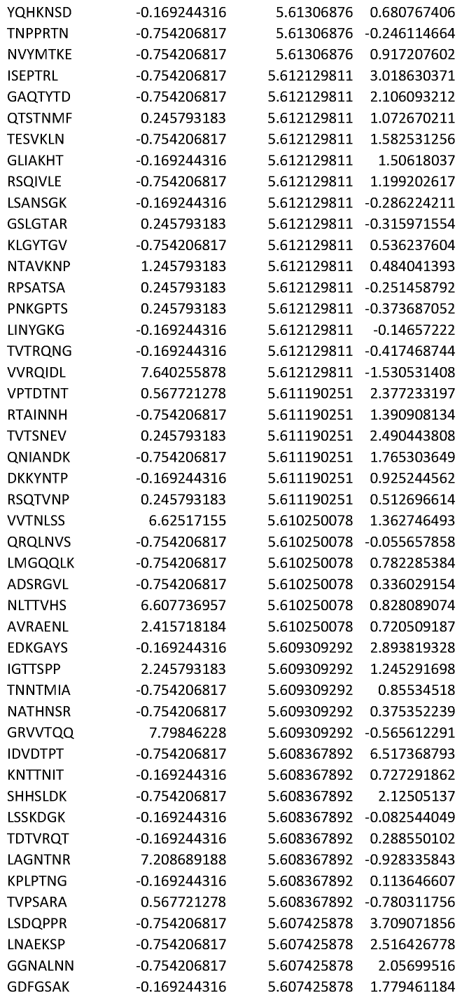

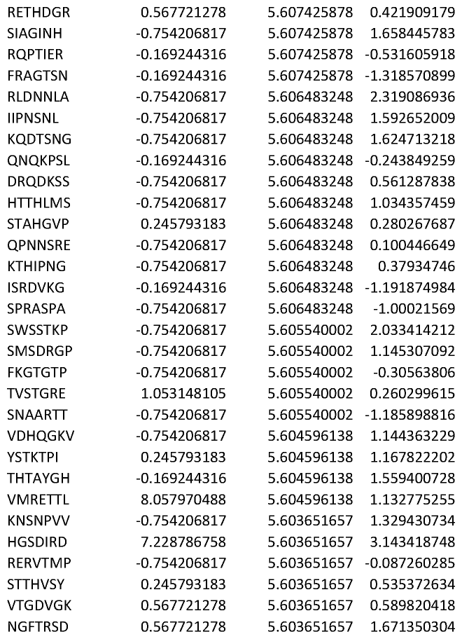

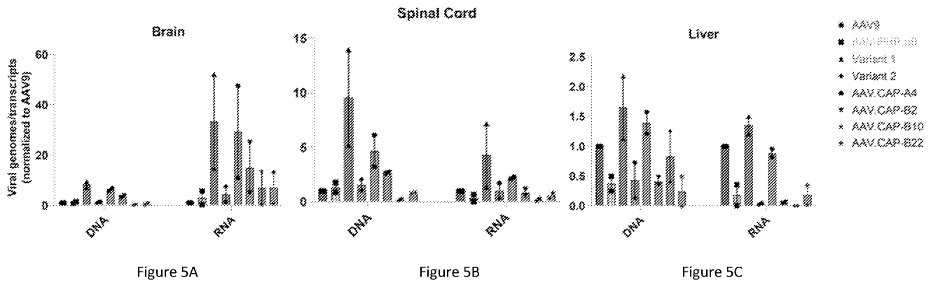

NGS data alignment and processing. Raw fastq files from NGS runs were processed with custom-built scripts (https://github.com/GradinaruLab/protfarm). For the pooled virus experiment, the pipeline to process the NGS results was similar to that of the first library experiment, with the difference that data was aligned to a hFXN-HA template containing the 12 bp unique barcodes. Read counts for each sequence were pulled out and normalized to the respective contribution of that barcode to the initial, injected pooled virus to account for small inequalities in the amount of each member of the pool that was injected into the monkeys. The distribution of the unique barcodes found within the DNA and RNA was averaged across the eight brain regions and represented as a single value for the entire brain. The DNA and RNA values for each of the variants, read out by their unique barcodes, was then averaged across the two animals, normalized to the value of AAV9, and graphed as viral genomes or RNA transcripts, respectively (FIG. 5).

Tissue preparation and immunohistochemistry. Macaques were euthanized (Euthanasia, VetOne) and perfused with 1×PBS. Each hemisphere of the brain was cut into eight coronal blocks, with one hemisphere, along with a sample of spinal cord and liver being flash frozen in 2-methylbutane (Sigma Aldrich, M32631) chilled with dry ice. The other hemisphere and pieces of spinal cord and liver were removed and post-fixed with 4% PFA at 4° C. for 48 hours. Each of the coronal sections of brain were sectioned at 100 m with a vibratome. Immunohistochemistry (IHC) was performed on floating sections with primary and secondary antibodies in PBS containing 10% donkey serum and 0.1% Triton X-100. Primary antibody used was rabbit anti-HA (Cell Signaling Technology, 3724S), with incubation performed for 16-20 hours at room temperature (RT). The sections were then washed and incubated with secondary Alexa-647 conjugated anti-rabbit FAB fragment antibody (1:200, Jackson ImmunoResearch Laboratories, Inc., 711-607-003) for 6-8 hours at RT. Stained sections were then mounted with ProLong Diamond Antifade Mountant (ThermoFisher Scientific, P36970).

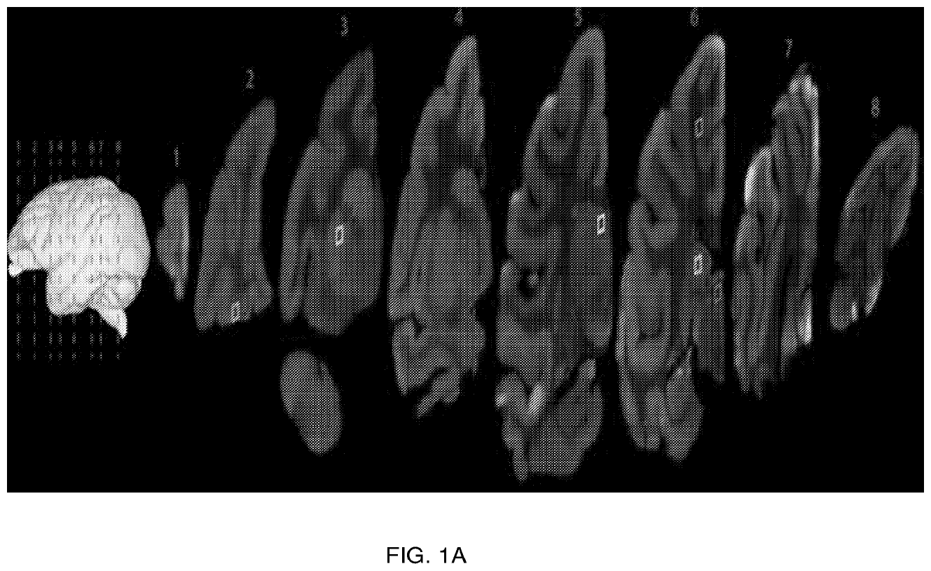

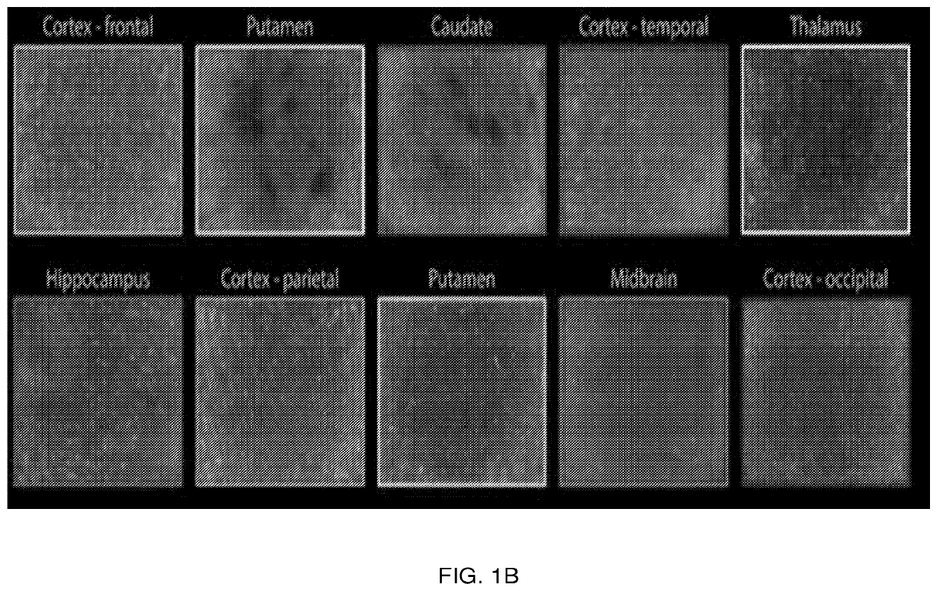

Imaging and Quantification. Macaque tissue sections transduced with the pooled viruses expressing CAG-hFXN-HA were imaged on a Keyence BZ-X all-in-one fluorescence microscope at 48-bit resolution with 4× and 10× objectives. Briefly, stained sections from each coronal block of the brain were imaged in their entirety at a 4× magnification (FIG. 1A). Across the eight coronal sections, sub-regions identified within various major brain areas, the four main cortical lobes, hippocampus, caudate, putamen, thalamus and midbrain, were imaged at a 10× magnification across a z-thickness of 25 m. A maximum intensity projection was then applied to those z-sections to produce a single image of representative staining in the area (FIG. 1B).

### Example 3

### Selection of AAV Variants with CNS Tropism in Marmosets

We performed two successive rounds of selection of our viral library based on the marmoset data described in Example 1, focusing on ability to transduce the CNS after systemic administration through the vasculature. Our original library, sized at 1.28 billion potential variants, was produced in HEK293 cells, which as a first pass removed many of the variants that were unable to produce functional viral capsids, and injected into a set of two adult marmosets. At the first round of selection, we performed a binary assessment of whether or not the viral sequences were able to be recovered from the tissue of interest. Any sequence found present in the marmoset brains, 33,287 sequences in total, was passed along to the second round of screening. In this second round, all of the capsid variants within the library were able to be produced. Thus, while the total dose injected into each animal is the same, each of the variants is present at a much higher titer than the original library, allowing for a much larger fraction of sequences to reach and transduce the tissue of interest, and thus a much more robust readout of the data.

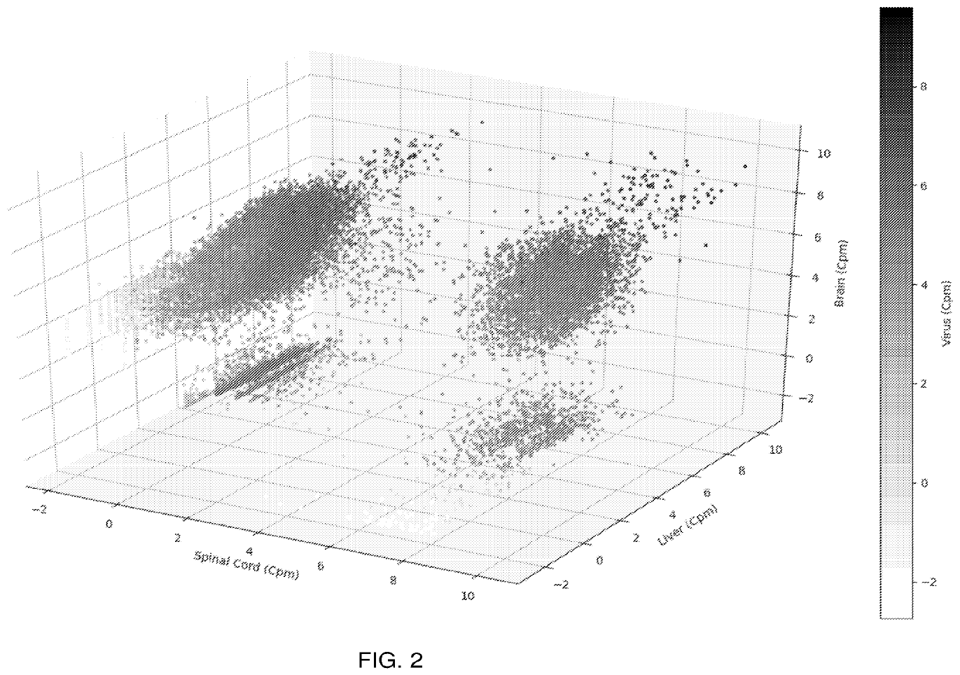

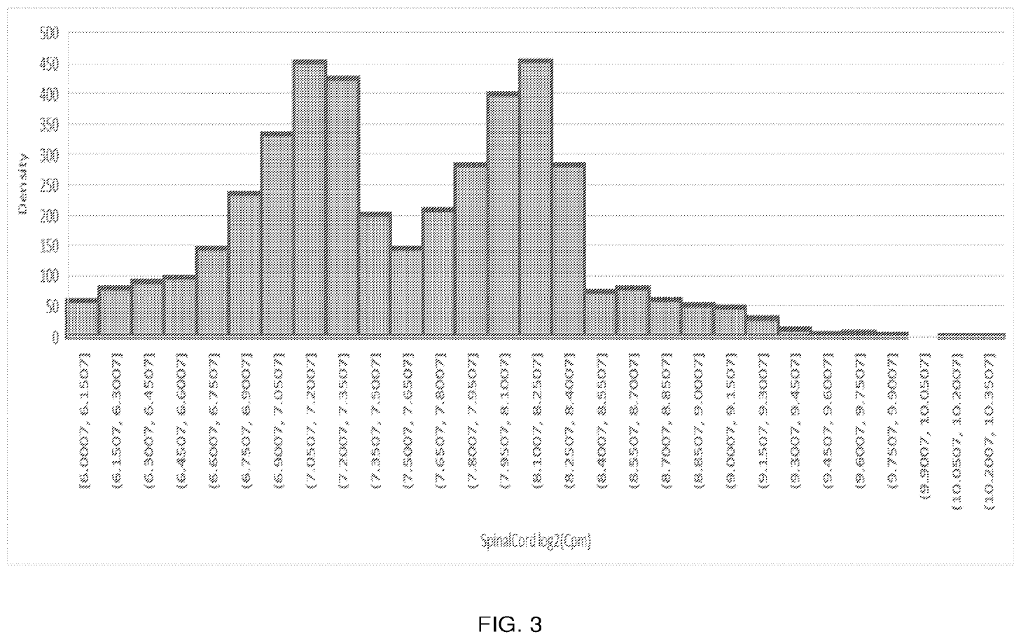

In the second round, a counts per million (Cpm) value was calculated for each capsid variant in three tissues, brain, spinal cord, and liver. A 3-dimensional scatter plot of the Cpm values in those three tissues was generated (FIG. 2). Five distinct variant groups of interest were identified from this plot: Brain+, SpinalCord+, Brain+SpinalCord+, Brain+SpinalCord−, and SpinalCord+Brain−. An additional five groups were found with the highest expression in the brain: MaxBrainCpm, Max Brain Cpm SpinalCord−, Max Brain Cpm Brain+Spinal cord+, Max Brain Cpm/SpinalCord+High, and Max Brain Cpm/SpinalCord+Low, Due to the selection of sequences found present in the brain in the first round of selection, a large percentage of the sequences from the second round fall into the Brain+group. Interestingly, though, the clear separation of the variants into these distinct groups is indicative of a mechanistic difference among the groups in the way they transduce the different tissues. A closer inspection of the SpinalCord+variants revealed a bimodal distribution of Cpm values (FIG. 3). Such a distribution identifies additional subclasses of SpinalCord+variants. As such, six additional variant groups were identified: SpinalCord+Low, SpinalCord+High, SpinalCord+LowBrain+, SpinalCord+LowBrain−, SpinalCord+HighBrain+, and SpinalCord+HighBrain−. Similarly to above, the appearance of this bimodal distribution of the SpinalCord+variants is indicative to us of the potential for a different mechanism of action of these viral groups. Even though the end result, efficient transduction of cells within the Spinal Cord, is the same, there may be two different ways these groups achieve it.

For these reasons, we separated all of these 16 groups for analysis of the top sequences. Two lists of specific variants of interest were designated within each of the eleven variant groups. In one list, the 10 variants within each group with the highest enrichment relative to injected virus (as measured by log 2([Tissue Cpm]/[Virus Cpm]) were assembled. In the other list, the 10 variants within each group with the highest enrichment relative to liver (as measured by log 2([Tissue Cpm]/[Liver Cpm]) were assembled. This resulted in the lists of variants identified in Tables 4-30, as described below.

Table 4 provides amino acid sequences of rAAV capsid protein insertions, having a greater enrichment in the BRAIN after two rounds of in vivo selection, as well as the DNA sequences encoding them. CPM is defined as counts per million.

Table 5 provides amino acid sequences of rAAV capsid protein insertions, having a greater enrichment in the SPINAL CORD after two rounds of in vivo selection, as well as the DNA sequences encoding them.

Table 6 provides amino acid sequences of rAAV capsid protein insertions, having a greater enrichment in both the BRAIN and in the SPINAL CORD after two rounds of in vivo selection, as well as the DNA sequences encoding them.

Table 7 provides amino acid sequences of rAAV capsid protein insertions, having a greater enrichment in the BRAIN over that found in the LIVER after two rounds of in vivo selection, as well as the DNA sequences encoding them.

Table 8 provides amino acid sequences of rAAV capsid protein insertions, having a greater enrichment in the BRAIN over that found in the SPINAL CORD after two rounds of in vivo selection, as well as the DNA sequences encoding them.

Table 9 provides amino acid sequences of rAAV capsid protein insertions, having a greater enrichment in the BRAIN over that found in the LIVER and SPINAL CORD after two rounds of in vivo selection, as well as the DNA sequences encoding them.

Table 10 provides amino acid sequences of rAAV capsid protein insertions, having a greater enrichment in the SPINAL CORD over that found in the LIVER and BRAIN after two rounds of in vivo selection, as well as the DNA sequences encoding them.

Table 11 provides amino acid sequences of rAAV capsid protein insertions, having a greater enrichment in the SPINAL CORD over that found in BRAIN after two rounds of in vivo selection, as well as the DNA sequences encoding them.

Table 12 provides amino acid sequences of rAAV capsid protein insertions, having a greater enrichment in the SPINAL CORD over that found in the LIVER after two rounds of in vivo selection, as well as the DNA sequences encoding them.

Table 13 provides amino acid sequences of rAAV capsid protein insertions, having a greater enrichment in both SPINAL CORD and BRAIN over that found in the LIVER after two rounds of in vivo selection, as well as the DNA sequences encoding them.

In addition to the sequences identified in Table 5, Table 14 provides other amino acid sequences of rAAV capsid protein insertions, having an improved enrichment in the SPINAL CORD after two rounds of in vivo selection, as well as the DNA sequences encoding them.

In addition to the sequences identified in Table 5 and Table 14, Table 15 provides yet a third group of amino acid sequences of rAAV capsid protein insertions, having improved enrichment in the SPINAL CORD after two rounds of in vivo selection, as well as the DNA sequences encoding them.

In addition to the sequences identified in Table 6, Table 16 provides other amino acid sequences of rAAV capsid protein insertions, having improved enrichment in both the BRAIN and in the SPINAL CORD after two rounds of in vivo selection, as well as the DNA sequences encoding them.

In addition to the sequences identified in Table 6 and Table 16, Table 17 provides yet a third group of amino acid sequences of rAAV capsid protein insertions, having significant enrichment in both the BRAIN and in the SPINAL CORD after two rounds of in vivo selection, as well as the DNA sequences encoding them.

In addition to the sequences identified in Table 12, Table 18 provides other amino acid sequences of rAAV capsid protein insertions, having improved enrichment in the SPINAL CORD over the LIVER after two rounds of in vivo selection, as well as the DNA sequences encoding them.

In addition to the sequences identified in Table 12 and Table 18, Table 19 provides yet a third group of amino acid sequences of rAAV capsid protein insertions, having significant enrichment in the SPINAL CORD over the LIVER after two rounds of in vivo selection, as well as the DNA sequences encoding them.

In addition to the sequences identified in Table 11, Table 20 provides other amino acid sequences of rAAV capsid protein insertions, having a improved enrichment in the SPINAL CORD over that found in BRAIN after two rounds of in vivo selection, as well as the DNA sequences encoding them.

In addition to the sequences identified in Table 11 and Table 20, Table 21 provides yet a third group of amino acid sequences of rAAV capsid protein insertions, having significant enrichment in the SPINAL CORD over that found in BRAIN after two rounds of in vivo selection, as well as the DNA sequences encoding them.

In addition to the sequences identified in Table 10, Table 22 provides other amino acid sequences of rAAV capsid protein insertions, having improved enrichment in the SPINAL CORD over that found in the LIVER and BRAIN after two rounds of in vivo selection, as well as the DNA sequences encoding them.

In addition to the sequences identified in Table 10 and Table 22, Table 23 provides yet a third group amino acid sequences of rAAV capsid protein insertions, having significant enrichment in the SPINAL CORD over that found in the LIVER and BRAIN after two rounds of in vivo selection, as well as the DNA sequences encoding them.

Table 24 provides amino acid sequences of rAAV capsid protein insertions, having a maximum expression in the BRAIN after two rounds of in vivo selection, as well as the DNA sequences encoding them.

Table 25 provides amino acid sequences of rAAV capsid protein insertions, having a greater expression in the BRAIN and low expression in the spinal cord after two rounds of in vivo selection, as well as the DNA sequences encoding them.

Table 26 provides amino acid sequences of rAAV capsid protein insertions, having the best expression in the BRAIN of the insertions expressed in the brain after two rounds of in vivo selection, as well as the DNA sequences encoding them.

Table 27 provides amino acid sequences of rAAV capsid protein insertions, having the best expression in the BRAIN of the insertions expressed in the one spinal cord group after two rounds of in vivo selection, as well as the DNA sequences encoding them.

Table 28 provides amino acid sequences of rAAV capsid protein insertions, having the best expression in the BRAIN of the insertions expressed in another spinal cord group after two rounds of in vivo selection, as well as the DNA sequences encoding them.

In addition to the sequences identified in Table 13, Table 29 provides other amino acid sequences of rAAV capsid protein insertions, having improved enrichment in the SPINAL CORD AND BRAIN over that found in the LIVER after two rounds of in vivo selection, as well as the DNA sequences encoding them.

In addition to the sequences identified in Table 13 and Table 29, Table 30 provides yet a third group amino acid sequences of rAAV capsid protein insertions, having significant enrichment in the SPINAL CORD and BRAIN over that found in the LIVER after two rounds of in vivo selection, as well as the DNA sequences encoding them.

### Example 4

### Characterization of CNS Tropism for AAV Variants

To assess how the top CNS transducing variants from our viral libraries performed compared to their parent, AAV9, we performed a pooled virus experiment in young Rhesus Macaques as described in Example 2. We produced a pool of viruses [AAV9 and AAV-PHP.eB as controls, AAV.CAP-A4, AAV.CAP-B2, AAV.CAP-B10 and AAV.CAP-B22 as variants pulled out of previous rodent engineering efforts that shouldn't translate well to NHPS, and AAV variants of the present invention, selected from our round 2 library analysis]. Each virus packaged an HA-tagged human frataxin (hFXN-HA) with a unique molecular barcode under control of the ubiquitous CAG promoter. We used hFXN because it is an endogenous protein expressed throughout the body. Each packaged hFXN contained a separate 12-base barcode on the 3′UTR to differentiate the contribution of each virus from the rest after NGS. The viruses were pooled at equal ratios and injected intrathecally in the CSF at the lumbar region of the spine into two young rhesus, aged roughly 5.5 mo old, at a total dose of 1.5×10¹² vg/kg (each virus injected at 1.875×10¹¹ vg/kg). Intrathecal administration, as opposed to intravenous administration, was used for this experiment to characterize the variants that performed better due to their ability to enter and express their cargo within cells of the CNS vs. the ability to more efficiently cross the blood-brain barrier, a characteristic more difficult in higher order primates. Following four weeks of expression, throughout which no adverse health effects were observed, the brains, spinal cords, and livers were taken for DNA and RNA sequencing, and immunohistochemistry.

As evidenced by staining against the HA tag on the hFXN, robust and broad expression was achieved by the pool throughout the macaque brain (FIG. 1). Expression was even throughout the areas assessed, all along the rostral-caudal axis of the brain, and in a variety of cortical and subcortical areas, including the frontal, temporal, occipital and parietal lobes, as well as the hippocampus, thalamus, caudate, putamen, and midbrain.

Following DNA and RNA extraction and NGS from multiple coronal slices per animal, as well as spinal cord and liver, we quantified the relative viral genomes and transcript expression levels of each of the barcoded viruses averaged across the two animals. At the level of viral genomes, a measure for the viruses' ability to enter cells, one variant had a cellular prevalence of roughly 8× higher levels than AAV9 (FIG. 5). Similar results were evidenced in the spinal cord, with roughly 9×AAV9 viral transgenes detected. Conversely, in the liver, that variant's viral genomes were not detected at meaningfully higher levels, roughly 50% higher than AAV9. At the level of RNA transcripts, the differences from AAV9 are more pronounced. One variant's transcripts were found at roughly 33× higher levels in the macaque brain than AAV9, with spinal cord and liver levels at 4× and 1.3×AAV9 respectively.

These results evidence two very important findings. First, the variants of the present invention are an incredibly potent viral delivery vehicle for targeting the primate CNS after an intrathecal injection, with significant therapeutic potential for gene therapy applications today. Second, that pooled variant testing in the macaques recapitulates the results of our library data analysis and validates the selection of top variants within each of the groups we separated within our data.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

All publications, patents, and patent applications mentioned in this specification are herein incorporated by reference to the same extent as if each individual publication, patent, or patent application was specifically and individually indicated to be incorporated by reference.

### Example 5

### AAV Variant Biodistribution Analysis

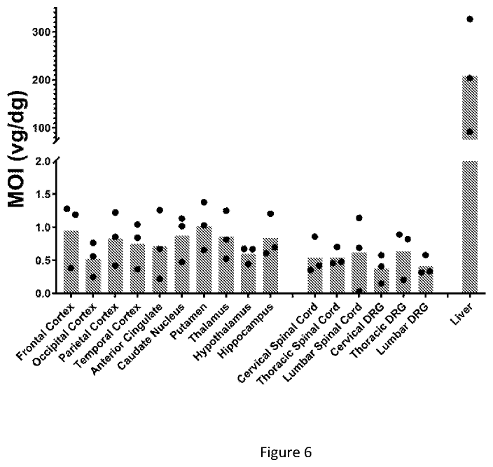

To further assess how the top CNS transducing variants from our viral libraries performed, we performed a virus bio-distribution experiment in young cynomolgus macaques. An AAV variant of the present invention [E] was injected intravenously into three young cynomolgus macaques, aged roughly 8 mo old, at a dose of 7.5×10¹³ vg/kg. The animals were sacrificed after 4 weeks in-life. The brains, spinal cords, and livers were taken for DNA sequencing. Viral genomes were measured by ddPCR of DNA extracted from the primate tissue and normalized to copies of GAPDH. A Multiplicity of Infection value were generated for each animal. See FIG. 6. Individual points on the graph indicate biological replicates.

TABLE 4
List of peptides that target the BRAIN with greater efficiency

| | | LOG2 BRAIN | | SEQ ID |
|---|---|---|---|---|
| SEQ ID NO: | SEQUENCE | CPM | DNA SEQUENCE | NO: |
| 38 | ISREFYK | 9.290656596 | ATTAGTAGGGAGTTTTATAAG | 385 |
| 41 | PSSNNPH | 7.733231578 | CCCAGCAGCAACAACCCCCAC | 386 |
| 44 | SQSIQKD | 7.651734329 | AGCCAAAGCATCCAAAAAGAC | 387 |
| 42 | NARSTGM | 7.067799511 | AATGCGAGGTCGACTGGGATG | 388 |
| 39 | GTDMRQT | 7.003731434 | GGTACTGATATGAGGCAGACT | 389 |
| 43 | SNRTLSI | 6.916671274 | AGCAACAGAACCCTCAGCATC | 390 |
| 45 | REDHNLY | 6.846548101 | AGGGAGGATCATAATTTGTAT | 391 |
| 40 | HLTSNQL | 6.686271274 | CATTTGACTAGTAATCAGCTG | 392 |
| 37 | AFGGIAD | 5.947520166 | GCTTTTGGTGGTATTGCTGAT | 393 |
| 46 | YQNDSGK | 6.342882968 | TACCAAAACGACAGCGGCAAA | 394 |

TABLE 5
List of peptides that target the SPINAL CORD with greater efficiency

| SEQ ID | | LOG2 SPINAL | | |
|---|---|---|---|---|
| NO: | SEQUENCE | CORD CPM | DNA SEQUENCE | SEQ ID NO: |
| 77 | EDNLSYV | 8.614299644 | GAAGACAACCTCAGCTACGTC | 395 |
| 78 | SDSTAFI | 8.220207773 | AGCGACAGCACCGCCTTCATC | 396 |
| 79 | SSNGPTD | 9.25282045 | AGCAGCAACGGCCCCACCGAC | 397 |
| 80 | EKTNEND | 8.303784906 | GAGAAGACTAATGAGAATGAT | 398 |
| 81 | SNTDSGT | 8.83731553 | AGTAATACTGATAGTGGGACT | 399 |
| 82 | GIGTSEA | 8.175051592 | GGGATTGGTACTAGTGAGGCT | 400 |
| 83 | AIVAAGY | 9.16465642 | GCCATCGTCGCCGCCGGCTAC | 401 |
| 84 | NLANIPN | 9.352356123 | AACCTCGCCAACATCCCCAAC | 402 |
| 85 | PLRTTQE | 8.057970488 | CCTCTGAGGACTACTCAGGAG | 403 |
| 86 | SDRRMNT | 8.247201377 | AGTGATCGTCGTATGAATACG | 404 |

TABLE 6
List of peptides that target both BRAIN and SPINAL CORD with greater
efficiency and specificity

| SEQ | | | LOG2 | | SEQ |
|---|---|---|---|---|---|
| ID | | LOG2 BRAIN | SPINAL | | ID |
| NO: | SEQUENCE | CPM | CORD CPM | DNA SEQUENCE | NO: |
| 301 | EQSHGSK | 5.711915931 | 7.887844876 | GAACAAAGCCACGGCAGCAAA | 405 |
| 302 | LLRDSNN | 7.115293002 | 8.297002124 | CTTCTTCGGGATTCTAATAAT | 406 |
| 303 | ILGNSRV | 7.249056472 | 7.982195114 | ATCCTCGGCAACAGCAGAGTC | 407 |
| 304 | VDKQREN | 6.287498829 | 7.199989493 | GTCGACAAACAAAGAGAAAAC | 408 |
| 305 | NDNQITR | 6.548276954 | 6.692876409 | AACGACAACCAAATCACCAGA | 409 |
| 306 | GTNSSTS | 5.818330199 | 6.420718865 | GGCACCAACAGCAGCACCAGC | 410 |
| 307 | LIKENRF | 6.751254772 | 7.237315029 | CTTATTAAGGAGAATCGTTTT | 411 |
| 308 | SSSTAMS | 6.65561285 | 7.211577468 | AGTTCTTCTACTGCGATGAGT | 412 |
| 309 | FQNSQTR | 7.295124395 | 7.292917095 | TTTCAGAATTCTCAGACTCGT | 413 |
| 310 | NTSQSQK | 7.643026804 | 7.408184512 | AACACCAGCCAAAGCCAAAAA | 414 |

TABLE 7
List of peptides that target the BRAIN with greater efficiency and
specificity over LIVER

| SEQ ID | | LOG2 BRAIN | BRAIN: | | SEQ ID |
|---|---|---|---|---|---|
| NO: | SEQUENCE | CPM | LIVER | DNA SEQUENCE | NO: |
| 47 | IDVDTPT | 5.608367892 | 6.517368793 | ATCGACGTCGACACCCCCACC | 415 |
| 48 | GASGEDL | 4.570199131 | 6.064162532 | GGTGCGTCGGGTGAGGATTTG | 416 |
| 49 | LDNLSVT | 4.829683675 | 5.738684576 | CTCGACAACCTCAGCGTCACC | 417 |
| 50 | TLMEGMK | 5.804422625 | 5.713423526 | ACTCTTATGGAGGGTATGAAG | 418 |
| 51 | VNEIIEK | 5.963805249 | 5.457768651 | GTCAACGAAATCATCGAAAAA | 419 |
| 52 | LHLGMID | 4.892900581 | 5.386863982 | CTCCACCTCGGCATGATCGAC | 420 |
| 53 | DHEVTDH | 4.79865678 | 5.292620181 | GATCATGAGGTGACTGATCAT | 421 |
| 54 | SYIPGHK | 3.612129811 | 5.106093212 | AGCTACATCCCCGGCCACAAA | 422 |
| 55 | NIEDNMG | 5.410871603 | 5.097480082 | AACATCGAAGACAACATGGGC | 423 |
| 56 | IFTLQSG | 6.5124935 | 5.836531899 | ATCTTCACCCTCCAAAGCGGC | 424 |

TABLE 8
List of peptides that target the BRAIN with greater efficiency and
specificity over SPINAL CORD

| | | | BRAIN: | | |
|---|---|---|---|---|---|
| SEQ ID | | LOG2 | SPINAL | | SEQ ID |
| NO: | SEQUENCE | BRAIN CPM | CORD | DNA SEQUENCE | NO: |
| 57 | TTISSTS | 7.971696369 | 7.140940686 | ACTACGATTTCTAGTACGAGT | 425 |
| 58 | KSSDKDS | 7.194899051 | 7.364143368 | AAGAGTAGTGATAAGGATAGT | 426 |
| 59 | NSNVPKN | 7.107651767 | 7.276896084 | AATAGTAATGTTCCTAAGAAT | 427 |
| 60 | AAAEVNK | 6.946403096 | 7.115647413 | GCTGCGGCGGAGGTTAATAAG | 428 |
| 61 | VLTTLSK | 6.906367628 | 7.075611945 | GTCCTCACCACCCTCAGCAAA | 429 |
| 62 | VTTNREL | 5.932554315 | 6.686761132 | GTTACTACGAATCGTGAGCTG | 430 |
| 63 | NPTVANT | 5.920468841 | 4.352747563 | AACCCCACCGTOGCCAACACC | 431 |
| 64 | TLNILNQ | 6.070194594 | 6.824401411 | ACCCTCAACATCCTCAACCAA | 432 |
| 65 | NNPLTGD | 6.02575774 | 6.779964557 | AATAATCCTCTTACGGGGGAT | 433 |
| 66 | LSTSGNE | 6.005162679 | 6.174406995 | TTGTCGACTAGTGGGAATGAG | 434 |

TABLE 9
List of peptides that target the BRAIN with greater efficiency and specificity over
LIVER and SPINAL CORD

| | | | | BRAIN: | | SEQ |
|---|---|---|---|---|---|---|
| SEQ ID | | LOG2 BRAIN | BRAIN: | SPINAL | | ID |
| NO: | SEQUENCE | CPM | LIVER | CORD | DNA SEQUENCE | NO: |
| 67 | QVDGPVR | 5.598919954 | 5.092883355 | 5.76816427 | CAAGTCGACGGCCCCGTCAGA | 435 |
| 68 | GDNGFYK | 5.576951978 | 5.070915379 | 6.331158795 | GGCGACAACGGCTTCTACAAA | 436 |
| 69 | APVTGEN | 4.556597864 | 5.050561266 | 5.310804681 | GCCCCCGTCACCGGCGAAAAC | 437 |
| 70 | SNDMTEK | 4.544836698 | 5.0388001 | 5.299043515 | AGTAATGATATGACTGAGAAG | 438 |
| 71 | CNEEMKA | 4.856890045 | 5.028925352 | 5.611096862 | TGTAATGAGGAGATGAAGGCG | 439 |
| 72 | ENQSAST | 5.681803339 | 5.005841739 | 5.851047655 | GAGAATCAGTCTGCTTCGACG | 440 |
| 73 | PHSEGDN | 5.782889464 | 4.95492477 | 6.537096281 | CCTCATTCGGAGGGGGATAAT | 441 |
| 74 | LSTETMV | 4.720654268 | 4.892689574 | 5.474861085 | CTCAGCACCGAAACCATGGTC | 442 |
| 75 | AGDYKEW | 5.688054581 | 4.860089887 | 5.442261398 | GCTGGTGATTATAAGGAGTGG | 443 |
| 76 | ALGEEST | 4.676423488 | 4.848458794 | 5.430630305 | GCGTTGGGTGAGGAGAGTACT | 444 |

TABLE 10
List of peptides that target the SPINAL CORD with greater efficiency and specificity over
LIVER and BRAIN

| | | LOG2 | SPINAL | BRAIN : | | SEQ |
|---|---|---|---|---|---|---|
| SEQ ID | | SPINAL | CORD | SPINAL | | ID |
| NO: | SEQUENCE | CORD CPM | LIVER | CORD | DNA SEQUENCE | NO: |
| 107 | STHDRDF | 8.398078025 | 6.985150831 | −9.670470997 | AGTACTCATGATCGTGATTTT | 445 |
| 108 | GEMKDMS | 8.19853443 | 6.785607236 | −9.364012198 | GGCGAAATGAAAGACATGAGC | 446 |
| 109 | MNDFVSL | 8.204345898 | 6.610846458 | −9.592216087 | ATGAACGACTTCGTCAGCCTC | 447 |
| 110 | QHDGSML | 8.347768854 | 6.51980416 | −9.735639043 | CAGCATGATGGTAGTATGTTG | 448 |
| 111 | HADLRDG | 7.972011342 | 6.465974744 | −9.359881531 | CATGCGGATCTGAGGGATGGG | 449 |
| 112 | GLEFTRH | 8.111526454 | 6.28356176 | −9.499396643 | GGGTTGGAGTTTACTCGGCAT | 450 |
| 113 | VDANGTW | 7.284712172 | 6.971320651 | −8.672582361 | GTCGACGCCAACGGCACCTGG | 451 |
| 114 | IEEKNGT | 7.12231013 | 6.294345436 | −8.287787897 | ATCGAAGAAAAAAACGGCACC | 452 |
| 115 | ARDTDDA | 7.103774178 | 6.790382657 | −8.37616715 | GCCAGAGACACCGACGACGCC | 453 |
| 116 | ETDKHGP | 6.85681798 | 6.350781382 | −8.244688169 | GAAACCGACAAACACGGCCCC | 454 |

TABLE 11
List of peptides that target the SPINAL CORD with greater efficiency and
specificity over BRAIN

| SEQ ID | | LOG2 SPINAL | BRAIN: SPINAL | | SEQ ID |
|---|---|---|---|---|---|
| NO: | SEQUENCE | CORD CPM | CORD | DNA SEQUENCE | NO: |
| 97 | DQTNSTH | 8.233057195 | -9.620927384 | GATCAGACTAATTCTACGCAT | 455 |
| 98 | MQMNSGA | 8.202895225 | -9.475288196 | ATGCAGATGAATAGTGGTGCT | 456 |
| 99 | NTMNSYP | 7.992307504 | -9.157785272 | AACACCATGAACAGCTACCCC | 457 |
| 100 | ILSNQAF | 7.925273282 | -9.197666254 | ATTTTGTCTAATCAGGCTTTT | 458 |
| 101 | GYSTSEV | 8.220207773 | -8.945112949 | GGCTACAGCACCAGCGAAGTC | 459 |
| 102 | ANSHDKI | 8.27783891 | -9.665709099 | GCTAATAGTCATGATAAGATT | 460 |
| 103 | GPGTSDN | 8.073929667 | -9.239407435 | GGGCCTGGGACGAGTGATAAT | 461 |
| 104 | TGFNNKI | 8.287452335 | -9.675322524 | ACTGGTTTTAATAATAAGATT | 462 |
| 105 | DIAGRNP | 8.160178315 | -9.432571287 | GATATTGCTGGTAGGAATCCT | 463 |
| 106 | KQSPSNY | 8.325277967 | -9.597670938 | AAGCAGAGTCCGAGTAATTAT | 464 |

TABLE 12
List of peptides that target the SPINAL CORD with greater efficiency and
specificity over LIVER

| SEQ ID | | LOG2 SPINAL | SPINAL | | SEQ ID |
|---|---|---|---|---|---|
| NO: | SEQUENCE | CORD CPM | CORD: LIVER | DNA SEQUENCE | NO: |
| 87 | NSEPDAN | 7.993986033 | 8.487949434 | AACAGCGAACCCGACGCCAAC | 465 |
| 90 | VQVGSMT | 8.23447787 | 7.55851627 | GTTCAGGTGGGTAGTATGACG | 466 |
| 88 | ELGTAEM | 8.013977508 | 7.507940909 | GAGCTGGGGACGGCTGAGATG | 467 |
| 91 | PTNMPPT | 7.761493021 | 7.4481015 | CCTACGAATATGCCGCCGACG | 468 |
| 92 | DAVSRVP | 7.946232901 | 7.118268207 | GACGCCGTCAGCAGAGTCCCC | 469 |
| 93 | CGKTILT | 8.306489115 | 7.100012798 | TGTGGTAAGACGATTCTTACG | 470 |
| 94 | MVNELTP | 8.294280057 | 7.08780374 | ATGGTGAATGAGCTTACTCCG | 471 |
| 95 | NIAEQPK | 8.228786758 | 7.022310441 | AACATCGCCGAACAACCCAAA | 472 |
| 96 | GREPSQY | 8.197077898 | 6.990601581 | GGTAGGGAGCCGAGTCAGTAT | 473 |
| 89 | STLEMPH | 7.430668526 | 7.117277005 | AGCACCCTCGAAATGCCCCAC | 474 |

TABLE 13
List of peptides that target both SPINAL CORD and BRAIN with greater efficiency and
specificity over LIVER

| SEQ | | | | SPINAL | | | SEQ |
|---|---|---|---|---|---|---|---|
| ID | | LOG2 | LOG2 SPINAL | CORD: | BRAIN: | | ID |
| NO: | SEQUENCE | BRAIN CPM | CORD CPM | LIVER | LIVER | DNA SEQUENCE | NO: |
| 311 | TQPTMEN | 4.777036738 | 7.578948533 | 6.265557012 | 3.463645217 | ACCCAACCCACCATGGA | 475 |
| | | | | | | AAAC | |
| 312 | ALVSGDV | 6.006592506 | 8.108430541 | 5.602393942 | 3.500555907 | GCGTTGGTTAGTGGTGA | 476 |
| | | | | | | TGTT | |
| 313 | SEYGTKH | 5.744843733 | 7.605542743 | 5.514543644 | 3.653844634 | AGCGAATACGGCACCAA | 477 |
| | | | | | | ACAC | |
| 314 | ENMTKNI | 9.470952651 | 8.157185171 | 2.673868649 | 3.987636129 | GAAAACATGACCAAAAA | 478 |
| | | | | | | CATC | |
| 244 | ENHIKTI | 9.508966742 | 8.342508337 | 2.324719085 | 3.491177489 | GAAAACCACATCAAAAC | 479 |
| | | | | | | CATC | |
| 315 | NNVSQEI | 4.595123386 | 7.081843538 | 5.768452017 | 3.281731865 | AATAATGTTAGTCAGGA | 480 |
| | | | | | | GATT | |
| 316 | TPEGPSN | 5.498842525 | 7.113071923 | 5.35910781 | 3.744878412 | ACCCCCGAAGGCCCCAG | 481 |
| | | | | | | TAAC | |
| 317 | LNDTNER | 6.337780092 | 6.860503027 | 4.496485433 | 3.973762498 | TTGAATGATACTAATGA | 482 |
| | | | | | | GAGG | |
| 318 | NSLVLNS | 5.969681816 | 6.709317556 | 4.158886838 | 3.419251097 | AACAGCCTCGTCCTCAA | 483 |
| | | | | | | CAGC | |
| 319 | FEPHTYA | 6.772421452 | 6.24014662 | 2.827219425 | 3.359494258 | TTCGAACCCCACACCTA | 484 |
| | | | | | | CGCC | |

TABLE 14
List of peptides that target the SPINAL CORD
with improved efficiency

| SEQ | | | | SEQ |
|---|---|---|---|---|
| ID | | LOG2 SPINAL | | ID |
| NO: | SEQUENCE | CORD CPM | DNA SEQUENCE | NO: |
| 137 | EGKNEVI | 8.325277967 | GAGGGTAAGAAT | 485 |
| | | | GAGGTGATT | |
| 138 | NSDNHNI | 8.380219503 | AACAGCGACAAC | 486 |
| | | | CACAACATC | |
| 139 | DQKLPAT | 9.112299395 | GATCAGAAGCTT | 487 |
| | | | CCGGCGACG | |
| 140 | TITPITN | 8.245793183 | ACCATCACCCCC | 488 |
| | | | ATCACCAAC | |
| 141 | ILTASER | 9.192699457 | ATTCTTACTGCT | 489 |
| | | | TCTGAGCGG | |
| 142 | IGTTQTN | 9.019756551 | ATTGGTACTACG | 490 |
| | | | CAGACGAAT | |
| 143 | SPATASH | 9.115387026 | AGTCCTGCGACT | 491 |
| | | | GCTTCTCAT | |
| 144 | SVDNRGN | 8.721526614 | AGCGTCGACAAC | 492 |
| | | | AGAGGCAAC | |
| 145 | NVSSRSN | 8.297002124 | AACGTCAGCAGC | 493 |
| | | | AGAAGCAAC | |
| 146 | KSQATQY | 8.131489556 | AAGAGTCAGGCG | 494 |
| | | | ACGCAGTAT | |

TABLE 15
List of peptides that target the SPINAL
CORD with significant efficiency

| SEQ | | | | SEQ |
|---|---|---|---|---|
| ID | | LOG2 SPINAL | | ID |
| NO: | SEQUENCE | CORD CPM | DNA SEQUENCE | NO: |
| 197 | THNDLLN | 7.295641732 | ACCCACAACGAC | 495 |
| | | | CTCCTCAAC | |
| 198 | PERAQVS | 7.457681478 | CCCGAAAGAGCC | 496 |
| | | | CAAGTCAGC | |
| 199 | YESLTQN | 7.251417732 | TACGAAAGCCTC | 497 |
| | | | ACCCAAAAC | |
| 200 | SERPDTL | 7.158682519 | AGCGAAAGACCC | 498 |
| | | | GACACCCTC | |
| 201 | TNDANTL | 7.330601571 | ACCAACGACGCC | 499 |
| | | | AACACCCTC | |
| 202 | SSNEYST | 7.24014662 | AGCAGCAACGAA | 500 |
| | | | TACAGCACC | |
| 203 | NTFSRNN | 7.217336737 | AATACTTTTAGT | 501 |
| | | | AGGAATAAT | |
| 204 | YNLQLNS | 7.088143526 | TACAACCTCCAA | 502 |
| | | | CTCAACAGC | |
| 205 | AGYPNSA | 7.094416123 | GCTGGTTATCCT | 503 |
| | | | AATAGTGCG | |
| 206 | NADKNNL | 7.140610946 | AATGCTGATAAG | 504 |
| | | | AATAATTTG | |

TABLE 16
List of peptides that target both BRAIN and
SPINAL CORD with improved efficiency and
specificity

| SEQ | | | | | SEQ |
|---|---|---|---|---|---|
| ID | SE- | LOG2 BRAIN | LOG2 SPINAL | DNA | ID |
| NO: | QUENCE | CPM | CORD CPM | SEQUENCE | NO: |
| 117 | SDIGKTH | 4.914387926 | 8.158682519 | AGCGACAT | 505 |
| | | | | CGGCAAAA | |
| | | | | CCCAC | |
| 118 | PNEGGHN | 4.943046689 | 8.654122924 | CCTAATGA | 506 |
| | | | | GGGGGGTC | |
| | | | | ATAAT | |
| 119 | AGNPGVI | 5.771160171 | 8.157185171 | GCTGGTAA | 507 |
| | | | | TCCGGGGG | |
| | | | | TGATT | |
| 120 | VVGSTVL | 4.455527483 | 8.25282045 | GTTGTTGG | 508 |
| | | | | TTCTACTG | |
| | | | | TGCTG | |
| 121 | GAITNNY | 4.556597864 | 9.423835512 | GGTGCGAT | 509 |
| | | | | TACGAATA | |
| | | | | ATTAT | |
| 122 | SLNNVTN | 4.875790251 | 8.914678167 | AGTCTTAA | 510 |
| | | | | TAATGTTA | |
| | | | | CTAAT | |
| 123 | EKTSVNT | 5.969681816 | 9.247904959 | GAAAAAAC | 511 |
| | | | | CAGCGTCA | |
| | | | | ACACC | |
| 124 | SLSQYEK | 4.773681631 | 8.346455522 | AGCCTCAG | 512 |
| | | | | CCAATACG | |
| | | | | AAAAA | |
| 125 | GAQFRSD | 4.035595932 | 7.867845002 | GGTGCTCA | 513 |
| | | | | GTTTCGTT | |
| | | | | CTGAT | |
| 126 | VASKSNH | 5.497826184 | 9.602795275 | GTGGCTTC | 514 |
| | | | | GAAGTCTA | |
| | | | | ATCAT | |

TABLE 17
List of peptides that target both BRAIN and
SPINAL CORD with significant efficiency
and specificity

| SEQ | | | | | SEQ |
|---|---|---|---|---|---|
| ID | | LOG2 BRAIN | LOG2 SPINAL | DNA | ID |
| NO: | SEQUENCE | CPM | CORD CPM | SEQUENCE | NO: |
| 177 | HGSDIRD | 5.603651657 | 7.228786758 | CATGGTTC | 515 |
| | | | | TGATATTA | |
| | | | | GGGAT | |
| 178 | ETPNHDG | 3.226839655 | 7.443009876 | GAAACCCC | 516 |
| | | | | CAACCACG | |
| | | | | ACGGC | |
| 179 | NDSGAAS | 3.720654268 | 6.980502803 | AATGATTC | 517 |
| | | | | GGGTGCGG | |
| | | | | CTAGT | |
| 180 | ETASVHF | 4.566326121 | 7.457681478 | GAGACTGC | 518 |
| | | | | GAGTGTGC | |
| | | | | ATTTT | |
| 181 | NDNANTK | 5.821583177 | 7.287452335 | AACGACAA | 519 |
| | | | | CGCCAACA | |
| | | | | CCAAA | |
| 182 | SSNALQV | 4.724134837 | 7.314571461 | AGTTCTAA | 520 |
| | | | | TGCGTTGC | |
| | | | | AGGTT | |
| 183 | SGANHFS | 4.187038647 | 7.161672562 | TCGGGGGC | 521 |
| | | | | TAATCATT | |
| | | | | TTTCG | |
| 184 | TGSPNIP | 4.932554315 | 7.007344415 | ACTGGTAG | 522 |
| | | | | TCCGAATA | |
| | | | | TTCCG | |
| 185 | VSNISRY | 4.954945272 | 7.257020438 | GTTTCTAA | 523 |
| | | | | TATTAGTA | |
| | | | | GGTAT | |
| 186 | NVDKTPR | 3.710161894 | 7.364734256 | AACGTCGA | 524 |
| | | | | TAAAACCC | |
| | | | | CCAGA | |

TABLE 18
List of peptides that target the SPINAL CORD with
improved efficiency and specificity over LIVER

| SEQ | | | SPINAL | | SEQ |
|---|---|---|---|---|---|
| ID | | LOG2 SPINAL | CORD: | | ID |
| NO: | SEQUENCE | CORD CPM | LIVER | DNA SEQUENCE | NO: |
| 147 | DNGVKEK | 8.444238224 | 6.938201626 | GACAACGGCGTCA | 525 |
| | | | | AAGAAAAA | |
| 148 | GTELVSR | 8.420718865 | 6.329719766 | GGGACTGAGTTGG | 526 |
| | | | | TGTCTAGG | |
| 149 | AIMKIDA | 9.003183192 | 6.287693228 | GCTATTATGAAGA | 527 |
| | | | | TTGATGCT | |
| 150 | AFAGANV | 7.585643186 | 6.272251665 | GCCTTCGCCGGCG | 528 |
| | | | | CCAACGTC | |
| 151 | MNFAGPI | 8.070751924 | 6.105283706 | ATGAACTTCGCCG | 529 |
| | | | | GCCCCATC | |
| 152 | GVSSIDK | 7.767393623 | 6.091432022 | GGTGTGAGTTCGA | 530 |
| | | | | TTGATAAG | |
| 153 | IVSEYAG | 8.314571461 | 6.05364736 | ATTGTTTCGGAGT | 531 |
| | | | | ATGCTGGT | |
| 154 | NPIAESR | 8.151180188 | 6.001287399 | AATCCTATTGCTG | 532 |
| | | | | AGTCGAGG | |
| 155 | NREDTKL | 8.40566452 | 5.992737325 | AATAGGGAGGATA | 533 |
| | | | | CGAAGCTT | |
| 156 | TGVIEGL | 7.871502026 | 5.973148004 | ACCGGCGTCATCG | 534 |
| | | | | AAGGCCTC | |

TABLE 19
List of peptides that target the SPINAL CORD with significant
efficiency and specificity over LIVER

| SEQ | | | SPINAL | | SEQ |
|---|---|---|---|---|---|
| ID | | LOG2 SPINAL | CORD: | | ID |
| NO: | SEQUENCE | CORD CPM | LIVER | DNA SEQUENCE | NO: |
| 207 | NHNDSVE | 7.191237019 | 6.10023792 | AACCACAACGACA | 535 |
| | | | | GCGTCGAA | |
| 208 | LEASNTA | 7.158682519 | 6.06768342 | CTCGAAGCCAGCA | 536 |
| | | | | ACACCGCC | |
| 209 | VDNDNPL | 6.804213896 | 5.838745679 | GTCGACAACGACA | 537 |
| | | | | ACCCCCTC | |
| 210 | VELGSSP | 7.330601571 | 5.824564972 | GTTGAGTTGGGTT | 538 |
| | | | | CGTCTCCG | |
| 211 | VNEKESV | 7.309188264 | 5.89626107 | GTCAACGAAAAAG | 539 |
| | | | | AAAGCGTC | |
| 212 | SAVDMSA | 6.474611873 | 5.798650273 | AGCGCCGTCGACA | 540 |
| | | | | TGAGCGCC | |
| 213 | RLDLQHD | 6.864178685 | 5.451251491 | AGACTCGACCTCC | 541 |
| | | | | AACACGAC | |
| 214 | HEDKSVA | 6.904004666 | 5.491077471 | CATGAGGATAAGT | 542 |
| | | | | CTGTTGCG | |
| 215 | RSPGQIG | 6.733633217 | 6.227596618 | AGAAGCCCCGGCC | 543 |
| | | | | AAATCGGC | |
| 216 | AKEMRYA | 6.692876409 | 5.727408192 | GCTAAGGAGATGC | 544 |
| | | | | GGTATGCT | |

TABLE 20
List of peptides that target the SPINAL CORD with improved
efficiency and specificity over BRAIN

| SEQ | | | BRAIN: | | SEQ |
|---|---|---|---|---|---|
| ID | | LOG2 SPINAL | SPINAL | | ID |
| NO: | SEQUENCE | CORD CPM | CORD | DNA SEQUENCE | NO: |
| 157 | IGNTDHD | 7.87878838 | -9.151181352 | ATTGGGAATACG | 545 |
| | | | | GATCATGAT | |
| 158 | LEISTTS | 8.025512538 | -9.29790551 | CTTGAGATTAGT | 546 |
| | | | | ACGACTTCT | |
| 159 | VSLAPSI | 7.95142557 | -9.223818542 | GTGAGTTTGGCT | 547 |
| | | | | CCTTCTATT | |
| 160 | GSKSTFF | 8.185372397 | -9.350850165 | GGTTCGAAGAGT | 548 |
| | | | | ACGTTTTTT | |
| 161 | NASNASA | 8.214459976 | -9.486852948 | AATGCTAGTAAT | 549 |
| | | | | GCTAGTGCG | |
| 162 | QQNNSSL | 8.305137644 | -9.693007833 | CAGCAGAATAAT | 550 |
| | | | | AGTAGTTTG | |
| 163 | MHTERGT | 8.714417226 | -9.78035932 | ATGCATACGGAG | 551 |
| | | | | CGTGGTACG | |
| 164 | KSRSVND | 8.176530521 | -9.56440071 | AAAAGCAGAAGC | 552 |
| | | | | GTCAACGAC | |
| 165 | GSLGKPT | 8.461326183 | -9.186231359 | GGGTCTCTGGGG | 553 |
| | | | | AAGCCTACG | |
| 166 | TTNRTVY | 9.066769876 | -9.13271197 | ACTACGAATCGG | 554 |
| | | | | ACTGTGTAT | |

TABLE 21
List of peptides that target the SPINAL CORD with significant
efficiency and specificity over BRAIN

| SEQ | | | BRAIN | | SEQ |
|---|---|---|---|---|---|
| ID | | LOG2 SPINAL | SPINAL | | ID |
| NO: | SEQUENCE | CORD CPM | CORD | DNA SEQUENCE | NO: |
| 217 | MVNVNVK | 7.109979328 | -8.382372299 | ATGGTTAATGTT | 555 |
| | | | | AATGTGAAG | |
| 218 | NTLASFS | 7.23163512 | -8.504028092 | AATACGTTGGCT | 556 |
| | | | | TCTTTTAGT | |
| 219 | IGAKGSP | 7.119237295 | -8.507107484 | ATTGGTGCTAAG | 557 |
| | | | | GGTAGTCCT | |
| 220 | NITSVTA | 7.12231013 | -8.510180319 | AATATTACTAGT | 558 |
| | | | | GTTACTGCG | |
| 221 | ITMRSMM | 7.125376433 | -8.513246622 | ATTACGATGCGG | 559 |
| | | | | TCGATGATG | |
| 222 | MDNQSNN | 7.170605687 | -8.558475876 | ATGGATAATCAG | 560 |
| | | | | AGTAATAAT | |
| 223 | YQSGLLE | 7.284712172 | -8.672582361 | TATCAGAGTGGT | 561 |
| | | | | CTTCTTGAG | |
| 224 | TGANIGY | 6.650934646 | -8.038804835 | ACGGGGGCGAAT | 562 |
| | | | | ATTGGTTAT | |
| 225 | QDNSKLS | 7.179483838 | -7.904389014 | CAAGACAACAGC | 563 |
| | | | | AAACTCAGC | |
| 226 | SSPAKPT | 7.452807503 | -8.725200475 | AGCAGCCCCGCC | 564 |
| | | | | AAACCCACC | |

TABLE 22
List of peptides that target the SPINAL CORD with improved efficiency and
specificity over LIVER and BRAIN

| SEQ | | LOG2 | SPINAL | BRAIN: | | SEQ |
|---|---|---|---|---|---|---|
| ID | | SPINAL | CORD: | SPINAL | | ID |
| NO: | SEQUENCE | CORD CPM | LIVER | CORD | DNA SEQUENCE | NO: |
| 167 | HNGVSIL | 8.228786758 | -9.501179729 | -9.670470997 | CACAACGGCGTCAGCATCCTC | 565 |
| 168 | NESSVTS | 8.204345898 | -9.47673887 | -9.364012198 | AATGAGAGTTCTGTGACTTCG | 566 |
| 169 | TGTEIGY | 7.823222011 | -7.548127187 | -9.592216087 | ACGGGTACGGAGATTGGTTAT | 567 |
| 170 | SLSDREY | 8.230211642 | -9.395689409 | -9.735639043 | AGCCTCAGCGACAGAGAATAC | 568 |
| 171 | GPGEHSP | 7.587867851 | -8.97573804 | -9.359881531 | GGGCCGGGTGAGCATTCGCCT | 569 |
| 172 | TSTSDIA | 7.786889798 | -9.05928277 | -9.499396643 | ACCAGCACCAGCGACATCGCC | 570 |
| 173 | ASRDSDV | 8.315914127 | -9.588307099 | -8.672582361 | GCCAGCAGAGACAGCGACGTC | 571 |
| 174 | YNSLQGQ | 8.012322092 | -8.284715063 | -8.287787897 | TATAATTCGCTGCAGGGTCAG | 572 |
| 175 | FIENKVA | 7.784951994 | -9.172822183 | -8.37616715 | TTTATTGAGAATAAGGTTGCG | 573 |
| 176 | IGTLPTM | 7.690808029 | -8.963201 | -8.244688169 | ATCGGCACCCTCCCCACCATG | 574 |

TABLE 23
List of peptides that target the SPINAL CORD with significant efficiency and
specificity over LIVER and BRAIN

| | | LOG2 | SPINAL | BRAIN: | | |
|---|---|---|---|---|---|---|
| SEQ ID | | SPINAL | CORD: | SPINAL | | SEQ ID |
| NO: | SEQUENCE | CORD CPM | LIVER | CORD | DNA SEQUENCE | NO: |
| 227 | QEGNLVS | 7.457681478 | 5.307788689 | -8.845551667 | CAAGAAGGCAACCTCGTCAGC | 575 |
| 228 | PDNTTTS | 6.777174644 | 5.101213043 | -8.165044832 | CCCGACAACACCACCACCAGC | 576 |
| 229 | WSGTLVH | 7.3673267 | 5.401858483 | -8.170234389 | TGGTCGGGTACGCTGGTGCAT | 577 |
| 230 | MLHGHHL | 7.423212721 | 5.273319932 | -8.81108291 | ATGCTCCACGGCCACCACCTC | 578 |
| 231 | VWHDQSA | 7.007344415 | 5.179379722 | -8.395214604 | GTCTGGCACGACCAAAGCGCC | 579 |
| 232 | IPFPGPE | 6.134536432 | 5.043537332 | -7.522406621 | ATCCCCTTCCCCGGCCCCGAA | 580 |
| 233 | SHHHPTT | 7.38790024 | 5.181423923 | -8.112805417 | TCGCATCATCATCCTACTACT | 581 |
| 234 | RYDERNA | 7.333256024 | 5.367787807 | -8.721126213 | AGATACGACGAAAGAAACGCC | 582 |
| 235 | IGNRYPT | 7.12231013 | 5.446348529 | -8.510180319 | ATTGGTAATCGTTATCCTACG | 583 |
| 236 | DEDRSGE | 6.306489115 | 5.341020897 | -7.694359304 | GATGAGGATAGGTCGGGTGAG | 584 |

TABLE 24
List of peptides that have highest BRAIN Cpm

| SEQ | | | | SEQ |
|---|---|---|---|---|
| ID | | LOG2 BRAIN | | ID |
| NO: | SEQUENCE | CPM | DNA SEQUENCE | NO: |
| 255 | GNTTRDY | 10.23051531 | GGGAATACTACGAGGGATTAT | 585 |
| 256 | GNMVKQV | 10.20952286 | GGCAACATGGTCAAACAAGTC | 586 |
| 245 | ENNIRSI | 9.748106238 | GAAAACAACATCAGAAGCATC | 587 |
| 260 | ENHTRNS | 9.664018467 | GAGAATCATACTCGTAATTCG | 588 |
| 261 | DNSIRNT | 9.67597426 | GACAACAGCATCAGAAACACC | 589 |
| 258 | GNNVKSI | 9.991778929 | GGCAACAACGTCAAAAGCATC | 590 |
| 257 | TNSVKNL | 10.1501166 | ACGAATAGTGTTAAGAATTTG | 591 |
| 243 | GNTTKSS | 9.869910491 | GGTAATACGACTAAGTCTAGT | 592 |
| 241 | LNTTKPI | 9.953648634 | CTCAACACCACCAAACCCATC | 593 |
| 259 | DNSTRSV | 9.977039606 | GACAACAGCACCAGAAGCGTC | 594 |

TABLE 25
List of peptides with highest BRAIN Cpm and
improved SPINAL CORD expression

| | | | LOG2 | | |
|---|---|---|---|---|---|
| SEQ ID | | LOG2 BRAIN | SPINAL | | SEQ ID |
| NO: | SEQUENCE | CPM | CORD CPM | DNA SEQUENCE | NO: |
| 300 | GNSTKAS | 8.937716356 | 9.604444384 | GGGAATAGTACGAAGGCGTCT | 595 |
| 292 | ENSTRYT | 9.112556777 | 8.356928853 | GAGAATTCGACTAGGTATACG | 596 |
| 291 | RRDMDPT | 9.129389098 | 8.163165262 | AGAAGAGACATGGACCCCACC | 597 |
| 296 | NNSTARI | 9.004626128 | 8.073929667 | AATAATTCGACTGCTAGGATT | 598 |
| 293 | MNSTRPF | 9.101894164 | 7.717468397 | ATGAATTCGACTCGGCCTTTT | 599 |
| 298 | TNATRPL | 8.97890735 | 8.220207773 | ACCAACGCCACCAGACCCCTC | 600 |
| 297 | LSNKAML | 8.982090602 | 8.302430898 | CTTTCGAATAAGGCTATGCTT | 601 |
| 299 | GNAVRGT | 8.954019221 | 7.765429436 | GGTAATGCTGTTAGGGGTACG | 602 |
| 294 | SNNVKQT | 9.096700992 | 8.539264832 | AGCAACAACGTCAAACAAACC | 603 |
| 295 | SNNSRPY | 9.017093941 | 8.263993362 | AGCAACAACAGCAGACCCTAC | 604 |

TABLE 26
List of peptides with highest BRAIN Cpm and
significant SPINAL CORD expression

| | | | LOG2 | | |
|---|---|---|---|---|---|
| SEQ ID | | LOG2 BRAIN | SPINAL | | SEQ ID |
| NO: | SEQUENCE | CPM | CORD CPM | DNA SEQUENCE | NO: |
| 282 | DNVIRPT | 8.47605794 | 7.265383911 | GACAACGTCATCAGACCCACC | 605 |
| 286 | SRTSISE | 7.984269352 | 6.918218525 | AGCAGAACCAGCATCAGCGAA | 606 |
| 290 | FSHTVKG | 7.638192108 | 7.023870313 | TTTTCGCATACGGTGAAGGGG | 607 |
| 287 | SNSVRND | 7.928222743 | 7.372497656 | AGCAACAGCGTCAGAAACGAC | 608 |
| 289 | QNTIKMT | 7.688500063 | 7.284712172 | CAGAATACTATTAAGATGACG | 609 |
| 283 | NVRDLNL | 8.354018084 | 7.338550324 | AACGTCAGAGACCTCAACCTC | 610 |
| 285 | LNTNRTN | 8.169114769 | 7.020580243 | CTTAATACGAATAGGACGAAT | 611 |
| 288 | IGNRPVI | 7.863020347 | 7.335905603 | ATCGGCAACAGACCCGTCATC | 612 |
| 281 | GNEVRRD | 9.076590474 | 6.987260169 | GGGAATGAGGTTAGGAGGGAT | 613 |
| 284 | TSRLPAL | 8.23311325 | 6.93229371 | ACGAGTAGGTTGCCTGCGTTG | 614 |

TABLE 27
List of peptides with highest BRAIN Cpm and
with greater specificity over SPINAL CORD

| | | | BRAIN: | | |
|---|---|---|---|---|---|
| SEQ ID | | LOG2 BRAIN | SPINAL | | SEQ ID |
| NO: | SEQUENCE | CPM | CORD | DNA SEQUENCE | NO: |
| 262 | NNRRPDD | 9.452317635 | 7.747092833 | AATAATCGGCGTCCGGATGAT | 615 |
| 263 | QNVIKPT | 9.207309576 | 7.502084774 | CAAAACGTCATCAAACCCACC | 616 |
| 264 | QNSTKLI | 9.091573272 | 7.145340371 | CAAAACAGCACCAAACTCATC | 617 |
| 265 | ANNTRNM | 9.07990516 | 7.133672259 | GCCAACAACACCAGAAACATG | 618 |
| 266 | SNTTRNL | 9.068997549 | 7.363772748 | AGCAACACCACCAGAAACCTC | 619 |
| 267 | ENSVRNN | 9.020990247 | 7.967842142 | GAAAACAGCGTCAGAAACAAC | 620 |
| 268 | NNSTKLL | 8.97662929 | 7.560911105 | AATAATTCTACGAAGTIGCTG | 621 |
| 269 | GNSVRAN | 8.954297099 | 7.008064198 | GGGAATAGTGTTCGGGCGAAT | 622 |
| 270 | SNSTRPL | 8.953555973 | 7.248331171 | AGTAATAGTACGAGGCCGTTG | 623 |
| 271 | GNSTMRV | 8.918381416 | 7.087625732 | GGCAACAGCACCATGAGAGTC | 624 |

TABLE 28
List of peptides with highest BRAIN Cpm and
with greater SPINAL CORD efficiency and specificity

| SEQ | | | LOG2 | | SEQ |
|---|---|---|---|---|---|
| ID | | LOG2 BRAIN | SPINAL | | ID |
| NO: | SEQUENCE | CPM | CORD CPM | DNA SEQUENCE | NO: |
| 279 | SNVIKNV | 9.188692888 | 9.242972664 | AGCAACGTCATCAAAAACGTC | 625 |
| 277 | SNSIRNN | 9.294539242 | 8.371206653 | AGCAACAGCATCAGAAACAAC | 626 |
| 239 | TNTTKNF | 9.545881929 | 8.384064983 | ACCAACACCACCAAAAACTTC | 627 |
| 275 | SNSVKDY | 9.434435075 | 8.958320183 | AGCAACAGCGTCAAAGACTAC | 628 |
| 273 | MKSGLSM | 9.529874824 | 10.21409998 | ATGAAAAGCGGCCTCAGCATG | 629 |
| 272 | GNSTKIG | 9.57604941 | 9.004849122 | GGCAACAGCACCAAAATCGGC | 630 |
| 278 | TDRMGLT | 9.191995691 | 9.147414341 | ACGGATCGTATGGGTCTGACG | 631 |
| 280 | YNSTRNQ | 9.136997965 | 8.057970488 | TACAACAGCACCAGAAACCAA | 632 |
| 274 | SNKMGNT | 9.476573925 | 8.737646279 | AGCAACAAAATGGGCAACACC | 633 |
| 276 | AVHKSDF | 9.344509257 | 7.088143526 | GCGGTGCATAAGTCGGATTTT | 634 |

TABLE 29
List of peptides that target the SPINAL CORD AND BRAIN
with greater efficiency and specificity over LIVER

| SEQ | | | LOG2 | | | SEQ |
|---|---|---|---|---|---|---|
| ID | | LOG2 BRAIN | SPINAL | SPINAL | | ID |
| NO: | SEQUENCE | CPM | CORD CPM | CORD: LIVER | DNA SEQUENCE | NO: |
| 127 | FGEITPG | 4.202092993 | 7.992307504 | 5.962708949 | TTTGGTGAGATTACTCCTGGG | 635 |
| 128 | ITDNRIV | 3.869517654 | 8.2556218 | 5.891604206 | ATCACCGACAACAGAATCGTC | 636 |
| 129 | AITPVAH | 5.819144131 | 9.065173974 | 5.777777662 | GCCATCACCCCCGTCGCCCAC | 637 |
| 130 | NGIERQE | 3.872657361 | 8.334581422 | 5.699261806 | AACGGCATCGAAAGACAAGAA | 638 |
| 131 | EWNNHES | 5.388014394 | 8.180958233 | 5.674921634 | GAATGGAACAACCACGAAAGC | 639 |
| 132 | DSMDGKK | 5.397854717 | 8.126907144 | 5.666674234 | GATTOGATGGATGGGAAGAAG | 640 |
| 133 | NDNNAGA | 3.724134837 | 7.505536447 | 5.607182425 | AATGATAATAATGCTGGGGCT | 641 |
| 134 | KDDHKEP | 5.346839431 | 8.309188264 | 5.5936983 | AAAGACGACCACAAAGAACCC | 642 |
| 135 | QADVGAN | 4.057834447 | 7.74164821 | 5.591755421 | CAGGCGGATGTTGGTGCGAAT | 643 |
| 136 | THISAVHH | 3.542867149 | 7.869674673 | 5.556283152 | ACGCATTCGGCTGTGCATCAT | 644 |

TABLE 30
List of peptides that target both BRAIN and SPINAL CORD withs
ignificant efficiency and specificity OVER LIVER

| SEQ | | | | | | SEQ |
|---|---|---|---|---|---|---|
| ID | | LOG2 BRAIN | LOG2 SPINAL | SPINAL | | ID |
| NO: | SEQ | CPM | CORD CPM | CODE: LIVER | DNA SEQUENCE | NO: |
| 187 | PRDLNDP | 3.236620676 | 6.629497475 | 5.423021159 | CCGCGGGATTTGAATGATCCT | 645 |
| 188 | GTQNDVM | 4.844150934 | 7.403140118 | 5.25324733 | GGTACGCAGAATGATGIGATG | 646 |
| 189 | KGVDGDI | 4.641877154 | 7.049924204 | 4.900031416 | AAAGGCGTCGACGGCGACATC | 647 |
| 190 | ENPSSNG | 5.379210736 | 7.265383911 | 4.852456717 | GAAAACCCCAGCAGCAACGGC | 648 |
| 191 | KGDVTFT | 4.312569529 | 7.265383911 | 4.805151002 | AAGGGGGATGTGACTTTTACG | 649 |
| 192 | PPNQDQH | 3.457619862 | 6.949696756 | 4.799803968 | CCCCCCAACCAAGACCAACAC | 650 |
| 193 | TPANELK | 3.440795239 | 6.620832614 | 4.792867921 | ACTCCGGCTAATGAGTTGAAG | 651 |
| 194 | GNEQITG | 3.393489525 | 6.935791154 | 4.785898366 | GGCAACGAACAAATCACCGGC | 652 |
| 195 | EVIKETG | 4.546803563 | 7.097542224 | 4.784150704 | GAGGTTATTAAGGAGACGGGT | 653 |
| 196 | ATVINGT | 3.638192108 | 7.341190206 | 4.665228606 | GCTACTGTGATTAATGGTACT | 654 |

1. An AAV capsid protein comprising an AAV capsid protein comprising an insertion sequence at least 71.4% identical to an amino acid sequence provided in Tables 1, 4-30 or FIG. 4.

2. The AAV capsid of claim 1, wherein the insertion sequence is at least 86.7% identical to the amino acid sequence provided in Tables 1, 4-30 or FIG. 4.

3. The AAV capsid of claim 1, wherein the insertion sequence comprises Formula I

| | (I) |
|---|---|
| (SEQ ID NO: 2) | |
| | X¹-X²-X³-X⁴-X⁵-X⁶-X⁷ |

wherein:
X¹ is an amino acid selected from I, L, M and V;
X² is an amino acid selected from A, S and T;
X³ is an amino acid selected from K and R;
X⁴ is an amino acid selected from D, E, N and Q;
X⁵ is an amino acid selected from F, W and Y;
X⁶ is an amino acid selected from F, W and Y; and
X⁷ is an amino acid selected from K and R.

4. The AAV capsid of claim 1, wherein the insertion sequence comprises Formula II

| | (II) |
|---|---|
| (SEQ ID NO: 3) | |
| | X⁸-X⁹-X¹⁰-x¹¹-x¹²-P-X¹³ |

wherein:
X⁸ is an amino acid selected from I, L, M and V;
X⁹ is an amino acid selected from D, E, N, and Q;
X¹⁰ is an amino acid selected from A, S and T;
X¹¹ is an amino acid selected from A, S and T;
X¹² is an amino acid selected from K and R; and
X¹³ is an amino acid selected from I, L, M and V.

5. The AAV capsid of claim 1, wherein the insertion sequence comprises Formula III

| | (III) |
|---|---|
| (SEQ ID NO: 4) | |
| | X¹⁴-X¹⁵-H-X¹⁶-X¹⁷-X¹⁸-X¹⁹ |

wherein:
X¹⁴ is an amino acid selected from D, E, N and Q;
X¹⁵ is an amino acid selected from D, E, N and Q;
X¹⁶ is an amino acid selected from A, S and T;
X¹⁷ is an amino acid selected from K and R;
X¹⁸ is an amino acid selected from D, E, N and Q; and
X¹⁹ is an amino acid selected from D, E, N and Q.

6. The AAV capsid of claim 1, wherein the insertion sequence comprises Formula IV
(IV)
(SEQ ID NO: 5)
X²⁰-X²¹-X²²-X²³-X²⁴-X²⁵-X²⁶
wherein:
X²⁰ is an amino acid selected from A, I, G, P, H, N, S, R and Y;
X²¹ is an amino acid selected from Q, N, S, T, F, L, A and E;
X²² is an amino acid selected from T, S, G, R, N, and D;
X²³ is an amino acid selected from D, E, S, T, G, I, M, H and N;
X²⁴ is an amino acid selected from I, L, F, R, T, S, N and Q;
X²⁵ is an amino acid selected from A, L, Q, G, K, S, P and Y; and
X²⁶ is an amino acid selected from D, K, H, M, Y, T, L, and I;
Provided X²² is not S when X²⁴ is R or S; further provided X²¹ is not S when X²³ is S or when X²⁵ is S; further provided X²⁵ is not S when X²⁴ is T or F or when X²⁶ is L; further provided X²³ is not T when X²⁴ is Q or when X²⁵ is P; further provided X²² is not G when X²⁰ is S or when X²⁶ is M; further provided X²⁵ is not L when X²³ is S or when X²⁶ is T or K; further provided X²² is not T when X²⁴ is S or when X²⁵ is P; further provided X²⁴ is not S when X²² is D or R; further provided X²⁵ is not G when X²² is G or T; further provided X²⁰ is not G when X²⁵ is P; further provided X²⁵ is not A or X²³ is T when X²⁶ is T; further provided X²⁰ is not Y when X²² is A; further provided X²⁰ is not R when X²³ is D; further provided X²¹ is not L when X²⁴ is L; further provided X²¹ is not T when X²³ is H; further provided X²¹ is not N when X²² is N; further provided X²³ is not G when X²⁶ is H; further X²² is not R when X²³ is I; and further provided X²⁵ is not Q when X²⁰ is P.

7. The AAV capsid of claim 6, wherein X²² is R.

8. The AAV capsid of claim 6, wherein the insertion sequence is selected from AFGGIAD (SEQ ID NO: 37), ISREFYK (SEQ ID NO: 38), GTDMRQT (SEQ ID NO: 39), HLTSNQL (SEQ ID NO: 40), PSSNNPH (SEQ ID NO: 41), NARSTGM (SEQ ID NO: 42), SNRTLSI (SEQ ID NO: 43), SQSIQKD (SEQ ID NO: 44), REDHNLY (SEQ ID NO: 45) and YQNDSGK (SEQ ID NO: 46).

9. The AAV capsid of claim 1, wherein the insertion sequence comprises Formula V

| | (V) |
|---|---|
| (SEQ ID NO: 6) | |
| | X²⁷-X²⁸-X²⁹-X³⁰-X³¹-X³²-X³³ |

wherein:
X²⁷ is an amino acid selected from I, G, L, T, V, D, S and N;
X²¹ is an amino acid selected from D, A, L, I, H, Y, F and N;
X²⁹ is an amino acid selected from S, T, M, E, V, L, I and N;
X³⁰ is an amino acid selected from P, G, L, I, V, E and D;
X³¹ is an amino acid selected from T, E, S, G, I, M, Q and N;
X³² is an amino acid selected from P, S, M, H, I, V, E and D; and
X³³ is an amino acid selected from G, L, K, H, T and D;
provided X²⁷ is not S when X³² is S; further provided X²⁷ is not T when X²⁹ is I or S; further provided X²⁷ is not V when X²⁹ is S; further provided X²⁷ is not L when X³¹ is N; further provided X²⁸ is not N when X³² is P; further provided X²⁹ is not V when X³⁰ is P; further provided X²⁹ is not N when X³⁰ is V; further provided X³⁰ is not G when X³¹ is Q; further provided X²⁹ is not S when X³² is P; further provided X³¹ is not T when X³² is S or V; and further provided X³² is not S when X³³ is K or L.

10. The AAV capsid of claim 9, wherein X²⁷ is I or L.

11. The AAV capsid of any of claims 9-10, wherein the insertion sequence is selected from IDVDTPT (SEQ ID NO: 47), GASGEDL (SEQ ID NO: 48), LDNLSVT (SEQ ID NO: 49), TLMEGMK (SEQ ID NO: 50), VNEIIEK (SEQ ID NO: 51), (SEQ ID NO: 52), DHEVTDH (SEQ ID NO: 53), SYIPGHK (SEQ ID NO: 54), NIEDNMG (SEQ ID NO: 55) and IFTLQSG (SEQ ID NO: 56).

12. The AAV capsid of claim 1, wherein the insertion sequence comprises Formula VI

| | (VI) |
|---|---|
| (SEQ ID NO: 7) | |
| | X³⁴-X³⁵-X³⁶-X³⁷-X³⁸-X³⁹-X⁴⁰ |

wherein:
X³⁴ is an amino acid selected from T, K, N, A, V and L;
X³⁵ is an amino acid selected from T, S, A, L, P and N;
X³⁶ is an amino acid selected from T, S, I, A, N and P;
X³⁷ is an amino acid selected from S, T, D, E, N, V, I and L;
X³⁸ is an amino acid selected from S, T, K, R, P, V, L, A and G;
X³⁹ is an amino acid selected from N, T, S, K, D, E and G; and
X⁴⁰ is an amino acid selected from S, T, K, N, Q, D, L and E;
Provided X⁴⁰ is not S when X³⁴ is A or N or when X³⁵ is N; further provided X³⁹ is not S when X³⁴ is T or L; further provided X⁴⁰ is not N or when X³⁵ is A or when X³⁶ is S; further provided X³⁶ is not S when X³⁹ is T or when X⁴⁰ is L; further provided X³⁵ is not S when X³⁹ is G or when X⁴⁰ is D or K; further provided X³⁸ is not S when X³⁴ is V or when X⁴⁰ is K; further provided X³⁵ is not P when X³⁶ is P or when X³⁷ is L; further provided X³⁹ is not T when X³⁴ is not L or when X³⁶ is A; further provided X³⁷ is not S when X³⁶ is A or N; further provided X³⁷ is not V when X³⁴ is T or K; further provided X³⁵ is not T when X³⁴ is K or when X³⁹ is K; further provided X³⁴ is not V when X³⁵ is A or when X⁴⁰ is Q; further provided X³⁴ is not L when X³⁶ is P; further provided X³⁴ is not A when X³⁸ is P; further provided X³⁵ is not N when X³⁶ is T; and further provided X³⁷ is not T when X³⁹ is N.

13. (canceled)

14. The AAV capsid of claim 12, wherein the insertion sequence is selected from TTISSTS (SEQ ID NO: 57), KSSDKDS (SEQ ID NO: 58), NSNVPKN (SEQ ID NO: 59), AAAEVNK (SEQ ID NO: 60), VLTTLSK (SEQ ID NO: 61), VTTNREL (SEQ ID NO: 62), NPTVANT (SEQ ID NO: 63), TLNILNQ (SEQ ID NO: 64), NNPLTGD (SEQ ID NO: 65) and LSTSGNE (SEQ ID NO: 66).

15. The AAV capsid of claim 1, wherein the insertion sequence comprises Formula VII

| | (VII) |
|---|---|
| (SEQ ID NO: 8) | |
| | X⁴¹-X⁴²-X⁴³-X⁴⁴-X⁴⁵-X⁴⁶-X⁴⁷ |

wherein:
X⁴¹ is an amino acid selected from Q, G, A, S, C, E, P and L;
X⁴² is an amino acid selected from D, P, H, S, G, V, L and N;
X⁴³ is an amino acid selected from N, E, Q, S, T, V, G and D;
X⁴⁴ is an amino acid selected from G, T, S, M, Y and E;
X⁴⁵ is an amino acid selected from P, F, T, K, E, M, A and G;
X⁴⁶ is an amino acid selected from V, E, D, M, K, S and Y; and
X⁴⁷ is an amino acid selected from R, K, N, A, T, V and W;
Provided X⁴¹ is not G when X⁴⁶ is S; further provided X⁴¹ is not S when X⁴⁶ is Y or S; further provided X⁴¹ is not A when X⁴⁵ is A; further provided X⁴¹ is not P when X⁴³ is N; further provided X⁴² is not P when X⁴⁶ is S; further provided X⁴² is not S when X⁴⁶ is D; further provided X⁴² is not H when X⁴⁷ is K; further provided X⁴³ is not S when X⁴⁴ is G; further provided X⁴³ is not G when X⁴⁵ is P; further provided X⁴⁴ is not T when X⁴⁷ is T; further provided X⁴⁴ is not S when X⁴⁶ is V; and further provided X⁴⁵ is not G when X⁴⁷ is V.

16. The AAV capsid of claim 15, wherein X⁴¹ is L, X⁴³ is T, and X⁴⁷ is V.

17. The AAV capsid of claim 15, wherein the insertion sequence is selected from QVDGPVR (SEQ ID NO: 67), GDNGFYK (SEQ ID NO: 68), APVTGEN (SEQ ID NO: 69), SNDMTEK (SEQ ID NO: 70), CNEEMKA (SEQ ID NO: 71), ENQSAST (SEQ ID NO: 72), PHSEGDN (SEQ ID NO: 73), LSTETMV (SEQ ID NO: 74), AGDYKEW (SEQ ID NO: 75) and ALGEEST (SEQ ID NO: 76).

18. The AAV capsid of claim 1, wherein the insertion sequence comprises Formula VIII

| | (VIII) |
|---|---|
| (SEQ ID NO: 9) | |
| | X⁴⁸-X⁴⁹-X⁵⁰-X⁵¹-X⁵²-X⁵³-X⁵⁴ |

wherein:
X⁴⁸ is an amino acid selected from E, S, G, A, N, and P;
X⁴⁹ is an amino acid selected from D, S, K, N, I and L;
X⁵⁰ is an amino acid selected from N, S, T, G, V, A and R;
X⁵¹ is an amino acid selected from L, T, G, N, D, R and A;
X⁵² is an amino acid selected from S, A, P, E, I, T and M;
X⁵³ is an amino acid selected from Y, F, T, N, G, E, P and Q; and
X⁵⁴ is an amino acid selected from V, I, D, A, Y, N, E and T;
Provided X⁵² is not S when X⁴⁹ is L or S; further provided X⁴⁸ is not S when X⁴⁹ is K, further provided X⁴⁸ is not S, when X⁵² is T or when X⁵³ is P; further provided X⁴⁸ is not P when X⁵³ is N, further provided X⁴⁸ is not G when X⁵³ is T, further provided X⁴⁹ is not S when X⁵² is M or X⁵¹ is N; further provided X⁴⁹ is not N when X⁵³ is T; further provided X⁵⁰ is not G when X⁵¹ is L, further provided X⁴⁹ is not N when X⁵⁴ is V, and further provided X⁵³ is not N when X⁵⁴ is A.

19. The AAV capsid of claim 18 wherein X⁴⁸ is E or S.

20. The AAV capsid of claim 18 wherein X⁴⁹ is D.

21. The AAV capsid of claim 18, wherein the insertion sequence is selected from EDNLSYV (SEQ ID NO: 77), SDSTAFI (SEQ ID NO: 78), SSNGPTD (SEQ ID NO: 79), EKTNEND (SEQ ID NO: 80), SNTDSGT (SEQ ID NO: 81), GIGTSEA (SEQ ID NO: 82), AIVAAGY (SEQ ID NO: 83), NLANIPN (SEQ ID NO: 84), PLRTTQE (SEQ ID NO: 85) and SDRRMNT (SEQ ID NO: 86).

176.-308. (canceled)
